SEQUENCE LISTING (698 sequences)

SEQ ID NO: 1 - DNA, 376 bp, Mus musculus
  misc_feature 34, 36, 38, 51, 54: n = A,T,C or G
gacttatctggctctactgatggcgagcaactcnananggaacccgacttnttncaggaagacgaagaggaagcgatgacattggccctacctgagggaccccaggagttagacatggacagtcccatggagagcagccaaggcccggagggctctgtcaagggttgtggcgaggagaaggagcctgagctggcgggcctgttcctgccagaggacaagtgcctcgtcctcactccatctgtgaccacatcagacctttccactcactccaccgcctctcttatcagcaacgaggagcaatttgaagactatggcgagggggatgacgtggactgtgctcccagcagcccatgccccgacgatgagaccagaacca

SEQ ID NO: 2 - DNA, 318 bp, Mus musculus
gaggtggggtctttcaaggaggctaaacgactaaaagaattcctagaagattatgatgatgatagagatgacccgaagtactacaggggtagtgctcttcagaaacggttgcgtgatagggaaaaagaaatggaagctgatgaacgagacaggaagagagagaaggaagagcttgaggaaatcaggcagcgcttactggcagaagggcatccagatccagatgcagagctccagaggatggaacaagaggctgagaggcgcaggcaacctcaaataaagcaagaaccagaatccgaggaagaagaagaagaaaagcaa

SEQ ID NO: 3 - DNA, 316 bp, Mus musculus
  misc_feature 257, 258, 268, 276, 289, 292, 294: n = A,T,C or G
tgaggttgaccctttgggtttgctgacatgagacttttaaacagcggtacctagtcgctgataaacactggcagagaaatggtggatcaatacttttctacactggtaacgaaggggacattgtctggttttgcaataatacggggttcatgtgggatgtggcctgaggagctgaaagctatgcttggtgttcgcctgagcatcgatacctattggggagtcccttccctttggtcaggactccttcaagattattnnatttattgantatgagancactgaacctgcnttnanacacatcacaagaaggcattgg

SEQ ID NO: 4 - DNA, 128 bp, Mus musculus
gactgagattatttgtataactttgcaagtgctgcaacaaagaagataactgaatctgttacagaaacagctcagacaatcaagaaatccgtggaagaaggaaaaatagatgatatccttgacaaggc

SEQ ID NO: 5 - DNA, 187 bp, Mus musculus
  misc_feature 79: n = A,T,C or G
tgaggataccctcaggcaagttcgctgcacccgaaagctgcggagctggatcgtggagcaggtggagagtggccatttnccaggggtgtgctgggacgatgcagccaagaccatgttccggattccctggaagcatgcaggcaagcaagacttccgagaggaccaggatgctgccatattcaaggct

SEQ ID NO: 6 - DNA, 367 bp, Mus musculus
agacaaaataacaaccacaggtgctactggagggcttaatgaatctactggcaaggcacctctggtaccaacgcagagagagcgtgggacgaagcctcccttagaggaactgtccacctcagcaacctcagaccatgatcacagagaacacgagagtacaaccactgtcaaagtggtgactagccactctgtggacaagaaaacaagtcaccccaatagagataatgcaggggatgaaacgcagacaacagataagaaagatggcttgccagtagtcaccctggttggaatcatagttggcgtcttgttagccattggcttcgtcggagggatcttcattgttgttatgaagaagatttctggaagg

SEQ ID NO: 7 - DNA, 232 bp, Mus musculus
  misc_feature 25, 129, 134: n = A,T,C or G
atgagccacagacagggggagtcgncccagagagtccggaatgctcagtgcttgtcggttccaacagaaaagaagcacagcagcattctaagtgtcccagagcacaggcccacatctaaggagcaggancaganggtggtggtgaggcccgtcctcttccaggtcccggtcctcaggccccgacccaccccagtatttccttgcattccccttttttttttttttttttttt

SEQ ID NO: 8 - DNA, 425 bp, Mus musculus
  misc_feature 129, 237, 254, 266, 297, 331, 370, 399: n = A,T,C or G
aaggcagcatgaatagtggccctggccctgtaggaggcaggcctgggggccgagggggaccagccgttcagcagaacattccttccaacctcctccaggaccatgaaaaccagagactctttgagcttnttggccgaaaatgctggacactggctaccacagttgttcagctctacctggcactgccccctggagctgagcactggaccatggaacactgcggggctgtgtgcttcntgaaggataaccctcanaagtcctacttnatccgcctttatggcctacaggctggtcggntactctgggaacaggagctgtactctcagctggnttatctcactcccaccccggtcttccacacttttgctgnagatgactgtcaagtaggactgaactttncggatgagagttgaaagcccaggcct

SEQ ID NO: 9 - DNA, 560 bp, Mus musculus
  misc_feature 35, 42, 57, 65, 174: n = A,T,C or G
actgagatgcccagtggaactatccctggccagtncacagantaaccagatgacatngccaggcnaaacccttacttctcattctccgcaagaagccacaaattctccgaagccacagccaagctatgaggagctggaggaagaagaacaggaggatcagtttgatttgacagntggtatcaccgatcctgagaagatcggggatggtatgaatgcctatgtagcctacaaagtcaccacacagacaagcttaccgatgttcagaagtaggcagtttgcagtgaaaagaagattcagtgacttcctgggtctttatgagaagctttcagagaagcactcccagaatggtttcattgtgcctcctccacccgagaagagcctaataggaatgacaaaggtgaaggttgggaaggaagattcttcttccgcagaatttcttgagaaacggagggctgctttggaaaggtaccttcaaagaattgtgaatcatcccaccatgttacaggacccagatgtcagggagtttctggaaaaagaagagctgcctcgagctgtccccc

SEQ ID NO: 10 - DNA, 542 bp, Mus musculus
  misc_feature 26, 470: n = A,T,C or G
gactgagccacagctccctgcttggnggcggaggggtggacggctgcattcatcgggccgcgggatccctgctcacggacgaatgccgcaccctacagaactgcgagaccggcaaagccaagatcacttgcggctatcggctgccagccaagtatgtcatccacacggtggggcccatcgccgtgggccaacccactgccagccaggcggccgagctccgcagctgctacttgagcagcctggacctgctgctggagcaccggctgcgctcagtggctttcccgtgcatctccacaggcgtgtttggctaccccaatgaggaggctgcagaagtagtgctggcttcgctgcgggaatggctggagcagcacaaggacaaggtggatcggctcatcatctgtgtgttcctggagaaggacgagggcatttaccgggagcgccttccccattatttcccagtagcctgaggntcccacatcccaccctgaacaggactgacccacccgggcctgtaggaattcgttcccagctttgagaggtct

SEQ ID NO: 11 - DNA, 248 bp, Mus musculus
  misc_feature 31, 97: n = A,T,C or G
actgaggctcggccatttctccatgacgacncagaagtacggcgctctcaggtccttacctcataatccacatgcaattggattgcagagcatttgngggacacaagaggaacaacagaccaagacccttgaacctcccacagagaagctccaggcagtcgcatccttcagggtgtctctccctgtgtcatttgctgcagctggcttttcagggctggggatggaactctgggactcctgcatgttgc

SEQ ID NO: 12 - DNA, 511 bp, Mus musculus
  misc_feature 39, 160, 161, 164, 165, 184, 185, 194, 202, 224, 247,310, 322, 329, 334, 375: n = A,T,C or G
cagtcagatccgcgcctgtacagccgcttcaccagcctngacaaaggggagaacgggactctcagccgggaagatttccagaggattccagaacttgccatcaacccactgggggaccggatcatcaatgccttcttctcagagggagaggatcaggctnnctnncgaggattcatgagaccannggctcattnccgacccnttgcggataccgaaaagagcangagatgtgaatggcccggaaccnctcaacagccggagcaacaagctgcacctttgctttcaggactctatgatttggatataagancacaagatctcnctgtgangagcntgttacaggtacatgcgaatgatggttggagtgaatatctnggatgagcagctgggcagtattgcggacaggaccatccaggaggctgaccaagatggagatagtgccatatcttttacagaattcgttaaggttttggagaaagtggatgtagaacagaagatgagcatccgattt

SEQ ID NO: 13 - DNA, 404 bp, Mus musculus
  misc_feature 17, 85, 158, 184, 190, 191, 194, 200, 213, 215, 221,227, 229, 232, 242, 253, 283, 296, 299, 300, 301, 303, 304, 305,317, 321, 336, 346, 360, 370, 388: n = A,T,C or G
gaaaaggtgtttgatgncatcatccgctgtttcaaacgccatggagcagaagtgattgatacacctgtgtttgaactaaaggaancactgactggaaagtacggagaagactcaaagccttatctatgacttgaaggaccagggtggggagcatgctngtccctccgttatgaccttacctgtncctttnnctncctgtntggttatgacttntnctgacnagcatntnacnctaccatatntcaaaggttgnatcacacgggacaccccagctatgacccgnggccgctaccgcngannntnnnactgcgatcttnacantgccggacaggactnaccccatgantcctgatgcagagngcctaaagantatgtgccgagatcctgngctttacttcagatca

SEQ ID NO: 14 - DNA, 317 bp, Mus musculus
  misc_feature 207: n = A,T,C or G
gactgagtttatcatgcaatctacttggcagaaatggttgcctcagaagttgctcgaaaacttgcctccgtgtttaatattcctttccaccaaattaaccaggtttacagacagggccccactggtatccacattcttgttagtgaccagatggttcagaactttcaagatgaaacttgttttttattctccacagtaaaagctganaataatgatggtattcacataattttgaagtgataccttaaacggactgaactctatccagtaccaaataaagtcatgcttaaaagtgtgtgaagactgaaaaaaaaaaa

SEQ ID NO: 15 - DNA, 505 bp, Mus musculus
  misc_feature 20, 400, 408, 420, 433, 448, 455, 473: n = A,T,C or G
tctccatacggttttcaagnatgggaaggtgacaaaaagctactcatttgatgaagtcagaaaaaatgcacagctgaacatcgagcaggacgtggcacctcattaggcttcatgtggccgggttgttatgtgtgcagtgtgtgtatacatacatgcacgtatgtgtgcacctgtgcgtatgtactaacatgttcattgtacagatgtgtgggttcgtgtttatgatacactgcagccagattatttgttggtttatggacatactgccctttttatttttctcccagtgtttagatgatctcagattagaaaacacttaaaaccatgtacaagattaatgctgaagcaagcttttcagggtcctttgctaatagatagtaatccaatctggtggtgatcntttcacanataacaaaccangaaaccttttatntataactaccagatnacataanacagatttgcataaaatnaccatgcctgctttatgtttatatttaacttg

SEQ ID NO: 16 - DNA, 161 bp, Mus musculus
  misc_feature 40, 49, 98, 104, 105, 109, 113, 114, 141: n = A,T,C or G
gaaaggcagcatgattgagaagtgggagcaggatactggngagcaaggncaaaggtgagaccagggccaaactaaaaaggtcacagagtttcggtgtngccagnnccancagnntcagacagatcctgctagagtggtgcngcagcaagactgagggctat

SEQ ID NO: 17 - DNA, 343 bp, Mus musculus
  misc_feature 28, 63, 73, 134, 159: n = A,T,C or G
tgcgcacacacgtcagccgctgtcaagngtgcatgagaagaacataactatgatgctcgcctntgccaccgtnacaacatggtgctactcccccccttgtcctcttctccctcccattgtatttgtaacagcantgaaccctagaattatatcctgtgnacctcactgtccactatgaaaaccataaagtgccttatagggattcgcgtaactaacacaccccgcttcgttggcctctgtttgctgaaggagaaggacaggcagcggagagctttccataatggcacataccaaatggcgctcctgatccaataaaagatgaattgcatttttgcaaaaaaaa

SEQ ID NO: 18 - DNA, 519 bp, Mus musculus
gactgagggaagagtgaggatacggctcgggtaaatcgagcatgtcatgtaccacgattggcgtttggtacccaagcacgaggaggaggccttcactgcgttcactgcgaaaccagaggacagattgaattctgtcccctatccacctctgctgcgagccatgatcctcgctgaacggcagaaaaactggggataccagcgtgcaggagccgctcctgaacctggagaggactcgaatgcgcccctgggactaccctgcaaaacaggagacgaaagggagggccaagggcaccccagtctaactgccaggactggcagagaggtcctgtgagtcatttgatggacgagtgcctttgactgtgctagatcttcacttcggtggcgagttatatcctacagccctgacacatcaggcaatgaataatggggcagatgacaggctgcttggtgtcattttcaggcgggcccctctctgattcctcggtctggaaataaacgccaggaaacagccaaaaaaaa

SEQ ID NO: 19 - DNA, 185 bp, Mus musculus
gactgagaatgagaaagaagtttgatgatggagaagatcctcttgatgcagagagtcaacaaggaggtggtggcaaccccttccacagaagttggaactcatggcaagggtttaaccccttcagctcaggcggaccgttcagatttaagttccacttcaattaaaccagctattttacttgctca

SEQ ID NO: 20 - DNA, 202 bp, Mus musculus
  misc_feature 32: n = A,T,C or G
gactgaggggctgtttttggagcattgcaagncattcttgggagccgctccatttcaaaggaatactataaccagacgggccgcatacctgactggtgctctctcaacaaccctcccctggaaatgatgtttgatgtggggaaagcgcaaccaccgctgatgaaaagggcattttccactgagaaataaacgcttgaataca

SEQ ID NO: 21 - DNA, 478 bp, Mus musculus
  misc_feature 34, 38, 39, 45, 46, 49, 51, 74, 84, 101, 112, 113, 152,346: n = A,T,C or G
gactgaggtcctggggtgggctcaggcgaagcgnggcnnagtcgnnctncncatgcatggggtctacacgcacntcctagcggnacttgtcctccacccanactggatatcnnacatgcgcctcaggaacacgtgaagttatgagagagatncaaaaactctatgaaaacaagtcatttcttttcttgggttgtggctggacagtggacgacaccactttccaggccctgttcctagaggccgtcaagcataaatctgacctagaacatttcatgttggttcggagaggagatgtagatgaattcaagaagcttcgagaaaacatgttggacaaggggataaaggncatctcctatggaaatgactatgctgatcttccagaatatttcaaacgactgacatgtgagatctccacaagaggcagatcagcagggatggcaagagaaggtcagctaaatggttcatctgcagcacatgg

SEQ ID NO: 22 - DNA, 477 bp, Mus musculus
  misc_feature 452: n = A,T,C or G
ctcgtgtgtgccagtgcagctatgagctgttctccagtcactcgaagtacgcacactcatccccgtggccagcgttcactgaaaccatccatgcagacagtgtgaccaagtgccctgagaaaaaccgaccagaagctttaaaggtgtcctgtggcaagtgtggcaatgggttgggccacgagttcctgaatgatggccccaagcggggacaatcgagattctgactatttagcagctcactgaagttcgtccctaaaggcaaagaagctgctgcctcccaggggcactaggacatggactgcgaatggcctgccccctgccccctgccccctgtcccctgccaaccgctccagcctcagtcaccgaatgatctgctctggtcaaatccttctatgccagccagggtggtgatgacccgtgacctttgaggaggggagtaggttgataccggnaactcttggatccttgccttctggc

SEQ ID NO: 23 - DNA, 192 bp, Mus musculus
cagtaatgaaagtaacagttgatgtccctcctcgacaaaaagagcaaagaagtgctgcagcaaaagccaaattcacagtggatttagactcggatgaagatttctcaggtttggatgagaaggatgaggatgaagatttcctcccattagatgctactccacctaaggccaaaatccccccaaaaaaaaaaa

SEQ ID NO: 24 - DNA, 497 bp, Mus musculus
  misc_feature 29, 35, 48, 53, 54, 58, 64, 65, 75, 90, 95, 96, 101,107, 112, 114, 121, 129, 130, 133, 137, 139, 140, 141, 149, 157,165, 180, 428, 475: n = A,T,C or G
atcttggcatgttggtctggtcacctaancagagnctttctgaagcanaaccnngcgngtacanntgtatagcanacagagaagcaccgncaacnncatgnagcagnctctngngatgctnattttggnntangcanannnttggaaantcattgancgacttgnccaacattcaccccntttccagatgagtggactgtagaagacaaagtcttatttgagcaagcctttagttttcatgggaaaacttttcataggatccagcaaatgcttcctgataaatccatagcaagtttggtgaagttttactactcttggaagaaaactagaactaaaacaagtgtgatggatcgccatgctcggaaacagaagcgggagcgggaggaaagtgaggatgaactggaagaaacaaacggcagtaatcccgntgacattgagattgacccaaacaaggaaagcaaaaaggaggtgcccnctacagagacagttcccatatt

SEQ ID NO: 25 - DNA, 247 bp, Mus musculus
  misc_feature 37: n = A,T,C or G
actgagttggtcgaattatgtttcagctcttctcagncatatgtccaaaaacatgcaaaaacttcctatgcctgtgctcaggggagaaaggccttgggaaaacaactgggaagaagttatgttataaaggttctacattccaccgtgtggttaaaaactttatgattcagggtggggacttcagtgaaggtagagataaaagttatattcctaataaatccatccatcagaatctcaaaaaaaaaaa

SEQ ID NO: 26 - DNA, 222 bp, Mus musculus
atacgctggaacaaggactttggagacaccactgtttaaaggaatggatgcctctgtgagcccatagaacatcgcacttcacaggaaacaagagctttggctacaggaacccagacttcgtttacaggacgttttagagttttcatttttgtgcaccaaacttgaagaggaacaagaagacagacctaaataaaatatgcaatatgaatggaaaaaaaaaaa

SEQ ID NO: 27 - DNA, 105 bp, Mus musculus
  misc_feature 17, 34, 37, 63, 78: n = A,T,C or G
ttggggcactgaggcanatcagcatctctgactntgnaggatggtggtgcggaggtgcaccgncctgaccttcacagnctcagaggaccgagtcttggtggctga

SEQ ID NO: 28 - DNA, 428 bp, Mus musculus
  misc_feature 39, 84: n = A,T,C or G
gactgagagacggcgagagggacccccacactctaatcngggtccagtatttcctggcacagcattatgataaacttgggcagnattttctggctttggaatatattaatgctgtaattgctagtactccaactctaatagaactattctacatgaaagcaaaaatttacaagcatatgggtaatctcaaagaagccgcacagtggatggatgaggcacagtctttggacacggctgacaggttcatcaattccaaatgtgccaaatacatgcttcgagcaaatatgataaaagaagcagaggaaatgtgctccaggttcacaagggaaggaacatctgccatggagaatctgaatgaaatgcagtgtatgtggtttgagacggagtgcatttctgcctatcagcgcctggggagatatggggatgcc

SEQ ID NO: 29 - DNA, 240 bp, Mus musculus
  misc_feature 51, 53, 56, 65, 72, 75, 140, 146, 207: n = A,T,C or G
gatatggagaaatgaagatgtctttggcaacaggatcatccaggtacattnantanaaaacatanagaattnttngaagccaagagttcaaaatgctattgccgacaaagtgaagtctcccaaaaaagttaagaacacanaattgngcctcatcaaagacacaagtgaacagtcacccagtgtcaaagccatgccagggaaagtgtngcaggcaaattctggatctgctacaaaaaccac

SEQ ID NO: 30 - DNA, 395 bp, Mus musculus
  misc_feature 198: n = A,T,C or G
gactgaggtgacttcattatctggagtgacacaaggtaagattcaactaaggggtcactgaatcctgaaaatcctgttcaagtaagcatggatcacctaacaacagcgatttatgatcttctcaggctccatgcaaattctagtaggtgttctacaggctgtccccgagggagcaggaacatcaaggaagcatggacntgcaacggagcagcctccagttcactgtctatgccgcacacggaatttccagtaactgggtatcaaagtaagtagctgcttaaaggacatccacagctttacccacacaagttacacttagtgttagtaactacttttatcttttttgtttaaatgaacaatttaaagatcactgggaacccttgtacctttgtggc

SEQ ID NO: 31 - DNA, 350 bp, Mus musculus
actgagttccttctaatgattctcagtcatgcccgagtgaagaaacacaggaggaggatctgctgaataagtgcatgcagtccatgtccatccaagaacaaggcccagctcacgccagcctgacggtctagtcctccacaccaagctcagccatcagctaacatccacatgctgagtgcacagggaagagtggaccatgtatgggatagtactagtagtaacggtatgttattttggttgttgagaaatttataatagccgccttcgtctgatatatttctgtgttaatgtgtaatgtatgcttgtttctagctagaacaattaaaattttacttgcaacataaaaaaaa

SEQ ID NO: 32 - DNA, 317 bp, Mus musculus
gactgaggtcctcaagggaagttgaagttgcctgtttgttctgggaggtagcccccacctcccacacctccacgcacattccaggcattcaatgtgtgtatggaaactgggtgagaacttggacctgccctttaaggacacaccgaaggtgtgaggatccagcacaatttatcccgaggcctttgcagcccagccttgccactaggtccaggcaggaatccaaaagggaggggcctcctgcctgcccgccaagcctgcgcacacctctgctctttgtctctggagcctaatcagcctccagatctagaaaaaaaaaa

SEQ ID NO: 33 - DNA, 189 bp, Mus musculus
  misc_feature 24, 28, 40, 46, 76, 99, 107, 113, 115: n = A,T,C or G
atttttaacctttcattaccagtnaccntgagcgtttgcntattanttgcttaatatgtacataagttatacatantgattactgaaccggaccatcgnaccatggngtgttngnggctggtgtcatccatgagcccaagccacccaagacgcagggccattagaggtctgccccagctgggatggggg

SEQ ID NO: 34 - DNA, 124 bp, Mus musculus
  misc_feature 20, 45, 57, 64, 75: n = A,T,C or G
tgccagccaggcacagccgngaacactggagccatgacaaccagncaccagcctcangacaggnataaggcagtntggcttatcttctttgtgctgggcctggggacactgctcccctggaatt

SEQ ID NO: 35 - DNA, 360 bp, Mus musculus
tgagacttattgcacatgtgctgtggtcctgagtccctgatgtacacacagaggcagaatttttcctcccaggatgccacatgaggtacctcagccagagccagagcctacctgaggagcttgggaacagcaaacctgaaggcatcgctaatgaccaggctgagggtccccagcaggaaggtggaccggaacacgcgccagatagccctgagtagtgggcccctctgactcctctctggctgcaggaaggcctctgtctcaggggcccccacactactgtgccctttgtgcctggaagagaggaaacatgggcacggggcccaagttatattcaatggctgtagcagaccaacaaataat

SEQ ID NO: 36 - DNA, 238 bp, Mus musculus
  misc_feature 67, 78, 92, 108, 118, 127, 176, 198, 209: n = A,T,C or G
tgaggtttatggacctgcgtcagtttcttatgtgcctgtccctgtgcacagcctttgctttgagcangcctacagaanagaaggaccgagtncaccatgagcctcagntcagcgatanagttcacantgatgctcagaattttgactatgaccatgatgccttcttgggtgcagangaggcaaagagttttgatcagntgacaccagangagagcatggaaaggcttggaatgattgt

SEQ ID NO: 37 - DNA, 183 bp, Mus musculus
gactgagtgattcacatctgagacatggctatggattggctaggcagtattgtgtccatcaactgtggagatagcctgggcgtctaccagggaagagtatcagcagtggatcaggtcagccagaccatttctcttactcggcccttccacaatggggtgaagtgtcttgtaccagaagtcacc

SEQ ID NO: 38 - DNA, 349 bp, Mus musculus
  misc_feature 170, 172, 175, 182, 192, 219, 226, 227, 234, 236, 242,243: n = A,T,C or G
gactgagaataccgctaccaagatgaagacacgccccctctggaacacagcccggcccacctccccaaccaggccaattctccccctgtgattgtcaacacggacaccctagaagccccaggatatgagttgcaggtgaacggaacagagggggagatggagtatgaggngntcncattaanaaggactaangagtcgtgtggatgttcatcttgagcntgggtannaccttcntncacatnngtgacgacccatccatctttatcaccaagatcattcctggtggggctgcagcccaggatggccgccttagggtcaacgacagcatcctgttttgtcaatgatgttg

SEQ ID NO: 39 - DNA, 322 bp, Mus musculus
  misc_feature 31, 32, 54, 58, 62, 77, 83, 85, 96, 101, 136, 138, 139,142, 145, 157, 221, 229: n = A,T,C or G
tgtgaagcgagctcgctggagcatggacganntgcccatgcagacgcgactgcncacntatnctgagcagctcactnagacanangccccggaacnggatntgaaccagcctgaagttatgccatcattgatggangnntanttntgcacccccganaggatcgagctggagaactaccgccgatctgtgaggcaggtggaggagctggaggaggtacccnaggagacncaggccgagcataacctgagcagcgtcttagacaaaggcaccgaagaggatgtggccagcagcagttctgagtcggagatggaggaggaggaa

SEQ ID NO: 40 - DNA, 410 bp, Mus musculus
  misc_feature 20: n = A,T,C or G
atgcttgagcgtctgctgcncagcggccgccgaagctgatgcaccagcgtcatcttccagattcctccctcccgacagacacctcctcatcgagaggtactacacctttgatgaggcctttgttcgggaggtcttgggcaagaagctgtccaagggtaccaagaaagacctggatgacatcagcaccaaaacaggaattactctcaagagctgccggaggcagtttgacaactttaaacgagtcttcaaggtggtggaagaaatgcggggctccctggtggacaacatccagcagcacttcctcctctctgaccggttagccagggattacgcagccatcgtcttttttgccaacaaccgctttgaaacaggaaagaaaaagctgcagtacctgagctttggggactttg

SEQ ID NO: 41 - DNA, 364 bp, Mus musculus
  misc_feature 287, 291, 319, 322, 327, 328, 338: n = A,T,C or G
gagccgacgtactggatgacccaagctctaaaggatcagcgtcctcagcccgggagcgccaccaccacttaaccccattaagatggcggcagcgcccgctcagccactcagggccgaagcctgcgtcacgtgacccgcacagctctcaccgcggtggctgatgggaggtggagatagaaccgagggtgtcggccattttgtgtctgtttcctgcgggacgcgctggtggcggttggtgtggagaactctgcgagtctcttgggttttttgttttttgttttttccancttntttccgcctctattctggctgttgtgtnggncgcanntatgggttgnttgaacatgatgcagttcaccagcat

SEQ ID NO: 42 - DNA, 513 bp, Mus musculus
atgatgcactctcctggctttgatggaggcagtagcctgagcttccagatgctgatgaatgcagacagcttgtacacagcggcacactgtgccctactgctcaacctgaagctctcccacggtgactactaccggaagcgaccaaccgtggcaccaggcatgatgaaagagttcatgaaacaggtgcagaccagcggcgtgctcatggtcttctcccaggcctggctcgaggaactgtaccatcaggtgctggacagaaacatgctgggagaagctggctactggggcagccctgaggacaacagccttcccctcatcaccatgctcaccggtcagtgagtaatgccaaggtcatagagtgtgcagtagttgggagaagctgacgttctgggcccttttagaaatgccacaatgcttgaaccggctttgagtaactgttccaggaaggcatcatgaaaacacccctcaagcatttgctctccttttgtcatggacagcctggtgtgctcacac

SEQ ID NO: 43 - DNA, 512 bp, Mus musculus
  misc_feature 188: n = A,T,C or G
gccggtccagcaatgacaagcagtgacctgccactgaatttccaaggaagcccttgttcccagatctggcagcacccgaggtttgcagccaaaaagcggagctccatggtgaagtttcactgctacacaaaccactcaggtgcactgacctggttccgaaagcgagggagccagcagccccaggaacntggtctcagaagagggacgcattgtgcagacccagaatggctctgtctacaccctcactatccaaaacatccagtacgaggataatggtatctacttctgcaagcagaaatgtgacagcgccaaccataatgtcaccgacagctgtggcacggaacttctagtcttaggattcagcacgttggaccaactgaagcggcggaacacactgaaagatggcattatcttgatccagaccctcctcatcatcctcttcatcattgtgcccatcttcctgctacttgacaaggatgacggcaaggctgggatggaggaagatcacatga

SEQ ID NO: 44 - DNA, 506 bp, Mus musculus
gactgagggttagcctggtccccatgaaggttcacggaccatcactgccactcctgaaggcagtccagaagctgtggacttgagtctgattggcctccctccgcccatgtcccagcggcccggcagtgcctctgccacaaggtctattttccgttccatgtctgtggctacaggcagcgagccaagaaaaaaggcactggaggccactggacctggaggcccccgagctatcaacaatcttcgaagatctaacagcactacacaggtcaaccagtcatggacgggctcccccagctcgcttcctccatggatcccaatcaaggaatggtgctggccacgtggggaagaaagagcgggcatgccttcccacctcaaattaacggatcacagagtcatgctgagagaccagttcctgaatgattgtagatcttgttaaactgacaaaacccagtgcagctccaacccttattagcttggtacccaaatacattgctctgaagtcacaa

SEQ ID NO: 45 - DNA, 414 bp, Mus musculus
  misc_feature 70, 147, 318, 372, 381, 386: n = A,T,C or G
gactgagatacctggagtctcctacctgacgaattttctttcgatggcattaccagagtataggctcaantttaaagaagcttttacacactggaaattctattaagataagatgtgaaggaatcagactatttcttttgtggcttnaagcacttcagacaaactgtgcagaagagcaggtgctgattttcgcttgcctagtgcctggtttcccagcggtcttgtcatccaggggtccctgcacactggagacactcatcaaccctagccctagtatagttgatgcaaaaatatacccagaggaaatcaccccacttntgccagccatatcaggggagaagattgctgaggaccaaacctgcttttttcttnaaatactgntgaantacatggttattcaggctgcaagcttgc

SEQ ID NO: 46 - DNA, 559 bp, Mus musculus
  misc_feature 24, 44, 79, 91, 165, 369: n = A,T,C or G
gactgaggctcggccatcttgatnttcttctcgttgccgtgcgntagacgtcttctgctcgatgctgctgatgaccctncaggaggagcgnggagccctcaccacagttcttgtaggccaccgacaggaggttccgttcctcattggacagtggttcgttcagcntcggtcacctggaggaggaaggacccgagcaccgttacagacaggtgatgacaatggggtgaagctatctttacatcaacccacaacaggtccatgcaccatggagaccgttagcaggcagaaacactgcactctggctgggaggaaggggagtgagtgacaggagcagcacaggtggcttctacagtctcaagcaccagtgtnctccacggaagaaccgtcagcctgtcctaaggacaaatcaaatcctctcccgtggcatggacacttagcactctcagcatgagatggccatgttctcaggaagagagcaggcatcctcaagctgaccagtgctcttacttgagtcatggtccctctgggaaagaatgagtcaccctctctcagtatgcct

SEQ ID NO: 47 - DNA, 106 bp, Mus musculus
  misc_feature 19, 44, 62: n = A,T,C or G
gactgagatcgagttccgngatgaagctgaactttctcctcggnccggggcagcagggcggnagttgacccaagacgagaagcttcagcttcgaaaagaaaagaaa

SEQ ID NO: 48 - DNA, 544 bp, Mus musculus
  misc_feature 390: n = A,T,C or G
tgaggtgctggaaagcaacacttctgaactacattttgacagaacaacataatagaaaaatagcagtgattttaaatgaatttggagaagggagtgcagtagagaaatccttagctgtcagtcaaggtggagaactctatgaagaatggctggaacttagaaatggttgcctctgctgttcagtcaaggacagtggccttagagctatagagaatctgatgcaaaagaagggcaagtttgactacatactcttagagaccactgggctagcagatcctggtgcagtggcttctatgttttgggttgatgctgagttagggagtgatatttatcttgatggtatcataactgttgtagattcaaaatatggattaaaacatttaactgaanaaaaacctgatggccttgtcaatgaagctactaggcaagttgcattggcagacatgattctcatcaataaaacagacttggtttcagaagaggagctaaacaacttaagaacaactatcagatcaataaatggactgggaaaagttttagaaaa

SEQ ID NO: 49 - DNA, 202 bp, Mus musculus
  misc_feature 29, 48, 50, 168: n = A,T,C or G
agcccaaggaactggaaaagaatgaaggncattcctaaaagggccccntncgacgtgggggatgacatgtccaacaaagtatccatgtccagcactgcccagggcagcaacatctttgagagaactgaggtcttggcagctctgatcgtgggcggcgtggtaggcatnctctttgccgttttcctgatcctgctgctggtgt

SEQ ID NO: 50 - DNA, 134 bp, Mus musculus
  misc_feature 32, 39, 40, 42, 46, 50, 53, 55, 87, 93, 100, 118: n = A,T,C or G
cctccatttagacttcacctaactcctgacantaatggnntnattngctnagnangaccagaaaaggagagggcccaccagacgccngcccanagccttncagacccttccacagtcngggctgtgctgaaagg

SEQ ID NO: 51 - DNA, 496 bp, Mus musculus
  misc_feature 33, 40, 65, 72, 170, 171, 177, 212, 231, 249, 252, 316,378, 387, 399, 403, 404, 448, 450: n = A,T,C or G
gactgagccgagcccgatcctggtgcgtctggncaccggnggccaggagggactgcagtaccagncaggggnccacataggtgtgtgcccacccaaccgtcctggcctagtggaggcactgctgagccgagtggaggaccctccgccatccacagaacctgtgaaatgannaacaantggagaaaggcagccctggtggccctccccccggntgggtacgggacccccggntacccccatgtacgctgnggnaggctctcacctacttcctggacatcacttccccgcctagtcctcgcctccttcgactgctcancaccctggcagaagagtccagcgaacagcaggagctagaggctctcagccaggacccccggngctacgaanagtggaagtggntcannatgccccacactgctagaggtgctggagcaatttcctttagtgntnctgcctgccccgcctgatcctcacccagctgcccttgctccagccc

SEQ ID NO: 52 - DNA, 489 bp, Mus musculus
  misc_feature 20, 26, 40, 154, 184, 197, 200, 387, 412, 413, 460: n = A,T,C or G
actgagctatcttcgacttncacgtncacccccaggccgngacgggtgaggagggtggtgtggcccgtttccagtgccaaatccatgggcttcccaagcccctgatcacatgggagaagaacagagttcccattgacacaggacgatgagaggnacacacctactgcccaagggagtcctgcanataacaggacttnggnctgaggacagcggcatcttacactgtgtggcctcaaacattgctagtgtgcgggtcagccacggggccaggctcactgtgtcaggctcgggctcagggacctacaaggagcccaccattcttgtggggcctgagaacctcaccctgacagtgcaccagactgcggtgctggagtgtgtcgccacggncaacccgcgccccattgtgtcctgnngccgcttggatggccgccccattggggtggagggcatccaggtgctnggcacaggaaaccctatcatctcagatgt

SEQ ID NO: 53 - DNA, 282 bp, Mus musculus
  misc_feature 20, 218: n = A,T,C or G
ggtggcatgtcacatctganagaggctccatgtgtaagacctgtgccaaccagcgccagccacgatttaagccatgaggatgagcctcctgccgcttcccatatggccactggagctggatgatggcggcatacagacagatcatcccagacatgcattttagaaagttatcctgttgctcaaccttggaatgcgtcacttggtagccaagcttccgntgatagtcctccaaagccatcctttccttgaaagctgggtagaaaggacggagtatggacactt

SEQ ID NO: 54 - DNA, 235 bp, Mus musculus
  misc_feature 128: n = A,T,C or G
actgagacttagcctgacagattcagatgggccccggagtggtggttccttccttcgctaggaactgatgtcccagaaccttcaggagacaagccaggcctacccccgacatcgacctggtagccacngcaggccctaaaagtctcaaagttaccccacgagcaaccatgtacacgtttcctgcctgacacaaagcaggtgctcaataaaagagagctattgaagtaaaaaaaaa

SEQ ID NO: 55 - DNA, 263 bp, Mus musculus
  misc_feature 23, 24, 26, 67, 95, 101, 111, 113, 114, 123, 128, 129,135, 138, 141, 152, 154, 158, 167, 178, 204, 206, 238: n = A,T,C or G
gctgcttcatgaaatgcaacttnnanacccaacagcctctttgatagcaaaagtggctacagcccangatgacataactggcgatggcacctacntccaangatcttatcntnngtgagcctnatcanncaggcngtnccntgtgcatttangnaggncttcacccnctgaataatanctgaaggttttgaagcggcaaaggantnggcactccaatttctggaacaagtcaaagtangcaaagagatggacagagaaacatt

SEQ ID NO: 56 - DNA, 110 bp, Mus musculus
accctgaaagatgagaccggagctatcttcatcgaccgggaccccaccgtctttgcccccatccttaatttcctgcggccaataaaacctgtcactgccaaaaaaaaaaa

SEQ ID NO: 57 - DNA, 457 bp, Mus musculus
  misc_feature 36, 39, 42, 52, 53, 55, 57, 58, 83, 84, 89, 100, 101,102, 266, 276, 289, 292, 296, 298, 305, 308, 313, 331, 332, 357,365, 398, 399, 404, 409, 410, 419, 420, 434, 436: n = A,T,C or G
gactgaggctcaaatctgatttccttctggggtagnggncgngcacgtaganntngnngaaagatcttgcgaggaggatcaanntgctngaatatgcttnnnagcaggaaagagccaaataccacaagttgaaatatgggacagaactgaatcagggagacatgaagcctccaagctatgactccgatgaaggtaatgaaacagaggtgcagccacagcaaaatagccagttaatgtggaaacaaggccggcagctactcaggcantgtcttactncggactggtatgnatncccntncacactncgntacangcctttgcagcacaccanngtaactctctggattcccatgattntgcacagngtggactaacatactgggactctataggggttnngatgncaggnngcagactanncctggagtcttggngncttgcatacagatttatgccg

SEQ ID NO: 58 - DNA, 329 bp, Mus musculus
  misc_feature 27, 48, 50, 66, 256: n = A,T,C or G
gactgagacatacatactaggcgaccncaggcagccgccctctgacantncatggacccaagactnctgcagctgcccctgagaagcagccagaagacatcaaaggccggattcccaagggtcttccaaagccttacaacagactacagagttgaagtgctggaagacggagtgttacccctaatcttattacaccccccccagactaatccccaagctgggagtcctaccacccgggtcctccctcccacaactntgtcttaccgaccctataaatcgtaataaatatctgtgttacagcagggaggcatatctcgcaccccgtggat

SEQ ID NO: 59 - DNA, 464 bp, Mus musculus
  misc_feature 16, 48, 49, 53, 70, 71, 72, 93, 97, 100, 101, 109, 124,160, 165, 167, 169, 210, 225, 350, 379, 414: n = A,T,C or G
ttactgcgtctggtgncagcatggcctacggcctgttgcgcgccttannaggntcaccagaagccctcannngtgccctcttctgcctcctcntgcncannccatccgnggatccgccagccancatggttggcttttgattggggagaacttccgcttnctgtntntnttgttcaagagcaaggatgtgatgattttcaatgggctggnggccctgggcacggngggcagtcaggagctgttctcagtggtggccttccactgtccctgctcacccgcccggaactacctgtatggactgacagcaattggcgtgcctgcgctggccctcttccttatcggagtcattntcaacaaccacacttggaacctagttcgncgagtgccagtaccggagggccaagaactgctccngcggcccccgaaccttcctcctcctaagctcaatcctgggcccgtgccag

SEQ ID NO: 60 - DNA, 132 bp, Mus musculus
tctgagctataaagacttgaagttgcccttccagtctaaagatggaaaacagtaccaccaccatttctcgggaggagcttgaagagctacaagaagcgtttaataaaataggtaaggttaaggaaaaaaaaa

SEQ ID NO: 61 - DNA, 259 bp, Mus musculus
  misc_feature 103: n = A,T,C or G
actgagcccattgctatggacagtgccatcactctgtggcagttccttctccagctcctgcaggagcctcagaatgagcacatgatctgctggacatctaacnatggggagttcaagcttctgcaggctggaggaggtggctcgtctttgggggattcgcaagaacaagcctaatatgaattatgataaactcagccgagccctgcgatactactacgtaaagaacatcattaaaaaagtgaatggtcaaaaaaaaaaa

SEQ ID NO: 62 - DNA, 519 bp, Mus musculus
gaactgagatgtatctggatattatgcatgcacttaaaaatgccagcacggataactcagtcataactgttttcacaggaactggtgactactacagcagtgggaatgacctaaaaaacttgattaatgacgctggtgaaatacaggacgtagtagccacgtctactaagatactgagggaatttgtaaactgttttatagactttccgaagcctctggttgcagtagtaaatggcccagctgtgggaatcgctgtcaccatcctggcactatttgatgctgtctttgcttcggacagggcaacgtttcatactccattcagccaactcagccagatcccggaagcatgctccacatacatgtttccaaagataatgggcccaacaaaggcagctgagatgcttctctttgggaagaagctcacagcaagagaggcctgggctcaaggccttgtcactgaagttttccctgagagcacttttgagacagaagtctggaccaggctgaaaacatattcgc

SEQ ID NO: 63 - DNA, 397 bp, Mus musculus
  misc_feature 27, 36, 37, 42, 44, 64, 160, 316, 320, 326, 330, 334,337, 351: n = A,T,C or G
aggattcttcacagcggatgagtgtgnggtcatgcnncagangntcggcgagattgtatctgtnatggatgtccctctgcactgccggacagaattttccacccaggaagatgagcaacttcaaacccagggcaagacagactacttcttgagcagcggngacaagatccgattcttctttgaaaaaggcgtttttgatgagaaaggaaatttcttggtccctcctgagaaatctatcaacaaaattggccatgctctgcatgcccatgaccctgtcttcaggagcatcacacattctcccaaggtgcaggctttngttngaagtntggncctncanatgccagtggtggngcagagcatgtatatgtttaagcaacctcactttggcggcgaagag

SEQ ID NO: 64 - DNA, 237 bp, Mus musculus
gactgagccacaaatgcagcccacggccaccatggccacagcggccgccactaccgccactgttgccctgacgacttcgtgggacaatgccaccagccgccccacggcggagcctgaccccatcttggacaactatgtgctgctggtggtggtgatgtcgctgttcgttggaggcacagctggtggtgctgtctggcgttcttctgctctgcaaacgctgctgggaggtgcaccaga

SEQ ID NO: 65 - DNA, 310 bp, Mus musculus
  misc_feature 185, 195, 264: n = A,T,C or G
gactgagcccttcaagctcctaggtattttagatgttgagaacactccatgtgctcgagaatctatactctatggctcattaggatctattgtgactggacttggacatttcttggtgaccagtagaattagaagatcatgtgatgttggagtaggaggatttatcctggtgactttaggatgcntggttccacntgtaggtataatttcgctaagcagaggatccaggaaagaattgccagagaaggaataaaaaataagatnttatatgaaagcacccaccttgatcctgaaagaaaaatgaagacca

SEQ ID NO: 66 - DNA, 362 bp, Mus musculus
  misc_feature 16, 33, 38, 69, 79: n = A,T,C or G
gactgagagggggctnggacatttgcttcatangaaanagaccacaattcttacggtcatcctgatctnaacgaaggantcttccgaggagccgccggagttgagtttgagctgcagttctgagacgatgcccagcaggtctgccttttcagcccgaaggcgcatcacctggatcttcagatgcctccacttcctgctccacctgggtcttcagcttctccacttcctcctccaaggctctgggatacctgtagccacctgtgaggtcctgggaagaccaggaatgaggcatgaggcgaatacaaaaatcaaacggaaggtctggctagtaaacttgatggccaagcatcacaaaaaaaaaa

SEQ ID NO: 67 - DNA, 138 bp, Mus musculus
  misc_feature 20, 32: n = A,T,C or G
tgaagaacttccaggacatnatgcgaaagcanaggccagaaagagtgtctcatcttcttcaggataacttgccaaagtctgtttctacttttcaatatgatcatttctttgagaagaaaattgatgagaaaaaaaaaa

SEQ ID NO: 68 - DNA, 510 bp, Mus musculus
  misc_feature 28, 40, 189, 190, 218, 221, 401, 478: n = A,T,C or G
gactgagatacacttttggcgtcatggntcggtttggagntatctcagaaggtgaagtagaccactctttctttgacagtgattttgaagatgcaaagaaatgtgaaagtaattccatttttgacaagcaaaatgatgatgaccttaaggaaggaataaataaggacaccaaaaatgtaaacttgaaanntggagtacaaaaatgatcatcttaaggnganaatagatcctaacacagaaaatgtgaacttgaaacttgggctacaaacaacagaaaattaccttactcagaaaggaaatgaaaggaaagcaaacttttcttcaaaggaacaacatatagaaaatgatcctacacaagcacgaagttcctcagtgttgacttcttcacgatcaaaaaaatngtgtgatgccacaaaaggacataaattaaacttgcccgttccagatagaattcctaagattgtgaaaggtgagggcnattactacacggatggggaggaaagcagcgac

SEQ ID NO: 69 - DNA, 227 bp, Mus musculus
  misc_feature 200: n = A,T,C or G
gactgaggtggggtctttaaaagcctgcccgccgggtacgagccctgccatccttgttctaaggagcctcccccagggcttggccgtgggcccctcgagatccaggaatcagcacttgggggtctggtgtgtaggagcacccctgcagcccggcctgcattggggacctctggaagatgagtccgtcctggagaagggcngagggaatgaagacaccaaaaaaaaaa

SEQ ID NO: 70 - DNA, 491 bp, Mus musculus
  misc_feature 118, 198, 208, 223, 451, 470: n = A,T,C or G
cgtgtgggcaaaagggctaccaggaaacggactctgtggtcagctcggtgacaaccaaagccaaaggtgtggctgtgaccaacacttctcagcttggattccggatctgggacgtggncgactatgtggtcccagctcaggaggaaaactccctcttcatcatgaccaacatgatctgtcaccgtgaaccagacgcanggtacctgtncagagattcctgatnagaccagcatttgcgattcagacgccaactgcactctcggctcctcggacacccacagcagtggaattgggactggaaggtgtgttccattcaacgcgtctgtgaagacctgtgaggtggccgcatggtgcccagtggagaatgacgctggtgtgccaacgccagcttttttaaaggctgcagaaaacttcaccctcttggtaaagaacaacatctggtaccccaagnttaatttcagcaagtaagngggggccgggcaataggcatt

SEQ ID NO: 71 - DNA, 431 bp, Mus musculus
  misc_feature 29, 62, 71, 82, 107, 112, 113, 126, 128, 129, 130, 137,141, 142, 145, 146, 148, 150, 155, 158, 163, 164, 165, 171, 176,178, 179, 181, 182, 186, 203, 205, 206, 219, 312, 318, 329, 352,390, 401: n = A,T,C or G
actgagatattggcatcaacctgtcagcncctatgttcagaggaatttataggggggtgcanaagcatcangatgacttacnagatgtaatagagagagctatccanattgnncgttaaaaagacntnnntttcagnacannggnntntnacccnacnagatnnnatgagnttgananngnnagcnctgatcattaccttatnannattgctgagcctngctgagaacaacaaaggaaaagttgttgcaataggagaatgtggacttgattttgaccgacttcagttttgtcccaaagatactcaactcaantatttngaaaaacaatntgaacttgtcagaacaaacacanttaccaatgtttcttcattgtcgcaattcacatactgngtttttagatnttatgagaagaaatagagaccggtatgtgg

SEQ ID NO: 72 - DNA, 520 bp, Mus musculus
tgaggtattagaaatggctactccacagtcagttttcgtctttgcaatctgcattttaatgataacagaattaatcctggcctccaaaagctactatgatatcttaggtgtgccaaagtctgcctcagagcgacaaatcaaaaaggcctttcacaaattagccatgaagtaccaccctgacaaaaataaaagccctgatgctgaagcaaaattcagagagattgcagaagcgtatgaaacactctcggatgccaatagtcggaaagagtatgacacaattggacacagtgcttttaccaatggcaaaggacaaagaggcaatgggagtccttttgaacagtcatttaacttcaattttgatgacttatttaaagactttaatttttttggtcagaaccaaaacactcggtctaagaagcattttgaaaaccacttccacacacgccaggatggttctagtagacaaaggcatcacttccaggagttttcttttggaggcggattgtttgatgatccccaa

SEQ ID NO: 73 - DNA, 324 bp, Mus musculus
  misc_feature 30, 35, 40, 51, 89, 106, 119, 141, 149, 150: n = A,T,C or G
tgtggtaggacacacatgggacacccaggntcatncccgnaccagatgatncgagaacagagtcagggagtccctactcctgctgcagnagccaccaccagcctgncaaggcaccccancactcacctgtggagaaccctnggccccgnnttcgtcccccacggaaaaagccccggcctccagcagagccaccccggtatcctcgggaccggttgtcagaagacttgccagcctggtcaactctgatctgccgcccgtccacagactgcaggagaaagttagggtcactgtacccgattccacaccagacctatccaaggtgac

SEQ ID NO: 74 - DNA, 102 bp, Mus musculus
  misc_feature 17, 34, 42, 45, 77: n = A,T,C or G
atctaccagctgaaggngctccactgtgccatgnatgcctgnatnaacctggggatgctggaggaggcgttgatctncgccatgcgcaccatggagccgtac

SEQ ID NO: 75 - DNA, 477 bp, Mus musculus
  misc_feature 38, 42, 47, 51, 56, 69, 71, 81, 83, 87, 97, 104, 173,396, 399, 425, 436, 451, 452, 458: n = A,T,C or G
tctgggccgaggccataattacttcggtgaggaacatntcancggantggnccagnaagcatgggtgantnccatatactntncatncaccgatctntaccagngtctcgagtccaccagctcaggtcgcaggcatgagcttcttgaggctctcgtagcccaccacgatgacngatggtggagggtgtagccgagatgattcttgccgagaggcccttcatgaggccccaaggcccctcctctgccatcagctgcctgaaggtcaggacgatggagctcttgccttcaacctgaaaaagtcaccacagtgaagagccaccaactgtgctgtcaccactgctcagcacaccctcaatagcacttcaggctcgaggagggactccgagggctagcctntgntttctgggcagcacagtgacttcctnctagctctggngacagacttcctggnnggactnaaccctgcctacccccagg

SEQ ID NO: 76 - DNA, 437 bp, Mus musculus
  misc_feature 18, 31, 70, 109, 111, 112, 116, 122, 128, 141, 222, 239,254, 257, 264, 280, 290, 292, 319, 323, 327, 330, 348, 357, 364,372, 419: n = A,T,C or G
ggagcatgtacggcggangtggagagctatnaagaaagcagtgccagcaaagtcatctctcctcactgtntctgaggggcaggatggaggagcgtccaggaggaatcancnncggnttcagnttctanaagcacgagcagnaggcctctgttcacgccatgatgaaggagaaccaggaacataatgaagaaagttcctagaacttggatgagaaattacaanactgttcaaaggaaaangaggagtctaagaancanggctcanagcacctccggaggcnctgaggaatntngggctcagcgttcatgattgcttgagnctanacangangcatgaagaggcatcggntgaagattnaattttnaagcttanagagctgtacaaggagcccacattcctgaaaggacagctcgctactnttgaagagactgtaactg

SEQ ID NO: 77 - DNA, 512 bp, Mus musculus
  misc_feature 38, 168, 206, 357: n = A,T,C or G
agccttttgccgcttgtctgagcagtacagaaatcagngccccgactgctcatcaatcgagacttggtggggccgttcgttctgagtcctcgaaggaaagatgtggtccagctaggggatgtagttcatggtgtggaaaggctggtggacctcctggggtggacacangaactgcatggatcttatgcagcggggaaccgtggcangcatggatggacaggacagataagactatggcttcttcacctggggaagtcacacagcagatcatcctatgtccagcaagacttcatgcctgaagacagctccaacacgtttacaaacatgaaccagaccacaacatgtggcctggacagnggtcctccgaggctgcctttggaaaggctgaccagggatgtctacccttggggcccctccatgtgtgcgccctgtccacctcatcactgctgaaggtgtagtgcaggtgctgctttctgcagcggcccttaagttatcacgagggcagcacagac

SEQ ID NO: 78 - DNA, 462 bp, Mus musculus
  misc_feature 366, 393, 401, 402, 406, 410: n = A,T,C or G
ccctgcttggtatatgtcaccttcaatcagaagatctacgtgtactgggaggtgcagctggagcgcatggagtccactaaccttctgaagctgctggaggctgagcccgagtaccaccgcctactgcaggagctgcgtgtggatcctgaagatctccctgcagtctgtaccctgcttcatcagactctctaccatccggaccagccactacagtgtaccccctcaagctttcaggaccccacctagctgggctcgaccacttagctggtgaaggatccttccgagcccccaaccctaccccagagcgatccatgaccttggaggatggggagtgtgtattacagaggaaggagcaggactgggttntcagccaggatggatgtggccccccgngagcctgnnatgnaganatccatccggctgcccctctgctcgcggctccttcttatgctccctgctctc

SEQ ID NO: 79 - DNA, 500 bp, Mus musculus
  misc_feature 24: n = A,T,C or G
gactgagtaagctcaacccagggncacctctgccctctgctctccacgcatgcgcctctggagaagaagtcttggcccagtacttacagcagcagcatcctagagtcttgagttcgtcacatctgctgctgactccctgcaaagtggctcctccatacccacacttcttctcaagcttcagtcagaaagggctggccatggacagtacccccaagggcgcagcagtacagagcatccctgtgtttggggccttgcgttcaacttcatccttgcaccgtacgctgggagacttggctgaagagctcagcagacttgacctgcggcgctgggctagcttcatggacgctggcgtggagcaggatgacatggaagagatgctgcacgagttacacaggttggcccagtgttaccaggagggtgacagtctctcgaactgaagtccaggggagggtagtgggaagaagaatttataataaaagctgccgtgcaggaaaaaaaaa

SEQ ID NO: 80 - DNA, 226 bp, Mus musculus
gactgaggaaaccattgaagatgttgaagagatgctcaacaatctccctggtgtgacgtccgttcacagtcgtttctatgatctctccaggcctaggtgatgagaaagggcacggaatgggaatggggacagggtagtctccacccagctccacctgagtggactttgtccagtgtggtctgagtgcttgctctaataaatgccttggcattgttaaaaaaaaaaa

SEQ ID NO: 81 - DNA, 353 bp, Mus musculus
  misc_feature 26, 49, 155, 172, 266, 279, 282, 284, 285, 303, 316,319, 323: n = A,T,C or G
caacaccgaggcttgagcagttcagngtggtgcctgccacagcggattnttccatcctgatagaaatagatgaggtcgaccagtagttcattacacgtgaagcagacaaagcaggacgggtgccagcatactccaggtcccgcacgggaggcgancacggacacctcccctncattcatctgcagctccacactgaaaacaaagacttgaagttaacaaacggtccttcacctaaattactgggttttcagcatttcccacttganaataacctatggntgngnnagggcagctttaatttcngtctactcctgcnggngacngaatggctatcccaccttcaatacaccctg

SEQ ID NO: 82 - DNA, 552 bp, Mus musculus
  misc_feature 29: n = A,T,C or G
actgaggccctgctgtccgtccttcgagnaaagccaccgttcaagaacagtctttaggaaagtgggagggtttgtgtacattacatccttgctcgttgctatggagaggtctttgagctctcctcccaagaacggctgggagaaggtgagccagagccaggtgttggagctcctgcacactgtgttctgcacgctgacggcagccttgcgctatgagccggccaactcgcacttcttcaagaccgagatacagtacgaaaagctggcggacgctgtccgcttccttggctgcttctcagacctcaggaaaataagtgccgtgaacgtcttcccgtcaaacacgcagcctttccagagactgctggaggaaggggcggtgtccgtggactctgtgtcaccgaccctgcggcactgcagtaaactctttatctatctctacaaagtggccacagattcttttgacagccatgcagagcagatccctccttgcctgacaagcgagtcctctctcccctctccctggggaacaccagctctgtccaggaaaagttc

SEQ ID NO: 83 - DNA, 554 bp, Mus musculus
  misc_feature 118: n = A,T,C or G
tgaggcatcctcgtgtcagggcacacagcaatcattgagtcctgccaggaagccttcatcctgcgcctcttccgccagaaaaacaagcgcgggttcatcaaagctttcaccgacaacngctgttgcctttgacactggcttttgtcacgtggaaagagtgatgccggaacctttttgtgaggaagctctacctgtggccaaggttccatgtagcagtcaactcattcttggaacaacacaagcctgaagttgtagagattcacgtgtccatgactcccgccatgcttgccattcagacggccatactagacatcttaaatgcatgtctgaaagaactgaaatgtcacaacccgtcacttgaagtagaagatttgtcactagaaaacgctcttgggaagccattcgacaaggtccacttcttttctttctcttaccccatagctggttacgttgttgttttaaagagtggaaggcatttcaatattcaccatgttctttgtaggaattaaactgtaggtgacttaaagtggggctgctttgcccactgggaaata

SEQ ID NO: 84 - DNA, 393 bp, Mus musculus
  misc_feature 17, 148: n = A,T,C or G
cattctgtctctctggncatagcaagccctaatgctatagacttggcagtggctgctgatgtggaacactgtgaatgtccccaaggctacactggggacctcctgtgaggcctgcctccctggctattatcgagtggactgggatacntctttggaggaatctgtcagccctgcgagtgccacgggcatgcatccgagtgtgacattcatggaatttgctctgtgtgtacacacaacaccacgggggatcactgtgagcagtgcctgcctggcttctatgggacaccttcacgtgggaccccaggagactgccagccttgtgcctgccctctctccattgactctaacaatttcagccctacctgccacctcactgatggagaggaagtggtt

SEQ ID NO: 85 - DNA, 213 bp, Mus musculus
  misc_feature 35, 37, 38, 64, 133: n = A,T,C or G
gcatctgctcccaagtgttgcttccagaagaaganannattatagtcgaagaaaccaagagcantgggcacgacagtgatagaggaaaagagcctcgtggatacggtgtatgccgttaaaggatgaagtccangagttaagacaggataacacgaagatgaacaagtccttagaggaggaacagagggcccgcaaggacttagagaagctgga

SEQ ID NO: 86 - DNA, 164 bp, Mus musculus
  misc_feature 137: n = A,T,C or G
gatgctgagtctgacttggtggatggccggcactccccaccaatggtcaggagtgtagctgccatccatcccagcctggagatcccgatgctgattcctaaagagaagcccccaatcacagttgttggtgatgttgnaggaagaatcgacatcattgtcgatca

SEQ ID NO: 87 - DNA, 139 bp, Mus musculus
  misc_feature 44, 49, 60, 70, 83, 88, 98: n = A,T,C or G
catacttctcatttaaactactcaagaaagaaggccccgttgcnaatgnacagaactctnccattcctgnaaaacttcctganccagngaaagccagngaagcagcatgtggaaaaagacccagccaaaggctcaggta

SEQ ID NO: 88 - DNA, 461 bp, Mus musculus
gactgagcgagtctccagttaataagtcacatcgaacaatttttggataccaatgaaacactgtattttatgaaaagtatggactgcatcaaagctttccgggaggaggccattcagttttcagaagaacagcgcttcaacagcttcctggaagcccttcgagagaaagtggaaattaagcaattaaatcatttctgggaaattgttgttcaggatggagttactctgatcaccaaagacgaaggcccaggaagctctatcacagctgaggaagccacaaagtttctggcccccaaagacaaagcaaaagaagatacaacgggacctgaagaagctggtgatggtggatgatttactggacatgatatagatcatggatgtgtggggaatccaagagtgccagctctgtgctgctgagagctctggcagcatccctgaggccgtgcaggggatgtgaagct

SEQ ID NO: 89 - DNA, 303 bp, Mus musculus
  misc_feature 78, 166, 188: n = A,T,C or G
tgagggggttccatcgttaagccaatcgtttatggcaatgttgccaggtactttggaaagaagagagaagaagacggncacactcaccagtggactgtgtaccgtgaagccctacagaaacgaggtgggcatcgcattttcccggaaggagtgtttgcggtgcagnctgtttagttgacagtaaactngaaattgtgtatagtaacgtttccaagtatgaggtgggtccattatagactcccaccactagccccatcagaaacaataaaatgaagcccctcatcttcatgtaaataaaaaaaa

SEQ ID NO: 90 - DNA, 466 bp, Mus musculus
  misc_feature 96, 205, 242, 294, 296, 304, 324, 326, 346, 359, 368,373, 406, 414, 429, 433, 435, 445: n = A,T,C or G
gggaggagttgaagatgagtttgatgctgcccaagagttgctggaatgcatgggctccaacgtggtatacctgtggagcgtgttggaacggggcantctgcaaagatctgcaacaacatgcctgttggctataacgtatgattggaaccagctgaagctatgaaccttggaatcaggtcaggccttgacccaaaactattggctnaaatcctgaatatgagctccggacgatgctggtccancgacacttacaaccccgtacccggggttatgcatggggtcccctcatccaananctaccaangtggatttggaacaacactnanggctaaggatctgggattgnctcaagactctgncactcagcnccaanaacccaattttcctaggaagcctggcccatcanatatacangatgatgtgttcaangggntnctcaaagaangacttctcatctgtgttccag

SEQ ID NO: 91 - DNA, 246 bp, Mus musculus
gtttgagatgaggtttgccaagatgcccgatgagcccatggaggcgcctgcgctgcccgctcccacggcccccatcgtgagcaagggggctgagagcagccgcagtagtgaggagagctcttcagattcaggcagctcagactcagaagaggagcgagccactcggctggctgagctgcaggagcagctgaaggccgtgcacgaacagctggctgccttgtctcaggccccagtgaacaaaccaaa

SEQ ID NO: 92 - DNA, 299 bp, Mus musculus
  misc_feature 255, 257: n = A,T,C or G
attgagcgttggtgagaagataaagtcagctctgaagaaagctgtgggaggtgttgccttatccctctcgactggcctctctgtgggtgtattcttcctgcagtttcttgactggtggtactcttctgagaaccaagaagccatcaaatcactgactgccctgcccactccaccaccacctgtacacctagactacaactctgattctcccctcttacccaaaatgaaaacagtgtgcccactctgtcgtaaaantngagtgaatgatactggtgtcttgccacctctggctatgtatt

SEQ ID NO: 93 - DNA, 275 bp, Mus musculus
  misc_feature 23, 26, 48, 51, 55, 57, 59, 60, 66, 70, 83, 255: n = A,T,C or G
tgagtggagcagccggcacctgnggncatcactcgcgctaggggcctntgncccngngnntgcagnaggngatgtttgccagngacacggactgtggttccatgtggatggctgactctgtggcctggaaccctcacaagcttctcgccgcagggctgcagtgctccgctcttcttctccgggacacctcgaacctgctcaagcgctgccatggatcccaggccagctacctcttccagcaagacaagttctacngatgtggctctggacaccag

SEQ ID NO: 94 - DNA, 215 bp, Mus musculus
tgagatggtgaagaaagctcagactatgatgaagaaagctcagaagaagatgaatcgcttggggaagaaaggggaagctgataggcatgtgtttgatatgaagcccaagcacttgctgtctgggaagaggaaagctggtaaaaaggacaggagataatgtcctcttcagttggaacagtttaagtagactcttggttattttgggtgtggtatgg

SEQ ID NO: 95 - DNA, 283 bp, Mus musculus
  misc_feature 31, 236, 253: n = A,T,C or G
actgaggtgcacaaggcaaatccaatacggnaagtacgcagcagtggaggtgaaatctagcgagctgcctctgactgtgccggtgtctcctaagttctccactcggttccagtagaagctgcatctatatcacaagcatggagcacgcggtcttcagactcacccgccatgtgtggaaacacacctgataactaatggatgccgggtgttttgaagatgactctacacagcaactntgaagctaatgagcctncactcatttagactttaaccagacttcttg

SEQ ID NO: 96 - DNA, 361 bp, Mus musculus
  misc_feature 91, 97, 99, 101, 104, 107, 111, 112, 116, 117, 128, 135,149, 156, 178, 204, 219, 275: n = A,T,C or G
tttgccaatgcaggaggagccccgaacatcgagactgcacctctgttcctgaaccctcagaacctctctctgctcaccagcaacccagaangcttgntntntgncgntgcnncctnnacagggaactnatgcacntacaggcagccttnatgcctnctccacctcaactgagtccatncagagctctctgttcacagtcgcctntgccagtgggcctgnttccaccaccacagctgcctccaaggcacagtgagctgagtgcagagctgggttgncacaggctttcctcactacagggtgataggctggctaccagctgcttaagaaaatgacacctgtcattggccttcctcccaccatg

SEQ ID NO: 97 - DNA, 210 bp, Mus musculus
cgtgtgagacatgaacttctcttggcgaaatgaatgcaaccagtgtaaggcacctaagccagatggcccaggagggggaccaggaggctctcatatggggggaaactatggagatgatcgacgtggcagaggaggatatgaccggggcggctaccggggccgaggaggggaccgtgggggcttcagagggggccggggtggtggggacag

SEQ ID NO: 98 - DNA, 315 bp, Mus musculus
gactgagacagaagaaaaaggaaaaacacaacaagacgcagttcttcgagcagcctccaagagagaatcagagtcacaggaaaacgcctcaagcagccgccggtaccatggcagttcacacacctcccagtgtccttgtgcctctgcttcaggcgcagccagacgcgccctgggcatccgccgtctctaccatagggatgctggtgctccgtgcatccgtgtacacgtgcaaggttttgctctgcttgggtggacagcagcccctgtaccactgaactcattttgtaacagcaatttcgcttgcaaaaaaaaaaa

SEQ ID NO: 99 - DNA, 213 bp, Mus musculus
  misc_feature 84, 162, 168, 174, 176, 183: n = A,T,C or G
ctgggagcaggactatcatgtttgagatcaaccgcctggatctaggcctgaccgtggaggtgtggaacaagggtctcatctggnacacgatggtgggtactgtgtggatcccgctgcggactatccgccagtccaatgaggagggcccaggagagtggctcncactgnactctnancccatcntggcagacagtgagatctgcgggaccaagg

SEQ ID NO: 100 - DNA, 248 bp, Mus musculus
  misc_feature 60, 85, 95, 102, 105, 116, 119, 127, 135, 150, 159, 176,184, 189, 214, 215, 228: n = A,T,C or G
accctgagatgctcaagtaccagcggcgacagcaacagcagcagcctggatcagaagaangccactactagggagacaagctcancatcctcagncacggcntgnggtcaatgggnaangtctagangatatcanaaagaatcttttgangaaacaagnggaaactcggacagcanatggccgnagganaatcacgcctctttgcatagcacannctggacactgggnactcactccttcaccaagct

SEQ ID NO: 101 - DNA, 366 bp, Mus musculus
tgaggtttccttaggaaacaaagaaagaagcaaaggcgtgttctacaaggccctccagagctgtccttgggcaaaggtgctatacatggacgccatggagtatttcccagacgagctgcaggagatcctggacgtgatgaccgagaaggagctccgtgtgcgcctgccccttgaggagctggagctgctgctggaggattagtgtgcagccaggcctgggccagcctgcagagcccaaagccgcagctgtgcaggctccactgtcctgctggggttggacgtattgacttacatattatatatgtgatctctatgtacaagtggagtcgtgccagtgataaaccttttgtggttgaaaaaaaaaaa

SEQ ID NO: 102 - DNA, 500 bp, Mus musculus
  misc_feature 32, 296, 482: n = A,T,C or G
gttttgagagaatgacagcttgaggcatcagncgtgtggtggtggaatacctgcgggctgtcatgcagaagcgtatttcctttcggagtgctgaagagcgcaaggagggcgccgagaagatggtcagagaggctgaacagctccgcttcctgttccggaagctggcgtctggatttggtgaggatgcagatggacacctgtgatacaattgttgcagtggcagaggtcattaagctcacagacccttctctgctctatctagaagtctcaactctggtcagcaaatatccagacancagagatgaccacattggtgcactgctggcactgcgaggagatgccagccgggacatgaaacagaccatcatggagacgctggagcagggccccatgcaagcaagccccaactatgtacccatcttcaaggagattgttgtgcccagtctgaacgtggcaaagctccttaagtagccctgtcctcnctgctgccctatgtgcct

SEQ ID NO: 103 - DNA, 258 bp, Mus musculus
  misc_feature 32, 33, 35, 37, 59, 60, 69, 108, 147, 178, 184, 188,195, 199, 206, 207, 216, 224, 226, 228: n = A,T,C or G
ggcatcaccatatccaccaccaaagcgccggnntntnccactctccacctcttaaacanngcaagcctncacagtcaccaagagacgcttcgccctccttatcttcanaacataggaaagggtcttccccaggccgatccacccggnaggccccgttcaccacaaccaaacaaacggnattcgncctnaccacgnccancgggctnntcagacctnaagccctncncntgtacgaagaggagcttcagcgccaccccc

SEQ ID NO: 104 - DNA, 521 bp, Mus musculus
actgagcattgagaggtgaatgtccggctgaccctcttccatgtcaggagctgtgtaccggggacgaatcctgtccccaggggcacaaatgctgcagcacagggtgtggccatgcctgccgcggagacatcgagggagggcgggatggtcaatgtcccagaatcctggtaggcctgtgtattgtccagtgcatgatggatgagaactgtcaatccggggaaaggtgctgtaagtcaggctgcggccgcttctgcatcccaggactccagcccctccaacagctcaaagactccaacctgactgatggctttaattctaaactggaggctcaagcgccctagctgacccgatttgcttggcgctccaggaagccaggaaggggtgacatcctggggcttgtgacactccctggagtatcaggcctttgccttgtatctactgctacttgcatgtagcatggaattgccctggtgctgggcgcttctctttcccaataaaatgctggcatggaaaaaaaaaaa

SEQ ID NO: 105 - DNA, 525 bp, Mus musculus
  misc_feature 18, 41, 48, 262, 293, 301, 365, 385, 386, 397, 401, 417,422, 426, 452, 468, 473, 478: n = A,T,C or G
catgaacgaaccgctggnggactgtgagggctatccccggncggatgnggacttgtaccaggtccgaacagcaaggcacaacatcatctgtctccagaatgatcacaaggcactgatgaagcaagtggaggaggccctgcaccagctgcacgctcgggacaaagagaagcaggctcgggacatggcgtgaagcccgagaagaggccatgaaccgcaggctggcctccaacagccccgccctgccccaggcctttgccagagngaacagtatcagccccggttccccagccagnattgcggncctgcaagtggatgactaaattgtggagttcggagtccgtgaacacccaaaacttccagtcagngcagaacgtgggcactgcgnngcagcataccngagnggaagcccctgaatgngacgntganccgcactaggagagaagcaccagctnagactgattccaaccngctgngcaangaaaaggactgctgatctgcacattattcctctccagagatgactgt

SEQ ID NO: 106 - DNA, 256 bp, Mus musculus
  misc_feature 17, 19, 198, 219, 224: n = A,T,C or G
acgtgtgaagggtctgnangcctagggaagttaccaatgttcaaaatgctctgtaaccgtgtatcaatattattgcccatcattttggccttttcctctgaacacccagctgtgaagatgtttttggaatagtgtgtgcatgccagagttggtgcatgacactttctttgtgaaccgggatgtcagtttggagaaagncttctgcagtcccgaggattngcttncatttccagaaggcaggtcagcctgattccca

SEQ ID NO: 107 - DNA, 540 bp, Mus musculus
  misc_feature 25, 88, 160, 163, 175, 184, 186, 200, 229, 242, 244,272, 472, 500: n = A,T,C or G
gactgaggtggggtctttccgtggncgcacttctggatcaggtggagcgcttccgtgagcagctgcgacgacaggaggaaggcgcttnggagacccagctgcggcaggaagttgaaagacttacaggagcggtctagaggaaaaacgagaggagagatgnttnagcctgatgatncactctcancntgagcaaagagaangaggtagtcttgcattcgtggaagtgtgnccgagaaggatananccagggccgccagtgatgttctgtgccnatccttggctgatgagacccaccaacctgcgcaggacattggcagccactgcccacatgtgccaacatctggccaaatgtctggatgaacgacagtgtgcacagggagacgctggggagaaaagccctgctgagctagagcaaacaagcagcgatgcttctggccagagtgttattaagaagttacaggaagaaaatcgnctgttaaaacagaaggtgactcatgtanaagaccctcaatgctaagtggcagcgttatgatgcaagta

SEQ ID NO: 108 - DNA, 114 bp, Mus musculus
  misc_feature 24, 31, 32: n = A,T,C or G
gactgaggataacaaccttcaggnaccagtnnaagaaggtgcttaccttggcgcctcatctcctgccggacagcctctagacctccctggtcctcaataaaatcgtagatgagc

SEQ ID NO: 109 - DNA, 517 bp, Mus musculus
  misc_feature 28: n = A,T,C or G
tgaggaagaaaaattatcactgactagntcactagatgatgttaccaggcaactgcacattacacaagagacgttgtcggaaaaaatgcaagaattagataagctgcggagtgaatgggcctcgcacacagcatcgctgacgaataagcacctctcaggagttaacagctgagaaggagaaggccttgcagacccaagttcagtgccaacagcagcacgagcaacagaaaaaggaactggaaaccctccatcaacggaatatccaccagctacaaagcagattgtccgagttagaggcagctaataaagagctcaccgagaggaagtataaaggagactcgactgtccgagagctaaaggcgaagctggctggcgtggaagaggagctgcagcgggccaagcaagaggtcctttctctgagaagagagaattgtactctggatactgaatgccacgagaaagaaaagcacatcaaccagctacaaacaaaagtggccgttttagaacaggagatcaa

SEQ ID NO: 110 - DNA, 205 bp, Mus musculus
  misc_feature 53, 66, 170, 175, 179: n = A,T,C or G
tcctgaaatgtattgcccagaggaggctgggaaattccagatcagaatactcncaggttatgtctnctgaaggcccatcatttgacctacagaaatccatcctctttcttgtcctcatggcagaagactgagggagctctctgggagtttcttatgagagcattgacccntgaangttncactctattatctgatcaccacatca

SEQ ID NO: 111 - DNA, 264 bp, Mus musculus
tgagggcataaaaccagccagcttcaataaagtcacagaccctgaagtgaaagaaataattgaaggatgtattagacaaaacaaatctgaaaggttatctatcaaggacttactaaaccatgcattctttgctgaggatactggactgagggtggagttagcagaggaagatgattgctcaaattcatccttggctctaagactctgggttgaagatcctaagaaattaaaaggcaaacataaagacaatgaagctattgaata

SEQ ID NO: 112 - DNA, 477 bp, Mus musculus
  misc_feature 31, 62, 112, 123, 126, 130, 134, 141, 151, 155, 227,260, 270, 280, 287, 293, 295, 309, 311, 312, 316, 325, 333, 335,352, 364, 379, 385, 399: n = A,T,C or G
agatggaaaaaagaacggctcaagagaaggngaaataccagccataactatgaaaccaccanccttaccagagtgttctgcatggcctgacgtcccctaccaggcagaacancaccccatccncangctncatnggctgtnccaacagctntggnctacagtgaaggtagcagctcacctaatcacccggtggagcccttacccctgggcagtggatcaccgtgctnccatcagcctgtattcaggatgcacagcagtgngcttgcatcnaaaccgcttntcacaantctgangnctctttgccagctncnnaggngctgacctnctgaagangnccagagatgatttgatncaggtttgtggncctgcatatgggatncgactnttcaatgccatcanaggcaggaatgtgaggccaaagatgaccatctatgtctgtcaggagctggagcaaaaccaactacccctgccacagtt

SEQ ID NO: 113 - DNA, 468 bp, Mus musculus
  misc_feature 28, 37, 51, 72, 98, 369, 370, 372, 377, 379, 380, 382,387, 395, 396, 425, 431, 438, 441: n = A,T,C or G
gactgagatggcgtcgtcgagccccgangccccttgntcctgcgactgttntgtctctgtgcccccggcctnagccatcccagctgtaatctttgcanagaactcagaccggccccgagatgaggtgcaggaggtggtgtttataccagcaggcactcacagtccctgggagccggcttcagtgtacctatatcgaggtggaacaggtggggaagacacacgctgtgattctgagccgcccctcttggctgtggggagctgagatgggggccaacgagcttggtgtctgcattggaaacgaagcagtgtggaccaaagagcctgtggggcagggggaagccctgctgggtatggacctgctcaggctgnntntggancnnancagcnctgctcannaggctgtatcacgtgattgtaggctctgntggacngctatgntcngcctggcagcctccgtaaaggatcctg

SEQ ID NO: 114 - DNA, 141 bp, Mus musculus
ggatttgcgaaagcttgaggcgttacgtacagcagtgatgacgattaattctacgcgatggaatcaaaggactttcttcctggatgtcttctgcattctcacattttaaagagtgggtgggagtgggcctgtttggcgcaa

SEQ ID NO: 115 - DNA, 240 bp, Mus musculus
  misc_feature 31: n = A,T,C or G
actgaggctgaatattctgaactggttgagncattactgtcaagtcaacaagacccagtcatttaccagagattagcagatgccttcaacaagctcacagcgagcagcactcctcctgccctggaccggaagcagaagatggccttcctaaagagtttagaagaatttatggcaaatgttggtggtctcctttgtgtaaaataaacaaacagagctataagcttaacttaaaaaaaaaaa

SEQ ID NO: 116 - DNA, 169 bp, Mus musculus
  misc_feature 16, 17, 20: n = A,T,C or G
gcttactacgaccganncgnttccttcatccgctctgctttccgggatcaggcctcgaggctgggagttcctgtgggggtgctatctgccaaagtgtttgctcgcagtgtgcagcaggtctgcgtggagcccagccaccccgtgctgctcagcccggaacaaagtgtga

SEQ ID NO: 117 - DNA, 156 bp, Mus musculus
  misc_feature 26, 30, 41, 50, 51, 86, 124, 135, 140: n = A,T,C or G
aggatgaagaggttctttctactgancatntttctccacangtcacaaannaggtgcttggtccagaactggtccatgtatcaganaagaacctgtcagagattgagaatgtccatggatttgnttctcattctnatatntcaccaataaagccca

SEQ ID NO: 118 - DNA, 375 bp, Mus musculus
tgagctacgccgcaggaatggggcacttaagtgcaccttctctgcagctggccacagcactggcctcctgcagggcctggctgccctccgtgcacagggccagctgctcgatgtggtcctcactgtcaacagtgaagcctttcatgcgcacaaggtggtcttggcggcctgcagtgactactttaggaagccagccccactgtcttccaaatgagtatgttctctggtgatctaaggtgtcccttgctgctcagagtcatggatgtcatcgttgtacggccgctcagcacatgagtaattaatcccagtcctttgctgcagcctctgctgatcagtcctttgatttaataaaggcattatggaggaaaaaaaaaa

SEQ ID NO: 119 - DNA, 332 bp, Mus musculus
  misc_feature 29, 265: n = A,T,C or G
gtactaaacccaggatcaacacaccctgntttccgaatctggctgacgagttacccatctccaaatttccctgtgtcggtcctgcagaatggagtaaaaatgaccaacgaagcacctgaaaggattgccgggcataatatcatcccgatcatacctcatggaccccatctctgaccctgaattctttgacagctgcaaaaagcctgttagtgatggctaagtcctgtcaccccagccctgtaaagcaattcaagtggcccctagngatcagggatgtgtgcatagagtaaaactacaaattaaatagaccccctgctctgcaaaaaaaaaaa

SEQ ID NO: 120 - DNA, 542 bp, Mus musculus
  misc_feature 24, 39, 237, 297, 331, 371, 380, 464, 515: n = A,T,C or G
ttttggggtaaccgcggggcgtgntttccagccatgaancgcaggtgccgtcccctctgtgcccactctgccgtttgcccttcgaccccaagaaagtggacaaggccacccatgtagagaagcaactctcatcctacaaggcaccctgtcggggttgcaacaagaaggtgacactggccaagatgagagcgcacatttcctcctgcctgaaggtccaggagcagatggctaactgtnccaaattcgtccctgtggtgcccacatctcagcccatccccagcaatatccccaaccggnctaccttcgcctgcccctactgcggagcccgcantctggatcagcaggagctggtgaagcactgcgtggaaagncaccgcagngatcccaaccgtgtggtatgccccatctgctcagcaatgccctggggggaccccagctacaaaagcgccaacttcctgcagcanctgctgcaccggcacaagttctcctacgacaccttcgtggactacagcatngacgaagaagcttaccaccacactgaa

SEQ ID NO: 121 - DNA, 192 bp, Mus musculus
  misc_feature 50: n = A,T,C or G
gggttctgtgttgccacagcatgctgctcttcatcttgctgctcatcacntgtagtcctggctacacgacatcgtcacagcggaggatgcaagacgtcggacaaaaaagctgggaagtcaaaggggaaggatgtgaacatggtggagtttgctgtagccacaagggatcaggcctccatataggactgagga

SEQ ID NO: 122 - DNA, 318 bp, Mus musculus
gactgaggtattgcagactcctgtgtacaagttggaaacatctctgtaagcctttatttacctgtctcattggaaatctcattgtctgcacaaggaatgcaatcaaagcagcagacagccttcccctcctgggcagctttcctgaatccaggcctacagctctcactgcacacagactgaggaatctaggaggaaagaaatctactagcaaatgcttggaatttttgaaaaatccaaatttttggaccaattctgagtatcattgaagagttactttgtaaatgcttccaataaaagttgaagataatgaaaaaaaaa

SEQ ID NO: 123 - DNA, 438 bp, Mus musculus
  misc_feature 111, 122, 129, 201, 238, 245, 266, 303, 316, 319, 323,327, 332, 338, 339, 348, 396, 404, 418, 419: n = A,T,C or G
ctgcacacagagcaatgccagctctgcccctggaccaactccagcatcacccacaaagacccgaagacaggacagagcgaagacttcagcagcgctgaaccctgagcaganggcagaccggnattttgngttggacaaaccgtcccctaaagacaacattcccgccctagtggaggagtacctggaacgtgccaaccttcncagctaatagacctcgactgggtcctggccttgcctnacgatanattctggtgccaggttatttntgatgagaccctgcagaagtgcctggactcctacctncactatgtacccncanagtntgangagtngggtgnncccgacccntgaggtggctgacatgcagaattcacctgcaccggagttgttttcctnaccacctncgggaagggcctgnntggcattgagaccttgtat

SEQ ID NO: 124 - DNA, 446 bp, Mus musculus
  misc_feature 51, 58, 130, 165, 186, 197, 198, 227, 229, 244, 252,253, 272, 297, 312, 313, 328, 333, 335, 348, 357, 384, 403, 406,411, 414, 417: n = A,T,C or G
aagaacagacgttcactgacatggccagcgacctagcactgtgccactcancagcgtnccccaggtcagggttctgaagaaaagctcggcagcagttccttgtgctcctccagggagcttctctgcttgnagcttcttccgcagaactcacacttgtacggcttntccactagaaggaagactcanggattatgtgnngtatatacacgtgagaacaagcatttggngngaccggagctgttgnaccccctnnagatggagctgaagttggntataaggcaacaagatatggatgtnaacacggtatcctgnncctctggaagagcantctgngntcctaaccactgngtcatttgnttcagcgcttacgccccacccctgttnatattttataattaatttncangctgnacntanttgagcattgtatagctttcctgaatagg

SEQ ID NO: 125 - DNA, 145 bp, Mus musculus
  misc_feature 25: n = A,T,C or G
gactgaggcaccaggcactctgggnccgctacatgggcaccctccgtggctgccttctgcgcctctatcatgattaaagcccttttccttcctccctccgacataccctgagaataaagttgccataagtttggaaaaaaaaaaa

SEQ ID NO: 126 - DNA, 512 bp, Mus musculus
  misc_feature 32, 70, 105, 111, 127, 139, 167, 238, 255, 269, 301,356, 365, 382, 386, 390, 422, 426, 435, 437, 442, 443, 447, 453,468, 469, 473, 480: n = A,T,C or G
tgtgtggagggcaagtgctgccacgggtaggngtaaacctcctggctcccagaggtgcaccaaacccagntgtgttccgccatgccagaacaggagggatgtgtntacggnctcagcctctgtgtgngcaagccagggncaaagggcaaagcctgcgagatcacagntgcccaggacaccgtgccagccagcctttggagggcagaatcctgggtcttcgtgggccccccttgagcangcagcaaaacatacgtnaaccaagaaggcanacaccttgcccagggtcagtccagtggcccanatgaccttgaccctcaagccaaagccatcaatgggactctcccagcagatacatnctcatttgnagtgctggctgctcttncaanaggncttatgggactcaattccctgcacctacgtgntcgntcacagttntntgctnnctcnaggtcnaggttggatctgtanncttnataggcntgcgcatggaccattgcatacacagcatggcc

SEQ ID NO: 127 - DNA, 196 bp, Mus musculus
gagctcctgctggacatcaggatgccattgccactgaaaagagtgacctactgaacgagctgctggagtctctgggggaggtgcccacggtggagtcctttctcggtaagaacagtattccagtcctccctcatttatttaaggggtaataactaccattgcctttttttttttttttttttgggaaaaaaaaaaa

SEQ ID NO: 128 - DNA, 405 bp, Mus musculus
  misc_feature 278, 280, 299, 302, 311, 317, 319, 344, 352, 366, 368,371, 373: n = A,T,C or G
gaggctggaggggtagcaggcgtctctgtggacttgatattatttcctctggacaccattaagacaaggctgcagagtccccagggatttaacaaggctggtggatttcgtgggatatatgctggggtcccttctgcagctgttggatcctttcctaacgctgctgcattttttctcacctatgaatatgtgaaatctttgctgcacacggactctacttcacacttcaaacccgtgaagcacatgcctggctgcctctacaggagaagtggtgagcnantggatacatgtatacagcntcncagaaatanaagagnangcctgtgtaatgattctttaaaganctttgcanaccttttctgactncnctntncgtgaaagaggttattcaaaggactgttcccg

SEQ ID NO: 129 - DNA, 399 bp, Mus musculus
  misc_feature 26, 34, 35, 46, 67, 72, 78, 79, 89, 137, 153, 161, 164,194, 202, 216: n = A,T,C or G
tgagttgcctcatggcaccgatgaanactggctnnagaagctgtgnaacacttttgtcaacaagaantcganattcgnnaagcctagantggcaaactcatccttcatcatccaacattttgctgacaaggtggagnaccagatgtgaagggntcctgganaanaacagagacacatgtctatgacatgcttgntggagatnctcagagcatgcanggtcagcagggtccccagggaggggtacaggctgctgctcattgtgtagctcacacaaggaagggtagagttggagagaggggagcttacttgagtaacagcagcacctccaagtggtgttttcatttccttctttttcccagttgaataaaattccagtatatccatgcactaaaaaaaaaa

SEQ ID NO: 130 - DNA, 460 bp, Mus musculus
  misc_feature 356, 379, 408, 429, 440: n = A,T,C or G
gactgaggtcttgatgcagagctttactatgtgagaaatgacctcatcagtcactacgctctgtcctttaacctgctagtgcccagtgagacaaacttcctgcacttcacttggcatgcaaagtccaaggttgaatataagctgggattccaagtagacaacatttgtggctatgggcatgccccaggtcaatatttctgctcaaggggaggtcccacgcactttatcagtgtttcgggtcgagctttcttgtaccggcaaagtcgactctgaagtcatgattctaatgcagctcaatctgacagtgaattcctcaaaaaattttacagttttaaattttaaacgaaggaaaatgngctacaaaaaacttgaagaagtnaaaacttcagccttggacaaaaacactancagaactatttatgaccctgnccatgtcgcgncaaccacttccacgcgtggg

SEQ ID NO: 131 - DNA, 533 bp, Mus musculus
  misc_feature 515: n = A,T,C or G
actgagtggctcagatgtcttcaattccaaaaacctggccgttcaggcacaaaagaagatcctgggcaaaatggtatccaaatccatcgccaccacgctgatcgacgacaccagcagcgaggtgctagatgagctgtacagggtgaccaaggagtacacccagaacaagaaggaggcggagagggtcatcaagaacctcatcaagacggtcatcaagctggccgtcctccacaggaacaatcagttcaatcaagacgagctggcgctcatggagaagttcaagaagaaggtgcaccagcttgccatgacggtcgtcagcttccaccaggtagagtacaccttcgaccgcaatgtgctgtccaggctgctgaacgagtgccgagagctcctacacgagatcattcagcgccaccttaccgccaagtctcacggacgggttaataatgtctttgaccatttttcagattgtgattttttggctgccttgtacaatccctttggaaagtttaaacctnacttacagaaactttgcc

SEQ ID NO: 132 - DNA, 295 bp, Mus musculus
  misc_feature 26, 31, 35, 49, 51, 54, 56, 62, 84: n = A,T,C or G
gggctgagggagagcctgcccagagnccatnatgncttacttgaggcantntgnangggcgnactggaaacatgaagtgtctgntccctatgccatgaatctgctcaacggaatcaaacttttcgggaggcctatcaaaattcagtttagatcaggaagcagtcacgcctctcaggatgccagtgtgtcatatccccagcatcatgttggaaatttaagcccaacctccacatctcctaacagctatgaaaggacagtgggtaacgtgtctccgacagcacagatggtccagagg

SEQ ID NO: 133 - DNA, 186 bp, Mus musculus
agcatctgcatcctcattctcctctacgccatgtacaagtaccagcaaccgcgacgagggctcctaccaggtggaccagagccggaattacatcagtaactcggcccagagcaatggggctggtggtgaaggagaaggcccctgctgcccccaagacgccaagcaaggccaagaagaacaaagaca

SEQ ID NO: 134 - DNA, 548 bp, Mus musculus
gactgagcctgacgtgtatccagagcctctaccgtccatagcgtgactccacccctgaagacattcgagaggatgagctttctctccagcctctctgagcccaggcggggtgagactgcatataaccaggtcacacactacgactttgtaaggcatactgtcatggtctagacatccagggaagcctctcaggggcaggctcctcagctgaccgtcccggctgacctcaggtgcagagcttccgagcgtacctcgcatgagtgtgagcacagtccctttgtagaagccgcggatcccgaactcctgatacagcttctttgcgcagtccaaggtgccactgtacttgttctcccctgaagaagcctgaatctgggagggatggaaggtcaccacatcaaaaacagcagtaatggaactgacagacccagcaggtctggggcttcctggctagctggcctagcttcatcactgagcctcaggctagtgagaaagcctgtctcaaacacaaggtgggtgagtccttagggacaatcaggcctccacatgca

SEQ ID NO: 135 - DNA, 270 bp, Mus musculus
  misc_feature 40, 60, 61, 65, 71, 79, 82, 89, 101, 116, 118, 130, 143,208, 215, 230, 242, 253: n = A,T,C or G
cctgacatcctcaagatcactggaggccaagctggctgantgccaagaccagctgcaggnnttcnagaggnagaacatntanctcacantcatcatttcanacctgcgcattcggntnaggtactggcanaagggatcccacnatctgtcccgatcaggggcccgcttaccgagatgagctgcacgccccccaagggaggaccacttnctttttnttggctgccgatttntgaaaggagtgngctatcatcantgctgtgaaataaaagt

SEQ ID NO: 136 - DNA, 292 bp, Mus musculus
  misc_feature 19, 25, 62, 222: n = A,T,C or G
gtctatgatcttttcccanggaagntccagttacctccacctagagaaacatctgtagcatnaatgagtcagacaagcggtggtgaggcaggctcgcctcctccagctgtagttgctgctggtatgcatttcattttacttcattaatgaaaactagggtactatctggccaacatgaacagtattggaaaagtttatatcaaaaagagtatgatttgaggnttttagacaggtctaaagactttaccctttacgttgaaaggttttcggtgtgtttttttttttttaaaaa

SEQ ID NO: 137 - DNA, 430 bp, Mus musculus
  misc_feature 24, 72: n = A,T,C or G
actgaggctccaaaaacaggtggncaagactttgtgaaagcagaaaagcaaaggttgcgggctgagaagaancagcggaaggcagaggtcaaagagcttaaaaagcagcttaaactccacagaaaggttagtttaccattagaatagatgggaagaaggctctgtcaagagttctaggtctcatttagtttataaagggaagctttgggtccagatggtgaaagaaaaggcaacacaacactaaagattttattcatgtttgagaaaaggtatctgaactccagctcaaaggggatagttaaaatagtattatttaacttagttggtatttataataaatggcgatcttaattactcataatatgtttatttacaatctaactcttgaatgcttctaaataaaccacaattcaaactgcacacaaaacaa

SEQ ID NO: 138 - DNA, 509 bp, Mus musculus
  misc_feature 244, 289, 291, 313, 343, 385: n = A,T,C or G
gactgagagcccgccatggagcctccgctgcccacgccgatgctactgctgttgctgctgctgctctccagttccgccgcgctccctgcacctcccaggaccccaagacactcagacggaatgttcaccagcgagctcagccgcttgcaggacagtgccaggctgcagcgcctgctgcagggtctggtggggaagcgcagcgagcaggacacagaaaatatcccagagaacagcctggcccggnccaagcccttagaggaccagctctgcttgctgtggtcgaacactnanaccctacaggactggcttctgnccaggctgtccctggatgggtccctgtctntctggctgcctcctggaccaaggtctgctgttgatcgttcanagtggactgaaacaaccaggccacccagatgagggaggaagagtctctaggaccctggctggagtagggatttggttgtccttggcatcaataaaaaaggaatttagaccctgaaaaaaaaaaa

SEQ ID NO: 139 - DNA, 489 bp, Mus musculus
  misc_feature 36, 151, 309, 452: n = A,T,C or G
gatatgaatgcctacatcaggccctctcctaccagngggactctaggaggagtgacaaggaccacaaatgaagccattgtgttgtgtgaaggagggggctatgacatcattcttattgaaaccgtcggtgtagggcagtcggagtttgcangtggcgtgacatggtcgatatgtttgttttattgctgccaccagcaggaggggatgaactgcagggcatcaaaaggggcatcattgaaatggcagatttggttgttataactaaatctgatggagacttgattgtgccagcccgcaggatccaagcanagtacgtgagcgcactcaagttgctccgtaggcgctcggaagtctggaggccaaaggtgattcgcatttctgccagaagtggagagggcatcactgagatgtgggacacaatgagagagtttcagcaccaaatgctggccagnggggagctggctgccaagagacagacacagcacaaag

SEQ ID NO: 140 - DNA, 554 bp, Mus musculus
  misc_feature 27, 146, 149, 151, 152, 161, 169, 190, 206, 230, 257,333, 343, 350, 358, 359: n = A,T,C or G
gactgagccgcggattgcaaagacagncccgggttcccagtgaagaggttaatacaagcccgcttgcccttcaaacgcctgaaccttgttcccaaggagaaagttgaggaagacacatcaccaaaagcagctgtggaaagcaaagnccngnntctacaatngtctttgngcacttttgaaagccagtgtnacacaggctctcacgnaggcattgagtacaaagatggcanggtggccagggccccattgatagcttnttgagggcaacaatcaagccagtaccgagtgtggtcattatcgacctgacggagaactgcagtgacattccagacngccctgaggnccacagngaactaanncctgacactgcctggtgtggtcaccacagtggaaggagctgccaagcagcaggatcactctgcagcagagctgtgtctcttagagaccccttcagacatcacttgtcacatggaagaggagcctgggagtccaggagacccaaagaggacgggtgactgtcaggctggttcactacagagctgtccggagctgac

SEQ ID NO: 141 - DNA, 270 bp, Mus musculus
  misc_feature 231: n = A,T,C or G
ctacctctggatgcggatgctctggtcttggtggccctgagtcgctatcatcacatcagcctcgtccagaacaaacaccttgatcttatttggggtcaatgaacttcagcttggagcaccagtccaagacggtcccgggggtgccaatgacaatctgctcactcaccttctgacctctctccactgtggaaaaggtaagttttctttacatgttccaaagagaaagccagntctggaaataaaaatcttctacattgtcaaaaaaaaaaa

SEQ ID NO: 142 - DNA, 196 bp, Mus musculus
gactgagttccctgcagtttgtgaatttttacagaacaacagtttattatcaataatcagagcccatgaagcccaggatgcggggtaccgaatgtataggaagaaccaagcaactggctttccgtcacttattacgattttctctgcccctaattacctagatgtctataacaataaaggtaaagaagtccagcaa

SEQ ID NO: 143 - DNA, 463 bp, Mus musculus
  misc_feature 22, 388, 428: n = A,T,C or G
tgcgtgactggctgaggatagncaactgtcatcatgccccactcatacccagctctttctgctgagcagaagaaggagttgtcaggatattgctctacggatcgtgacccccggcaagggcattctggctgcagatgagtccgtaggcagcatggccaaaaggctgagccaaattggggtggagaacactgaggagaatcgccggctgtaccgccaggtcctattcagtgctgatgaccgtgtgaaaaagtgcattgggggggtcatcttcttccatgagacactctaccagaaagatgacaatggtgtccccttcgtccgcaccatccaggataagggcattctcgtaggcatcaaggttgacaagggtgtagtgcctctagctggnaccgacggggaaaccaccactcaagggctggatgggcctnttggaacgctgtgctcagtataagaaatgatgcag

SEQ ID NO: 144 - DNA, 471 bp, Mus musculus
  misc_feature 32: n = A,T,C or G
actgagataaagagaagaagaactcaccaagnatttgtgacgagctgatcgccaagatgggaaaaagctaactctgggccaagtgcgtggacttggccgttgcgtgcaatacgagcgtcggctcaactgtttgtccttccgattccgtgaacagcgtctcctttcacttccgtatgcactactgtgtcctctttctaaataaggttggtttgaccgtatgcagtgccgagacctaccaggattccttgctatccgtttgcattttctagtataattcatagcaagttgacctcggagttcctgtatcagggagactgtcagattctctaatagactacaaattgctggccttggccttggcctttggcgagcagcatctgatgggacgtgaacatatgcatcgggactcttgccttaaggagtgtacaattgatccacatttgctggtttggttttttaggaaaaaaaaaa

SEQ ID NO: 145 - DNA, 548 bp, Mus musculus
  misc_feature 83, 87, 90, 98, 99, 103, 104, 127: n = A,T,C or G
gactgagatgctaaagaaggaagaagcaattccttggcctggaccttagccattgttcattcctacatcgcctacaaagcagnaaangantagcgatnnaacnnaagctacttaaggaggagttcanatttaaaacaggagcgggaacaactggagcagaaggtcaaagagctcagcagttctataagtaaatgtatggagatgaagagcagcatcctggcgcggcagaaggagatgcgcagctccctggacaaggtgaagcggctcatccgccttgtgcatggcgtcgacctctgcagacctgtggactcagaggccacggcgggggccctctccaatggcccagactgcactccccctgccaacgccgcctccacacccgccccttccccctcctcgcagagctgcacagcgaactgtaaccagggggaagagactaaataacagagccctcgaggcggacatgccacagtcctggtggcgaggagacgctagaagagcaaagccggatttcttctggaaagtacagagttcttttggttctttgg

SEQ ID NO: 146 - DNA, 383 bp, Mus musculus
  misc_feature 361: n = A,T,C or G
gactgaggaggtgagtgcttttggggaagaggtgagggtgatgacctggacctgtggacagtccgatgttctgggcaacactgggagcgagaggccagtgtccgcttccagcacgtgggcacctctgtgttcctgtcggtcactggtgaacagtatggtaaccccatccgtgggcagcatgaggttcatggcatgcctagtgccaatgctcacaacacgtggaaggcaatggaaggaatcttcatcaagcctggagcagatctctccacgggtcacgatgaactctgagaaccagatgagtgatgggagggtggctgagtggcaatctcggggtcactcttgtgtaagactttgtttgtangggccctcaagtgcctttatga

SEQ ID NO: 147 - DNA, 266 bp, Mus musculus
  misc_feature 18, 23, 36, 200, 204, 235, 248: n = A,T,C or G
atgatatgcctacaattnctcantaggtagataccnctccctctacattgtctatgagtttctgatgtctggttggcatgagtacctacacctggcgctgtgaccccatagacttttccaatagccctgaagcacttctggatggttcgagtggcctggctcttcatgctttccaaggtcattgagctgatggacacagngatntttatcctccggaagaaggacgggcaagtgnccttcctccatgncttccaccactcggtgct

SEQ ID NO: 148 - DNA, 163 bp, Mus musculus
  misc_feature 36: n = A,T,C or G
tgagagtacgtcggcaggaattcaacgggcacagantcaaaaggagctctggagaattcaagatgtcatggaggggttgagcaaacacaaacagcaaagaggctcttcagaaacaggtctggcaggatcaaagcctttctcatcagttaagtacaagagcgag

SEQ ID NO: 149 - DNA, 409 bp, Mus musculus
acctcttccctccttgcctccccgaaacttacctggcattgcacctctccccatgctgtccgacttcctcccgtcattccctttggttccagagggctcttctgcagccagcgcaggggagccgctgtcttcccttcctgccatgggcccaccttctgaccctgtcatgactactgcaaaggcagacgcctcttccctcactgtggatgtgacgtctcctgcttccaaggtccccaccactgttgaggacagagtcagcgactgcaccccggccgtcgagaagcctgtgtctgatgcagatgcttcagagccttcttaactgggaaccaatgttcagaaccgacgtggtctgtttagttaaccacggggtcaggtctggaatcgcaaactaccattgatttcatactag

SEQ ID NO: 150 - DNA, 484 bp, Mus musculus
  misc_feature 34, 37, 39, 44, 51, 52, 68, 70, 97, 100, 101, 128, 135,149, 153, 177, 204, 210, 216, 220, 225, 230, 231, 350, 439, 442,448: n = A,T,C or G
tcgatttgcctacgacaggcctaggggtcgtccnatngncaaancgaaganncccagggacatggctntntcccagcacaaagctgacagacaagancgnnggctttacgatgcgtgcagatgatggncgtgganggaaggccatggcnctgngctcccttgggaagggcatccganagcccgtcagtgtgggtgctctttcanaagganaggtcntganagcangatgnncctgagcagaaagaagacacctttgacgtcttccgtcagcgcatgatgcaaatgtacagacacaagcgggccaacaaatagatcaaagccactggcgacagagataagccttgaagcanccttgcccttcccgtcaccctgccccggacgcctgcagcctgagcctcgttccagggtcacatccacaaggaccatggattccaaaagntgngctggnctggcctgccctggctccagtttgttcctgttcctg

SEQ ID NO: 151 - DNA, 256 bp, Mus musculus
  misc_feature 107: n = A,T,C or G
tgaacatggctgggttcatgaaagcaaatgagagtgagagtggtctagggaaaagacggtgatacatcgctgacacctgcccgtccatcggcactgctctctgcagntttttgcagtatggcacgggcatacccaccatctctgccgaaggaagatggcgcatacagggattgttggagaactgtatccagtgtgagctttggggaggataaagttggtgtgcaaataaattgatactgaagcttaaaaaaaaaaa

SEQ ID NO: 152 - DNA, 565 bp, Mus musculus
  misc_feature 137, 191, 279, 316, 328, 362, 365, 380, 397: n = A,T,C or G
aactgagcctccctctggtctgcttctcgaggttcaattcacagagcgcttcatatccacaaggatatgttcctaagacctccctgagctcaccaccctgtcaagaagtggttctgccggaccctgttgaagagacnagacaccatgcggaggttgtgaagagggtgaatgagctgatcgccacgggccantatggccggctctttgctgtcgtgcactttgccagccaccaatggaaggtgaccgcagaggacctgattctaattgaaaatgagttgnatatcaagtgtggagagaggattcggctggagaaggncctgctggttgnggcagacaacttcacactcctgggcaagccactnctncgaaaggagcttgtncgagttgaagccacagncattgaaaagacagaatcatggcccaaaatcaacatgaaatttaggaaaaggaaaaacttcaggaagaaaaaaatcattgtgaaccctcagaccatcctccgcattaacaccattgagattgcacctcgtttgctgtgactgcagagccgatgtctaagaagttctga

SEQ ID NO: 153 - DNA, 491 bp, Mus musculus
  misc_feature 26, 32, 34, 40, 45, 48, 51, 63, 64, 76, 77, 89, 93, 95,101, 104, 113, 122, 133, 148, 152, 167, 169, 173, 174, 180, 188,192: n = A,T,C or G
ggccctgacagacctgactcggaggngcaggncnagatancgaanctnggnctggagctggannttgcgtctcctnngatgctgactgngacntngagagntgngacgatgangagaactgngatctgatcancaatggacggcatanatgnccaggatggcctatncnctgnntttatntacgtagnaggngaggggccactgaaaacattcccaggacttaattcacttcctagaggtttttgctgcagagggattaaagcttacttcaagtgtacagtcattttcaaaacagctaaaagatgatgacaaacttatgcttctcctggaaataaacaaacttattcctctatgccaccagcttcagacaataactaagacatctttgcagagtaaagtctttctaaaggttgacaagtgtattacaaagataagatccatgatgactcttgtggtccagctcctctcactttgctataaactactgaaga

SEQ ID NO: 154 - DNA, 152 bp, Mus musculus
gactgagaccccaccaacaactgaacgttttggccaagctgcaacaatggaaggaattggagcaattggtggaactcctcctgcattcaaccgtccagctccgggagctgaatttgctccaaataaacgccgccgatattagaaaaaaaaaa

SEQ ID NO: 155 - DNA, 183 bp, Mus musculus
gactgagacctcctatgccccttcccagctgtacaagtgcagggccagcctagcagttctcagccttctccagtcagtgcgtccagtcagcatgcagatccagtgagaaaacctgggcagaacttcatgtgtctgtggcagtcttgtaaaaagtaaatggcggggttttgttaaaaaaaaaaa

SEQ ID NO: 156 - DNA, 402 bp, Mus musculus
tgaggtttgggaggttgccgcaccgtacatcgagaaatacagaatggagcacattccagaatccagaccgctttctccaacagcgttttcactagaatctctccgaaagaactcagccacaatcccagagtctgacgatctttaacgggcttcctcgaggacaggcacatcagatgggcctgagcttagcacagcactggtgtgttgatggagagtctcaggaactgcctgtaatgtgtaaaactcatctcttttcaaacatactctggataggccatggcagcaatgcagtggctgaacccagaaccctgatcttgttttcttgttactatggtttgaggagtactgggtgccttctggttgtgggagttgtctttcttgataatataaacaggctactta

SEQ ID NO: 157 - DNA, 532 bp, Mus musculus
  misc_feature 50, 77, 79, 96, 391, 440, 468, 480, 490, 493, 515: n = A,T,C or G
gactgaggtgaggacgtgagtctgagtctgtggaatgtctacagcagganggaccctgcgtctctggagaacatgcntncagaaattccctgcggnggcactggcggcagcccaggatctagcagcagctccggctcctcgagggaggactcagtgccggtgactatggtggccaccgctgggcaagtgcaacagcagcagcgacaccaccagcagggaaaagtgacagtgaaatatgaccataaggagcttcgtaagcggttggtgctggaagaatggatcgtggagcagcctgggtcagcctgtatggctgcaaggaagaagaaatgccagacgtagaaattgacattgatgatcttcttgatgccaaccgcaaggaggaaagagcttntaaattacaggaagctctcgtagtctgctataaaccaacagaggaattnatcagagagctgctctcccgaataagancatgaggaagcngaacccttcncanaagaagagcgtttgattttgcnacagggtaaaattctct

SEQ ID NO: 158 - DNA, 151 bp, Mus musculus
taaaaaagtggcaagcctggccctttcttgctatctggttccagagacaaaactattaagatgtgggacgtcagtactggcatgtgccttatgactcttgtgggtcatgataactgggtaccgtggagttctgttccattctggggggaag

SEQ ID NO: 159 - DNA, 134 bp, Mus musculus
  misc_feature 40: n = A,T,C or G
actgagagaaggcccgcttaccatttctgcacaggagagngtaaaagtggtatattacctgagcgctgtacccctttgaatccagaagtcaccgatgagatcaccatccagccaggagatatagtcatgctggc

SEQ ID NO: 160 - DNA, 404 bp, Mus musculus
gactgaggtatggaagcctttgagcaggctgagcggaagctaaagccgaacccaagcctcctcttctccgatgtgtaccaggagatgcccgcccagctccgcaggcagcaggagtcgctggcacggcacctgcagacttacggggagcacgtaccccctggaccactttgacaagtgaggccctagccagcacaagggggtcctgctctgagtggatgctgccccctgtgctagtcagctgtgcgaacatgctgagcagccagggtggcattccagggtgacttttgctcctgttcctgctcccccaggctacttcactggggcaccatgtttgcagccattgctgcggctacttcactcccttcacctgttacagtgccttttaggggtgggaagggaccagg

SEQ ID NO: 161 - DNA, 225 bp, Mus musculus
  misc_feature 77, 89, 95, 129, 133, 178, 184, 197, 201: n = A,T,C or G
atggggaagaagaagaatgtcccgaggagggtcatccactttgtcagtggagagacaatggaggagtacagcaccgncgaggacgaggnggacgnactggacaagaaggacgtgctgtccacatgtcgntccnacaaaactgtacctggggcccctacctatggttttacatgcctgngggccngccacatcaaccntctnagtgtgtgactttcttggagagaa

SEQ ID NO: 162 - DNA, 269 bp, Mus musculus
gaggtctggatgaacgcacaggcctggtggtgagtagcccgacgcaggagacgaagcacatagcggggaaaccgatcctggataacaacgccgtgaatctcttctcctccgtcttggtggccctcctgctccccacagccgcgtcggggttttggccccgagagtgaactttcttttccttcccaagcagtcgatactggtaaggccagccagacttgcaatgtgaaaggttttttttcagtgaataataaattcagggaccttgggaa

SEQ ID NO: 163 - DNA, 241 bp, Mus musculus
  misc_feature 38, 55, 68: n = A,T,C or G
gaccaataaggggagaactaagctgcctccgggtgtgnaccgcatgaggcttganaggcatttgtcancagaggacttctctagggtcttcgccatgtctcccgaggagtttggcaagctggccctgtggaagcggaacgaacttaagaagaaagcttccctcttctgacatccctgccggctcggctcgattcggctcgccctgtccgtgacagtaccttccttctgctgcttcgaggcc

SEQ ID NO: 164 - DNA, 111 bp, Mus musculus
  misc_feature 34, 44, 47, 70, 74, 91: n = A,T,C or G
gactgaggtgcgcgtgtcgacgaggccgccaggncggagcgatnctnacggaggaaggagcacgccaacngggnctgactgcgtacagaantgccccccctcaataacaat

SEQ ID NO: 165 - DNA, 408 bp, Mus musculus
  misc_feature 285: n = A,T,C or G
actgaggcgtggaaggttcatctgatgggccctgctggctgcacaatgaacctgcgcaagacacatgttctctacaaggctacagccccgcagcaagggaaaggcccagcagcagcaaagtcaagcacctcagcacccaaggttggaagcaagatccccaccccaaagggaggccttagtaagagctccagcaggacatacatgctgacggtgacagagtttccagttctgtctctgacaggctgtgcaaggttccccgctctaagccatgtgtgttggcatgcntgatgcagggctccatgcccactgtcccttggcctctggtgtcccagtgtctactctcacaggaacagtacatgttatgaccaaagagccctttccagtgtggccccagtcctgggcctgtgt

SEQ ID NO: 166 - DNA, 520 bp, Mus musculus
  misc_feature 21, 394: n = A,T,C or G
gaggggcgaaagcctgtctgnacagcaaagtacgtgagcaggaagaaaagtatgaagttcctggaggtccacagaggagcaggctcaacagagagcagctgttgccaaagctgtttgatggatgctacttctttttggggggaaacttcaagcatcatccaagggatgacctccttaagctcattgctgcagcaggaggcaaagtcctgagtcgcaagcccaagccagacagtgatgtgactcagaccatcaacacggttgcataccatgccaagcctgagtcggatcagcgcttctgtacgcagtacatcgtctacgaagacctgtttaactgtcacccagagagggttcggcagggcaaagtctggatggctccgtccacctggcttatcanctgtataatggcctttgaattgcttcctcttgacagctgaatgtcataccaggtgaacattttaagttggacatgcatggtttgtaagaacaacggccattgtgatatttctgggacattcatgta

SEQ ID NO: 167 - DNA, 137 bp, Mus musculus
gactgagatccttcagtgttttactgatcctctggccaaagaagaagaaaatgttggcatagatattactcaacctttgtacatgcaacaacttggagagattaatattacaggagagccatttttaaaaaaaaaaa

SEQ ID NO: 168 - DNA, 503 bp, Mus musculus
  misc_feature 435, 451, 457, 458, 482, 486, 487: n = A,T,C or G
tgaggctttctgtctcaactggtctctgacaagcctctgactgagtgcattcgcgctggccactacgcagcaagcgtcatcattagacgaactggctgtacctttcccgagaagccagacttccactgatggaagaaagaaaactcaggctgtggagtggagactgcagtgaccacatcctgagcgttcctccataagaaagaatagagttatcctctgtctttgcccaccatggttctcattagtaacttagagggctcagtgctacttctaggacctttagtctctgaaatctgggaaaaatgtttatttgcatagtttgttagtgcctcttaaatgtcaaacaggaatacatttcagtagaaactttaatttcattttcaattgtttgtaaattcatcagtatttagtaaattgatcttttttttatatttntgcttccaatgcaggngcaatnnaatataatagactttttaaagaanttanngctaacatcattcttg

SEQ ID NO: 169 - DNA, 154 bp, Mus musculus
  misc_feature 101, 103, 106, 111, 123, 127, 128: n = A,T,C or G
actgaggtgctacctcatactctatcatgccatttttgtgttctttgcatggacctactggaagtctatcttcacactcccacagcagccaaaccagaagntncanttgtnctatacatgacnaggnncagctatcaaaatgaacgagagacca

SEQ ID NO: 170 - DNA, 218 bp, Mus musculus
aggctcacctgcttgaagtccaaggcctgcaagttcggattagagatttggaggctgaggtgttcagactactaaagcaaaatgggacccaggttaacaacaacaacaacatctttgagagaagaccatctcccggggaagtctcgaaaggagacactatggagaatgtggaagtcaagcaaacatcctgtcaggacggcttgagccaaggattgctg

SEQ ID NO: 171 - DNA, 309 bp, Mus musculus
  misc_feature 258: n = A,T,C or G
acagaggatctgcaaaaggacaggagattctgcactgctatgggcctcacgagagccggatgggcgttgctgagaggcagcagaggctgagttctcagtacttctttgactgccgctgtggggcctgtcacgctgagacactgagagcagctgcagctcccagatgggaagccttctgttgtaagacttgcagagcgctcatgcaggtaaatctctgctgttcccaggggcagggctccagctaaaggttggcagtcngccaggagaaccattcctgcttcccttcttgtaactcctccctacatgtcg

SEQ ID NO: 172 - DNA, 192 bp, Mus musculus
  misc_feature 43, 72: n = A,T,C or G
ttcttgtggggggaagcacaccatcttcaatggaagaatgatncatctaatctggatccatctcaggctccnggcctggctcagcctctggtgtttagtaattcgaagcagaatgccatatcacagccgtgcttcagggagtacattttctcaccatagcatggtgagcatgttggggaaaggatttggtga

SEQ ID NO: 173 - DNA, 205 bp, Mus musculus
tgaggtggggtctttcactgcgaagaactgggtgagatgtggtctgagcaatctgccaaagataaacaaccgtatgagcagaaaggggctaaactaaaggagaagtatgaaaaggatattgctgcataccgtgccaagggcaaaagtgaagcaggaaagaagggtcctggtaggccaacaggctcaaagaagaagaactgaacca

SEQ ID NO: 174 - DNA, 307 bp, Mus musculus
  misc_feature 43, 88, 91, 239, 242, 243, 244: n = A,T,C or G
gactgagaacgtcaagaagaggagattcgatcccactcagcanggccccatctgaatcgtctgcactggaggaatgccaccttgcacnggngggttctgggaatgacatgttttcagccatgctgccggaactcttaagtctgtaaagaagacccttgggtggtaatgacatacagagcctcagatctgagaattgggatggggtacaagacaaaggtcccttttggaaataacttcanggnnnccccccccatcacctgtcaataacggggaggggttaaagaccatgtcattaaaaaaaacaaca

SEQ ID NO: 175 - DNA, 191 bp, Mus musculus
  misc_feature 110, 118, 161: n = A,T,C or G
tttgtggctttccagacggccatggcctcttctacctggttgcctgccttactagtgggagctaacacctttgcctcccacctcctcttcgcagtaggcatgcccactgntcctgctntggcccttcctgtgtgagcgtcaagggccaaagagaaggcagnccctcccggcaagtgagtctgaagccagag

SEQ ID NO: 176 - DNA, 462 bp, Mus musculus
  misc_feature 159, 160, 161, 163, 165, 170, 171, 174, 175, 177, 179,187, 194, 196, 198, 209, 216, 227, 260, 301, 331, 392, 410, 433,441, 444: n = A,T,C or G
attgaggtaaagtgctgtttgtacgcaccttgccaacacggtaacagaagaaattttagaaaagtcatttagtcagtttgggaaactggaacgagtaaagaagctaaaagattatgctttcattcattttgatgagagagatggtgctgtcaaggcatnnngnanaactnnttnnangngactcctngggagantncntcgcaattgtntttgctncgccaccgtancaggaggaggagagaatgaatccctcagaggcnggcaccgaaagaatcaaatgatatgatgattactacatatnatggtccacctcatatgcctcccccaacangaggtctgagggctgtggaggtagaggtggcgtatgtgatatcctccagattattatgganacgaagattattatgatnattatggttatgacttaccatanctatcggnggngggatatgaagatcccat

SEQ ID NO: 177 - DNA, 202 bp, Mus musculus
  misc_feature 18: n = A,T,C or G
ggatagtgtaactgattnccaaatcgaagggaaatcctttcaaggaggccttcctacaggaaaattttgaatgacttatcttctgatgcaccaggggtgccaaggattgaagaagaaaagtcagaagaggagacttcagcccctgccatcaccactgtaacagtgccaacccccatttaccaaactagcagtaaaaaaaaaa

SEQ ID NO: 178 - DNA, 384 bp, Mus musculus
  misc_feature 50, 81, 90, 116, 120, 128, 133, 140, 143, 151, 173, 174,184: n = A,T,C or G
ggactttcgaagtaagcatgcaatccatattcccaccaattcccaagtanccatgagtcatccgaggagtgggaggaagtnctgaagtgnaaatgacacagcactgggcagactcnggancaacgggntgcangaatggnagntttggaancaccatgtctgagaaagactgnngcgactcctntctaggaacacgtgccgatggccggcaaacccagaggccacagcatcaccgtggctgtggctgtggcacacggtcggaatgttttcctaaagctaagacaacaattgaattacctagaagttctcagaagggacctggaggaaatgttgttgtttagaacaataaaccatgtttcatagtcacaaaagtcgggggggggg

SEQ ID NO: 179 - DNA, 523 bp, Mus musculus
gactgagttagaagacctgaagatctaaagtactaacacctgaagagcaattagcagataaactccggctaaagaaattacaggaagagtcagaccttgaattagcaaaagaaacttttggtgttaataataccgtttatggaatagatgctatgaacccatcttcaagagatgacttcacagagtttggaaagttactaaaagataaaattacacaatatgaaaaatcactatattatgccagttttttggaagccttagttcgggatgtctgtatttcattggaaattgatgacttgaaaaagattaccaactcattgactgtgctctgcagtgaaaaacagaagcaagaaaagcaaagcaaagccaaaaagaagaagaaaggcgtggttcctggagggggattaaaggccaccatgaaagacgacctggcagattatggtggatatgaaggaggctatgtacaagactatgaagacttcatgtgacactttaccttctgctgtcttctttctttgccagc

SEQ ID NO: 180 - DNA, 419 bp, Mus musculus
gactgagacatggctctttatgccgacgttagtggaaaacaatttcctgtaacccgcggccaggatgtgggtcgatatcaggtgtcctggagcctggagcacaagagcgctcatgcgggcacctatgaggtcagattctttgatgaagagtcctatagcctcctaaggaaggctcagagaaataatgaggacgtttctatcattccacccctgttcacagtcagtgtggaccatcggggtacctggaatgggccttgggtctccacagaggtgctggctgcagtaattggcatagtgatctactacctagctttcagtgcaaagagccacatccaggcctgagggcagcaacttcagccgtccattgcttctttcaataaacagtcactatttgacatgagtacattcaaaaaaaaaaa

SEQ ID NO: 181 - DNA, 520 bp, Mus musculus
  misc_feature 192: n = A,T,C or G
gactgagttgccgccatcatagccgacaatgcgggctatgacagtgcagatctggtggcacagctccgagctgctcacagtgaaggccatataactgctggactggatatgaaggaaggtaccatcggcgatatggcagtactgggtataacagagagttttcaagtgaagcgacaggttcttctgagtgcnggctgaagcagcagaggtgatcctgcgagtggacaacattatcaaagcagcaccaaggaaacgtgtccccgatcaccacccctgttaagcattcccatttgctgatgaactctgggccagttcatagcaaagttgtacttggaagacttcaacctttaaagaagactggtggaattgacctgtccatgatagccttaagtttgaacattagctgaccttctgtgttaaacatgggtctaatttatttactgtttcattttccatacaattcagttgatttacaagttcatttctcatactgtgtattaaaataaaaatccagttactc

SEQ ID NO: 182 - DNA, 177 bp, Mus musculus
  misc_feature 109, 129, 152: n = A,T,C or G
tgagcgtgaagcccttctcacaggcaccgttctccgtgtcactgacgcccccagcctctgacctaccagggtacgagccgcccccagccctctcaaccctgccaccttnctttccccaagagtgatggnttatggttgaaggagaataaagntatttttaccaaatgaaatacaata

SEQ ID NO: 183 - DNA, 338 bp, Mus musculus
tgagaagggtttttctatgtagccctcatctgtgggcttcaggtgggcctcaaattcacagagctcctccaccccctgcctcctgagtgctggaattaaaggacggaggctctggactcccacattctgaagagagacccatctgccagagggctgaaggtggccttgagggccatgcgtctgggtctcaggatgcccctgcctgagcccagtgagcaggactgtgagagcctgagagctggccaggagccatctcttggagccaggaagcctcaagagtcctccaatctggtcccggccagggataaagagagacccatttttctaaaaagaaaaaa

SEQ ID NO: 184 - DNA, 389 bp, Mus musculus
  misc_feature 120, 233, 334, 338, 339, 362, 366: n = A,T,C or G
actgaggctgttatcagaaggtgcagatgtcaatgcaagacacaaacttggctggacagcactgatggtggcatccatcagccacaatgagagtgtggtgcaggtcctgcttgctgctgnagctgacccaaaccttggggatgagttcagcagtgtctacaagactgccaatgaacagggagtccattctctggaagtcctagtcactcgagaggatgacttcaacaaccggntaaatcaccgtgctagcttcaaaggctgcacagccctgcactatgctgtcctggccgatgactacagcatcgtcaaggagctgctggacagagtacatggncaannataggaagctgaaggcagaggancggngccgcttcccactggagcagcga

SEQ ID NO: 185 - DNA, 511 bp, Mus musculus
  misc_feature 487, 489: n = A,T,C or G
tgagggattatctggaacacataacggccattggccccaggactacaggaagcacagaaaatgaaattctgacggtacagtaccttttggagcagatcaagctgatagaagcacaaagcaacagccttcacagcatctccgtagatatacagcggcccaccggttcctttagcattgacttcttggggggcttcacgagctactatgacaacattactaatgtcgtggtaaagctggagccccgggatggagccgagtctgctatcctggctaactgtcactttgactcagtggcaaattcaccaggtaggtacttgtatttagtcatttaaagtcacaaagatagaacccagaaagtttattaacttagccccatataaggaccatttttctttgaccagcattttctcattgtgttagaagttaagctaattaatatgtttagagtctagcaatgtaatattcctttaagttcattattatcagngntattgtaattattaaatagtag

SEQ ID NO: 186 - DNA, 329 bp, Mus musculus
gactgagggcgattgtggtggctctggctgtggaaaatgtgactgtcatggcgtgaagggacaaaagagcagcccaagggaagcaaagtgttgaacagcgttctgagcttggttatcccgattcgcagcgcatcgccttctatcgccttcttgacgagttcttctgaggggatcaattctctagagctcgctgatcagcctcgactgtgccttctagttgccagccatctgttgtttgcccctcccccgtaccttccttgaccctggaaggtgccactcccactgtcctttcctaataaaatgaggaaattgcatcgcaaaaaaaaaaa

SEQ ID NO: 187 - DNA, 420 bp, Mus musculus
  misc_feature 331, 368, 371, 373, 392: n = A,T,C or G
gagcacacacctcccttactgaatatttcctcaatgataagtaccaagaagagctgaattttgacaatcccttaggaatgagaggtgaaatagctaaatcatacgctgaactcatcaagcagatgtggtctggaaagtttagctacgtcactccaagagcattcaagacacaggtagggcgcttcgcacctcagttctctggatatcaacagcaggactgccaagaactactggctttcctgttggatggactgcacgaggacctgaatagaattaggaaaaaaccctatatccagttaaaagatgctgatggcaggccagacaaggtggntgctgaagaagcctgggagaaccatttaaagcgaaangantncatcatagtatacatattnccatggcctttttaagtctacgttagtg

SEQ ID NO: 188 - DNA, 263 bp, Mus musculus
  misc_feature 67, 87, 152, 174: n = A,T,C or G
ggctctaggaaaccaagaatgaagtggcagatatgggccatcatctttgagggtgtgaaagaagatnggagaccctatcttggtggncttctttagcttggaccatgtgggtctcactccataatggtgtgaaagccacctcaaggcagggnagggtgcctgcaaccttagctntgttgccataaacccaggagcagaggaccacagaggaggaagtcctgcctagaggaacagaatggactgttggcggccgagctttgaga

SEQ ID NO: 189 - DNA, 328 bp, Mus musculus
  misc_feature 30, 31, 40, 43, 45, 48, 53, 63, 64, 67, 68, 76, 91, 92,98, 123, 124, 258, 276, 286, 287, 289, 295, 304: n = A,T,C or G
taacactgcagtactatcgagggaggcgtnnccaaagagntcntncanctgantgcggaggannttnntaatccanatatgagatagtgtnnacactnggatcccagcatacagaaattattnnggtttaatccatcttcttttgaaactgagggtcagtttacctctgattggcatagtcctgggtctggctatttacaataattgtatacgtggatgtccattttcccatggttgtatacaggaagcctaactgcnggaactttaggaaccttncgtgacttgnnanactctnacccagganctatatcaagagttttaaaggatt

SEQ ID NO: 190 - DNA, 390 bp, Mus musculus
  misc_feature 35, 192, 228, 358, 368, 369: n = A,T,C or G
gtctatgatttgaggtgtgaccctgctagtgcagntgaaggcataaaaccaaggaaaagaaagacttttgctttgcctggaataattaaaaaggaaaaggacgcagaatctgtggaatgtcctgatgcagattcgcttaatattccagatgtagatgaagaaggtttcagtattaaaccagaagcaaatcanaatgataccaaagagaaccatttctactcatccagngactctgactctgaagatgaagaaccaaagaggtaccggatagagatcaagcccgcgcatccaaataacttgcatcacacaatggcttctttggatgaactgaaagcatccatagggaatataacattgnccccagcagnnatctggtttccttccatagct

SEQ ID NO: 191 - DNA, 423 bp, Mus musculus
cggccatatactaacaaggtcattactttgtggtatcgtccacctgaattgctcttgggagaagaacgatatacaccagccattgatgtatggagctgtggatgtatccttggtgaactcttcactaaaaaacctatatttcaagcaaaccaggaacttgcacagcctagagctaataaggaaaaaagaggctcctctctgtctcttacatatacaaggttagcttcctgctttctgggccaggctttgaccacactgatgaaatcagcatccagtccctccatggaacaagagcaaagcagttttatgcttgttgttgataaatgaggaaaacgattagcttaaaaaatgtttacagttttaaaaataatgtttcaagctataaatgttgaaatggaaacctattttcttaaatgttgaata

SEQ ID NO: 192 - DNA, 512 bp, Mus musculus
  misc_feature 139, 175, 186, 487: n = A,T,C or G
tgagcttgtatacttttgatatgcgcgcactgggcactcctgttatggtgcacatggatcacgtgtctgcagtacttgatgtggactactctcccactgggaaggaatttgtgtctgccagctttgataagtctattanaatctttcctgtggacaagagccgaagcagggaagngtatcatacanaaacagaatgcaacatgtgatgtgtgtgaaatggacttctgacagcaagtacattatgtgtgggtctgacgaaatgaacatccgactgtggaaagctaatgcgtctgaaaaactgggtgtgcttacatcacgagaaaaagcagcaaatgattataaccagaagttaaaggaaaaatttcaataccatccacatgtaaaacggattgctcgccaccgacatctcccgaaatctatctacagccagattcaggagcagcgcgtgatgaaggaagctcggcgccgaaaggaaatgaatcgtcgnaacatagcaagcctggatcattttt

SEQ ID NO: 193 - DNA, 543 bp, Mus musculus
  misc_feature 359, 489: n = A,T,C or G
aactgagatgcccgaagaacaggcattcagtgttctggtcaagatcatgtttgactatggtctcagggaacttttcaaacaaaattttgaagatcttcattgcaagttctaccagttggagcgccttatgcaggaatacattcctgacctgtacaaccacttcctggatatcagccttgaagcacacatgtacgcctcccagtggttccttacacttttcactgcaaaattccctctctacatggtcttccatatcatcgacctgctcttatgtgagggaataagtgtcatctttaatgtcgcccttggattactaaagacttccaaggatgacttgctgttgacagactttgagggtnccttgaagttcttcagggttcagctccctaaaagataccgctcagaagaaaatgcaaagaggctcatggagcttgcctgtaacacaaagaatcctgctgtggctgtggagcctctgaaccatgtatctgnggaagccgacatttcaggcatgaaatgagtataagaaaagaaaacaggagagcc

SEQ ID NO: 194 - DNA, 430 bp, Mus musculus
  misc_feature 37, 43, 48, 64, 73, 76, 100, 124, 134, 135, 145, 152,172, 179, 198, 204, 205, 219, 224, 233, 234, 241, 248, 260, 269,273, 282, 285, 298, 305, 307, 311, 316, 323, 332, 351, 354, 364,383, 387, 388, 404: n = A,T,C or G
gatactaagctagagaactgctgcactccagcctccntggtanatcangatcccaaacaagacntagtctccntgntggaagaagaagagccatcatcgngaaaggggaaaaaagccagcccangtagtctgannaaaatagcanggcccangacagcaggaacaagtgcanaactccnacaagatgatgagcatagngaggannagcagaagtcccanagcanacttactanngaagtgncactcangaagaaaagttncaacagcangaanagcaggatnccncactggtgagaanccctatnantgtnttgantgtggtnaagccttcntgcaaaatgcctcttttancanacatatgatnattcactcataagaacaanctgnngaagaagattaatgcnggaaacctgccgactgccctggtttg

SEQ ID NO: 195 - DNA, 106 bp, Mus musculus
  misc_feature 17, 40: n = A,T,C or G
tcttcatgcaaaagtgngcgcagaagtggttacaggaaangaaaaacagatttttttgccctcctccttgtgtgtatcttatgggcagtggttggaaaaaaaaaaa

SEQ ID NO: 196 - DNA, 365 bp, Mus musculus
  misc_feature 30: n = A,T,C or G
actgagagtacctgaccgtgttctcccagnatgatcgccttccatgatccagagctgagtaatcacctcaacgagattggcttcattcccgatctctacgccatcccttggtttctcaccatgtttactcatgtatttccactgcacaaaattttccacctctgggatacattactgctcggaaattcctctttcccattctgcattggagtagccatcctccagcagctgcgggaccgtcttttggccaatggcttcaatgaatgcatccttctcttctctgacttaccaggtatgtgtctaaatgaaggaatcaccgaatgcaattttgactataaaatgatgtgctctctcaaaaaaaaaaa

SEQ ID NO: 197 - DNA, 369 bp, Mus musculus
  misc_feature 21, 26, 38, 70, 71, 81, 90, 124, 133, 134, 159, 161,169, 183: n = A,T,C or G
gaactgagatttaatcactantgctncgcctggggatnctgtatagccctgctgcgggcttcattctcannagagcccagntctgacaanaacaactggacgccctcgtggcaaaggggaatgncttgaccanngagcaacaggaaaggaaggaagccntnctgagctntccgcagttacctngctgagagcttcatgggtgtttgtccccggcccgtacctgtctcaaatgctccatctcccggtaagagagttgatgaaactttaggttttgtttccacatgtctgcctttattcttctagcctttttgccatcagcccataacatctgtattcctgccccattaaagtggaggacagaaaaaaaaa

SEQ ID NO: 198 - DNA, 549 bp, Mus musculus
actgagggctccttgtgtacgggctgagcctattgtgcttttctgcgcttcggcccttcggcgagccgaggcgggaagtggagatccaccggcagtacgtggcccagtctgtgcagcgtcttcatcctctacttctttaacctggctgtgctttccacctatctgccccaggacaccctcaagctgctcccgctgctcaccggtctctttgccatatctcggctgatatactggttgacctttgctgtgggccgctccttccgaggctttggctatggcctgactttcctgccactcttggccatgctggtgtggaacctctattacatgtttgtggtggagccagagcgcatgctcactgcctccgagagccgcttggactaccctgaccatgctcggtctgtttccgactacaggccccgctcatggggctgagccacctagagctgcacagggggctttcggcacttccctggaacagttgtaggcctggcctttgcccaggcaatggacctttgatgcgaggaaccttgctacttccccactggg

SEQ ID NO: 199 - DNA, 313 bp, Mus musculus
caagtcaagtccattgaacgctttcttcgacgactggaattccatgccagtaagattgacgagttgtatgaagcatactgtgtccagcggcgtctcagggatggtgcctacaacatggtccgcgcctaatatattcttttgttaccactggggttggcagaatgcctgaaatgaagtgcctcccagccgcggttctgcacagtctttgccacacacagtgtcggaggcctggccatttggtcagcttgccttgcctcatcacatcccttgggtttgtattttatttacagagttttacagaaaataaaagaaa

SEQ ID NO: 200 - DNA, 140 bp, Mus musculus
gctctgctgcctgcatgcagacatgctgagctctctgggccccaaagaagccaagaaggccttccttgacttctatcacagtttcctggagaagactgcggtgagatacctcccagctgcccctgtcccaaaaaaaaaaa

SEQ ID NO: 201 - DNA, 122 bp, Mus musculus
  misc_feature 100: n = A,T,C or G
gactgaggacttcccgtttctgttgattttcaagtacgtctttgagtcgagtggaagacagaagaagtggtcctgaagaagctagagtatatttatgcanaacaacaagcactagaagcaca

SEQ ID NO: 202 - DNA, 255 bp, Mus musculus
  misc_feature 118, 147, 197, 208, 224: n = A,T,C or G
cctggagtggctgtgctggagtggcaggagaagcgtagacccttctcggactccacagctttaatactggggagtagggtgcagtttcactagtagaagccacccctcctcctgcctnccaggtcagctgcttctgagcaggggctnctggcgactgtacctgcccagcaacctggaggacagcgtcctgaggtctnccaaggatggnccttgcccctgtatgnttccctaataaagccttttaacccaaaaaaa

SEQ ID NO: 203 - DNA, 141 bp, Mus musculus
actgagggtaggaggcagagaggtccatgcacaatccccgggactggccttacgcatcgcatcagagctttgtttgaaaatgcgattcggagcgacaagggcaaccagtgtcccctactgtggaggatgtatttgaatttt

SEQ ID NO: 204 - DNA, 367 bp, Mus musculus
gactgagggtggccctgactccatgtaggccccagctctgattctgtcattgtggatgacgaggactgactcccacgatatcgcctggaccttgcttggcctttcagtatggggaggattggcatttcctgtctctttcctgcctcttggcattttagcatctctccagtgggctgtcctcgaattctgaacaccaatttacgacaaatcgttgtcattagcatcctggctgcagctgtctcccttttatacttctctgttgtcataatccgcagcaagtatgggtggctgtcaaaggacaagaaatttcaaagaggtaactcacggttagactcacagcaactgaccatcggaggagagaacggtc

SEQ ID NO: 205 - DNA, 387 bp, Mus musculus
  misc_feature 18, 52, 90, 120, 125: n = A,T,C or G
gactgagaagaccatgtntggcccagtgacgagacagcaggagacctgccgntgaaagataccggtctaaacctctttggagttggaggnttacaagaaacctcaactgcgctgcactgntgaanactcgccaggcagtgtcactgtgtcagccgtgaagaactggaagacagatttttgctgtttgcatgatgagaacattttacttaaacagcatgcccgcaagcaagaggataaaattaaaaggttatgataaaaagcttttagctttttttcttaactctgtaaagtttgaaggatatagtttcttataaacttaattgaaagccccaccttcaatcttttctggaacaggcaggttataaataaatagaatttttttaaaaa

SEQ ID NO: 206 - DNA, 508 bp, Mus musculus
  misc_feature 243, 421, 424, 441, 443, 444, 458, 459, 464, 468, 470,474, 483: n = A,T,C or G
actgagaatgttgttggaaaaggaaggaggaggcgcaacaccatctctgacagaactgggtatgactgcaaacacttgacccagaactgtgtgactggcattgtggagaaatcagctctcgccgcgtcgctgttgggttcaggaccatcaccccgagcagagggacaagtcgcctaccatttgcaatcctgcactgccgcagcacctcgttgaagccctcacagagcttgacatcgctctggntctgagcacactccagaaactgcttgatctctagagagcaaggtccaaaagactgctggttctgcagctgggctccctgaggctcctgcaaagacacagcatcttgtgttgagatgagaaagcacagggcaccacccaagggcagcgctcacacactgggagctggggaacaactggnganaggtgcaagtttaatgncnnccaggaccatgccnntgaanacgntncaangaacacccnatcccctcccccaactcacaaccac

SEQ ID NO: 207 - DNA, 511 bp, Mus musculus
  misc_feature 31, 63, 76, 83, 96, 130, 151, 161, 170, 182, 246, 276,287, 298, 344, 377, 394, 410, 414, 432, 441, 458, 479: n = A,T,C or G
tctgcacagctacccacgatggggaggaaanaaatccaggatctcactgaattctagaccaanggaacaggcaggngactttnaccaagcgcaagnttggactgatgaagaaggcttatgagctgagcgngctttgtgactgcgacattgncctgatcatnttcaacagngcgcaacgcctnttccagtatgcgagcagcgacatggaccgggtgctgctcaaatacaccgagtacagcgagccgnacgagagccgcaccaacgcggatatccttnagacacttaanaggaggggcgngggccttgatgggcccgagctggatatggaggaggggccagaaggncctggagagaagctgctgaggacactggggggngacagaggctcggcctngcccccgccttggatntatnccgtggcccctgcaatgncggtttcanagctgtcctaccgggtnccaccagccactccaggctgngatcctggcgggctgggggaggccccgtatgt

SEQ ID NO: 208 - DNA, 476 bp, Mus musculus
  misc_feature 29, 40, 43, 44, 45, 46, 59, 61, 71, 229, 234, 277, 284,310, 337, 338, 347, 361, 367, 385, 392, 414, 417, 428, 437: n = A,T,C or G
actgagggcagttcctccagaagtgcagnctctgcgagcntgnnnnctgagctggtcgnanaggctgaggnccgatgccttcgagtgattaatcctgatgtgggctctggcctcagtcacttgctaccaccagccatgtctgcccgcttccgcctggccatgagtctggcccaggcctgcatggacctgggctatcccttggagcttggctatcagaacttcctctatnccagngagcctgaccttcgagatctgcttcttttcttggctgagcgantacccancgatgcctctgaggacgcagaccagnctgcaggtgactcggctattttccttnnggccattgngagccaaatccggnaccagntggccctgccctgggtcncacctgnccttcgaactcccaaggtgcantgnctccagggctnaaccctccngcagccgttccactccagcaggttggttctacctgagat

SEQ ID NO: 209 - DNA, 316 bp, Mus musculus
  misc_feature 32, 49, 81, 116, 130, 162, 168, 170, 172, 178, 179, 181,183, 184, 193, 195, 199, 203, 283: n = A,T,C or G
gactgagggaagctggcacggcggcgcgtggngggcggctcaccgcggnacttcagcactgaaggacatgtccaacacganatctgggaccacttcctctctccaggctagcctgnctagaaactaagtnatagaaactaagggtaaaatacgcatcttcgnatatgnananacatanngntnnaggagatcncnggtncatnttctgagccatcgatggagacacaggcaggctgccatgccatgccaacctggcacttctgtgggttctggggcggggggngaattacgctccttaccggctccagcaccacag

SEQ ID NO: 210 - DNA, 272 bp, Mus musculus
  misc_feature 25, 40: n = A,T,C or G
caaaccccgtaccaaggaactagcngagttccaaaccagncagagatacaaagggagaggtgagatcctgtttgaagaaacctccaaacctaagacacacccaaagaaaagcactacccccaccaccatcaacaacaaaatgtttgaatgatctgggccatccagttcctttctggaaccaaaaccagctaatattgatattgacgtttatcacttaacccctctccctgatttcgaaatgaagtctgttccactcagccgaaaaaaaaaaa

SEQ ID NO: 211 - DNA, 103 bp, Mus musculus
tggtcgacgtctcctctgcactcaaacgcttcctcagggacctgcccgatgggctcttcacgcgtgcgcagcgcctggcctggctggaggaaaaaaaatgaaa

SEQ ID NO: 212 - DNA, 492 bp, Mus musculus
  misc_feature 276, 466: n = A,T,C or G
gactgagatagacctcttggccccccgcacctagtacaatggagtagacaagacttcgaggggaaagaggcttcccaagatgacccagctcattggcttgactcccaacgccacccacttacacaggccgccccgggactgccaggaactcttccaagaaggggagccggcacagtggacttttccagatccagcctctggggtctccaccatttttggtcaactgtgagatgacttcagatggaggctggacagtgattcagagacgcctgaacnggctctgtggacttcaaccagtcctgggaagcctacaaggatggcttcggagatccccaaggcgagttctggctgggcctggaaaagatgcacagcatcacagggaaccgaggaagccaattggctgtgcagctccaggactgggatggcaatgccaaattgctccaatttcccatccatttgggggggngaggacacagcctacagcctgcagct

SEQ ID NO: 213 - DNA, 397 bp, Mus musculus
  misc_feature 28, 58, 147, 237: n = A,T,C or G
aggcctggcccttctctgggtagcaggnctaggggatactgcccctaaccttccccgncttcggctctcctttcaagaattacaggcccggcatggtgtccgaaccttcaggctggagcggacctgctgttatgaagccttgctggnggatgaggagcgtggacgcctgtttgtgggtgctgagaaccacgtggcttccctcagcctggacaacatcagcaagcgagccaagaaggngccccggacccctaaccttactcagcatcagtgctggctcaggaaaaagccttagggtcccaatccagtctgtctagactccagagaatctccaagtggtgggcaactgaatagagaaacccccaaaataaagagcgccccaaatccctaaaaaaaaaaa

SEQ ID NO: 214 - DNA, 510 bp, Mus musculus
  misc_feature 77: n = A,T,C or G
tgaggtccatttcacgtcaacaacggtgccggaggataacagccacctaccagcccagagccgccagagctctctgngatggcaagtggcacacactccaagcccacaaaagcaagcaccgcatcgtcctgactgtggacgggaattccgttagggctgaaagcccccacacccattccacctcggcagacaccaacgatcccatttatgtgggtggctatcctgcccacatcaaacagaactgcctgagcagccgggcctcattccggggctgtgtgaggaacctcaggctgagcaggggctcacaagtgcagtccttggacctgagccgagcctttgacctacaaggagtcttccctcattcctgccccgggcctgagccctaaactgtcgccagcctctgcccttggaatcatcgccaacgcatggaagagagcagtttgtgaactcaagcagctcagctcccattcccatcccattgccatctcaggttatgtttccagaggaaaa

SEQ ID NO: 215 - DNA, 368 bp, Mus musculus
  misc_feature 193, 195, 196, 231, 292: n = A,T,C or G
ccctgatttgccccgagaaggaagcagcaggcaatgttaccattcactgggtgtactctggctcacaaaacagagaatggactaccacaggaaacacactggttctgagggacgtgcagctcagcgacactggggactatttatgctccctgaatgatcacctggtggggactgtgcccttgctggtggatgntnnaggaaaagggacaccgctcggcctgtgcgtgtggncagccagctgtggaagccatctggcagatttaatagccctggactcccagccaggcacctnagcttagcggctgctaaccccctcagattcttttggcaaaataaagtgaactttattactaagttaaaaaaaaaaa

SEQ ID NO: 216 - DNA, 268 bp, Mus musculus
gactgaggtggggtctttcacgaccaagaccagaagagctgttggaaagctgtggactccgcgccgtcgttcaccacctccacattggtgtagttcgggtaacccttggcctggatgaagccctgggacacagctctgtcaggcctgaggctcaaaggcccttggctgtgctccatccccagagacacccccaaccagtggccacaaggatgcccaagctctgaactgagttaattaaccaaacttgtgacccatttaaaaaaaaaaa

SEQ ID NO: 217 - DNA, 375 bp, Mus musculus
  misc_feature 30, 31, 54, 82, 120: n = A,T,C or G
agctagaacaagctcagagtcggatcttgnncctggaaaaccagctggaggagncaggtcgccactgggcacgagagaagcnaaacctggccattcgtcttcaagagcaacaacacggcntttggacagccaccaaactccatcatcatagaccagcctaatgctggagcatccaagaatcgccagcagctctcgaaactggaaacctccctgcccagctcagacaagaagctcaacagaccctcagactctcagattgagatctctaacaaccagaagacctgagaccgggagcagatctccttcgccatgtgctggggattcacgtgcccccaaagacgcctttaataaaagctgtcagtcaataaaaaaaaa

SEQ ID NO: 218 - DNA, 548 bp, Mus musculus
tgagccctatggggcacccgagtgtgcgctcaggaaccggagttcagctatggctgcgcagaaggcagctgctaccctgccactggcgaccttctcatcggccgagcgcaaaagctctccgtgacttcgacatgtggactgcacaaaccagagccctactgtattgttagccacctgcaggaggacaagaaatgcttcatatgtgactcccgagacccttatcacgagaccctcaaccccgacagccatctcattgagaacgtggtcaccacatttgctccaaaccgccttaagatctggtggcaatcggaaaatggtgtggagaacgtgaccatccaactggacctggaagcagaattccatttcactcatctcatcatgaccttcaagacattccgcccagccgccatgctgatcgagcggtcttctgactttgggaagacttggggcgtgtacagatacttcgcctacgactgtgagagctcgttcccaggcatttcaactggacccatgaagaaagtggatgacatcatctgtgactgggactg

SEQ ID NO: 219 - DNA, 339 bp, Mus musculus
  misc_feature 31, 38, 45, 53, 59, 60, 67, 78, 93, 99, 103, 105, 107,122, 124, 134, 136, 138, 143, 157, 171, 204, 243, 273, 275, 295,296, 308: n = A,T,C or G
tgaggaatgcctacctgttggttctaataangatgaangtgatgngatcttgnagaagnncatgatncatgcttcggncgcttgctttggcanggaggnttcntnancacaggcatcagagncngtagactcanancngagantgagaaacaccganggcttctgtgacancctccacatttcccacctgggtatgctcttcanagtcgttcacagttgatagcattctgacaggggtggtgnaccccccgaacggagaagatcgaagttaancntgttgggaaaactgatcccnncactgtcttcgngatgaataaaaacatctcaaccccttacagt

SEQ ID NO: 220 - DNA, 227 bp, Mus musculus
  misc_feature 40, 43, 44, 61, 63, 77, 96, 111: n = A,T,C or G
ctggttacctcatatacatgttcgcctgatgccatatccnctnncaaccatcgggtaacgnanggactttggagaanccaagcctatgggtacccnctcctggaggctgtncttggggagagcccaggctggcctttccttcagaagtcttaccttctgtgtgtttggcttgatgcatctgacaaagaaaggatttgaggagcttagtgttgccattaaaaaaaaaa

SEQ ID NO: 221 - DNA, 226 bp, Mus musculus
actgagaattgcttcatcatgagaaatctgaagttgcatcggaccctggagtttcgggacattcaggcaccagggaaacctcagtgtttctgtctgcgagctgagcagggcacggtgctgattggctcagaacgtggactgacggaagtagatcctgtgagaagagaagtgaagactgaaatttctttggtggcagaaggttttcttccagaggatggaagtgcct

SEQ ID NO: 222 - DNA, 541 bp, Mus musculus
  misc_feature 197: n = A,T,C or G
tgagagagtgatggtcttcaaaatgaaaactctggaaaattttaggttctctttaggaactacaaaaattgaaggacagcaattttttgaaaggaagttgttctgaaagcatgtttacaattcactgacactgttgactgttctctttaaaataataagacgctttgagaagattgtatttatggtaaaaggaaacntggactaacaatgaggccaaagacttttcctgccacaacttactctggaaatagccggcagcgattgcaagagattcgagaggggctgaagcagccatccaaggcttccacccaggggctgctggtgggaccaaacagtgacacttccctggatgccaaagtcctggggagcaaagatgcctccaggcagcagcaaatgagagccaccccgaagtttggaccttatcaaaaagctctcagggaaatccgatattccctcctgccttttgccaacgagtcaggcacttcggcagctgcagaggtgaaccggcagatgcttcaggagttggtgaatgcgggatgtg

SEQ ID NO: 223 - DNA, 433 bp, Mus musculus
  misc_feature 145, 264, 312, 316, 330, 338, 343, 352, 393: n = A,T,C or G
gactgaggtattggacaatcagataaagaaggacctggccgacaaggagagcctggagaacatgatgcagagacacgaggaggaggcccacgagaagggcaagatcctcagcgagcagaaggcggtgggtattaagacaagccgngccatcttcccaggaagaggttatgtacctgcaaggccaagagtcaccccgtttcatctggtgacatggtctctccatgatatcctacgaggtttataggtaccaggaagctttctcgnaaacagcttcctgtcccaaaagaacagggtcggatttataaacccgantggnaaactgaaagctgnaagagcanctggngatgatcanccaccaagatcacagtgacaagagtcggctgctggagctgnaaacaaggctgagggaggtcagcctggagcacgaggagca

SEQ ID NO: 224 - DNA, 112 bp, Mus musculus
  misc_feature 31, 48, 49: n = A,T,C or G
cagacgacatcatggacctgttctgtatcgncaccggcctgcctcggnncaccaccatctcccttttaaccacagacgacgccatggtctccatcgatcccaccatgcctgc

SEQ ID NO: 225 - DNA, 343 bp, Mus musculus
  misc_feature 26, 204, 209, 233, 244, 247, 274, 276, 280: n = A,T,C or G
actgaggacttggtgacctttgaggntgtggctgtgaacttcacccaggaagagtggactttgctggatccttctcagaagaacctatacattaatgtgatgctggagacctgcagtaaccttgcttctgtgggaatcaaatcagaataccagaccattgaagaccagtataaacagcctgggaaaaatataagactcttgaancaaantagaatgtttccaagaatctcatnctagaagcaanggncgaggaaaaccagaggaggaccttgtntntggnagacttgtctaagtctgttttcccgccatactgtcaagacaagagctgaaggtagggaggtgg

SEQ ID NO: 226 - DNA, 232 bp, Mus musculus
  misc_feature 160, 166: n = A,T,C or G
attgagctttgggattctgacatactgtagggcaaatgagacatccagcctagtcgactgaacaactgctgattcttggacgttgggagacagccattgttgaaatagctggaccacagcctggacttcaaaacatgacctgcattacagaagggaagcnggaggntaacaccacactcgaggagttgttactctattttattttcttaataaatctatgcatatgtaagta

SEQ ID NO: 227 - DNA, 420 bp, Mus musculus
  misc_feature 35, 77, 255, 262, 267, 311, 329, 331, 350, 354, 383,387, 389: n = A,T,C or G
cgtcgtgggtcaccacctgcccaggggccctagtncaagctgcctggaacacgctggctccatcaatgaagaggctntccaccccgacgagcccatcatcctctcagcatccagtgacaagaggctgtacatgggtgagatccagtgaagatggggaatggaaggctcccggctcccagccctgaggctcagactgcggaattggccagagtccgatggtgtctgggctgacctgcctcagatgaccatcattancaagcanctggnggtgatggccgtattccaacaaccacttgtaccacactcaccanaacaactgtggcctctgntnccgcctgccagattgcagngatntgctttcttctttttgaaaaataagaatncttnanaaggacaaacattggataagataatttcctg

SEQ ID NO: 228 - DNA, 288 bp, Mus musculus
  misc_feature 42, 56, 65, 70, 86, 87, 90, 91, 95, 100, 103, 105, 109,140, 156, 203, 258: n = A,T,C or G
acagcagtgtgggagtgccatggccttgcatccagaccctgnggatgaagactcgnacgactatnatggngaggagtatgacgcgnnggnncacnaacanggncncggngacatccctacattttatacctatgaagaangactatcccatttaanaccggccagggcatcgccaccctggagagattagatgggacgctttntcagtctgggagcagccaatataatatggcaggcgtccgaacagaagacatcagntacattttatacctatgaagaaggactatc

SEQ ID NO: 229 - DNA, 198 bp, Mus musculus
  misc_feature 37, 45, 57, 103, 106, 120, 139, 142, 157, 177: n = A,T,C or G
aaagacaacaagccttctgggtaccaagccttctctncaaacctncaaatacttctntcctcccccaccggccaggagctgcagagcagagctggccgcacgngtncccctgctcccggntctccatctctcctgccancantgccagcttctctcncacacacctcacggactcanacattgaacaaggaggcagca

SEQ ID NO: 230 - DNA, 317 bp, Mus musculus
  misc_feature 16, 31, 102, 143, 160, 181, 224, 251, 261, 277: n = A,T,C or G
agctttcgtaaaggantatgaagagctggcntatggcgagtcctgctgccgcttctgtgagaccgcccaggcccaatgaaagagccgtatacgctggtcatncccaagaatgcggccgaggagcagaagctcaagctggagcngctcatgaagaacccgnacaaagcagttccaattccanagaaaatgaatgaatgggctcctcgagcgcctccagaatttgnccgagatgtcatgggttccagtgctgnggctggcagnggagagttccactgtntataggcacctacggcggagagagtaccagcggcaggaga

SEQ ID NO: 231 - DNA, 146 bp, Mus musculus
  misc_feature 94, 105, 118, 128: n = A,T,C or G
ttgcctctactacctgtccgctctgaaattggaacacttggctctcatctacttcggaggcctgtctggctcctttctctacacaggaggaatngaatgcaagtntgagggcctgacngaccatggtnatcctcataacttacgga

SEQ ID NO: 232 - DNA, 256 bp, Mus musculus
gactgaggaattacacagtcgacaaaaaaagccatttttcttgggagctgaacaaaggctgaaggactggcataacaaggaggctatcaggagggacgcacagcgcgtaggatatggagaacaggggaagccttaccccatgaccgatgccgagagagtggaccaggcgtaccgggaaaatggatttaacatctacgtcagtgataaaatctccctgaatcgctctctccccgatatccggcacccaaagtgagtg

SEQ ID NO: 233 - DNA, 480 bp, Mus musculus
gactgagggcttggctatgtcgcctttcgggagcctgtttccttacccctacacatacatggctgcagcagctgcagcctccaccgcggccgcctccagctccgtgcaccgccacccgttcctcaatttgaacagcatgcgccccaggctgcgttacagcccctattccatccccgtcccagtgccggatagcagcagtttgctggccacagctttaccatcaatggcttccgccgcggggcccctagacggcaaagcggccgccctggcagccagcccagcctcggtggctgtggactcggggtcggaactgaacagccgctcgtccacgctgtcctctggctcagtgtccttgtcacccaaactctgctccgagaaggagacggctaccagcgaactgcagagtatccagcggctggtcagtggcttggaagccaagccagacaggtcttgcagcgggtccccttaaaaacaagaaaa

SEQ ID NO: 234 - DNA, 520 bp, Mus musculus
ttgagccacgcgttgaagtgtttgaatgcagtggtatagaggactgctacaagcctaagacttgctccagccagagcctctactgcttgaccaactggtatacaccgccaggtcagcaaacgactgtgacaaaaacatgtgcatatacctgccctgacattaatcatgtcacagccaactccaagtcctcctgctgcaacacggacctctgcaatagtgccaggaacctccacgtcagttggggattgctggcccttggcctctgctatatctatctcagccagtaggtgccgagctcccacaggggctcacaagctgcagagttaagagttggagtttggtgccatcatgcaaccggaatgtgtagccatgggtggggcacttcttgaactccaatcagaaggattccaagaggcaacctcacagatgtggctgatagagaccgtggcccctgtccagagtgctgcattcagaactcttgtctgagaggcatgtggattttctgagttcgaggccaagg

SEQ ID NO: 235 - DNA, 193 bp, Mus musculus
gactgagcgatacaactgcctaaactcagggttactgccgtggcagctgctgggatcctggagagagttcttctcaccactgcaatctgtgctttgtatctgcctttgtaaacacagaggaatagtgcaaccctgccatggacagcatgccctgaatagcttcttcctctgtgctcgtcgagaaagaaaaaaa

SEQ ID NO: 236 - DNA, 467 bp, Mus musculus
  misc_feature 21, 25, 44, 50, 53, 56, 57, 59, 60, 72, 73, 75, 78, 85,86, 90, 94, 109, 122, 123, 124, 127, 128, 172, 193, 417, 434: n = A,T,C or G
gagctgatgactggtcaggtntggnctacatccagggacaggangcccgntgnggnnannaacacgaagccnntntgnacaggannactncaanctctcacttcctcancacaccttcccgnnntcnncatcatggggctgattccttgcctccaaaccttcctgtaaaatnacaggagaccctgcagttctnctggcagtcccgatggcttcctccacgctttggatcttccatccacaagaggactggcaccctaccggatactagccttttcctgggggggaggggacggacacacagtctaaactgactctcaccttcactgggaaaatatccaaggaatcttccggtcctttcagattcatatagggcgagaagctgctggggcaggaccgcctcttctccagaagctccgncttaatctcctctcttnccaccagatgttccctgggtagaacaacaatgg

SEQ ID NO: 237 - DNA, 284 bp, Mus musculus
  misc_feature 138, 226: n = A,T,C or G
ctcttggggcattggtgaacctcagtaatgcacatcttggctccatcaaaactcggtttgaaggcctgtgtctgctgtccttgttgataggggagagccccacagagttattccagcaacattgtgtttcttggcttnggagcatccagtaggtgctgcaggttcttctgaattttccagaaagtcctgaaacccctgagttccagggtgggtgtatatcaaactnggagagatgaggcagagaaatgcagtatgggagaataaagattattgtaatctcctaa

SEQ ID NO: 238 - DNA, 183 bp, Mus musculus
  misc_feature 17, 19: n = A,T,C or G
ttttggtcggcaggtgntngcaccaggaatcacccacaggcctgctgtggccaagtccgtagacacagcatcggaagaacaagggggtggtcggagctaattccctggtgctggatccagtttttcttcctgagaggccaaagctaggatgggagtctcatctccaccattgaaaaaaaaaaa

SEQ ID NO: 239 - DNA, 374 bp, Mus musculus
gcctgtccctgagaccgtcagatcggccctcctttgaagaaatccggaaccatccgtggatgcagggtgacctcctgccccaggcagcttctgagatccatctgcacagtctgtcaccgggatccagcaagtagcagcctttctgctgctgtctgtcagaccctccagggagggagagcttgtcttctggcctccaacaggaccctgcgtgacgatgcagggacagcaatgacaactcattccagactccaggtccctggaggagcctcccacaagggaaagagactacttcactggtcctgggcccctctttgcgcgccccgcccccagactcagcgtctagtgttgctgggctcccctgaatccatccctgg

SEQ ID NO: 240 - DNA, 334 bp, Mus musculus
gactgaggcctgtattgatgaaaatttggacatggtgaagtttctggtggagaacagagccaatgtaaaccagcaggacaacgagggctggacacccctgcatgcagcagcttcctgtggttatctcaacatagcagagtattttattagccatggagccagtgtgggtattgtgaacagtgaaggtgaagttccttctgaccttgcagaggagccagccatgaaagaccttcttctggaacaagtaaagaaacaaggcgttgatctagagcagtcaaggaaggaggaagaacagcagatgttgcaggatgcccgccagtggctcaacagcgga

SEQ ID NO: 241 - DNA, 357 bp, Mus musculus
  misc_feature 299, 312, 320: n = A,T,C or G
actgagataattagcaccatggagcctcaggtgtcaaatggaccgacatccaatacaagcaatggaccctccagcaacaacagaaactgtccttctcccatgcagacaggcgccgccacagatgacagcaaaaccaacctcatcgtcaacatatttaccccagaatatgacccaagaggaattcaggagtctctttgggagcattggtgaaatagaatcctgcaaactcgtgagagacaaaatcacaggacagagtttagggtatggatttgttaactatattgatccaaaggatgcanagaaagccatcancactttanatggactcagactccagaccaaaaccataaaggtgcc

SEQ ID NO: 242 - DNA, 172 bp, Mus musculus
  misc_feature 17, 97: n = A,T,C or G
gactgagatgaacacantgatgtacagaagtaaacctttaccaaatggataaacgctcgattttccaagagtgggaaaccacccatcagtgatatgntctcttagtttcactacgtgtacagagcagagacgatgacccggagtggaagttggtagaccaaattaaaaaaaa

SEQ ID NO: 243 - DNA, 235 bp, Mus musculus
  misc_feature 16, 19, 24, 42, 58, 76, 81, 85, 128, 157, 169, 205, 211,214: n = A,T,C or G
gaccaggtcaggacanggngtctntgttgatgtgggcacaantcttcaaaccagtggntgtaatctcaacagccgncacgncttncatgtggtgcctccaccttggaaaagcaacaacagtgcatggncactaaagatcatgaaaaacatcatccgngactgcctgaanaccaccgagaacctgtctctacaatcgattgcattntcagcnatnggaacggcaaattcgaggctt

SEQ ID NO: 244 - DNA, 469 bp, Mus musculus
  misc_feature 17, 18, 49, 234, 343, 429: n = A,T,C or G
cctttgttcaggccagnntgttcacaggaagatctgggtatctcagtanccctgagtacccgcagccataccccaagctctccagctgcacctacagcatccgcctggaggacggcttcagtgtcatcctggacttcgtggagtccttcgatgtggagacgcaccctgaagcccagtgcccctatgactccctcaagattcaactagacaaggctggaacacggcccattttgngggaagacgctgccctccaggattgaaaccgacagccacaaggtgaccatcacctttgccactgacgagtcggggaaccacacaggctggaagatacactacacacctnacagcacggccctgccctgatccaacggcgccacctaatggcagcatttcacctgtgcaagccacgtatgtcctgaaggacaatgntttctgtcttctccaagacaggctttgagcttctgcaagg

SEQ ID NO: 245 - DNA, 454 bp, Mus musculus
  misc_feature 32: n = A,T,C or G
actgagctgcagcaatacccgcgtctgcgggnggagcatggagcgaattgtgaccacccacatccgggaacgtgagggccgcaccaaggagcaggtcatgcttctcatcgacattgagctggcttacatgaataccaaccacgaagacttcataggctttgccaatgctcagcagagaagcaaccagatgaacaagaagaagacttcagggaaccaggatgagattctggtcattcgaaaggggtggttgaccatcaacaacatcggcatcatgaagggaggctccaaggagtactggtttgtgctgactgctgagaatctgtcctggtacaaggatgatgaggagaaagagaagaagtacatgctgtctgtggacaatctgaagctgcgtgatgtggagaagggcttcatgtcaagcaagcatatttttgccctcttcaacagacataaaaaa

SEQ ID NO: 246 - DNA, 378 bp, Mus musculus
  misc_feature 17, 28, 36, 52, 64, 70, 72, 84, 88, 91, 99, 106, 107,108, 113, 157, 173, 175, 183, 184, 189, 206, 223: n = A,T,C or G
ccagctgtctctcctanccaggaaatgntggagctnggactcattcagaaangatcggacccangacagncngcgctgatgcangctnccnccaccccntcttccnnncatgngcgtcaaccctgcccttgtagggaccactcgcttctgagcgttngtagcggacctatganangagagttnngcagnctattttgaccacattnggcaaaaaacactaacnggttgctacaccccctgagggcagcgtgacggcagcatcatgaatgagacggacttggcagggccagtggtgttctgccttgcctttggggccacactgttactggctggcaaaatccagtttggctacgtatacgggatcagtgcgattggatg

SEQ ID NO: 247 - DNA, 256 bp, Mus musculus
  misc_feature 29, 145, 162, 193: n = A,T,C or G
gtagaccgtactttcaccacaatgagctncagccccttcaggaacagctcgctgtacgccgtcacccttctacacttgaccagactggcgctctccacctgccccgccgccgccttcgcttctcaccatgtgtaaggcggcgggnagccccgcctgagctgnccctccctgctcacacttgctcagatgctgnagtgcaggggcttcagcattcttgagttcctccgtgttaataaaaacatcctgtgacagttaa

SEQ ID NO: 248 - DNA, 368 bp, Mus musculus
  misc_feature 16: n = A,T,C or G
gactgagggctaagtncttgtgatatgtcgaagcagccaatttccaacgtcagagccatccaggcgaatatcaatattccaatgggagcctttcgtccgggagctgggcagcctcccagaaggaaagagagtactcctgaaactgaggagggagctcctaccacctcagaggaaaagaagccaattcctggaatgaagaaatttccaggacctgttgtcaacttgtctgagatccaaaatgttaaaagtgaactgaaatttgtccccaaaggtgaacagtagtcgaaaggacacaaaagcgttgacgtgtttttccaagggagaaaaaacaatgggttgaaataaacaacttcctgaacattttaaaa

SEQ ID NO: 249 - DNA, 528 bp, Mus musculus
  misc_feature 181, 183, 188, 190, 195, 197, 201, 203, 266, 267, 274,280, 293, 295, 302, 304, 305, 311, 312, 315, 322, 327, 332, 337,340, 343, 344, 357, 358, 378, 393, 394, 397, 398, 399, 415, 426,428, 429, 435, 439, 442, 447, 448, 450, 454, 457: n = A,T,C or G
  misc_feature 469, 485, 486, 488: n = A,T,C or G
gactgagtgttccccggacactgggccccaagtagcccctgggcagccagagaacaacttctccaaccccaaagcaagcatctgctactgctcctggccgagagagcccccgagagacaagggcccaaggcccatcaggccaggaggccgaaagtccccgcaggacgctgcagattgaccntnttacnangagancnctgngnccaatatgcgagacctcggacctcacatcaacctcccagccagccaatacttctgcacctgcnntccgagngtcatnaccaaggtctgcnangcaatgngnnttcaannccnaccctcncctancccanaggantgnggnncagttcctagcanncacagagtggctcacaatgntctgtattccatgcnnagnnnctccaatgccttctcnacccaggatgnanngagacntgcntgnaggannanaacnatncttatacatatnaatacttttaacctgnngngctccaatgccctctccacccaggatgcacagagacatat

SEQ ID NO: 250 - DNA, 385 bp, Mus musculus
  misc_feature 33, 46, 48, 51, 63, 65: n = A,T,C or G
tgtcatgggccagaacttaattttgaacatgantgatcatggattngnggnctgtgctttcantnggacagtctccaaagtcgatgacttcttggccaacgaggcgaagggcaccaaggtggttggtgcacagtccttaaaggacatggtctccaaactaaagaagccccgccgggtcatcctgcttgtgaaggccgggcaagccgtggatgatttcatcgagaaattagtaaggccagctcggttctcggatgccgcccgcaaatccctctaactgtctcacttctgagatggtggtcttcagtgtctgagtacagactgatgggttctgataaagtgcttgagtgtttcaggcttggttgccttgacaggcacttatggaaaa

SEQ ID NO: 251 - DNA, 511 bp, Mus musculus
  misc_feature 34, 292: n = A,T,C or G
gactgagcgcattggtgaggactacatctcagancctggaccagctgcgcaagctgctctcctatgtggatgatgaagcctttatccgtgatgtggccaaagtgaaacaggaaaacaagttgaagttctctgcatacctcgagagggaatacaaagtccacatcaaccccaacatcgcgtctttgacgtccaggtgaaaaggattcatgagtacaagcggcagcctcctcaactgcctgcacatcatcaccttgtataaccgcatcaaaagggagcccaataggtttatggngccgaggactatcatgattggaggcaaggctgcacccgggtaccacatggctaagatgatcatcaagcttatcactgccattggggatgtggtcaaccacgaccctgcggtaggagaccgcctccgagtgatcttcctggagaactaccgagtctcccttgctgagaaagttattcccgccgccgacctctcagagcagatctccaccgcgggcaccga

SEQ ID NO: 252 - DNA, 513 bp, Mus musculus
  misc_feature 96, 319, 340: n = A,T,C or G
actgagccgggttcccggccgggtcaatggctggccaagctcccacagcggttcccggcagtgttactggagaagtctcacgatggcagaacctangacctgcccagcctgcacagaaagtagcccagccccaaaacctggttccagatgggcaccttgagaaagccctggagggaagtgaccttctacagaagttgggtggctttcacatcgccattgcttttgcacacttggcctttggtggctacttgatctccacagtcaagaatctgcacctggtggtgttgaagtgctggtatccactctggggaactgtctnttttctcgttgcagggatggnggccatgacaacagtgacattccctaagacctctctgaaggttctctgtgtgatagccaacgttatcagcttgttctgcgcactggccggcttctttgtcattgccaaggacctcttcctggagggtcctttttcatggccaatctggagaccataccctgaacccacaacct

SEQ ID NO: 253 - DNA, 445 bp, Mus musculus
  misc_feature 329, 330: n = A,T,C or G
aaactgtgggaaacaagagtccacacataacattttcggaaagtacacagaaaacaaaggtctctgaaggctgtaacagagaaggctgtagacgatctcctccctaagtcggcttccacacaagagctggcagtgctgaacacccgccgcaactctcaccccgtgggaaccttacttacattgtaggtctccagcttgaccctgattctgaacgtaaatgaccggaagttggcattctggaaaacctcttcaaacgcctgctcgttctgcaaaaaacaagacatgggaactactggaaaacacccattagactgctgaagagactgagnntattgtcatcttgaagggttgaaatcactgaggaatggctaatgacaaaactgcttaaggagaattttattaaaaaacaaaactccttctggaggagtactgtgaaaaaaaaaaa

SEQ ID NO: 254 - DNA, 393 bp, Mus musculus
  misc_feature 59, 146, 220, 308, 309, 312, 321, 322, 348, 363: n = A,T,C or G
tgaggccccgctgtggaggtggccctgtgtcgagcggttctagagcccctgaagcccgncctgtggaccaaactccgaactctcagagcccaggaactacagacgactgctgcaggccgccaaatagccttgagggtaggagcggngcctgagggacagagcccggccttgcggaaccaggaaccgtatccacgcacgccttgctcacttgcatgccggngtgcgccccgcgccgcaaggtggcgctgttgctggcggtgtgcagcgacgtctacgcgggcctaggcggtggcgagaataaagagccnntgngggttgacnnctttttacctgacctgaccgaggagntgatctggagtccanatattggggaaacatagcgtggatgtggag

SEQ ID NO: 255 - DNA, 375 bp, Mus musculus
  misc_feature 20, 23, 42, 51, 62, 70, 80, 98, 110, 142, 152, 161, 165,173, 177: n = A,T,C or G
tgagacagacggagctggtncgnactcaggttgcctttacgnagacatcgntgccgggtcangagttacnagcattgctncactgcatcggaggctgnggactggctgcncagagcttctgcggaacagtcaaaactttggnccctaggtancctctcaancaancagtccanctgntgggaaaattcctgaagaatcatgttattgaggacatcaaggggaagtggggccaggaggattttgaagacaaccggcacctctacaggtaatggcagggtgtaaactctgaagaaggaacacggggcaagtgagtgcatcgggccagcatctggaaacttcccgaagtgaaagaatttgctgtgtccactgaagcag

SEQ ID NO: 256 - DNA, 352 bp, Mus musculus
gctgagggcaccgttctttatcaacaagacaaagattaaagactttcctcgattccctcaccggggcgtactgctggatacatctcgccattacctgccattgtctagcatcctggatacactggatgtcatggcatacaataaattcaacgtgttccactggcacttggtggacgactcttccttcccatatgagagcttcactttcccagagctcaccagaaaggggtccttcaaccctgtcactcacatctacacagcacaggatgtgaaggaggtcattgaatacgcaaggcttcggggtatccgtgtgctggcagaatttgacactcctggccacactttgtcctgg

SEQ ID NO: 257 - DNA, 197 bp, Mus musculus
actgagggacacagctggtatcacttacattgacaagagactccttgaacttgatgtgaagcctgtaattagaaagagcaatgatggcctcctcagcacggcccacatactctgtgctctctccatgaagttcaaggaaaggaacctgaaaaacagaagaagaaacctttcagagttccagagcagaaaaaataaaa

SEQ ID NO: 258 - DNA, 180 bp, Mus musculus
gactgagctgaagcctgtagtcgatatattcatgacttattcccttcactcatcaagaatctggatcctgtgccacttagacatttacttaatctggtctcagctcttgaaccaggtgttcatactgaacagacactgtacttggcatccatgttaattaaagctttgtggaataatgca

SEQ ID NO: 259 - DNA, 372 bp, Mus musculus
  misc_feature 100: n = A,T,C or G
agtgaacacatcgtttgctgcctgtgcgcgggctacttcgtggatgccaccaccatcacagagtgtctccacaccttctgcaaaagttgtatcgtgaagnacctgcaaaccagcaagtactgccccatgtgcaacatcaagatccacgagacgcagccgttgctcaatctcaaactggatcgggtcatgcaggacatagtgtataagctagtgccaggcttgcaagacagtgaagagaaacggattcgggaattctatcagtcccgaggcttagacagagtctcccagcccagtggtgaagagccagccctgagcaaccttggcctccccttcagcagttttgaccactctaaagcccactattatcgatat

SEQ ID NO: 260 - DNA, 300 bp, Mus musculus
  misc_feature 41, 45, 57, 79, 104, 121, 134, 135, 138, 139, 150, 162,170, 185, 194, 196, 200, 201, 204, 209, 214, 220, 272: n = A,T,C or G
tatgtagcatgcagtgacaacagcattgccccatcaattgncacnatgggcctcggngccacagaatatagaggaagcncccaagttgctggcctttacctgcngccttctgtgcggggcnctccatacactgnnctnncagagcctggncaccgcctgcgnggcatccntgcccgtctgtaagntccaggtgntnaacnnganttccngaggnccccanaccccaggcgttgcccttctgtaagcttagcctcggaatgtggcacctgacnctgcctgactgctcacctttgcaggtcg

SEQ ID NO: 261 - DNA, 335 bp, Mus musculus
  misc_feature 17, 24, 38, 40, 41, 44, 45, 62, 69, 86, 113, 121, 137,138, 145, 149, 151, 156, 171, 172, 177, 180, 183, 218, 237, 239,248, 251, 254, 262, 299, 302, 319: n = A,T,C or G
catggggatgacaggancccctgntgctgtccatgagnannctnncgctggagaggctgacnttgaccncagaggctctggaccantctttatagcttcctctatgagatcancacttgantggcacagcccttgcnnccattanaagntngaatnggtgatggatgactnnggccntgnacngacgaagagacacagacatcagaagatcctgcccnctgcgtcggaaggaaggcncnagtttcttngancanagagaacnaggcttggactggaagattttgagtctttaaaagtcntangccgaggagcatttggngaggtaaaaaaaaaaa

SEQ ID NO: 262 - DNA, 213 bp, Mus musculus
  misc_feature 16, 27, 60, 78, 99, 100, 147, 171, 186: n = A,T,C or G
gactgaggtagagcanatcctggcggngttccagctgcgggaggaagacctgaagaaggngatgagccggatgcaganggagatggaccgtggcctgannctggagacccatcacggaggccagtgtaaagatgttgcccacctacntgcgttccaccccagaaggctcanaagttggagactttntctccttagacctgggaaaaaaaaaaa

SEQ ID NO: 263 - DNA, 376 bp, Mus musculus
  misc_feature 341: n = A,T,C or G
actgaggttaaatgtatctttagaatacagtatgaagaggtattatatattcaaagacctattgaagaagatcgaagaaaatttttccaagaacttattctccatcaggcgtcaatggcaccaccacgaaggaaacatactgctcttcgtgctatggaagtgcttccgcttgcactaccatcacctcctcgtcagttatcagaatcagaaaaaaatcggatggaagaccaggaggaaaatacgttacgagagttgcggttgttcctcagggacgttaccaagaggctggccacagataaacgctttaacatcttcagcaaaccggtggatattgaagaggncttgtttcagtagtgtgcaaacagtgaagttgaac

SEQ ID NO: 264 - DNA, 387 bp, Mus musculus
  misc_feature 138, 173, 258, 266, 317, 324, 329, 334, 336, 343, 356,366: n = A,T,C or G
agatttgccaaagaggattcccaaggattcagcgcgttaccatttctttctgtacaagcattcccacgaaggagactacttagagtccgtagtttttatttattcgatgcctggatacacgtgcagcataagagagcngatgctgtattccagctgcaagagccctctgctcnagattgtagagcgacagctacagatggatgtgatcagaaagattgagatagacaatggggatgagctgactgcggatttcctgtncgatgaanttcaccccaggcagcatgcacataagcagagttttgctaaaccgaaaggancctgcgnccaangaggngnatccgangattgatctaggngcccagtgcnaccagaagccactactgacta

SEQ ID NO: 265 - DNA, 477 bp, Mus musculus
  misc_feature 17, 34, 64, 209, 214, 359, 361, 376, 381, 382, 392, 396,404, 405, 406, 413, 446: n = A,T,C or G
gggtaccgctgacatgnccactggcaaagacaancccatatatccttcggcgaacaaccatggnaaccaggaccagcccccgctttgctacacagaagttagtgggatcttccccatctctgggcaaagaaaatgttgtagagtcctccaaaccaacagctggtggcagcagatcacaaaaggtcaaggttgttcaggagagctcagcnggatncacacactgccttccaagaactcccagcaaaatctctcacagccagtaatattcctgggagaaactctacagagagccccagggagggcctgagggccaagccgggcctaccctgcctccagcccagctgctgggcctgatcccncnaacaacgaaaactgncgggnnccagtgaagntgtncactcagnnnatctggnaggtggcattctcaaaggagcctgcctgtgggnacttgaacttgagccaaggacacattatgta

SEQ ID NO: 266 - DNA, 117 bp, Mus musculus
  misc_feature 40: n = A,T,C or G
gcatgtggcagtcagctaacctcacacctcaaccccttgnctatgaccacaagctggaaggcttcttcgctcagaaattacctgacctagagacggtgatcaagaacccctgtctca

SEQ ID NO: 267 - DNA, 525 bp, Mus musculus
gactgagaggagaagatccgggagagtttgtggaagccttgaagacagagtttgctggcaaggggctgcggttctcccgaggaggcatgataagcttcgatgtcttccccgagggctgggataagcgctactgcctggacagcctggatgaagacagctttgacatcatccacttctttggaaatgagaccagtcctggcgggaatgactttgagatctatgcggacccccggactgtcggccatagcgtggtctcccctcaggacactgtacagcgatgccgtgagctcttcttcccagagacagcccacgaggcgtgactgggcccacagctgagcactgggaccttcaaagagctctgaccaggcctgaagatagaaccattgggtgccacggttcccatcccactggcccaacacgtagcatcctgtgggggcactgcagggtcacccacacaggaccaacgtctgtgtgggaactgtccccagccaggtggctcctgacgagacgcatacctgctctcta

SEQ ID NO: 268 - DNA, 293 bp, Mus musculus
gactgagaatatgtgctgtgtctcgaactctaagaaagaaattggccgctgttttaaactgattttgaaagctctggaaaccagcgtggatctgatcacaactggggacttcatgtccaggttctgctccaacctttgcctccccaagcaagtgcagatggcagctacacacatagcccgcaaggcagtggagctggacttggttcctggcaggagcccgatctctgtggcggcagcagctatttacatggcttcccaggcttcagctgagaagcgaacacagaaagaaatcg

SEQ ID NO: 269 - DNA, 273 bp, Mus musculus
  misc_feature 105, 159, 161, 177, 186, 203, 233, 235: n = A,T,C or G
cagaaggaggagatgggcatttcctgtgagctgctggaatcagacttcctcaagtgcagtgtgggatttcctttcatgaggtcaaagtctaagtatgaattcagngtcatctttgatacaagccacctgtctggggaagaggaaattctcagcttcatngngactgctcagagtggnaacttggancgctctgaagccctgcntgacaacactctcacactgacagtgccccntngtgcatgaagtggacacgttccatcactggaattgtgt

SEQ ID NO: 270 - DNA, 501 bp, Mus musculus
  misc_feature 318, 375, 408, 442, 480: n = A,T,C or G
tcctttcggcaacaaccccatacttccgctacctcttcggctggatggtgcctcccaagatctccctcctgaagctgacccagggcgagacgctacgcaagctgtactgagcagcaccacgtggtgcaggacatgctggtgcccatgaagtgcatgtcacaggccctgcataccttccaaaatgacatccacgtctaccccatctggctgtgcccattcatcctgcccagccagccaggactagtgcatcccaagggagatgaagcagagctctacgtggacatcggggcatacggggagccacgtgtgaagcacttngaggccaggtcctgcatgaggcagctggagaagtttgtgcggagtgtgcacgggttncaaatgttatacgccgattgctatatgaaccgngaggaattctgggagatgttcgatggctccttgntccacaagctgcgcaagcagttgggctgccaggacagnctttcctgaggtgtacgacat

SEQ ID NO: 271 - DNA, 208 bp, Mus musculus
  misc_feature 23, 24: n = A,T,C or G
actgaggactgcagcatccgagnncaaaatagacttagagatccgcatgcgagaaggaatatggaagctgctctcgctgagtacgaaaaaagaccaagttttgcatgcagttaagaacctcatggtgtgcagtgctcggatccaggcgtacacggcggagcctgcagaagtcaaaagaagagattgcaaatcaaaccggagcacgggt

SEQ ID NO: 272 - DNA, 256 bp, Mus musculus
  misc_feature 127, 132, 135, 178, 179, 219: n = A,T,C or G
aactgagtttatcagagaagatatgaaatataaagatgctacgaataaacatagtcacctgcacagagaagataagcacataactgttgaggatttgtggaaacagtggaaaacatcagaaggtaanactgngcngctgctctggagccttggcttgtctgcttctgccctgcttggnnttggcagccctactaattgcatataattatagagaagatnatgcttcatgtgagaatatttatatttaaaaaaaaaa

SEQ ID NO: 273 - DNA, 497 bp, Mus musculus
  misc_feature 21, 118, 163, 255, 299, 300, 391, 399, 436, 481: n = A,T,C or G
agatgtggtgcagacccaatngatgggaacaggctccagagcctctcagcatcccagcctgcactgctccttactgcacttgaacaacacgttttgtatttacagccttttttagcanaacttcagtctttaatgaaagaaaactcaacagctacagagatangacagacagaagcagagaccaagagtgaactaagagccatacatcccaccgaggacctacaagatgaagggaagcccaaagactgtgatgtnggagatgtgaaaaagacacaaaacctttttgacccagaagtagnncagatcaaggctggaaaagcagaaattgacagacgaatatctgcatttattgagagaaaacaagctgaaatcaatgaaaacaatgtcaggnaattttgnaatgctattgattgtaatcaagaaaatagtttgtgcnagaactgatgctgtcttttactccttatcctggatttaaaagtcntgtaaagggtttctag

SEQ ID NO: 274 - DNA, 199 bp, Mus musculus
  misc_feature 26, 40, 143, 152, 170, 182: n = A,T,C or G
ggagttccattgtgactggaagtctngcaggcatggtttncacgattgtcacataccctacagacctcatcaaaacccgattgatggtgcagaacgtgctggaaccatcatacagggggctaatccatgctttttctactatntatcaacangaaggggtcctggccctntatcagaggggnctcccctcagtgtttta

SEQ ID NO: 275 - DNA, 417 bp, Mus musculus
  misc_feature 17, 234: n = A,T,C or G
ggacacgcacaaggacntgtagaaagcatggtagagcttttggacttgtatcagatggaggatgaagcctacagcagccttgcagaggccacaactgaactctaccagtatttacttcagccattccgagacatgcgagaactggccatgctacgaagacagcagatcaagatttccatggagaatgattatttgggccctcgaagaattgagagtctacagaaagaagatgcntgactggcagcggaaagctcacatggctgttttgtctattcaggatctcaccgtcaaatattttgaaataacagcaaaagctcagaaagcctttccctaacaggattttgctttgtatttattgaagagcaaagaaacatgatatgagaaagaaataaaggattctattctacaaaaaaaaaa

SEQ ID NO: 276 - DNA, 288 bp, Mus musculus
  misc_feature 17, 33, 191, 193, 224, 236, 255, 261, 266: n = A,T,C or G
gccagcagcggacccgnaggcagctcgcccggnaaaagaagctctatgccaacatgtttgagaggctggctgaggaggagcacaaggtgaaggcagaagtggcagcaggagaccatcccactgatgctgagatgaagggtgagcggaacaatgtggccgagaaccagttctcgggtggagacagaagcagnancctgcctcttccgctgcccacctctgcagcntgctccctgctnccttattactccaccctgntagttntgcanaaactgaagaattttgagtgaa

SEQ ID NO: 277 - DNA, 295 bp, Mus musculus
tgcaacacccaacacatataagaggaagaacacagaaacagctttggacaacaagccttgtggaccacagtgttaccagcatctggagggagctaaggagtttgctgctgctcttactgctgagcgtataaagacaccacctaaacgcccagggggcccgcagaagaggaagacttccgaataacagtagcagacccagcacccccaccatcagtgtgctggagtcaaaggatacagacagtgacagagaagcagggactgaaactgggggagagaacaatgataaagaaaaaaa

SEQ ID NO: 278 - DNA, 123 bp, Mus musculus
actgaggattgcctgaactagaattaggaagcgatcgataaccagtttggacaaccaggagcagggggatcagattccatgggcaaataatactttgacaacaataaatcagaataaaccaga

SEQ ID NO: 279 - DNA, 390 bp, Mus musculus
  misc_feature 219, 312: n = A,T,C or G
aggttggcaaactgtatgtttgggatttagaagtagaagatcctcataaagccaaatgcacaacactgacccatcataaatgtggcgcggctattcgacaaaccagtttcagtagggatagcagcatcctcatagctgtctgcgatgatgccagcatttggcgatgggatcgacttcgataaactattttttcctagcaaaaattagtctgttgtctgnaaaatagaagtaatgtatcttgctagtaagggcacatagagcatttagacttgtttttcagcattcgatcaggctgagctgaatgtagccgangttcacattgtttacattctttgtactgtcttcctgctcggactttactgcttttaataaaaatttatttttgtaaaa

SEQ ID NO: 280 - DNA, 472 bp, Mus musculus
  misc_feature 440, 455: n = A,T,C or G
cctgtgtgtgaggagtcggcctgagcacttccaggacgccacaaaaccttttctacgtttcgggcttcatttcacagtgcacgcacggccgccgggaggagtgcaacagacaggcagtttttcttcatcaatcagaggccctgtgacccagcaaaggtctctaagcttgtcaatgaggtttatcacatgtataaccggcatcagtacccatttgtcgtccttaacgtttccgttgactcagaccccagactctgaacttaactgctgtcaatgaagctgtactgatagaaaatctggaaatattcagaaagaatggctttgactttgtcattgatgaggatgctccagtcactgaaagggctaaattgatttccttaccaactagtaaaaactggacctttggaccccaagatatagatgaactgatctttatgctaagngacagccctggggtnatgtgccggccctcacg

SEQ ID NO: 281 - DNA, 519 bp, Mus musculus
  misc_feature 17: n = A,T,C or G
ctgagtttttcgtaatnagctaccaaggaacaacgcattctactcctatgagcccccttctgaaacagaagctttgggtacggaatgtgtgtgcctccagctcaagtctggagcccatctctgtcgggtttgtggttgcttggcccctatgacatgctctaggtgcaaacaggcacattactgcagcaaggagcatcagacattagactggcggctgggacacaagcaggcttgtacacagtcagacaaaatagaccatatggttccagaccacaacttcctgtttccagaatttgaaattgtaacagaaacagaagatgaaattttgcctgaggttgtggaaatggaggattattctgaagttacaggaagcatggggggaattcctgaggaagaactagattccatggcaaagcacgaatccaaggaagatcacatattccaaaagtttaaatctaaaatagcccttgaaccagagcagattctcaggtatggaagagggattaaacccatctggca

SEQ ID NO: 282 - DNA, 526 bp, Mus musculus
  misc_feature 36, 91, 416, 440: n = A,T,C or G
gactgaggtactgcagggtgctcagctcatagccgnggcctcttcagaccctgctgctacaggagtagatgggtcgcctctccagggcagngacattcaggttcagtatgtccagctggcgcctgtgagtgaccacacagccgcagcgcagaccgcagaggccctgcagcccacatctgcagccctgacatgcagcgttgaacatggggccatccagatccagtgaggccaggcactgcaggagcaccgagtcacagctgctcgctgaccctgccccactcgtgccctgctctcttgatttcagcaagcaactgcaggttctgctgggcatctgagagctgctcctcccaggggaaggtcctggccacccctgctggaaggcgcctcagggttggagtctcactactggtcgtctncaaaggagaagcatagtgcagagngttgagtgcattcagacagacaagaactacgatattttgtttaaacagcttttttaatttgctatggtgtttataacaaaaaagat

SEQ ID NO: 283 - DNA, 482 bp, Mus musculus
  misc_feature 16, 26, 28, 46, 48, 55, 56, 64, 81, 83, 94, 95, 98, 106,111, 137, 152, 165, 174, 176, 215, 216, 227, 245, 271, 284, 286,295, 299, 302, 312, 314, 315, 319, 351, 379, 400, 448: n = A,T,C or G
gactgagaagagtaanaatgacatangnaaactcatcttcaaaagngngtgactnncaatatgntcccatacaccgatgtncncaagaatcctnnctngcaagagnggggncgagaagccattgaaatgcacgagancaggaagcaccaaanacctcctgcagangacagacgngnacatactcgcccacaatctgtaaggaactcctgaacatnnaacgcatcagnactgtacccagcctacantgaacatgcaccgcgattagagacanctcttgcattttncngagcacagnatanccnatctttcatnanngacnagagcaggaggagagactacttgtgagtccangagacagaggtgaggccccctgtttctntgagccacttccccggacatntggctcagagcactacacccccaaaagggagcaccatgcactagctgntcctggtactccgtagagccacgcttgaggggcg

SEQ ID NO: 284 - DNA, 244 bp, Mus musculus
  misc_feature 16, 31, 60, 107, 165, 175, 176, 183, 191, 198, 199, 200,219, 226: n = A,T,C or G
ggtacctgctcaggcncacctcgtgatccgngaagactgccgagggaacattctgattcnaggcctgacacagaagcccatcaccagcttctctgacttcgagcagnacttccttccagccagtagaaatcgagctgtaggagccacccggcttaaccagcgctnttcccgtagnnacgcagngctctagnccaaggnnnatcagcgtgaacgtttgantccatcncggcagtgggaaggaaca

SEQ ID NO: 285 - DNA, 499 bp, Mus musculus
  misc_feature 373, 439, 449: n = A,T,C or G
tgagcatctgaagaggatgtgagtcagtttgattcaaagtttacacgtcagacacctgttgacagccctgatgactccactctcagtgaaagtgccaaccaggtctttctgggttttacatatgtggctccatctgtacttgaaagtgtgaaagaaaagttttcatttgaaccaaaaatccgatctcctagaagatttattggtagtccacgaacacctgtcagcccagtcaaattctctcctggggatttctggggacgaggtgcttcagccagcacggcaaatcctcagacccctgtggaatacccaatggaaacaagtgggatagagcagatggatgtgacagtgagcggggaagcatcagcgccacttncaatccgacaacccaactccgggccgtacaaaaaacaagcttttcctatgatctccaaacggccanagcacctgcnggatgaatctatgatgaaacaatgcttttattaatgcaaaatcatatagg

SEQ ID NO: 286 - DNA, 451 bp, Mus musculus
gactgaggttatttgtatctaccccgcttatttaaataataagaagaccatagctgaggggagacggatccctataagtaaggctgtagaaaaccctacagctactgagattcaagatgtatgctcagcagttggactgaatgcctttctggagaaaaacaaaatgtactccagagaatggaaccgtgatgttcagtttctaggcagagtccgggtacagctcaaacaggaagatggcagcctctgtcttgtacagttcccgtcacgtaagtcagtgatgctatatgtagcagaaatgatacccaagctaaaaacacggacacagaaatcaggaggtgccgacccaagtcttcagcaaggagaggggagtaaaaaagggaaaggaaagaagaagaaatgacgtggtgtgaacgtggagcacagtcctgctgggagagacatggcatccttg

SEQ ID NO: 287 - DNA, 381 bp, Mus musculus
  misc_feature 23, 179, 210, 246: n = A,T,C or G
gtttgagcttcctgttgagcganttatgaacctttctacaagggtgggaaagcacagctggaccagactggccattacctcttctgtgtctgtggtaccaaagttaacatcctggatgtggcctcaggggctttgcttcggagcctggagcaggaggaccaagaggatacacacagctnctgtggcctccatggcttttgatgccacctntacactgttagccacaggcggctgtgatggggctgnacgtgtttgggagcaggacactgcaagcagccaccttcttggacttcctgtggcacaacatgaagctgtccccatgccctgctgcagctcccccagccctatgaaacagaaaagttaactttctaaaaaaaaaaa

SEQ ID NO: 288 - DNA, 546 bp, Mus musculus
gtttgagagacagtggagatcctgttgttcctcagtgccattgtgatgatgaagaaccgcagatccatcactgtggagcaacatgtaggcaacatctttatgtttagtaaagtggccaacgccatccttttcttccgactggatattcgaatgggtctgctatacctcacactctgcatagtgttcctgatgacctgcaagcccccgctgtacatgggtcctgagtatatcaagtacttcaatgataaaaccattgatgaggagctggagcgagacaagagggtcaccttggattgtggagttctttgccaactggtctaatgattgccaatcctttgctcccatctatgcggacttgtccctcaagtacaactgttcagggctaaattttgggaaggtagatgttggacgctacactgacgttagcacacggtacaaagtgagcacatcacccctcaccagacagctccctaccctgattctgttccaaggcggcaaggaggtcattcgtcggatcgtagattgacaagaaaggacgagctgtct

SEQ ID NO: 289 - DNA, 405 bp, Mus musculus
  misc_feature 249, 282, 285, 347, 354, 362, 375, 380, 388: n = A,T,C or G
attgaggacacaatcacaaagaaaggcaggtggcgggagtgcaccaccatgtgacagcattgttgtggcctactatttctgtggggaacccatcccctaccggaccctggtgaggggccgtgctgtcaccctgggccagttcaaggagctgctaaccaagaaggggagctacagatactactttaagaaagtgagtgatgagtttgactgtggtgtggtatttgaggaagtacgggaggatgaggccgncttgcctgtctttgaagaaaagatcatcggcanggnggaaaaggtggactgagcactgggcagcacacccggagcacaccatcactactgtgcattgncatcagncaggtggncagccttgtcctnaggancctggtgnggggaacaacacaagat

SEQ ID NO: 290 - DNA, 303 bp, Mus musculus
  misc_feature 20, 26, 40, 41, 53, 58, 59, 182, 207, 227, 253, 257: n = A,T,C or G
cgccaaccctgagtaacagngacctnaagtattcaggcanngacggcgctctncatanntgtcagtctgtcctcccctgccgaagtcacatcctccgtgcgacaggattcttggtgtgctctggccctggcctgagctggtgctaccaagaggcaaggagggctctgaaggcctcataccanttgtgtctgatattgtccagcagtncagatggcagcaaaaatgcngacataccattccttcgatgcgtganttcngtgcctttgttttgaaagagatatatttctcaagaagcttactgaa

SEQ ID NO: 291 - DNA, 318 bp, Mus musculus
  misc_feature 43, 217, 253, 270, 284: n = A,T,C or G
atgaccgctcttctcacgcgcataccaacaacattcgcaaagnctgtggtcaaccttgtagattctatttacaaggaacagctcaacaccagggttgtcctggtggcatgtcgagacctggaccgagaaggatcacattgacatcaccatcaaccccgtgcagatgctacatgacttctccaagtaccggcagcgaatcaaacagcacgctgacgcnggtccacctcatactcgcgcgtgacattccattatnagagaagcagtctgagntactttggaggcgngtgttctcgaataagaggggctggtgggaatgag

SEQ ID NO: 292 - DNA, 453 bp, Mus musculus
  misc_feature 19, 20, 26, 269, 279, 282, 305, 307, 310, 373, 422, 425,436: n = A,T,C or G
cttggggctggacaggtannacgganggttgcatagccccacctttggatcacatggagaatcagcaggaccttcaggactccagagtttgaagaatttaactgggaagccccgactccctctttttcaccgatggccagaggcgaatcgacttcatcctcgtgtatgaagatgagagcaaaaaggagaacaataagaaagggacaaatgagaaacagaagaggaaaagacaagcatacgaatctaaccttatctgccatgggctgcanctggaagcancangatctgtttctgatgacaagctngngtncgtaaaagtgcacgcgccctgggaagtgctgtgcacctatgctgagatcatgcacatcaaacncccgctaaagccaaacgacctgaaaacgcgctcgccctttggcaacctnaantggttcaccanggccctccgggtgaacg

SEQ ID NO: 293 - DNA, 511 bp, Mus musculus
  misc_feature 456: n = A,T,C or G
ctacaacgttgtcagtgacgaagcgctctgggggagacatcctccagtctggctgctgaggttaagccccagtgtggatgctgttgccaagactacagaccattgccttgcttccttgcccacgcccaggtgaagttcaggaaggctcttgggtcctaggcgccaattcaaggtgctgtcctaaggccaccgggtccctgggatcttgtgggtaggaggtggcaggtcgaaggaggctgcagcatcgcactggggtcaccatgacagactcagactgacatctggcagagcatcacaggcatgcgtccatgaagtcactggcctcaagcccaagtggtgggcagtgacagaagagctgccgggtctgttgagctcaaccttttcctgtagattgtcttagtctcactttcaagctgtctaatgtcaattctgtttttcttttttcctccatggggntaatgatactagagatagggaatattagcaatcagtttttgtcatggctggtccc

SEQ ID NO: 294 - DNA, 278 bp, Mus musculus
  misc_feature 26, 31, 110, 140, 159, 168, 169, 170, 173, 184, 193,196, 205, 216, 222, 223, 224, 225, 228, 230, 232, 233, 247, 258: n = A,T,C or G
agaagattaaagtctatgagtatggnacagncatccaggatgcagtggatattcattaccctgagaatgaaatgacctgcaattccaaaatcagctgtatcagttggagnagttaccataagaacctgctagccagcagngattatgaaggcactgttntactatggnnnggnttcacaggacngaggtcaanggnctatcaggngcatgaaaaanggtgtnnnnagngntnnctttaatctgatgnatcctaacctntctggcttcattgttctgat

SEQ ID NO: 295 - DNA, 371 bp, Mus musculus
  misc_feature 29, 34: n = A,T,C or G
tgaggtatacaatgtttacatggctgggnggcanctgtgttctaagcggtaccgggagtttgccatactgcaccaaaacctgaagagagaatttgccaactttacatttcctcgacttccaggaaagtggccattctcattatcagaacaacaattggatgcccggcgtcgtgggttggaagaatatctagaaaaggtgtgttcaatccgagtcattggtgagagtgacatcatgcaggaattcctgtctgagtctgatgagaattacaatggtgtgtctgatgtagaactgagagtagcactaccggatggaacaacggttactgtcagagttaagaagaacagtactacagaccaagtgtaccaggcta

SEQ ID NO: 296 - DNA, 334 bp, Mus musculus
  misc_feature 48, 78, 93, 99, 100, 117, 132, 136, 140, 156, 157, 168,174, 181, 188, 194, 214, 215, 218, 230, 237, 270, 278, 298, 311,314: n = A,T,C or G
ggcgtggacagcacagggaccaggctgctctttatggctaatgaagcngacttggatgaagagctggtcattaagggntccatatctgcaagnagcacnntatgatacactagcacncaggcacgttgttgnatcncccntcctctacatgcctgnngaagatacacntggggntaggatnaatgganaatggngcatgacagctctttatcanntanccagcttgtatncataccnacgacgcatctgaccttgccctccctatgatgnctgctggnctgctttgacgaggagacangatggaaagattnggntagagcacaggtccgatgct

SEQ ID NO: 297 - DNA, 236 bp, Mus musculus
  misc_feature 94, 130, 156, 189: n = A,T,C or G
ctgcatataacctaactggcttcaacattttttattcgatcaattcctgtcctaacaactgctctggtcatggaaagtgtacaaccagtgtctntgttgcaagtcaagtgtattgtgaatgcgacaaatnctggaaaggggaagcatgtgacattncttactgtaaagccaattgggggagtccagatnatggctactgtgacctaacaggagagaaactctgagtctgcaacgat

SEQ ID NO: 298 - DNA, 530 bp, Mus musculus
  misc_feature 155, 161, 167, 170: n = A,T,C or G
gactgaggtacccgtcccgcgtgtttgtgggagacaccttctgttactttgcgggcatgacttttgccgtggtggggatcttgggacacttcagcaagaccatgctgctcttctttatgccacaagtattcaatttcctctactcactgccttanacttgncatatnatnccctgccctcgacaccggatgcccagactcaacgcaaagacaggcaaactggaaatgagctattccaagttcaagaccaagaacctctctttcctgggcacctttattttaaaggtagcagagaacctccggttagtgacagttcaccaaggtgagagtgaggacggtgccttcactgagtgtaacaacatgaccctcatcaacttgctacttaaagtctttgggcctacacatgagagaaacctcaccctgctcctgctgctcctgcaggtcctaagcagcgccgccaccttctccattcgttaccaactcgtccgactcttctatgacgtctgagctccctgacagctgccaccttaa

SEQ ID NO: 299 - DNA, 505 bp, Mus musculus
  misc_feature 33, 188, 197, 200, 205, 220, 228, 240, 271, 272, 274,275, 281, 294, 295, 301, 305, 306, 307, 312: n = A,T,C or G
actgaggaggaagaccatgtgcggcacgctggnctatctgcccccagagatgattgaggggcgcatgcataatgaaatggtagatctatggtgcatcggggtgctctgctatgaactgatggtggggaacccacccttcgagagccctagccacagtgagacgtatcgtcggattgtcaaggtgcganagcctttgngtnctgtnggagccagggtctgncgacactnaaagtggatgtncacggcatctgcaaaaatgttcctgctttgnnannactttntatttattctatnnttgaantccnnnccaantcaaggagggttctgcctccctctgccctttagcctgctccttggttttttgtccctgtcatttttcagtgttctttgtatgtctgtgtatgtgttctgagaaggggtgggaactggaaactattcctagctccagttctaggggatctgatctctcttctgacctctacaggcaaaattaggcacccctgtt

SEQ ID NO: 300 - DNA, 562 bp, Mus musculus
  misc_feature 23, 34, 42, 60, 61, 63, 66, 67, 73, 75, 77, 88, 97, 100,101, 102, 110, 114, 116, 119, 120, 127, 137, 146, 153, 154, 156,160, 172, 173, 174, 177, 189, 195, 202, 216, 241, 242, 243, 248,312, 362, 416, 444, 463, 492, 515: n = A,T,C or G
acctaggacgcagcttttcttgngaactggccgngatggcgnagggtacacagaacaggnnantgnnctatcngngntccggagcccnactgccagnagnnngatgaccntgancncannacatacnggcatgaganggtgactgntactgannangacnttcatgctcacnnnagnccatgcatggangagctncgtatgncatgatcacattgnttatacacacgcgtggaaagactgnnnaatcntggtattcccagagctttgggttacatttttcactgagggaagaacactggcaagaagactggncattcatactctctctagctagtcagcctggagcaagtttggaacagacncacatttttgtacttgcacatattcttagacgaccaattatagtttatggagtnaaatattataagagtttccggggagaanctttaggatatactcgggntcaaggagtttatctgcctttgttgcggnaacagagtttttgttggaaaagnccaatcgcactgggctatacaaggggccacttctctgctttggttgc

SEQ ID NO: 301 - DNA, 305 bp, Mus musculus
ggttattgaaaatgcaagagagaaaatattaagcaacaaatctcttcaagaaaaactagcagaaaacataaataagtttttgactagtgatagtagtgttgctcaagtacctaagcagacagacagcaacccgacagagccagaaacgtcaattgatgaactgctaggccttccgagtgaaatccatatgtctgaagaagccatacaagatattttggagcagacagaatcagaccccgcctttcaggcactctttgatctttttgactatggtaaaacaaagaacaataaaaatatgccacaaa

SEQ ID NO: 302 - DNA, 371 bp, Mus musculus
tgaggaaaccaagtatcttttaccaagtctggaaaaagaattattcttggcagagcacagtgatcttgaagaaggtggactggatctgaatgtctccttaaaacctgttagtttctatatatcagacaaaaaggaaatgctgcagcaatgcttctgtatcataggagagaagaagttgcagaagatgctccctgatgtacttgaaggtactttcccagtgagctggctgcacacgcggagaaacagccttcatctgtagcagtcatgcctttgaatgtagtgtgtgattggatgttctaatctgaaacaagttacttgaaagaatttaaggaagaaaatttagagaaccttaaataaatggccttatatat

SEQ ID NO: 303 - DNA, 232 bp, Mus musculus
  misc_feature 176, 215: n = A,T,C or G
gactgagagtacccctgcgaaagagctcctaaggaaggagctgcaaattccggactgtccattctactggagcggatcatttgtgatcttgaaaaatgccttgggacagctttatcttcaatattagaaacagaaatgaaaattgcttttggaaatctttggatggaggtgcctgnatttgaaaccaccatggactctaataaatctattacagngctttaaaaaacaaaaa

SEQ ID NO: 304 - DNA, 356 bp, Mus musculus
  misc_feature 18, 26, 44, 50, 52, 54, 57, 75, 76, 78, 92, 96, 99, 134,145, 240, 260, 277, 303, 311, 316, 320: n = A,T,C or G
gttacataacgtactatnagacaatnactggaatgagttctganggaggngncnagnaaaccgggtggctcattnnanaccgcatgaccctnatcngcngagaggaaaaacagcttgaaaagaatttacaaganttggctacagnattagctccactttacaagcagatggctccagttgcttatcaaaattaggcggaatatgaagaagttggtggagactgtccacttggaaatgaanaggggcgtcctttctctggngtcacctgttgcatggntttttgtgcccattctcacaaggacnttcacatnatgcncaantggaagcacccgcggtgtgtactgttgattcgagca

SEQ ID NO: 305 - DNA, 204 bp, Mus musculus
gagaagccaaccttaatgcagcacattctcagatgtgactacgaggcctgccgacagtatctcgtgaatctggagcaagccgtggttttagagcagaacctgcagatgttacagacgttcatcagccacagatgtgatgggaaccggaacattttacatgcttgtgtatcagtttgctttccaaccagcaataaggaaactaaa

SEQ ID NO: 306 - DNA, 560 bp, Mus musculus
  misc_feature 208, 507, 511, 513, 515, 525, 528: n = A,T,C or G
actgagatcgagatggcacagaagctgctgactccgacttaggcgagctcatcagcaagatgaagctggcgcagcagtacgtcatgaccagcctgcagcaggagtataagaagcagatgctgacagctgctcacgccctggctgtggatgccaagaacctacttgatgttattgatcaagcaagactgaaaatgctagggcagacacnggccacactgagcccctggccattgaacgtggctttctgccctttgggagatcttctctagcattccaccagcagcgaggaagtaaccttagcctctgtagcccgtgctcacggctccaactgaatgacagctagctgaaagtctctcttgtataagtttcatcacttatacatggttttgattttgttatttttctaaatagtgttcagaaaactctggatccaaaatgtggcatttttctgagaatgaagattatacattaagaagcttttaaaatatacttggtgttcaaattggnatgngngntcctagaagnggnaattcatactgcctccaacgtctgtacaagag

SEQ ID NO: 307 - DNA, 102 bp, Mus musculus
  misc_feature 19, 20, 37, 73, 74: n = A,T,C or G
cttccctgaggctacgtgnngacctgccggagagcanacaggacagctcagcgcagatccgcagacgaaaggnntccggagacccatactgggcgtattctg

SEQ ID NO: 308 - DNA, 395 bp, Mus musculus
ggtctttcaggcacactcttccgtgtccatgctcggccagacctcaaccaccgcttccaggtgttccgcggtattctcgaggaccagtgccgccagctggaccccgacccatagttctggtgacctgcttctagaccattctctgctcctggccagcccgtgtcccctgcggctccatggcctgggggcctcaccctctgtgatgatactgatcacctgtgccagactgtgggcctctcagtttccagactgcagcttctctgctggctgcgacgacagtggggctcagctctgcttacaagcccagtggctgtcttacagtccccaggtcctccaggctcggggcagggagcagccatcgagtgtgaaaataaaacacctcaaaccaaaaaaaa

SEQ ID NO: 309 - DNA, 527 bp, Mus musculus
  misc_feature 475: n = A,T,C or G
ctccttactatgacaacgtccgtccctctcttacccagattctgatgcagtgctcatttgctttgacatcagtagaccagagactctggacagtgtcttaaaaaagtggaaaggtgagatccaggagttttgtcccaataccaagatgctgttggtgggctgcaagtctgatctgcggacagatgtcagcacattagtggaactctcaaatcacaggcagactcctgtgtcatatgaccagggggcaaatatggccaagcagatcggagcagccacttacatagaatgctcagctttacagtcagaaaacagcgtcagagacatttttcacgtcgccaccttggcgtgtgtaaataagacaaataaaagcgttaagcggaacaaatcgcagagggccacaaagcggatttcgcacatgcctagcagaccagaactctcagcagttgctacggacttacgaaaggacaaagcgaanagctgtactgtgatgtgaggcttcgccgtctataaggaggacgcgggaatat

SEQ ID NO: 310 - DNA, 318 bp, Mus musculus
  misc_feature 25, 36, 109, 113, 132, 149, 153, 160, 162, 163, 165,167, 170, 173, 184, 187, 197, 199, 203, 208, 210, 211, 221, 222,229, 233, 239, 259, 267, 286, 290: n = A,T,C or G
ctcttttaccgaagtttatcctcanagttgaagaanacacaggatcagctttccgcgtatccagagaaacacagagaagccacatgagggtgctgtgaggctggttganaagngcagtgaggtggacctgancatcgtcatcctatggnaggnatacctntnngnanatnccntgcagcgttanttnacaaggccancntcanggttntnnatcgcacttnntgggaangaancctttncacactctcagaaagagganccaccatnaaagggaactattaaaganttgntcaggccacaaaacactacagtttccac

SEQ ID NO: 311 - DNA, 269 bp, Mus musculus
  misc_feature 34, 38, 46, 61, 83, 89, 95, 106, 108, 117, 120, 163,178, 180, 182, 183, 194, 195, 206, 207, 220, 225, 231, 246: n = A,T,C or G
gtagcaaatgtctgttattaatcagactgcaggngatnccacagtncttgatagataatgntccaaaaaggtcttccttaatngaaganacaaangaatggccttngnaaagctttngtnaacaactttgcaatgacctttgtaatccctcctgggaggttcnacatggggcaccgtncngnnatttaaggctnnacaaatatgtnncatgcacaccctnacccncaccgnaaaacacaaatagtnttatgaaaaggaaaaacaaaaaa

SEQ ID NO: 312 - DNA, 444 bp, Mus musculus
  misc_feature 33, 100, 104, 128, 129, 133, 138, 140, 152, 161, 169,179, 184, 191, 193, 194, 199, 201, 202, 215, 220, 222, 248, 255,297, 300, 315, 332, 346, 352, 358, 361, 362, 363, 364, 375, 380,381, 392, 397, 400, 401: n = A,T,C or G
actgaggtgactgtttgaattttggaataaagnctcttgaggaaattaaatcaaagaaaatgaaggaaaaatccaagaagcaaggtgaaggctcctcggnagtntccagtgttttacagcatcctcannccantccangncctgagaagganaatgttccnactgtggngaggatggcnacttntatccancnnaccanannagcccttgtttanactgngnctttctgagagactggggaaacgaanactttcngtgggtggtgatagtgaccctccattaaagcgaagccttgcncanaggctgggaaaaaangtggaatcagccataanctaacattgataangcaccnaagaanggnnnngtctttgcttnttacnnacatggcctgnctgtntgnncttatatccggtgtggtccccgttcttcgctcaggtgaacgtt

SEQ ID NO: 313 - DNA, 402 bp, Mus musculus
actgagggattcgatcagtgagcttctatgaacacattatcactgtgggcacagggcagggctccctgctgttctatgacatccgagctcaaagatttttggaagagaggctctcagcttgttatgggtccaagcccagactcgcaggggagaatctgaaactaactacctggcaggggcgtggcttgaatcatgatgaaacctggaggaattacttttcggacattgacttcttccccaatgctgtttacacccactgctacgactcgtccggaacgaaactctttgtggcagggggccccctcccttctgggctccacggaaactatgctggactgtggagttaatgacaactaactttctcccaaaatgcagatttacactaacttccaaaaaaaaaaa

SEQ ID NO: 314 - DNA, 105 bp, Mus musculus
  misc_feature 62: n = A,T,C or G
acggaacacgaggagatgatgccttcagagcaaatgtgtatggtgactctatagttgtgcancaacacattagcgtggatggaagtcgatcttacaaaaaaaaaa

SEQ ID NO: 315 - DNA, 280 bp, Mus musculus
  misc_feature 26, 204: n = A,T,C or G
cattcccaggtttgatctgccagcangacacgcctgactgctccagtcacaggaacttaatgaaggcaacctttccaccatccagcggcggtgccgtgaccactccaaagggcagctccaggatgaagctgtccagacctaaagggagtgttggaacttgccaggcggttagggttgagggcggggtcgatagggggcgctctnaccctcaggcttacaacaggaaggaagccaccttcctcaccccacttgtacctcaaactttttaaaaaagaaataa

SEQ ID NO: 316 - DNA, 517 bp, Mus musculus
  misc_feature 92: n = A,T,C or G
aactgaggcactataagactgacttcgacaagaataagatctggtatgaacatctggctcatcgacgacatggtggcccaggtgctcaagtnttccggtggctttgtgtgggcttgcaaaaactatgatggagacgtgcagtctgacatcctggctcaaggctttggctccctcggcctgatgacatctgtgctggtctgccctgatgggaagacaattgaggctgaggctgctcatgggacagtcacccgccattaccgagaacaccagaagggccggcccaccagtaccaaccctattgccagcatctttgcctggacacggggtctggagcatcgtgggaagctggatgggaaccaggaccttatcaggtttgcacagacgctggagaaggtgtgcgtgcagactgtggagagcggagcgatgaccaaggacctggctggctgtatccatggcctcagcaatgtgaagctgaacgagcacttcctgaacaccacagacttcctggacaccattg

SEQ ID NO: 317 - DNA, 360 bp, Mus musculus
aactgaggatgaattgatcctgagttaaagcagacactaaatgctatcaaactagaagaaaaaggagtttatgtccaggcatctacactgggctctttggaagctcttcttgaatttctgaaaacatcagaggtgccatatgcaggaattaacattggcccagtccataaaaaggacgttatgaaggcttcagtgatgttggaacatgatccccagtatgcagtaattttggcctttgatgtaagaattgaacgggacgcacaagagatggctgatagtttgggagttagaatctttagtgcagaaattatttatcacttatttgatgcctttacaaaatacagacaggactacaaaaaa

SEQ ID NO: 318 - DNA, 524 bp, Mus musculus
  misc_feature 464: n = A,T,C or G
tgagccttttggccataaccagtcccagcaagtatattctacaagagaatactattcttaaggctactgaagtacagttcccgccaaagccagtagtaacacctgaagcaaaggcatttatcaggagatgtctggcctatcgaaaggaagatcgcattgatgtgcagcagctggcctgtgacccctacttgttgcctcacatccgaaagtcagtctccacaagtagccctgcaggagctgctattgcatcaacctctggggcatccaataacagttcttcgaattgagaccaactcctaaggccacaaactgttgagcacacacagtggacatacggcattcagcagtgggtttggaacatagtgaatccgaatggatctcatgaaacctgtaccaggtgcttttattttcttgctttttttccccatccatagagcatgacagcatcgattctcactgagganaagccttgggcagctctggccaggcctcataggaaaaaggtcccgcccaaagttccttgc

SEQ ID NO: 319 - DNA, 153 bp, Mus musculus
taatgaaaaaaaggatcagccctggagcaaaatgagtcacagaccttcaaagtcgaggtatcgtatatggagatctataacgagaaggtccgggacctcttggaccccaaagggtgagtgagtgctttgaagtaaagtgtaaaattaaaaaaa

SEQ ID NO: 320 - DNA, 199 bp, Mus musculus
  misc_feature 16, 129, 134, 160, 171: n = A,T,C or G
gcccagtttgcagtcnagtagtgtgctgagagacacatcatagacaacttttttgtgaaggacaccactgaagttcctagtagtacagtagaaaagtctaatcaggtatgtacaagcctgtgaagacantgcanaagctaatgggttttgtgtagagtgngtttgaatggntctgcaagacatgtattagagctcacca

SEQ ID NO: 321 - DNA, 219 bp, Mus musculus
  misc_feature 167, 195: n = A,T,C or G
ctcccggctgcgagaggcgcagcctgcgccactccagacagctggaggtgcctcggccctacatcgcgcgctcgattctccatcctgccagctgtcttccatcctgggaaccagaagcaatatggtggctttgacaacaggggcttggagccaggccaccgctatgncctctttgtgcttgctgtgttgcagaanaatgagcctgtatgtccaaaataa

SEQ ID NO: 322 - DNA, 516 bp, Mus musculus
  misc_feature 475: n = A,T,C or G
aactgaggtacaactgatgagaaaaaaagagcaagagaggaaaggagagacttctgctgccaacatgcagagaagtattatgtcattttttcaacccacaaaagaaggtaaagcgaagaagccagagaaggagacacccagcagcatcagagagaaggaaccccctccaaaggtggccgcctgaaggagaggaatcaagtggtgcccgagagtgattctccagtgaagaggacaggaaggaaggtagcccaggttctgagctgtgaaggggaggacgaagatgaagcccctggcacccccaaagtccagaagcctgtgtcagactctgaacagagctctcctcccagccctgacacatgtcctgagaacagtcctgtcttcaactgcagctcccccatggacatctccccatcaggattcccaaagcgtcgaactgcgcggaagcagctccccaaacggacaattcaagacactnttgaggagcagaatgaggacaaaaccaaaacagctaaaaaa

SEQ ID NO: 323 - DNA, 354 bp, Mus musculus
  misc_feature 192, 202, 208, 221, 224, 234, 235, 236, 239, 245, 255,257, 269, 289, 296, 302, 333, 337: n = A,T,C or G
gactgaggggctgactagaggagacagcaaaacacgaagaaacttgacgccatgaccctcattaaagaagacatgtccatcttcgggcactgccctgcccatgatgacttctatttggttgtatgtaaccactgcagccaggtggtgaagccccaagccttccagaagcattgcgaaagaagacatgggccnctcagcaagntttatncccgggccccacnccnacctgcagcnnntgncagaangtcaaatacngncggtgcacgcanactggctatgtgtacaaaanggactancgatanggtataattctattcatctgtcatcggaagnattnttctttgcaggattctt

SEQ ID NO: 324 - DNA, 516 bp, Mus musculus
  misc_feature 28: n = A,T,C or G
gactgagccacccttggggaagtctggntccagaagacatctgaaatgaatacagataaacagtatttttgccgaactcacttgggacatcttttaaatcctggagacctggtgctgggatttgatttggccaactgtaacttaaatgatgagtatgtcaacaaaatgaactcagacagagttccagatgtggtgttaattaagaagagctatgaccgcaccaaacgccagcgccgtagaaactggaaactgaaagagcttgcaagagacagagaaaacatggacacagatgatgaaaggcaatatcaagattttcttgaagatcttgaagaagatgaggcaattagaaaacatgtcaacatttacagagatgcaaccatccctgtggaaagtgacacagatgatgaaggagcacctcgaattagtctggctgagatgcttgaagaccttcatatttctcaagatgccactggtgaagaaggtgcatcaatgatgtcatgatgagtcatattattt

SEQ ID NO: 325 - DNA, 312 bp, Mus musculus
acactcccaaaagggaaagcttgtcccacgaggagcacccccacagccaccctctctatggacatggcgtatgcaagtggccaggctgtgaggcggtttgtgacgacttcccagcctttctaaaacatctcaacagtgagcatgcgctggacgatagaagcacagctcaatgtagagtacaaatgcaggttgtacagcagttagagctacagcttgcaaaagacaaagagcgcctgcaagccatgatgacccacctgcatgtgaagtctacagaacccaaagctgcccctcagcccctgaatctggtatcaa

SEQ ID NO: 326 - DNA, 153 bp, Mus musculus
  misc_feature 19, 44, 88, 97, 105, 109: n = A,T,C or G
tttaacggagctgcgattntcatttctaacacgttcaatccggntaagtcggacgcggaggatggggaagggacggtggcttcctggnagctccggntagaaggncggnctcctggaggacgccgccttgtccaaatatgacgccaccaagca

SEQ ID NO: 327 - DNA, 518 bp, Mus musculus
  misc_feature 96, 129, 146, 158, 342, 447, 450, 456, 470, 480, 489: n = A,T,C or G
tgagataaccctggagcagatatgctgtcaacaacactggcgaacatgctgatgaccaggacggtgactccaggttctcggatgttattctggcancaattttggcaaccaaatctgaaatgtgcaggncaaaaatttgaactgangattgataatgnacaggtttgttgggcacccaacactgctgcagcatgctctggggcaggtctccaaaacagatccatctccaaagcgggaagctccgaccatgattctttttaacgtggtatttgcactgagggccaatgctgatccatcagtgataaactgtctacacaacttgtcccgccgaattgctactgncctgcagcatgaggagcgccgctgccagtacctcactcgagaggccaagctgatcctagcacttcaggatgaggtgtctgctatggcctgatgcaaatgaaggtnctnagtccncattccagcacatnctacccaagngcaagctgnctcgggacctcaaagaagctttatgacag

SEQ ID NO: 328 - DNA, 231 bp, Mus musculus
  misc_feature 62, 107, 116, 128, 154, 155, 163, 166, 179, 196, 198,199, 200, 211: n = A,T,C or G
acagagaagtcccttgtcagaaacactagatgagaagccttctgatatgactctacccttancgctttcacgttgggactgaagaccaccagatgctcctgcctgtnacggacagncagtctcctgcntcagacacgctgctgaggtccacggnngaatgggnacntcggaaccgcagntgatttttttacatcantnnnactcgactttnagggcctggacttgcataga

SEQ ID NO: 329 - DNA, 495 bp, Mus musculus
  misc_feature 280: n = A,T,C or G
gactgagtgatgcggcccagggcagcctgcggctcatcctgaacacgaagctgtgggcacagatgcagatggataaggccagtgaaaagagcatccgcatcacagccaccgacgctgaggaccagggtgtcaaggttttcctgatctcggccagctccaaagacacaggccaacatgtatgctgcactgcaccaccgcatcctggctctgcgtagccgggctgaacaggaacaggaggccaaggcgcccccacctgagccaggagccacccgggccaccngaggaggaagacagtgatgagggatgctgttctggctccctccggtgtcacgggggctggcacaggtgatgaaggagatggccaggctcctgggagcacatagcggcggagccactgcaccaggctgctgctttgttgtctgtctgctgcccgtccctccctgcaggccgcatcatgggtggtgtctattgccggggtggccacaggctggatcc

SEQ ID NO: 330 - DNA, 414 bp, Mus musculus
agcatatctgaaaatgaaagaaggctggtttctcaggaggcttggagcaagctgcagcagtacttcccaaaggctcccgagttcccaagttacagagagtgctgctcgcagtgcaagatactagaacgagaaggtgaagaaaacgaagccttgcataagatgatagcaaacgagcagaagacctcacttccaaacttgttccaggacaaaaacagaccatgtctcagtaactggccagaggacacggatgctctctatattgtgtcgcacttctttttagatgaatggcggaaatttgttagaaagcctgcaaggtctgctcctgtgtcgtcaattgggaatgctgcccttctgtgtccacatggaggactcatgtttacatttccttccctcaccaaagaagactcgaaactt

SEQ ID NO: 331 - DNA, 169 bp, Mus musculus
gactgaggtgagccggctactttacagttttgccaccgccttccgccgctctgccaagcagaccccactctcagccacagctgcaccacagactcctgacagcgacatctttaccttctctgtgtctttagaaataaaagaagatgatggtaaaggttaaaaaaaaaaa

SEQ ID NO: 332 - DNA, 528 bp, Mus musculus
  misc_feature 29: n = A,T,C or G
actgagctacaggtacaagaaagaagagncaagggaaggccagaggcgaagaagttgataagttagccaggggccaccaggcacttgagcggaggcgagaaggaggcagggaggaccaaaggcaccaggaagggaggactgagagagcaaggtctgaaagacgcagagctcagaactcacgagatgcagactggagagatcccctagcaaagcacatagacgacagaagccatgagaatagtcacagcagagttggcaatggccgagaacaaggctcacacagagagccagaagacaggcatggccgagccaaagaagcggagagagagaagagattctttttctgaaaatgagaagcaaagaagccgaaatcaggacagtgataatgttagaagaaaagatcgctccaagtcaagggagaggagtagaaggcattctggtcacaagggagacgacgccaggtgtcagaacagtgcagagaggcgctgggaaaagcctggcagacgccctgaacagtctagagagtca

SEQ ID NO: 333 - DNA, 141 bp, Mus musculus
tgaggagcttgacaattatgcagaatgggacttaagagacattgattttgttgaagacgactcagacatcttacatgctctgaagatggctgtggtggatatctatcattccaggttaaaagagaggcaaagaagaaaaaa

SEQ ID NO: 334 - DNA, 409 bp, Mus musculus
  misc_feature 307: n = A,T,C or G
tgtgtagtggagctggtcttcaaggtggccacagggagagctaaagaatggctttgctgtggttcgtcccccaggacaccatgccagaggagagcacacccatgggtttctgctactttaactccgtggcagttgcagccaaacttctccagcagaggctgaatgtgagcaagatcctcattgtagacctgggatgtacatcatgggaatgggacccagcaggccttctacaatgaccccaatgttctctacatgtccctgcaccgctatgacgatgggaacttcttcccaggaagtggagcaccanatgaggtgggcacagggccaggcgtgggtttcaatgtcaacatggctttcacgggtggcactcaaaccccccatgggagacgctgagtacttgccatccctt

SEQ ID NO: 335 - DNA, 480 bp, Mus musculus
  misc_feature 321, 335, 386, 423, 437, 438, 450, 454, 459: n = A,T,C or G
actgaggttcagctcctaatcagcagcacagtgtactcagtgatgaagcgtctatcaacagaaagaacagggatgtaccaccaaaccattctcagctgaagcatgattctcatttggagataacaattccaaagtccttgaaactgaaagactcagagaaagtggatgaaaaacagcgtggtcatagatgcaggacacaaacgatttggagcagtgtcctgtaatatctgtggaatgctctatacagcattcaaatccagaagatgaaacacagcacctgcttttccacaaccagtttataagcgctgtaaagtatgtggnctggaaaaaggaangaattcttgctgaatatcctgatggtagaataataatggttcttcctgaanacccaaagtatgccctgaaaaaggttgatgaaattanagagatggttgacnntgatctgggttnccancaggntccttctgttgaggtattcca

SEQ ID NO: 336 - DNA, 293 bp, Mus musculus
  misc_feature 28, 72, 222, 265: n = A,T,C or G
gtaacgactgctggctttttgcagagancttattctgcctacaaccccaatatccttagtttctttaattgntgagagactcctccacatcatcagagatgataatgaaaggatacagattgtcacagaaattatctcagagattcgtgcacccattgttaatgttgttgttgatccatctgatacaagaaagaaagaactcaagatggctgaaataaaagntaaacttattgaggctaaagaagctttggaaaattgcattgcntgtacaagattttgatcaggcatcaaaa

SEQ ID NO: 337 - DNA, 272 bp, Mus musculus
  misc_feature 92, 101, 105, 107, 108, 111, 113, 123, 148, 149, 150,151, 153, 154, 163, 186, 197, 214, 216, 221, 229: n = A,T,C or G
agacactagtgcactgcagtctgaatgagctagaagatgcagcgctcacttttccagtccttttccaagatgtcatttatcaggtggaggcnaggaacacngctncnnatncncaagaacaangttgcgcactgttaagacatgggannnngnntttattttnccgcataatatccaggtaataanaataatgaaanatgcacaatacataaancntaaancgcagcgnggtgagcgccaccaggcttttccgatgaacattgaagtgtgtg

SEQ ID NO: 338 - DNA, 397 bp, Mus musculus
  misc_feature 54, 232, 237, 249, 257, 259, 271, 275, 285, 291, 311,312, 313, 314, 327, 332, 335, 339, 340, 349, 359, 363, 366, 375,376: n = A,T,C or G
catgcaagcaaaaccaagcaggtcatctccaagatcatggacacagtaaccttnccaacgtgccagctcatctcccgaagccactactcccccatttacctgtcattaatcatgctcttggctgccctgagctggcagtacctgagcaccctgtcccaggtcacagaagactatgtccagagagaacacctgattttgtttatccggatgtggaagttcccgtggagtgggnttttncttttcctccancctggatntngacttcccttgntttncctcctactntaaaanctttgggaaaactgagtgtnnnnacctttaggaacntggtnagnaggnnaggatgccnttgccctcancagnccnggtgctggnntgggacttgattactctgact

SEQ ID NO: 339 - DNA, 452 bp, Mus musculus
  misc_feature 23, 34, 38, 39, 57, 64, 70, 78, 79, 85, 118, 141, 144,149, 153, 157, 158, 163, 164, 196, 198, 204, 207, 221, 223, 225,239, 243, 266, 267, 275, 277, 291, 302, 303, 309, 310, 315, 321,326, 354, 425: n = A,T,C or G
catggccacatgcacagatcacngactttggctngtgnnaagagggcatcaggaganggagccnccatgnataccttnngtggcnccccggagtacattggcagcctgaggcgctagnagatacaaatgacctatatgtcngancagtngtantgtnnagagnntggtatgtagcctatgtatgatgatgatgatncnggcgtntgnccttctacaaccananancactgagcggcctntttnagctcattcttatggaggagatnnacattccngntcacacgacggacnaaaggccaagnncctgcnngatgngcctgntgganaaggacccacagcagaggctcggcggangtcccagtgatgcgaaggaggtcatggagcatagattcttcctcagcatcaactggcaggacgtggtacanaaaaagctcctgccacccttcaatcct

SEQ ID NO: 340 - DNA, 516 bp, Mus musculus
  misc_feature 21, 28, 42, 92, 93, 94, 108, 109, 113, 120, 133, 140,145, 154, 167, 174, 175, 178, 179, 184, 186, 207, 208, 213, 217,220, 221, 225, 235, 236, 247, 258, 266, 275, 276, 305, 311, 322,326, 355, 366, 368, 369, 394, 404, 423, 449, 454: n = A,T,C or G
  misc_feature 493, 498: n = A,T,C or G
tgagccataccccggaaaggngtctgantttggaattagaancggcagccggcttcaagcagacgacttcctgcaggactatactttgctgnnnaacattcttgatanngaanatcttgnaaaggatgttgantttgaantaagntggtgacangccctgaataagnggggccnnaannagctnangatgctgccaaaagcattgcnnacggnaggnatnncggngcccagcctnncacctgcacancacaagagcangatgatgngctcatagnngattgagacgaagaaggcccttcaaatancaccanactgcagcggngacnacaaggcccgcatgcggaagctggaaganaatgaggctgncnntaccaagaagtgccggttggagcanatggaagatncagatgacgtgatagcganagactgagcaggctcctgcccttggnctggncggaaccaaaagttagattctttgtgtggctttgatggntccgntcagcgttcattcgctga

SEQ ID NO: 341 - DNA, 285 bp, Mus musculus
  misc_feature 264: n = A,T,C or G
aagtggccatgcaggaaatggaagaatgtccaataagtaagaaaagagcaacatgggagacaattcttgatgggaagaggctgcctccatttgagacatttctctcagggacctacattacaatttactcttcgatggacaggagaaaccaacgataagtctacagctcctgttgccaagcctcttgccactagaaattcagagagccttcatcaggaaaataagcctggttctgttaaacctgcacaaactattgctgttaangagacgctgactacagagctg

SEQ ID NO: 342 - DNA, 274 bp, Mus musculus
  misc_feature 131, 171: n = A,T,C or G
gactgaggaaaacttgatcggagcactcttggccatttttgggcacctggtggtcagcattgcccttaacctgcagaaatactgccacatccgcctagcaggctccaaggacccacgggcctacttcaagncccagccttgtgctgagaacaccggtgcttttccagtggnctcacctgcagcacagatgaagcctggggaatcgacggtaatgcggcactgatgcttgccaataaaaccaaccaaccaatcaaaaacttgaaggaaaaaaaaa

SEQ ID NO: 343 - DNA, 370 bp, Mus musculus
  misc_feature 24, 28, 34, 45, 58, 83, 86, 134, 173, 188, 195, 207,235, 260, 262, 266, 282, 338, 347: n = A,T,C or G
tgagggcagcagcagcatgaccgngcanacgttnctggcaaacancctgactatcagngggctgagccccacccagatccagntgnagatggagcaggctctagccggagaggcccaccctggggagaaacggnggcagcatggcctgcacgtgtcccaactgcaaggatggngagaagaggtctggnagagcanggcaagaagaancactgtgtgccacatcccggactgcggnaagactttccggaagacatccttgntncggncccacgtccgcctgcncactggcgagcggccctttgtctgcaactggttcttctgtggaaagagattcactnggagcgatnaactccagcgacatgctatgcat

SEQ ID NO: 344 - DNA, 479 bp, Mus musculus
  misc_feature 51, 91: n = A,T,C or G
actgaggcaaggcatctttacatttgtgactatcataagaacttaattcanagcgttcggaacagaaggaagaggaacggaagcgatgatnatgggggagactcgcctgttcaggacatcgacactccagaggttgatttgtaccaattacaagtaaatacacttagaagatacaaaagacacttcaagcttccaaccagaccaggtctaaataaagcacaacttgttgagatagttggttgccactttaagtctattccagtgaacgaaaaagacaccttaacatgtttcatctactcagtgaggaacgacaagaacaaatcggatctcaaggccgacagtggtgttcactaggagagatgctgttcacactagcccatagatgtctgcctggaaagaatgtttactggttttcagatgtagaaatgttcttgtgtattttttctaccgaggatttactttgactttttttttttttt

SEQ ID NO: 345 - DNA, 122 bp, Mus musculus
actgagccactggacggactgttgaactacccgaggctgaagttgttagaaatccccgaagtataactgcaaaacaagcaccagagacagacaagaaaaatgaaaagtcaaaagaaaaataa

SEQ ID NO: 346 - DNA, 507 bp, Mus musculus
gactgagattctgcttcgggaaggctgcaagaaaatcctcgtagctgactcattgtcgttgctgaaaaaaatgcaccgaacccaaatgaaaggcgttctggttgatgaagagaatatctgtgaatcatgcctttctcccatcctcccaacagatgcagcgaagcccttcagcgtggtggtcttccactgccggcacatgttccacaaggagtgcctgcctatgccgagcatgaatgctcctgcacaatactgcaacatctgtagtgctaagaaccgtgggcctggaagtgccatcctggagatgaagaagtaggcctgcctgcctgcccaccctttgccactctccactgagtgactgctggaccacagcagctgtgtcaacgccagtgctgttgagtgacgtgttcagggctcctcggagctccccgtcagttgttttctccagaccttccttccagaagctgctccttgtcaccgatggagaagtcgaagctcttcccatgccat

SEQ ID NO: 347 - DNA, 122 bp, Mus musculus
  misc_feature 24, 56, 64: n = A,T,C or G
gactgagattgaacctgctctgangataatacactgaaaatctggcgcttgaatanaggcctanaggagaaaccaggtgataaacattccatagtgggttggacctctcagaagaaaaaaaa

SEQ ID NO: 348 - DNA, 497 bp, Mus musculus
  misc_feature 43, 46, 74, 83, 103, 104, 121, 122, 126, 127, 132, 135,137, 138, 139, 140, 141, 143, 144, 145, 149, 151, 161, 162, 170,175, 176, 177, 179, 181, 188, 189, 192, 199, 202, 205, 210, 215,216, 219, 220, 226, 228, 230, 232, 255: n = A,T,C or G
atggagggaatgacaccagacgactagcgccagccgctccttnctntggttcttgcgctggacagatggggacnagagggccnacgcatgcatccacctctcnngagtgggcaggccatcnnaggnngcaanctntnnnnncnnnttcntnaggccattcnnaatgcctnactannncnangtgtacnngcncttaaanatngtnaactnacatnncgnnaggagncntnanactgactccaaaaccagagtcgncaccagcaccgtcttctaatggccagctctgccgttacttccctgcttgtaagaaaatggaatgtcccttctaccacccaaaacactgtaggtttaacactcagtgtacgagacctgactgcacattttatcaccccaccattactgtgccaccaagacacgccttgaaatggattcgacctcagagcagtgagtgatgccctagtcctacctggcagaagatcatgcagtttgaaagctta

SEQ ID NO: 349 - DNA, 168 bp, Mus musculus
  misc_feature 90, 124, 127, 128, 136, 148: n = A,T,C or G
tgaggaaaaacaagagtggtggtggctttatattgcagataggaaagaacaaacattaatatccatgccatatcatgtgtgcacacatgnaggatacagaagaggtaattagccaacaatcttngcnntaatatcntacctttatgangtttcattaaaattttaata

SEQ ID NO: 350 - DNA, 394 bp, Mus musculus
  misc_feature 92, 181, 232, 325, 326, 368, 370, 373, 375: n = A,T,C or G
tgttagcatgaccaatgagtatggagcagagggttgagccacataatgattacttcagcacccagttcctgttgaactttgctgtccttggnacgcacagcatcacagtggagtcatcggtgagagatgccaatggcatcgtctggaagactggccccagaactactatgtttgtcaaatncctggaagacccctactcccagcagattcgcctacagcagcaggcccagcngcctttgcagccacagccacagccacagccgcagcctcgcagtgcctacacccaggttttagctgtgctacaagcatactactgccacactgnncagccatcagactacaagaagagtcttccgcatgtgcattangngtnantgttacagtgtgaaatctc

SEQ ID NO: 351 - DNA, 209 bp, Mus musculus
  misc_feature 183: n = A,T,C or G
agagctgtcggccatggagccgagtgatagcgccagtaccgctatggaggagcctgacagcctggaggtactggtgaagaccctggactctcagactcggacttttattgtgggggcccagatgaatgtaaaggagtttaaggaacacatagctgcctctgtcagcatcccttccgaggcggnagaggaggagggggagacttcacgcc

SEQ ID NO: 352 - DNA, 308 bp, Mus musculus
ttgagatgccgtgcgccacaacctcagcctgcacaagtgtttcgtccgtgtggagaacgtcaagggggccgtgtggactgtggatgagcgggaatatcagaaacggagaccgccaaagatgacagggagtcccactctggtgaagaacatgatctcgggcctcagttactggggcacttaacgccagcctacgggtccccgaaatggggtcctctcaccccgccccctcacctcctcctgtccttccctctcccagttaaaacggatgaccaaagacctttcttatgccggaagcaaaaaccaaaaaa

SEQ ID NO: 353 - DNA, 493 bp, Mus musculus
  misc_feature 427, 460, 470: n = A,T,C or G
gtgtcctaacgtgcttaaacttactccacaacgaagaaccagaagaaaaataattccttctcccttgaaagtacaacatactcagaagtctgcactgtctgaaagtacacagctcaatgattctttcagcaaagaacttcagcctattgtttatacgccagaggactgcaaaaaagctcaagacctgtttccatccttaacgagaacttcatcgcagtctgcaaatgtaatgaatgacaattcccagaaagcactgtgtagaatagagtctcctctcagcagaactgagtgcaaacagggtttgtactctacatctactctgtgtgatagcatcagaggcttgaagaataaatggcctgaacaagaaccactagcaagcagcaaaagcagtgtgcacaggatagagtcttcttcattttcaacaaangatttctatgcctgaatcagcaggtgtgccgtncctacatggnaatgactactgctgcaaagagga

SEQ ID NO: 354 - DNA, 383 bp, Mus musculus
  misc_feature 53, 65: n = A,T,C or G
caaggaaaaagcaagttaagaagataacagatggactgcatggccttcaagangcaagcaaaaancgatgctgaagctttggctgctgcacagcagcacttcaatgctgtttctgctggcttgtccagtaacgaagatggagcagaagcaactctggctgggcagatgatagcctgtaaaaacgatatcagcaaagctcagactgaggccaagcaggctcagatgaagctgaaacatgcccaacaagaactaaagagtaagcaagcagaggttaagaagatggacagtggctacaagaaggatcaagacgcttttgaagctgtgaaaaaagctaaagaaaaacttgaaactgaaatgaagaagctaaattacgaaaaaaaaaa

SEQ ID NO: 355 - DNA, 236 bp, Mus musculus
  misc_feature 17, 18, 29, 43, 48, 72, 73, 81, 82, 87, 93, 164, 175,206: n = A,T,C or G
atttttttttaacttanngcaaaagttgncgctcctgccctgnggtgnggtcctcacagcacctgtttctanntttgcctnngttgnaaaacncgtctggaggactggttggggaagacagtgcctggcagtgactggacacccaatgccaggttcattatacnttcctggcttngagatggcctcaagccacgatgcatcaccanagacctcaaatggggaacgcctgtgccctt

SEQ ID NO: 356 - DNA, 514 bp, Mus musculus
actgagcttgtgggctttttgctcattggcgtggctgcctggggcaagggcctcggtgtggtgtctagcattcacatcattggaggagtcattgctgtgggagtcttccttctcctcatcgcggtggcaggactggtgggcgccgcgaaccaccatcaagtgctgctcttcttttatatgatcatcctaggtttggtcttcatcttccagtttggaatctcttgctcatgtctggctattaacagaaacacacaggcagatgtcatcaatgcttcgtggtcggtcttgagcaacagtacccggcatgaactggagagaagttttgactgctgtggcttgttcaaccttacaaccctgcgcctacaagatgatacttcctgtagtgcggtgtgcaaaactaagagctctacatgccagatgtgtggggagaggttccttaaacattcagacaaagccctcaagatcctaggaggtgttgggctcttctttagtttcacagagatccttggtgtac

SEQ ID NO: 357 - DNA, 460 bp, Mus musculus
  misc_feature 208, 420, 423, 432, 433: n = A,T,C or G
gagaagcaaagcaaatgcctccttgtgttatattcatcgatgaattagattctgttggtggaaagagaattgaatctccaatgcacccgtattcaaggcagacgatcaatcagcttcttgctgaaatggatggtttcaaacccaatgaaggagtaatcattataggtgccacaaatttcccagaggcattagataatgccttaatacngtcctggtcgttttgatatgcaagttacagttccaaggccagatgtgaagggtcgaactgaaattttgaaatggtatctcaacaagataaagtttgataaatctgttgatccagaaatcatagctcgagggactgttgggttctctggagcagagttggagaatcttgtgaaccaagctgcactaaaggcagcagtctgatggaaaagaaanggntaccatganngaactagagttttccaaggataataat

SEQ ID NO: 358 - DNA, 513 bp, Mus musculus
tgaggtggggtctttcaattccaagagcccttccagaagttgaacggcatcgccaaagggccaggcttgtccactgaaccttctgtcccagatgcccaagtgtctaccaggcccagtcagtctgctgggaatgctgacgtgaacagcaaacgaaagaggtctgagaagctgaagcagcggggacctaagtggaagccctctaaggaggctgctgtcccaaagccgagcgccccttccagggttgaggactcgagaacacctcctgtgccgactccctctgaaatccgtgctgccccaaggcctaaggactgtgctccatcccttggggaagccaaaaagaaacagaaaggaaagcagcagttagcacagcagcctgctaatggagctgctcccctgaaagaagatgctgtttccaagggaccctcagccccctttgtgtctccccacagttccaccaggcccccaccagcaaaaaggaggaaatcaatgacaaagggcaacagccagcctcta

SEQ ID NO: 359 - DNA, 192 bp, Mus musculus
  misc_feature 21, 24, 27, 30, 39, 67, 93, 105, 107, 115, 121, 122,129, 144, 159, 167: n = A,T,C or G
ctctttgatgatgaagatcanacngcnaancgagaaatncaggcgccatttgtctaagattttgtcntaacaacatataaggcctcagcggcnccttcttcatgntntggaatgngcgganncattgcntcaagctcagctggnctccaagatcccacncattctgnaggagatgtatgatgccgacctgtt

SEQ ID NO: 360 - DNA, 117 bp, Mus musculus
  misc_feature 41, 42, 43, 50, 56, 73, 75, 93: n = A,T,C or G
gatgacaaaagtatgcaagagccaccttctcaactgttggnnnaacgaangccttnctggaacaagaatggancntggagaatgcttgaaagnccatagacctggctttaagagccg

SEQ ID NO: 361 - DNA, 514 bp, Mus musculus
tgaggtgcacaagcttctgatcgtggtagagctcctgctgggggagatcccagaccgcctgcagttccgccagccctccctaaagcgctccctcatgccctacttccttctcacccaagctgttaggacaggaaatcttgccaagttcaaccaagttctggatcagtttggggagaagtttcaaacggatgggacctacaccctgattatccgactgcgacacaatgtgatcaagacaggtgtgcgcatgatcagcctgtcctattcccggatctccctggccgacatcgcccagaagctgcagctggatagcccagaagatgcagagttcattgtcgccaaggccatccgggacggcgtgattgaggccagcatcaatcacgagaagggatacgtccaatccaaagagatgattgacatctattccacccgggagccccagctggccttccaccagcgcatctccttctgtctggacatccacaacatgtctgtcaaggccatgcgctttcct

SEQ ID NO: 362 - DNA, 354 bp, Mus musculus
  misc_feature 331, 338: n = A,T,C or G
ttgaggagttgaagctgtctttgaagagcaaagaagctttaattcagtgccttaaagaggaaaaatctcagatggcatctcctgacgagaacgtgtcatctggagaactccgaggactttctgccactctgagggaagaaaaagagagagacgctgaggaatggcagaaggagagaaaccactttgaagagaggatccaggcacttcaggaggacctgagagagaaggaaagagaaattgctacagagaagaaaaataacttaaagagttataaagccattcagggtctaaccatggcattaaaatcaaaggaaagagaggtatgtctgangagtgangtgggaaaaaaagaaa

SEQ ID NO: 363 - DNA, 396 bp, Mus musculus
actgaggagacagactggacagagccatggctcctaggactccttgccttccacctgctgtgcctccttctcacttgcttctcctcacagaggtataagctacagatcgggcacttcctgtgtctggtagtgttagtctatagtgccgagtacatcaacgaagtggctgcagtgaactggaggtaaagttggcttctcatgctctcgcctctgcctcagtcttcctctacagtggcactttcacatggcactttctaaagaaaagatgaagctcacagggaaattgaggacgccaggaatcgtggccacataaatattgaacagcctatacatatggggttaggccttaagaaataaattagttaaatattttcttgtttttgaccaaaaaaaaaa

SEQ ID NO: 364 - DNA, 391 bp, Mus musculus
  misc_feature 186, 189, 196, 198, 204, 206, 209, 211, 212, 216, 217,220, 222, 228, 229, 231, 243, 244, 249, 250, 257, 263, 265, 268,269, 271: n = A,T,C or G
tgagttcttgtgtctgaagaatattcgcaccttcctcaaagtctgccatgacaaatttggactaagaaacagtgagctgtttgaccctttcgacctctttgatgtccgagacttcgggaaggtcatctctgcagtgtcccgtctgtccctgcacagcatcgcacagagcaaagggatcaggccatnttnatcagtnanagacanangananngatnnccncntgtatnngngcctgaaccagnngcttnnacactgnactgtnantgnntntggtcagcagccaggaggtgtgtgggagagtgttcacgctggggaaatcttgcaagcatgttatgatgggggcaaaattgctttcgatttaggtaataagatcagcaaattaatcctaaa

SEQ ID NO: 365 - DNA, 501 bp, Mus musculus
  misc_feature 351: n = A,T,C or G
actgaggcatatatagggagggctggacgctagctcaccaccgcactgcccagaggctcctgtctgactcactgccagccttcggtccctgcatcaccaaatagaagatggacgtcaccattcagcatccttggttcaagcgtgccctggggcccttctaccccagccgactgttcgaccagttcttcggcgagggcctttttgagtacgacctgctgcccttcctgtcttccaccatcagcccctactaccgccagtccctcttccgcactgtgctggactcgggcatctctgaggtccgatctgaccgggacaagtttgtcatcttcttggacgtgaagcacttctctnctgaggacctcaccgtgaaggtactggaggattttgtggagattcacggcaaacacaacgagaggcaggatgaccatggctacatttctcgtgaatttcaccgtcgctaccgtctgccttccaatgtggaccagtccgccctctcctgct

SEQ ID NO: 366 - DNA, 512 bp, Mus musculus
  misc_feature 208: n = A,T,C or G
gactgagcttgcaaccatattgctaatagaaaaaagtgtcagtatgttggcaactgttcctttgctcatagtccagaggagcgggaagtgtggacttacatgaaggataacaaaatacatgatatagagcaattttatgaactctggctgaagaaccaaaaaactgaaaaaagtgatgagacagccagtcactgtagcaaagaaaacngggaagcagatccatatgccaacagactatgctgaagtcactgtggacttccactgctggatgtgtgggaagaactgtaacagtgagaagcaatggcaagaccacatctcctctgagaagcacaaggagaaggttttccacactgaggatgaccagtactgctggcagcaccgcttccccactgggtacttcagtatttgtgagaggtatataaatggctcctgcacagaaggaagcagctgtaaatttgcacatggaaatgctgaacttcatgaatgggaagaaagaagaaaggccctacagg

SEQ ID NO: 367 - DNA, 172 bp, Mus musculus
  misc_feature 22, 49, 61, 96, 123, 126, 139, 142, 149: n = A,T,C or G
ggagactcacgggagccccggncttcacctgagctacctcagtcgccangagtggcaaganaaggaagaagaccctgacttgggggggaccatagnactcaaacccagagtaggggaacattnacnttacctcgcttgnccnggaaatnttgatgggctttgcccactactt

SEQ ID NO: 368 - DNA, 294 bp, Mus musculus
  misc_feature 22, 26, 46, 53, 56, 132, 157, 199, 203, 257, 268, 273: n = A,T,C or G
tgaggcaaagcagtagtagcanaggntcttcagtccacgcagtggntcttttntgnacagcaacagaacaaacagataggaggcaaaacccagagagtggacagtgatacaagtaaccctgaaacgctgagngaccctcctggcacctgtccggaanaagtagaagaaaagccaccccctggacccaccataaccaccnaacntgttagaactggaccaatcaaacctcaggccatcaaaaccgaagaaacaaaatngtaaaggctgnggttnattggggatgggtgggtaggg

SEQ ID NO: 369 - DNA, 175 bp, Mus musculus
  misc_feature 94, 96, 97, 99, 105, 106, 109, 110, 112, 125, 126, 131,143, 146, 153: n = A,T,C or G
gtcagatattcctaagagcaccaaaggcgatggagaagataacagaccaggaatgagagggggccatcaaatggtcattgacgtccagacagananngnctattnnctnngngggtgggatgagnncacangatcttgctganttnacgggcntaccacagttgaagcgagaacc

SEQ ID NO: 370 - DNA, 346 bp, Mus musculus
  misc_feature 40, 41, 95: n = A,T,C or G
gcaccttgttccacccattttcagctacagaatcaccaannggatttctctatcccgagggcccaggccccggccagtccgtacaatgactacanaagggcgcaaaggcgacagctaagtgttgggaagagtgggacaaccagaggcaggtagcaggtccagccaggaatcctgctgatgtgaactgaactcagggcatcccggggaaacagtacagagaggcttgcaacctgcccagcacaccgccgtcttgcctggggctgctaagcctaacaaactgtcttcaaagagctccagggtttcatttgccccattcctaggacacttctgggaggtgggagttcta

SEQ ID NO: 371 - DNA, 181 bp, Mus musculus
actgagggaccttagaagattgggatctagccattcagaaaacggagacgcgcttggctcgtgtgaatgagcagagaatgaaggccgcagagaaggaagcagctcttgtgcagcaggaagaagaaaaggccgagcagcggaagaaggtgcgggcggagaagaaagccctgaaaaaaaaaaa

SEQ ID NO: 372 - DNA, 135 bp, Mus musculus
  misc_feature 28, 30, 37, 39, 42, 56, 59, 64, 65, 75: n = A,T,C or G
tgtctatacggccccgaccactatctcncnctgaagncnggnttgacaaacccggncanctggnncacctttgangatgaccctgcttttcaatcttctcagaaaagaaaggatttttctctggaaaaaaaaaaa

SEQ ID NO: 373 - DNA, 129 bp, Mus musculus
gctaaggattacatgcccaccagcaaagaggagccaatccgcctgttacgggaggtggtgttgctgaccgatgatcggaaccttcgtgtaaaggcgctgacaaggaatgtgcctgtgagggacatcccg

SEQ ID NO: 374 - DNA, 234 bp, Mus musculus
  misc_feature 34, 35, 212: n = A,T,C or G
tgaggttacttccttgtcctgggccaaacatganncccattgctcttgggagccgctggcttgcttatgcagaaaataagctgattcgctgtcaccagtcccgtggaggagcctgtggagacaacattcagtcttacactgccactgtccttagtgctgctaaaacacatgaaaagtggcctgacgatggttgggaaagtggtgactcaacntgacaggcacattgccctcagg

SEQ ID NO: 375 - DNA, 429 bp, Mus musculus
  misc_feature 314, 320, 384: n = A,T,C or G
aagcggcagtttcctctgcagccgaagggaagacgacacacgcccttacccgcaccaaggaagccttcactcctccagacctgtgtccatgtggccctgtgaagacgccgactccatcccatttgaagaccggccgctctccaaactgaaggagtcggacaggtgctcggccagtgagaacctgtacttggacgctttgtccctggatgatgatccaggggatccgccacccctcaggaactgccttgccgaggtcagcctgggtttgtcccccgaggttcctaatggctggagggagctgtcattaagtgcanctagtnttgagcagagtacccctctcatcaggttccccagacaacctgtgcattgagccacgtgaaccgntgccattggcgatgtgatgactttatttggtgaacctgaccccgg

SEQ ID NO: 376 - DNA, 302 bp, Mus musculus
actgaggatatggttactggacttagtcctctgctcttcaggaagcttagtaatcctgacatatttgcacccactggaaaagttaaactccagcgacaacttagtcaggatgactgtaagttacggagaggaagcctggcaagttctctgtcgggtaagcagctgctccctttgtccagcagtgtacacagcagtgtgggacaggtaacttggcagcctacaggagaagcatcaaacctggttcgaatgagaaaccaatcccttggacagtctgcaccttcccttacggctggcttgacagg

SEQ ID NO: 377 - DNA, 221 bp, Mus musculus
  misc_feature 29, 34, 41, 48, 51, 54, 114, 152, 153, 158, 174, 195: n = A,T,C or G
aatcttcaagagaaagaccttcaagcagnctggngagatgnctcatanacnaanagcacccctagctcctgtaagttcagtccccagcacccacatggtggctcaccatccatnactccagttctaggagagccaaggacctctttcacctnnatcangaccatactggcatgngctgcacaggcatccttgcangcaaaaacactttatcacgtgaaata

SEQ ID NO: 378 - DNA, 257 bp, Mus musculus
tggaagagtcagcatttactgggtcagctgaagatcattaaagacttttacctgctgggacgaggagaactgttccaggcctccacctccaccagcccagctttactgtctaaagaaacagtggcgtgacctccctcacacaagtcactgtggagccaagaactatgtctttatattgcttatctcagagtttacatttcttttttaaaaatatttcatttacttgtaaataaaaaatgattctcagaaaaaaaaaa

SEQ ID NO: 379 - DNA, 542 bp, Mus musculus
  misc_feature 501: n = A,T,C or G
ggctccgcagccttgtttctagcagcaaaagtagaagaacaaccaaagaaattggagcatgtcatcaaggtggcacacacttgcctgcacccacaggagtcacttcctgacacgaggagtgaggcttacctacaacaagttcaagatctggtgattttagagagtataattttgcagaccttagggtttgaactaacaattgatcacccacatacacatgtggtaaagtgcacccagcttgttcgaggatctcagactgaacagtagagaggtgaccaagcatcagccggccggcagtcagccctctgtttcctgactcctcgaagtgtgacagctgcttcatgttcctgctgttgggccttctccaccctggtggcttggactgtgagccatacaggaaaaggaactaaaagtcaacttttgtctggttggggtacttcacaaagtgcaaggacagatttggaaaagccaccaagtcacatataagttagcatttttcanaaaatcttgaaagttgctggcgttgggccagcaagtgggtc

SEQ ID NO: 380 - DNA, 458 bp, Mus musculus
  misc_feature 327: n = A,T,C or G
gaggcgcgatgatgcctattggccagaagctaaacgggctgctctggatgaccgttaccactcagacttcagccgccaggaccgctttcacgacttcgaccacagagacagaggccgttatcccaaccattctgtggataggagagaaggttcactggtcgatgatgggagacagagaaggacagcattaccctgaacgccatggaggacctgaacgccatggccgggactctcgagatggctggggctatggttcgaacaagagattgagtgaaggccgggggctgccccctcctcccaggcgtgactggggggagcatggccggngacttgaagatgaccgggcatggcagggcacggctgatggaggcatgatggagcgtgatcataagaggtggcaaggtggtgagaggagcatgtctggccactcggggcctggtcacatgatgaacccgttg

SEQ ID NO: 381 - DNA, 189 bp, Mus musculus
  misc_feature 17, 19, 36, 42, 44, 45, 46, 88, 160: n = A,T,C or G
cacagaatcgatctgananaagtggagagcgcatgnagtatntnnncaacaccaagatcaactccatcaacagggggctgagacaganaagcaagcgtgaatgaagtcttacacaatttttggaagcgctaccttcagaagagcaagcaggcctattgtnaaaacccagtttcatacaagtggaaggaa

SEQ ID NO: 382 - DNA, 463 bp, Mus musculus
  misc_feature 249, 369, 383, 394, 440: n = A,T,C or G
gagtctttaaaattcactggttgatggccgcccttcctttcaccaagtctctctcgttggtgttccatgcaatcgactaccactacatctcctcgcagggctttccgattgaaggctgggctgttgtatactacataactcacctgctaaagggcgcacttcctgttcatcaccatcgccctcatcggcactggctgggccttcatcaagcacatcctgtctgataaagacaagaagatcttcatgatngtcatcccgctccaggtcctggcgaatgtggcctacatcatcatagagtccactgaggagggcacaactgagtatggcctatggaaggactctctgttcctggatgacttgctttgctgnggcgccatcctctncccagtggtgnggtcaatcaggcctttacaagaagcctccgccacagatggaaaagncaagtgtctgctgctccccgtgc

SEQ ID NO: 383 - DNA, 489 bp, Mus musculus
  misc_feature 36, 57, 64, 72, 137, 143, 171, 188, 189, 204, 231, 239,242, 261, 269, 273, 284, 402, 425, 437, 440, 448, 451, 456, 459: n = A,T,C or G
ccacggcctttggattcagcgtgcatattttatganccctacttacaggatgggatngagccgntctctggncgtgcaaagggtctacaccctgctaggactatgctatatcatgagcgacatgctgtctccttgcnctgcancctgaacgagcacaaccatcacctcatnaatgacttcactatganncaaatgaggcaaggngcattccttgtgaatgcagcccgaggnggcctggnggntgagaaagccttagctcangccctcangganggcaggatacnaggagctgccctggatgtgcacgagtctgagcccttcagcttcgcccagggcccattgaaggatgctccaaatctcatctgcacaccacacacagcctggtacagggaacaagcattnctagagatgagggaagcagctgncacttgagatcngcncgagcaantcncaggncgnatcccatcaaagcttaccaaactgtgtcaa

SEQ ID NO: 384 - DNA, 537 bp, Mus musculus
actgaggatcgcatggtccgctactgcctttaccaagtcgttatagaatgggctctagagatacacctgaacttgttgcttatccattaccacaagcgtcttcctcttacatgcatggaggcagtccgtctggctccgttgtaatggttagtggattacatcagctcaaaatgaattgttcaagagtcttcaacctgttctgcttgtatggaaatattgaaaaggtaaagtttatgaagaccattcctggcacagcactcgtagaaatgggtgacgagtacgcctgtggaaagagctgtcacccaccttaataatgtcaaactctttgggaaaagacttaatgtttgtgtgtctaaacagcactcagttgttcccagtcaaatatttgagctagaggatggtacgagcagctacaaagattttgcaatgagcaaaaataatcgctttacaagtgctggacaagcatctaagaatataatccagccaccgtcatgtgtgctgcattattacaatgttccgctgtgtgtcacagattac

SEQ ID NO: 385 - DNA, 426 bp, Mus musculus
  misc_feature 31, 58, 83, 105, 108, 126, 159, 226, 227, 228, 231, 236,240, 254, 273, 287, 346, 349, 374, 378, 379, 383, 408, 410: n = A,T,C or G
tttgggtaactttaggtgtctgagaggaaanacactttatgaagatctatctttacantcctgcattggtgaaaaggatatgngaccttttgcaaagcggagccntantgaataaatgttaccagnctcatgaagcgcatattccctacctcctacagntcttcattgactacaatctttatggaatgaatttaataaacctggctgctgtcaagtcccgaaaagnnnganggaanggtnatgcatcacatgcnactggattatttaagcatnaattgtcaggaaantctcctgctggcactttatttaggtgggaagaggatgaaataccaaggttagcctctgnggncaaagaaatgaacattttatatatntatnntcgngattagccagcagtgggcatcatantnaaaacttaacttttac

SEQ ID NO: 386 - DNA, 245 bp, Mus musculus
tgaaggccctggtctactatgatgtccaggcctgcagattggatattggtaatgagaaaggagacacagccctgcacattgctgcacgctggggttacgagggcatcatagagacactgctgcagaatggagcccccacagcagtccagaacagactgaaggaaacaccactcaagtgtgcctctgaactcgaaggtagctctttccatctcggagcaaccctgtgactcttaaaaacaaacaaa

SEQ ID NO: 387 - DNA, 121 bp, Mus musculus
gactgaggataccatacttgcagaacttcttcagatacataaagagcacattgtgggctatcatgaacatgattctcttttggatcacaaagaagaagaagaactaactgaaaaaaaaaaa

SEQ ID NO: 388 - DNA, 155 bp, Mus musculus
gactgaggttgtggcagcattgcctgagaatgagagcagactttaatcccagtggtttttcgttggaattggcagtatgtgtgctttccgttggactgcttgccgtggtcttgtttctgtggcgaggcttccgatcaattcgaagcaaaaaaaaa

SEQ ID NO: 389 - DNA, 408 bp, Mus musculus
  misc_feature 17, 161, 205, 208, 314, 317, 383: n = A,T,C or G
ttgtgtttgataacctnttgctgcttgatcgcaatgtggatctcctgacgccactcgcctcccagctcacgtatgaaggcctcatcgatgagatttacggcattcaaaacagttatgtgaagttgcctccagagaagtttgcacccaagaagcagggtggnttttggtggtgggaaggaccttcccacggaggccaagaagcttnagntcaactccgcagaggagctctacgccgagatccgagacaagaacttcaacgctgtgggctctgtgctcagcaagaaggcgaagatcatctctgcagcgttcgaggncgngagtcagctctggcatacctgacggagagagacactttctgctaactattggcccctctgcctggnactgaaatgtttagatcttacagct

SEQ ID NO: 390 - DNA, 548 bp, Mus musculus
tgagagagaaaacggttggaagccaagcaacgggaagacatctgggaaggcagagaccagtctgtggtttgaatatcagttcctgaaaggaatgaatccatgattgggaagtggttgatattcttgagacaagaggatctttccagagctgtgcacatgcagatgagcatgttaaagaaaacatgaacaaaacagacgacttggagaacgagggtccagtcctcactcttgaattcagacctgtccactgtgacagagacgttaaaaggaactgtcatgtgggaaggaaaatggaagtggcttctaagcgtcctgggtggagcaggtcatcttcatgtgggtcaggcctccaggaagtctgaagcaaatgtaaagcaaaacaggattcaagcctgcagtgataggagtcggaggtccgtggcagggcagggcgctccttagtacttctcagagttgagaactgcaggatgaggggcagcagtaagacagggagaggaaacagctcacagacaggagtgcctggtgcactcagcccgtacagagactac

SEQ ID NO: 391 - DNA, 112 bp, Mus musculus
  misc_feature 25, 34, 42, 79, 91: n = A,T,C or G
ctctgcaacagcagcatgagggacnggggccttnttgctacngggtttgatccaggatcgagcagggaggatggctggngagacacctganctgaccttggagcagtcgacc

SEQ ID NO: 392 - DNA, 244 bp, Mus musculus
  misc_feature 48, 52, 63, 69, 90, 92, 135, 140, 143, 159, 165, 176,198, 199, 210, 218, 221, 226: n = A,T,C or G
tggtgaaggacccaaacaggagacattcacctttgccttaactttggntttnatccagtgtgngaaaanggctatgtttgccaagatctngnggatcgcactcggacctggcatctatgctgcctgttctgtctnctatntgngtgccatggtctccancaactnagccctgcagnttgtcaactatccaactcatgnnctcggtaaatnctgcaagncanggggnaggtatgctcctgcaagt

SEQ ID NO: 393 - DNA, 496 bp, Mus musculus
  misc_feature 31, 34, 50, 65, 66, 73, 84, 86, 87, 88, 92, 94, 100,107, 119, 127, 159, 166, 168, 169, 197, 201, 204, 210, 217, 220,222, 229, 235, 244, 258, 259, 262, 270, 326, 329, 342, 344: n = A,T,C or G
gactgagaaagcagggcacctgccccagcgnggncatacccaagctcggnctctgtgaggaccanngtcaggnggacagccagngnnntggnancatganatgctgncgcaatggatgngggaaganggcctgcaccacacccaaattgctgagcgttnagcctcnannaccctgaggaaccggagagaggttgttnctgncgnactgtncatgtgnagncnatcctgnggcctnctttctctntggtctttgcattnnttnctggcccnacgaaagcatctcctttttctaaccaataaagtgatcgttttcatcaatggacaanctnctgaagaacggananaggttgtttctgccggactgtgcatctggagtcgttcctgtggcctcctttctctctggtctttgcatttcttcctggtccgacgaaagcatctcctttttctaaccaataaagtgatcgttttcagcaatggagaagctataaaaaaaaaa

SEQ ID NO: 394 - DNA, 514 bp, Mus musculus
  misc_feature 478, 491: n = A,T,C or G
tgagttcctggcccagaacgaagagaagccgtatgtggttgcccacttccacgaatggttggctggcgttggtctgtgtctgtgccgtgcccggcgcttgccggtggcaaccatcttcaccactcatgccacagctgctggggcagctacctgtgtgctggcgctgtggacttctacaacaacctggagaatttcaatgtagacaaggaagcagggggagggcagaaggtgtgtgattggagtgtggagcgtgcagcagctcactgtgcccatgtcttcactaccgtatcccagatcaccgcaatcgaggctcaacacctccttaagagaaaaccaggtagggtctgagctgtggtccctacatgtggctgaggtaagaggctctatgtcacactcaacaccaatgccattttactccagagagtagaacaacttccctaagtcctggattgagatggtaactcgtaaatgaagctgnctgactctgtatnctgaaggataataggagctatag

SEQ ID NO: 395 - DNA, 498 bp, Mus musculus
tgagaacgtgtattgaactcagagcctgctgcaagccagtgtgcacgcgcttactcgctgacatttccgcaacccaagactcttcaattcatgaataatgacttgttgtattgatcaagacagtttggggcaaacatttcaagacgccattgacatattgatccaacaatcagctggagaaagtcaatactcctcagaaaatagaaattacatggctatcattaacccttatcctcacgtcagaggcaacgctaactactatggaatgtcacccacagagaaccccctctatgactggagaggtgtaacaaatggttctgcagacctgtatttggaaggaggcttccaccaatccgtacagaacatagctgaaagccagctggtacaaccccccttcttccagcaaaagggaggaagaggcaggagaaagcttcggctgtttgaataccttttcgaatccctgtgcaattcagagatggtgtcttgtattcagtgggc

SEQ ID NO: 396 - DNA, 210 bp, Mus musculus
ctgtcagcggtgtggagcttctcatgtttcctcagccggccttcatcggagaacgtcttcccacacgcctggcaggcgatctgctctcgatggtgaccgtagagcaagtactcgaacttcatgtcgctggccgccgtggtccagcccggagtctgttcatcttttacttcgctcatcccatcattaaaggttaaggcctgaaaaaaaaag

SEQ ID NO: 397 - DNA, 389 bp, Mus musculus
  misc_feature 78: n = A,T,C or G
gatttgagatcttgccattcgtctgcattacagagcagaatttcatggtggaagatgagacggttttgtgcaatattncatacatgggtgacgaggtgaaggaagaagatgagactttcatcgaagagctgatcaataactatgatggcaaagtccacggtgaagaagagatgatccctggatctgtgctgatcagcgatgctgtgtttctggagctggtggatgccctcaaccagtactctgatgaggaggaggacgggcacaacgacccctccgatggaaagcaagacgacagcaaagaggacctgccggtaacaagaaaacggaagcgccatgctatcgaaggcaacaaaaagagttccaagaaacagtttccaaatgaaaaaaaa

SEQ ID NO: 398 - DNA, 478 bp, Mus musculus
  misc_feature 205, 215, 220, 223, 224, 242, 247, 248, 253, 261, 265,267, 282, 324, 326, 330, 415, 418, 461: n = A,T,C or G
actgagggtcctctgccggagagtctgtccttgtcacagccagtagtggcgtcaggaaggactccagggacctgcctatattttagtggtgttccaaaagtcctgccctctagccaaccacctatatcctgctcagaggagggagttggaaatgctaccctgagcccagtgatgggtgaggagtgtgtcagggtctggagccacnagcgccttgngctcnccnngctccttacttcagaggngctgnntctgngtgtcagnggantnctggtcttgggcttnttcctggttctgctctgtggcctcctctgctgtaccactgcngnctgnttccatccgcgcccagagttccactgatctagaacacggctttgagggcgacagactgcgcctcactctgctgtcaggtacaggncangttcttggggcccacttctgagtttagaagacctattacaaanagtgaagaactagactg

SEQ ID NO: 399 - DNA, 548 bp, Mus musculus
  misc_feature 291, 492: n = A,T,C or G
ctattgatgagtatgtctgtcccagtgccagtcgacagaggacgccatgacagtgctcacaccactgacagagaaagattatgagggcttgaagagggtgctgcgctccttacaggcccacaagatggcgtggcctttccttgaaccggtagaccccaatgatgcaccaggattattacggtgttattaaagagccaatggaccttgccaccatggaagaaagaatacaaaaacggtattatgaaaagctgacagagttcgtggcagatatgaccaaaatttttgataacntgccgttattacaatccccgtgacacccctttttaccagtgtgcagaagttcttgaatcattctttgtacagaaactaaaaggattcaaggccagcaggtctcataacaacaagctgcaatctacagctccttagaactcagcgtgtctgtcacctaagctagacacagcaagtctggcgctctgaactatttaaactaangcgccagatattttcagtcaggctttcctgacaagaccgtaacctcgttcatattg

SEQ ID NO: 400 - DNA, 536 bp, Mus musculus
gacctgctgcttaccccataccccaggctggctaccaagggcctccgggcccctatccagggccccaacctggctacccagtcccaccaggaggttatgcaggtggtggccctagtggctttcctgtccaaaatcagccagcatataatcatccaggtgggcctggggggaccccatggatgccagccccccctcctccactgaactgtccaccggggctggaatacttagctcagattgatcagcttctggttcatcagcaaattgagcttctggaagtcttaacaggctttgaaacaaataacaaatatgaaatcaagaacagcctcgggcagagagtttactttgcagtggaagatactgactgctgtacccgaaactgctgtggggcgtctagacctttcaccttgaggatcctggataatctgggccgagaagtcatgactctggagagacctctgagatgcagtagctgctgcttcccctgctgcctccaggagatagaaatccaggctcctcctggggtaccagtaggg

SEQ ID NO: 401 - DNA, 560 bp, Mus musculus
  misc_feature 61, 120, 321, 340, 497: n = A,T,C or G
aactgaggacaggttgcttggaaacgagctgtcaaaggagttagagaaatgtgtgatgtgngtgacacaaccattttcaacctgcactgggtgtgccctcggtgtgggtttggagtatgngtagattgctaccggatgaagaggaagaactgccaacagggtgctgcctacaagactttctcttggataaggtgtgtgaagagtcagatacatgagcctgagaacctgatgcccacacagattattcctggcaaagccctctacgatgttggagacattgtgcattctgtcagagcaaaatggggcataaaggccaattgnccctgctccaacaggcagntcaagctcttctcaaagccagccttaaaggaagacctgaaacagacatccttgtctggagaaaaaccaactcttgggaccatggtccagcaaagttcccctgttttggagccagtggctgtgtgcggggaagcagcctccaagccagccagcagcgngaagcccacctgtcccaccagcacttcacctttaaactggctagctgaccttaccacatgagg

SEQ ID NO: 402 - DNA, 457 bp, Mus musculus
  misc_feature 19, 25, 41, 53, 99, 100, 109, 112, 127, 135, 141, 143: n = A,T,C or G
gcagcttgatggtaccaangcattnctcacttgcggcagcntctgtgaactgnctccggcatgagcgtccatgatgtgcacccgtgcttgattatcctnntacgccagnggnagagagagctgggangcatgtgnataaantntcttgggcccttgacagagatccatggaaggggtgcgttcaggcagaccagcagctcatggaaaagaccaaggccaaggtgttctctgcattcatcacagtgttacagatgaaggagctgcgagtcggtgatatcccccagtactcccagctggtgctgaacgtctgtgagactctccaagaagaggtgattgcactctttgatcagacccgccacagcctggcatccgacagcgctgccgaggacaaggacagcatggagacagatgactgcccctcggccccggcacaaggaccagcgtgatggggtgaccc

SEQ ID NO: 403 - DNA, 213 bp, Mus musculus
gaggatcagggtatcgttgatgcctaccgtcaagccctgccccaagttcgtctctatggccccaccaactttgcacccatcatcaaccacgtgaccaggtttgcagcccaagctgcacagcagaggtcggcctcggtaagagctgtggacggcagtggtgccaagctggcaggaccgtgtagagggcatcttggcagtgggagtgtgcacctg

SEQ ID NO: 404 - DNA, 402 bp, Mus musculus
ctgcctccaaggccatgaagatgggcgactggaagacctgccacagtttcatcattaatgaaaagatgaatgggaaagtgtgggaccttttccctgaggctgacaaagttcgcaccatgctagttcggaagatccaggaagagtctctgaggacctacctttttacctacagcagtgtctatgactcaatcagtatggagacactatcagatatgtttgagctggatctacccactgttcactccatcatcagcaagatgatcattaacgaagaattgatggcttccctggaccagccgacacagactgtggtgatgcaccgtactgagcccactgcccagcagaacttggctctgcagctggctgagaagcttggcagcctagtggagaataatgaacggg

SEQ ID NO: 405 - DNA, 146 bp, Mus musculus
  misc_feature 27: n = A,T,C or G
ctgctgtatttgtttgccttgaaaatnctttaccccaaaacactgtttttacttcgcggaatccatgaatgtaggcacctcacagagtatttcacgtttaaacaagaatgtaaaataaagtattcagaacgcgttaaaaaaaaaaa

SEQ ID NO: 406 - DNA, 437 bp, Mus musculus
  misc_feature 386, 406, 414, 415: n = A,T,C or G
gactgaggagaacaggggagatcaagtcagaagtccgagaacagatcaatgcaaaggtggctgagtggagagaggagggcaaggcagagatcattcctggggtgctgttcatcgacgaggtccacatgctggacattgagagtttctctttcctcaaccgggccctggagagcagacatggcgcctgtcctcatcatggccaccaaccgagggatcacccggatccgaggcaccagctaccagagtccccacggcatccccatcgacctgctggaccgcttgctcattgtatcaacgtcaccctacagtgagaaggataccaagcagattctgcgcatccgctgtgaggaggaagatgtggagatgagtgaggacgcctacacagngctgacccgcattgggcctngagacctnncctgcgctatgccatccactca

SEQ ID NO: 407 - DNA, 292 bp, Mus musculus
  misc_feature 57, 80, 87, 102, 103, 144, 154, 161, 169, 173, 174, 185,198, 216, 231, 234, 235, 239, 246, 248, 256, 260, 262: n = A,T,C or G
cttaccacgtgcacttgtgcttttgaccgtggcctggagatgaagaccatattggcngagaatgatgaagtcaaattcangtcctantgcccaaagcacagnntcacacaggaaacccgaggagggcctgggcgaaggagcagnccaggagaanggggccnctgagagntctnnccagagccctntggagccctatgntagtctggaagcccgagncgagaggaggcccancgnntganttgttcncnagcagaancctncngcagcatggaggatgagttctacacctttg

SEQ ID NO: 408 - DNA, 226 bp, Mus musculus
  misc_feature 129, 131, 132, 145, 173, 208: n = A,T,C or G
tcacggagccatgaaccagcagcacatgatgccttcccaagccttcccgatgcggcgccctctgcctccagatgacatccaggatgactttgattgggattcaatagtgtaggcttgtttctgcaaganannaaactccaggatnctttttgttcagtgaaaggacggattanaggcccggttgagaaaacaaagctatgttaccagngctatctaaattcctttc

SEQ ID NO: 409 - DNA, 507 bp, Mus musculus
  misc_feature 126: n = A,T,C or G
tgaggcctggttgcttgttacccaggcaacggacaggctatgtccgtcacgttgataacccaaatggagatggaagatgcgtgacatgtatatattatctaaataaagactgggacgccaaggtangcggaggtattcttcgaatttttccagaaggcaaagcccagtttgctgacattgaacccaaatttgatagacttgctgtttttctggtctgaccggcgtaaccctcatgaagtacagccagcatacgccacaaggtacgcaataactgtttggtattttgatgcagatgagcgagcgagagctaaagtaaaatatctaacaggtgagaaaggtgtgagggttgaactcaagcccaattcagtcagcaaagacgtctagtggggccttgggtccggcagtacccacgtcacctacagcctctcagttgccttctgtggactcgtggacaggatggacagagagacacctgcctggtatttcagctgggagccaggcgacttc

SEQ ID NO: 410 - DNA, 376 bp, Mus musculus
  misc_feature 123, 232, 329: n = A,T,C or G
gaacatggctgaggcacaccaggcctcgagcttgctgtcttcactgagttccgatggggcagaagtagagctcagctcgccagtgtggcaggaaatttacctctgtgccctgcgctcctgganaaggcatctctggcgtgtctgggtgaggagaactattggcctggtttccttctgggaattcagatttcggggcttcctgaaaccgattctgaagatatggatgtccagncccaggctacggttaactatggcttcctctgggatgctctgtgctgcccactggcctgccctgctcagcatccagactcactccactcccctgctgncctccccatgcagctcctcccccaataactttagtatagtcttctta

SEQ ID NO: 411 - DNA, 309 bp, Mus musculus
  misc_feature 33, 34, 40, 43, 44, 49, 64, 70, 92, 105, 107, 109, 115,129, 135, 139, 141, 155, 158, 179, 204, 211: n = A,T,C or G
cttctctgatctgtttccaggaagcatggatgnngccttncgnncgatngaagccagaaatgcngccatngccaatcactgcttcacctgtnctctcaaccgtgngnancagctnaccatgacctcggntacttntatngntcgagctatgtggnagntccctatggcagccggacccncggcctctcccgtaatcaggatggnctgctgnccacggagctcaacctcaatctctgccagcagatcaatgatttctggactttcaagatgacgggacgacttgagatgtatgcccggcaacttgccgaa

SEQ ID NO: 412 - DNA, 209 bp, Mus musculus
  misc_feature 39: n = A,T,C or G
actgagtgggcaagcaccgagttgaccggccaaaggagngactctgatgccgaagctcaaggaaagccctcagacgtctcccttacctcatctgtcacctccctcgactcctcccctgtcgacgtagcgtccaagaccaggaagtcatacaatagaattttttgaaatgtgtgcaaatctaattaaaattcttgcacagtaaacctaaa

SEQ ID NO: 413 - DNA, 357 bp, Mus musculus
  misc_feature 38, 51, 54, 197, 202, 204, 207, 212, 215, 282, 302, 307,331, 340: n = A,T,C or G
ccttagtggatgataatggtagtgaggaagacttcttngtatgaggagctntgncaggccaaccctcggtacctgcagccaggaggggaacagctggccatcaatgagctcatcagctgatggcagcagtggtctgtgcagaagcacttgtgggaccatgtgaccatggacgaccaggagctgggcttcaaggctgnttttntnatncaagntcnggaagcttctaacaaggactggtggtggggccggaatgaagacaaagaggcctggctccctgcaagntttgttaggcttccagcccnatcangaggagctgcccgagaactgcagnagctccccntggggaagagcaggatg

SEQ ID NO: 414 - DNA, 530 bp, Mus musculus
tatccagagactaagtccttgatgaaacctgaccacaatctgatctggattgtagccatgatgcttctcgtccagctggcttcattttacttagtcaaagatttggactggaaatgggtcatattttggtcctatgtctttggcagctgccttaaccactccatgactctggctatccatgagcttttcccacaatttcccctttggccaccacaaggccctgtggaaccgctggtttggaatgtttgctaacctctctctcggagtgccatactcgatttcctttaagagataccacatggatcaccaccggtacctcggagcggataagattgatgtagatattcccaccgatttcgagggctggttcttctgcaccactttcaggaaatttgtctgggttatccttcagcctctcttttatgcttttagacccctgttcatcaatcccaaacccattacctatctggaaatcatcaatactgtgatccagatcacctttgacattatcatttattatgtgtttggag

SEQ ID NO: 415 - DNA, 249 bp, Mus musculus
  misc_feature 44, 91, 93, 96, 97, 104, 105, 110, 111, 112, 132, 145,160, 174, 176, 183, 187, 188, 199, 202, 214, 221, 222: n = A,T,C or G
cgccccagtctccaaagcaaaaccacatccctttttcctatcanaaatggttccaaagagaatgggatccatgaggaccaaggccaccagncnctnnctttgtnncgctnnnttcatatgcactgatctttntactggatggacnctaacctgaagcccnctgccttgcatgancntatactnttcnnttttgcgcccncangacaacaatgtntccagannacctctaaccaagcatattgaggagcg

SEQ ID NO: 416 - DNA, 143 bp, Mus musculus
  misc_feature 34, 92, 109, 110, 117, 120: n = A,T,C or G
atgaccccattccagagaatgcgcgaaaccactntgaaggggcgcacagccatggagaagggccctagggggactctgaggagggacttcantcacataaacgtggagnntgagtcnatnttgggaagaagaagaaaaggttt

SEQ ID NO: 417 - DNA, 546 bp, Mus musculus
gttccttgatgtaagccagctgtccttcatccatgatctgggaccaaagggcctggaaggaatgatcatgaaaaggtctgggggacacaggataccaggtgtgaattgctgtggccatggacgagcctgctaccggtggtcaaagaggtggttgatagtaaaagactctttcctgctctatatgaaaccagatagcggggctattgccttcgtcctccttgtagacaaagaattcagagtcaaagtgggaaggaaagagacagaaacgaaatatggacttcggatcgataatctttcaaggacactgattttaaaatgtaacagctacagacacgcccggtggtggggaggcgccatcgaggagttcatccgaaagcatggcgctgacttcctcaaagaccatcgcttcgggtcctacgccgctctccatgagaacacgttagctaagtggtatgtgaacgccaaaggttactttgaagacatcgcaaatgcaatggaagaggcatcggaagagatttttatcacagattggtggttagtattgcc

SEQ ID NO: 418 - DNA, 332 bp, Mus musculus
  misc_feature 97, 119, 125, 134, 154, 157, 163, 202, 223, 231, 247,298, 309: n = A,T,C or G
gactgagaagtctagggagttggagtggaggtgagcaccgcgatcgcgaaagccctggctccctccgtccccgtcttccgccgatcgctctctgatnagttcctatccagcagcagatngaagcnggagatganatcttctcaaggtttgtgtnagnagcttnacgttctaggcagaaggcttcatgaaccaagtgaccgtnaaccaacaaggcttctctctntctcctcnctctaacaatgaaagntgctggtaacaagggaaaaaagagcgagagaattgtttataccatttcagntccaataatanatgacctatcagctctaaaagga

SEQ ID NO: 419 - DNA, 270 bp, Mus musculus
cactaaccgacagctaggcagtatggtggaaatcgccaagcagctccagacggtcaagcggaagatcagagatgaaagcctctgctcccagcaccacgaacctcttagccttttctgctatgaagaccaggaggctgtctgtctgatatgtgcaatttctcatacccaccggccccacaccgtcgtgccgatggatgatgctacacaggaatacaaggagaaacttcagaagtgcctggagcccctggaacagaagctgcaggagatcac

SEQ ID NO: 420 - DNA, 524 bp, Mus musculus
  misc_feature 22, 25, 33, 114, 136, 183, 222, 268, 281, 302, 305: n = A,T,C or G
actgagggatagagaaggctgngtnaggctganggactccgtcgatgggctccagtgtctgaccatgactgcggaaaacccaccctctggagacctgattcccgccccgctcgncacttgcaagctctgcctgtgngaacagtctctggacaaagatgaccatgctccaagagtgccagcgtntcttttgtacaccctgcctgaaacagtatatggtgctgncaatccgagaaggctgtggatctcctatcacttgtcctgacatggngtgcctaaaccangggaccctgcaggaaaccganatngcctgtttggagcctctggatgagtttcagctgtatcagcggctcaagtttgaacgaggtacacactcttctgtttatcccggtggcgaatgtagctttgacgcagaccgtctttacacgagcttcatgaaagattaaggccactgtgggctgtcgacagaaccacaaggggactgcaaggagtggaaacagcaattggcacatccgatgtcaactcct

SEQ ID NO: 421 - DNA, 251 bp, Mus musculus
tgagggaaaatgaccgagttgcaatggttaacggagtttcaatggataacgttgaacatgcttttgctgttcagcagctaaggaagagtgggaaaaacgcaaaaattactatccgaaggaagaagaaagttcagatccctgtaagtcacccagatcctgagccggtgtctgataatgaagacgaaaatgaatgtcacatttctttttcctctaatatatctattaaaatcttacaaattataaaaaaaaaa

SEQ ID NO: 422 - DNA, 511 bp, Mus musculus
tgaggcttgagcagcacaggaacaggagaagctggagcgggagatggcactgctgcgtggtgccatcgaggaccagcggcgacatgctgaactgctggagcaggctctgggcaatgcacaaagccgtgccgcccgggctgaagaggagctacgcaaaaagcaggcctatgtggagaaggtggagcggctgcagcaggcactggggcagttgcaggctgcctgtgaaaagcgagagcagttggagctgcgtctgcgcacgcgcctggagcaggaactcaaagccttgcgtgcacagcaggtaagcccagaagacctgtgcagctggcagtaccgggcacaattcaaagagtccctccctgctctctagtctttgtcctctttgaaagatgattgcttcatgttccccttggcgctgcttggtccctctcaggtgctttgtatagcaaaggctctgcctcgagccctactgtggctaataacagaaatttggtaccttagtaccagtcagtaa

SEQ ID NO: 423 - DNA, 298 bp, Mus musculus
  misc_feature 20, 28, 59, 63, 70, 75, 78, 85, 88, 102, 105, 106, 109,111, 112, 137, 155, 159, 160, 179, 192, 197, 220, 233, 246, 250,282: n = A,T,C or G
actgagaggagccccaggantgcttatnggttggctcagcagcctttacctcactgtgnagtnccaccgngcagnccntgcgctncgngcactgcaagctgnacnnatngnntgaagcttgcccgataccttgcatnctagatgccacatatgcntgcnnaagagcaccagcaggcctntggacatccttgncatgnaggagccaattaacagaaggctntttgagaaatacntgaaggatgacantggntcgagagacccttccagtgactgggagatgtnacagtcttccgtgaga

SEQ ID NO: 424 - DNA, 213 bp, Mus musculus
gactgaggctgcatttgccgcagctttacaacgtcttctgctctgctgaccagaacccatattaattatggcgtcaaaggggatgtggcagttattcggattaactcacccaattcaaaggtacttgaactttctttagttaatttctaccaactacagttatgagtcgtatcctacatagaaataaaatctccctgacttgaaaaaaaaaaa

SEQ ID NO: 425 - DNA, 512 bp, Mus musculus
gactgagggcgctgtgaggttatgcgcatgcttctagctgaccagggccagagctggaaggaggaggtggtgaccttggatgtttgggagcaaggcacattcaaggcttcctgtttgtttggacagatccccaagttccaggatggagagctcaccctgtaccaatccaatactatcctaccggcacctgggccgctccttcgggctctatggcaaagaccaacaagaggcagcactggtggacatggtgaacgatggactggaagacctattcagacgcattgcccggcagtatcgccatatcctgaaggagggcaaggaccagtatcagaaggagcttccgggacacctgaagccatttgaaactctcctggcccagaacaggggtggccagtccttcattgttggtgaccagatctctttcgcggactaccgcctgctggacgtgcttctgaatctcgagctcctgtttcctggttacttgaatgactttcccctgttctaatctccaa

SEQ ID NO: 426 - DNA, 178 bp, Mus musculus
  misc_feature 38, 79, 95, 98, 119, 133, 142: n = A,T,C or G
gatctgagaggcaaggtcggtgaaatggaggcctcggngatattgcccattctgaagaagaagctggccttcctttcangaggaaaggacagacncanctggactcatcttgacaattncgctgggccttgancagacaagnatggatgagcatgagtgtcactttggattatctact

SEQ ID NO: 427 - DNA, 493 bp, Mus musculus
  misc_feature 136, 143, 146, 149, 150, 155, 161, 167, 168, 170, 171,172, 176, 191, 195, 196, 198, 200, 201, 205, 211, 212, 228, 250,268, 270, 287, 395, 474: n = A,T,C or G
gactgagggtcgcccaaaccgtaggtgcgggacaggttcgcctggagctgagctttgtcaactcagacctgcagatgctgaaggaggagctggaggggctcaacatctccgtgggagtgtaccagggcaccgaggnagcagancancanntcttngattcnttttgnncnnnaggngaccatgcgaagtangatnntncnntcgnacttcnnccgaaatgtcaaaccngggaggctcatagcagaagaanctgaagtgtgacttatgngnctgcagtgtctttaggngccacgactcaaatgtcaactgacgtttgcaaggtgaccctgggacctcacttggccacttacaaggagatttttgtgtattcgctcctctgaaactcttcctgtgtntctgatggcttctgacaccctttcattgtgccggggatggaaccgggatcttgggtgtgcggggcaagtgctctacagngacctatgttcttgtctgt

SEQ ID NO: 428 - DNA, 507 bp, Mus musculus
  misc_feature 65, 69, 211, 220, 223, 242, 243, 256, 266: n = A,T,C or G
actgaggtgttgtcttacattaatggagtcatgacaaataaacctggaacagccctggtccactntatntcttcacggaatgtgtccctgcgcctgggggcactccaggagggagactctgggacttaccgctgttctgtcaatgtgcagaatgatgaaggcaaaagtataggccacagcatcaaaagcatagagctcaaagtgctggttnctccagctnctncatcctgtagtttacaggnngtaccctatgtcnggaccaatgngaccctgaactgcaagtccccaaggagtaaacctacatgctcagtaccagtgggagaggctggccccatcctcccaggtcttctttggaccagccttagatgctgttcgtggatctttaaagctcactaacctctccattgccatgtctggagtctatgtctgcaaggctcaaaacagagtgggctttgccaagtgcaacgtgaccttggacgtgatgacagggtccaaggctgcagtggg

SEQ ID NO: 429 - DNA, 461 bp, Mus musculus
  misc_feature 428, 432: n = A,T,C or G
tgaggtgacccaggcaatagtttcttcaatcggcctatgtcgctatggaggcaggattgactgctgctggggctgggcccgccagtcctggggacagtgccaacgactctagactggtccagcactgaccattgttggtagtgttttgggtttcttcctttacccttcattgaactggatattaatgcatctccaagggaaatggctcaggttttgtggttagttcttgttgctattgacgaccacagcagccacgcaagaagacagagccacggaagggaagaagacaaactttaatgtgtttttctcaaagctggagagaaaagggaacgtgtggcttatgagaaaaaaaggtaagtgaatggtcatgacgtacgagctaccagcctgggtacggattgagccaaagttggtcacttttttttttncacntgctttatccaaaattgattaaaaaaaaa

SEQ ID NO: 430 - DNA, 516 bp, Mus musculus
  misc_feature 40, 156, 175, 190, 216, 291, 344, 370, 431: n = A,T,C or G
gaggcctgtcctcatagagatttccagcaccccaaggaanctcaatgagactcttgatgaaaatttcaaaaaattccataaggatattatccatgagctggagaagaagacagaacttgatgtaaagtatatgaaccgctactcttaaaagatatnaagcagagcacaggaatanattaagattctttgnagaaatctcaggcaggagctgaagangatcagaagaaaaagtcaaggtggacgaaacgcactcaaatacgagcacaaagaaattgagtatgtggagacggngacgtctcgccagagtgagatccagaagttcattgccgatggctgcaaagaanccctgcttgaggaaaaaaggcgcttntgctttctggtggacaagcactgcagctttgccagtcacatacactactatcacatgcagnctgcagagttacttaattccaagctgccccggtggcaggaaacctgttgtgatgccacaaaagtcccagagaaaatcatgaacat

SEQ ID NO: 431 - DNA, 531 bp, Mus musculus
  misc_feature 40, 52, 79, 130, 149, 151, 163, 167, 174, 195, 205, 223,246, 248, 251, 259, 273, 286, 287, 288, 292, 294, 297, 298, 314,316, 317, 325, 327, 341, 343, 379, 380, 390, 392, 398, 399, 406,409, 411, 416, 426, 441, 443, 457, 469, 472: n = A,T,C or G
  misc_feature 474, 482, 489: n = A,T,C or G
actgaggcttagccggcacgggctaaggccctggtgctgntcaccgtgccgnagcatccaaaccctgcatgtgctcagnggagatatgagtcagctgccggctggagttcgagactttgtggcgcgcagngcgcatctgtgccaaccananggcatccacatngtgncgatggnaccgaggcatgagaacactgncatacatggnccatgctggaagaacagngctcttatccgcaaactccccangnatnagaactgngtggcgtggccctntcagacgcccaannntgtngnacnntatagaaagcaagcananntgattganangtccttcgcatggngncacagagcctctcctggctggctggggccagggcannagctgggcancnggatgnncccagangancnccatnagtagctgcngacgattagattacnangatgcatgcatggnctgcattatgtntgngnttccattnagaatgnagtcccgtgggctcctccactctcccgtattggagtgcgaat

SEQ ID NO: 432 - DNA, 229 bp, Mus musculus
  misc_feature 26: n = A,T,C or G
gactgaggaatgtatcctgtcggggntaatgtcagtgaatgggaagaaggttcttcatatggatcagaacccatactatggaggagaaagtgcgtctataacaccgctggaagatgtaagtctctgacgcacacgcttacatgttggctcatttgggactgtccacttttggactcgctcttggttttggcttcctattactatatttttttgtagggtaggaggaggg

SEQ ID NO: 433 - DNA, 164 bp, Mus musculus
gactgaggttttgtccaaggatccgaaccactttgcagtctacgtactgtgacaccagtgcccagcccacctgtgattatggatatggaacttggaactctgggacaaatagaggctacgagaactatggttatggctatggctatggccaggataacaccacc

SEQ ID NO: 434 - DNA, 520 bp, Mus musculus
gactgagataccctattttagtcttatgaagattttaatgtctgcctccccaactatgcatagtatatcacagtttcatcaaaggacaccagcaatgtgcagttgtcgtcaaactcagagtggtgagaaatacagtgatccttttaaacttggctggagagacttgaaaggtctgtatgaagacattagaaaggagcttgcacatctccaccagagaactaaaggacatgtccgaatactacttcgatggcaaaggaaaagcctttagaccgattattgtggtgctaatggcccgagcgtgtaatattcatcataataatgcccgagagatgcaagccagccagcgctccatagccttagttgcagaaatgatccacactgctactctggttcatgatgacgttattgatgatgcaagttctcgaagaggaaaacatacagttaataaaatctggggtgagaaaaaggctgttcttgctggagatttaattctttctgcagcgtctgtagctctggcacg

SEQ ID NO: 435 - DNA, 351 bp, Mus musculus
  misc_feature 25, 38, 43, 45, 56, 91, 96, 155, 158, 180, 183, 187,192, 194, 201, 208, 211, 213, 215, 254, 288, 324, 332: n = A,T,C or G
ccatggcaaccgagagctggatgantattttacacttngccancngatcagatggnaatcatagacggtattgatccgagagatgaggcanatggnccagaagacctcaatcggacctgtattgatgaaaagctaggtggcgtccttaatgtgtngcnctttgaaggatccttgtgtgtngancagncagtnantgccacnagttganagntngnatactgaacactatcctttcactatataagtaaagcaantatattaggccaggatctcactgaggaatttgtnttgtcatcttttagagtaaactaaaaattgtatagncgtctgcncacactaccctacctaaat

SEQ ID NO: 436 - DNA, 201 bp, Mus musculus
  misc_feature 16, 35, 40, 44, 87, 124, 152, 169: n = A,T,C or G
ctttcatggggcaggnaaggaaagccaatgcgcangtgtnccancattcgaccaggagagactggaatggatgtaacaagccggtgnacccttggagaccccaacaagctgccataaggggttncccaacctgctcgcatgccctatatttnagacaagcaccctaggnaaaccttggaagtgatcaaccttttgagaaag

SEQ ID NO: 437 - DNA, 517 bp, Mus musculus
aactgagggatatacatcagaagaaaccaaaagccagctggaatagttcctggatgagattttattcctgtttgcagttgttttgaggggggataccctctggatcagaattaaaatgctctggtttcaaggaaagagcctgcagattgccaaatcctcctttggactcttgagaaatctctctgcctctaacagagctctgcctgtgttgaccttattcacaaaggccccatgcccgctgtgtgatgaagctaaagaagttctccagccgtataaagacaggtttattttacaggaggtggacatcacacttccagagaattctacttggtatgaacggtataaatttgacatccccgtcttccatttgaatggccaatttctgatgatgcatcgagtaaatacctcaaagcttgaaaaacagcttcggaaacttgagcagcaggtgctggctacaaactgatattttctcattgatgcaagtacccagatgagaagtttacttctaggcatcttg

SEQ ID NO: 438 - DNA, 178 bp, Mus musculus
ctgagagtatcatggccttaggcacacagctgatgcttctgctgtggaaaaattacacctatcgacggagacaaccgatccaactactagtggagttgctttggcccctcttcctcttcttcatcctagtggctgtccgtcactcccacccccctctggagcatcacgaaaaaaaaaa

SEQ ID NO: 439 - DNA, 115 bp, Mus musculus
  misc_feature 31, 37, 69: n = A,T,C or G
cttccaaaatctgaacacagaaatatgtgangagccncatccgcgccgttccccatgaccgccggctgnagtacacagagtaccaacagttggagggttggcggtggagtgtggc

SEQ ID NO: 440 - DNA, 466 bp, Mus musculus
  misc_feature 358, 361, 362, 367, 370, 389, 396, 414, 425, 426: n = A,T,C or G
tgaggacagtgatggggacaagagtgatgacaacttagttgtggatgtgtctaacgaggacccttcttctccacatgcaagccccacacactcaccccgggaaaacggaattgacaaaaaccgtctgctgaagaaagatgcttcaggtagcccagcatccacagcctcctctggaagttcctcttccctgaaatccaaagaagtgagcctgcatgaaaaagccaacactcctgttctgaaatccagcacaccgacgcctcggagcgacatgccaaccccaggcaccagcgctactccaggcctccgtccaggtctcggcaagcctccagccatggaaccccttgtcaaccaagcaggncannacccnttnccctactgtgctttgtttnttctgtnccagtgttgatgtgctgnctgctaatctnntgacagttagcccctgaatgtgaagcaccttggcgctgtt

SEQ ID NO: 441 - DNA, 132 bp, Mus musculus
  misc_feature 42, 48, 49: n = A,T,C or G
gggatgacagtggaactgttgccccccggcggagggggcagnaggctnngcccttctgcgctggtgggcctggctcttggggtaccagctgctccttccttctttctcatgttctaaatggaaaagaagagt

SEQ ID NO: 442 - DNA, 512 bp, Mus musculus
gattgagcctctaggtcgccatggcaactgggcagaagttgatgagggctattcgagtgtttgagtttggtggaccagaagtgctgaagctccagtcggatgtggtcgtgcctgttccacagagccatcaggtcctaatcaaagtccaccgcctgctggtgtcaacccagtagagacatacattcagctctggggcctacagtcgaaaacctgccctaccctacactccaggctctgatgtggctgggatcatagaatccgttggagacaaagtgtctgctttcaagaaaggcgacagggtcttctgctacagcaccgtctctggtggctacgcggaatttgctcttgcagccgatgacactatctaccccctgcccgagacactgaacttcaggcagggggctgccctggggatcccgtacttcacggcctgccgagctctgttccacagtgcccgtgctcgagctggcgaaagcgttttggttcatggggccagtggaggagttggatta

SEQ ID NO: 443 - DNA, 252 bp, Mus musculus
  misc_feature 28, 37, 39: n = A,T,C or G
atttgaggatacactattatcagcctcnggccctggnanaaacatgcagacagcatcctggcactggcttcagtcttctggtccatctcttattactcctctcccttcgccttcttctacttatacaggaaaggttacgttgagtttgtccaaagtggtgccattttctcactatgccgggacattgctgctgctacttgcaggggtggcctgcctccgaggcatcggccgcatggaccaacccccagtacc

SEQ ID NO: 444 - DNA, 199 bp, Mus musculus
  misc_feature 37: n = A,T,C or G
gactgagggtttttaacatgttcctgagcatcgaggntcaattctgtcaatgtacggaacttccttacctgggggagcaaacaggaagaaaggaatttgggaaacatcactgcatagtccagcatccctttacagtgttagcactgccctccaccacactgagttcaatgaagaaaataaagcgacaacatagattata

SEQ ID NO: 445 - DNA, 367 bp, Mus musculus
  misc_feature 18, 234, 329, 348: n = A,T,C or G
ctcctttggcatctgtgnggtgaaggatgacactaaaatcagcatcgaacctgatggagctctgcaatgtagcatgctccagtgtgatgccaggagggggcaccaacctggagcttgccctgcacatgcctgcatgatgcccagggcagtgttcaggttgccctggagactctgttactcagaggaccccagaagccccggactcacccactggttgactatcgatacacaggntcagacatttggacccccatggagaagaggctcttcaagaaggcattctgtgcccacaagaaggacttttacctgatacacaagacgatccaaantaaaagtgtagcacagcgnaagcgagtactactacatc

SEQ ID NO: 446 - DNA, 180 bp, Mus musculus
  misc_feature 157: n = A,T,C or G
ggacccagggccagttcgcggagctgtcggctgcgaggacaccgcttccctagagcccggcacggaggccttgtggtcaccagccacaggctccagatagctcgacccacctttgctgggcgaaagaggtgtgggcgttcatctgcccattacgggntgagaaggtgaacgaaaaaaaaa

SEQ ID NO: 447 - DNA, 295 bp, Mus musculus
  misc_feature 37, 40, 61, 63, 83, 85, 128, 130, 154, 169, 179, 191,202, 204, 211, 230, 249, 252, 263: n = A,T,C or G
aaccctcgaagacttcaggcacacattgaacagtggnacngcggaaggcagacagccttcngngacacctcattcctgagtcngncacgggggcggtccgcctcccgagcctggagccccactcgggngnggccccacattctgccgttccacnctgcagccccacagncccctgtgtncttgtgcctttncctgcggaccngntgctgtntttccaccctggacctggnccccctctatgctggacgnggngttaggccgtnattgggacgccggagcgcagcgagaacaccag

SEQ ID NO: 448 - DNA, 308 bp, Mus musculus
  misc_feature 234: n = A,T,C or G
actgaggacccagcctcgatgcccacttgaaaggccggaagcaccgggatttggtgcaactccgagctaccaggaaggcacagggactccgaagtgtgtttgtcagtggcttccccagggatgtgggttctgctcagctctctgagtacttccagacatttggccctgtggccaatattgtcatggacaaggacaaggtagagtgaatggctctgggattaatgtgtttgcttnatatgcagagagagaaacctgcagtctgttttggcttgcccttggtagacaattcagaacattaagtagctcag

SEQ ID NO: 449 - DNA, 417 bp, Mus musculus
  misc_feature 89, 181, 312, 345, 347, 350, 399, 400: n = A,T,C or G
cagaaaaactatggaatgcggtgtaccactgactgaccgtaaccaaacgaactgtaccgacagtagtttgaaagccagcagttagcagnttcttcagacttcaacactgtccagcgctttccagagcagactcactgtttacaagaactcttggcctcccgaagttagcaacctcagattntgtttatttaaagaatttctgtgaaagaaaaggaccccagagcccattttccaaaatggccatggacgggtatcctgtggatggtcattttgggtttcattattgctttcatcctggcgttttcagtcggngccaacgatgttgccaattccttcggaaccgcngnggnctctggcgtggtgaccctgagacaggcctgcattctggcttccgcattnnaaaccactggctctgtg

SEQ ID NO: 450 - DNA, 421 bp, Mus musculus
  misc_feature 223: n = A,T,C or G
gactgaggtggggtctttcaatcctagtcgactatgacgagaaaggctacctcctacagatcttcaccaagcccatgcaggaccggcccacactcttcctggaagtcattcaacgtcacaaccaccagggctttggagcagggcaacgttcaactctctgttcaaggcgttcgaggaggagcaagccctacggggcaacctcactgacctggagcccaatggngtgaggtctggaatgtaagcccctatccacacatggaccacgcaaagtgctggacacatcagtcatctccaactggctgaaaggctgaacctcagggctccacccacgtcatggccacaccccctctattacaagagtccgccttgcctgagtcctccctgctgagtaaagctaccctcccaggtccaaaaaaaaaaa

SEQ ID NO: 451 - DNA, 408 bp, Mus musculus
ctgagcaaagcatcccaggttgtcttcaccaatggggaagaaataactgtggagcatcccgatatgaaacaagtttactcttttatttacgatgtttccttttggtcttttgatgaatgtcaccctggctatgccagccagacaactgtatatgagacactagctgcaccactcctggacagagcttttgaaggctataacacctgtctctttgcttatggccagactggctctggaaagtcctatacgtaagtaatattggtcatatgactaactaaacacgtttataagtaatatcggtcttataactaactaaacacgtttatgttttctttcgtcagaaacctcatggttgtagaaagaataccactgttgttttattaaaatttacaaactataaaaaaaaaa

SEQ ID NO: 452 - DNA, 109 bp, Mus musculus
  misc_feature 52, 55, 81, 93: n = A,T,C or G
tgccttgatgcagtggtctgtgaaggtggcaatgacatctcgccacaacggngcnttgttgagtgaccgaaggatctctgncatggagcaggnccagcccaggcccact

SEQ ID NO: 453 - DNA, 178 bp, Mus musculus
  misc_feature 129: n = A,T,C or G
gactgagggacttctcccaatgttgcccgtaatgccattgtgcaactgtgctgagctggtgacctatgacctcatcaaagatactctcctgaaagccaacctcatgacaggtgtgttagcgccacattnaagctggtagggggccgtctagttgtctaaagactgcatcttgttaaaa

SEQ ID NO: 454 - DNA, 317 bp, Mus musculus
  misc_feature 31, 32, 40, 42, 46, 53, 54, 56, 71, 84, 89, 107, 117,122, 124, 129, 140, 145, 152, 157, 165, 183, 193, 213, 240, 275,289: n = A,T,C or G
ctgcacttgctgaacgtaacatacatgaagnncatcgagntntgcngagccgnntntgagaacaatactcnaacagaacagganggccntgctcctgcccgacagcntatctacacngaaangnactgnccaatctgatnacatnaagcctnctttnctgttganacaaactgtggacatctnttttgtggtncctgcataattgcatactgncgatacgggtcctggcttggtgcaatnagttgtcctatctgtagacaaacggtagtgttctntagatataaagtancacttatggtatttcatttcataaatat

SEQ ID NO: 455 - DNA, 490 bp, Mus musculus
  misc_feature 21, 39, 46, 66, 70, 85, 98, 112, 146, 148, 150, 151,185, 187, 189, 193, 218, 244, 247, 248, 285, 288, 326, 339, 345,355, 370, 384, 399, 413, 428, 431, 432, 434, 435, 440, 442, 469: n = A,T,C or G
ttgcaggtttgctccagctcngccgggccatctcccagngatgtcnccgtcccagatccactcagncccncgatggtgctgtctnaaggattcctganccttctcacctggnaccctgagaatgaatagtttcctgaaaccctagngncnnacagaccccggctgcaggagcctggagtcgcagnanangcanctaaccatccttgcctctgtcttgntggtggccaggagcttctctgacagnggnntgtttagctcaccccagttcgtagacaagctgaaacnaancacgaagtccctggtggaggatttcaactccaggcccnaggaagtgatgcngtcagngagtgagcangtggtggaggaagtncaccagggtctcgngagcatggggttgancgctctgagcagcnagaatacggggtccntgnngnnccagntncagaacattgccaagaaggaaaactgngtccgcagcgtcattgaccag

SEQ ID NO: 456 - DNA, 549 bp, Mus musculus
  misc_feature 53, 65, 66, 79, 82, 86, 87, 90, 92, 100, 103, 116, 131,133, 137, 152, 174, 175, 188, 191, 195, 198, 221: n = A,T,C or G
gagagccccaccacctgccccctgcggaaacacaggtaccatccgggaagccncgcacacgcctnnaccacatcgccanctnctannctngnagctgcanganccagacagaaacngtacctgtaccatancnagancaggtacttgagttngtccagctctctgaaccttacnnagacccaaggtcnaantcanggntccagaacctgaagggcttagangcagaaaagactgcaagaggcaggaactggaaaagctgaaaatggctgccaagcctatgctgccctcaggcttcagtctgcccttccctatcaactcacccctgcaagcagcatccatatacggcgcatcctaccccttccacagacctgtgctccccatcccgcctgttggactctatgccacgccggttggatatggcatgtaccatctatcctaaggaagaccagatggaccagactccaggatggatgcttgcataaaagcatccccctccctctccgagaaggtggtgccaactctgctcctgaatgcgagccttgcattgtc

SEQ ID NO: 457 - DNA, 454 bp, Mus musculus
  misc_feature 41, 65, 78, 82, 91, 134, 147, 151, 152, 199, 222, 284,367, 369, 374, 384, 387, 389, 390, 394, 413, 435: n = A,T,C or G
gccgagatggtgctggagacactcatgatggagctggctgngcagatgagagaggtggagctggnatcagagggagcngtcncatccgcantcaggaatatcatgcactggtgtggacatacagcatggttggnaaacacgcccagncccnnctatgacatcagccctggtgagcggctgcagttggaggatgtatgtnccaagatccacccatcctactgngggcctgccatcctcaggttcaggcagctgctggctgaacgagagcccgaggtgcaggaggnggcgcggctgttccgctccgtgttgcaggaggccctggagaagatgaagcaggaggaggaggcccataagctgacacgttagngnagctngaggccccgnggnannctgncgtgcttcaagacccgcgntcgcatagtaccctttgccagngacatacgaaccatctagg

SEQ ID NO: 458 - DNA, 518 bp, Mus musculus
  misc_feature 21, 48, 53, 78, 83, 107, 109, 113, 127, 133, 153, 249,292, 294, 301, 339, 384, 388, 436, 442, 472, 481, 493: n = A,T,C or G
gactgaggtgggtgtctttcnagcagaagagaagagaagagaagagangagangagaatagaagagaagagatgagangagangagaaccagaagaggtgagcgagnancctnacacaaagccaggncgtcancctgctggccaagggcctgnagacaccggccttcccgttccaggctacgagacagtgccagcatctgccgaggacggtgaaggctctgatgggcccggaggcaaggtgaccgacgnctgctggtagcccgctgcaccggctgcgctcgcctctacattngngtccagnctccccagccagtggatctttctgcctggagcccccgngcttgaggcgcagtctggatgaggacgaaccgccgccctcgccantcgntcggtaccggcccctgcacaacgccgcctcgcatgagggcctggccgncacctncggctcgccaccacgctctgcaccctcctncgacagctnccccagcttcgngcgtcgttaccctcgagcagagccg

SEQ ID NO: 459 - DNA, 180 bp, Mus musculus
  misc_feature 71, 75, 87, 90, 129, 142, 147: n = A,T,C or G
cttttggggtctgttccgagccagtgatcaaagacaatgtggtggtggtgtacgaggtgttggaagagatnctgnacaactgggttnccnttgtctacggagtcaaacatcctcaaagaactgataaancctcccactattnttcgnacagtggtcaacaccatcacaggaagcaccaat

SEQ ID NO: 460 - DNA, 393 bp, Mus musculus
  misc_feature 33, 59, 73, 83, 89, 107, 110, 124, 132, 142, 158, 159,170, 171, 180, 181, 186, 191, 202, 206, 210, 211, 215, 216, 218,219, 223, 239, 241, 250: n = A,T,C or G
gactgaggtgactgggggcctgggcctgtcagnggggatctacgcagaggcagagcggnagaaatacaaaacntctggagagngccttnctggcccccgccatcatnctnatcctcctgggggnggtcatgntcatcgcctncttcatcggggtgctnncttccctccgnnacaacctgnnccttntgcanacgtttatgtntatnctgnngatnngnntggncatggagcttattggngncataggggntttgatcttccggaaccagactattgactttctgaacgacaacatccggagaggaatcgagaattactacgatgatctggacttcaagaacatcatggactttgttcagaagaagttcaagtgctgtggcggtgaggactaca

SEQ ID NO: 461 - DNA, 415 bp, Mus musculus
  misc_feature 21, 33, 35, 38, 39, 42, 44, 46, 55, 56, 57, 77, 88, 89,109, 128, 190, 203, 212, 214, 226, 228, 229, 237, 238, 244, 252,262, 264, 293, 299, 320, 375, 383, 396, 399: n = A,T,C or G
ccccgggctccccgcttacgntgtggcaggggncntanntgntnantcgaggacnnncgtgcgccgctgggacgacnggctccatgcnnctgctcatgagattattganactatccaggtgggtctgngaggaaatcccagatctcaagctggccatggagaactatgtcctgatcgacttaccgacacncaaaagtttcagnaagcatgcntngggtgtgtgacnannacaagcgnnccatcnacagcatncaccagctgngnaaggccactacacagcccatgaagctgantactcntacgtgcaatggacttctgcngcacatcctgcagataagcatataaccactctgtcactgacccggagaagcttancaactacnagcccttctcccntgnggtgtatggggagacc

SEQ ID NO: 462 - DNA, 296 bp, Mus musculus
  misc_feature 68, 107, 111, 127, 128, 130, 135, 149, 159, 197, 218,237, 241, 253, 263, 270, 280: n = A,T,C or G
acacttacacgagtgcccccgcctattcttcccaggccatcccagtcagacaggggagcaacagctgnaaacacgttctcagacctgcattacagctcattttcttntggntatggagcctacgganntncattntatggaagctatancccttatagntatggatacaatggttggggctttaaccgcctccgtgnagatgatcttcctcccagtgnggtttgttcagcaagctgnaganagccgcacaggngcatttgagnccattgnaagcattgcncatgcatttgcctctg

SEQ ID NO: 463 - DNA, 457 bp, Mus musculus
  misc_feature 38, 84, 92, 104, 141, 145, 162, 188, 204, 205, 210, 259,274: n = A,T,C or G
gactgagaggccagtatgaggaagctgtggtggatcanggtgggaccagcacaattctcaacattcactatgagaaagaagagntggaaggngagtcccagggncatcaggggctgtctggcttggaaaaaaacagaccangggntcacagccccgactttntttgtggctgtgaaactctgtggggnaaaataccttaacctnnttagnctcgtcttccctggtcttgaaaaggaaaaaagctgaggtgatgaacccnacccgtccagtcagntcatcagagacctgagacagtgctcagagaactgggaagctcgacataaatactaacaaagaccttttccttgaggacgtgaaatactcatttagctccctgaagttttttgttttgtttttttataacctcatggagatgtaaagtatcaacacatattcacaagcaaataaactatcagtc

SEQ ID NO: 464 - DNA, 146 bp, Mus musculus
acagttctgtgtgtgcctccaacgaaccatttcgtaaagttgattacaccaaaaacgttaatccgaactggtctgtgaacatcaagggcggaaccacccgcaccctggccgtggcctcttcgaagagtgaggtgaaagaaagcaaa

SEQ ID NO: 465 - DNA, 345 bp, Mus musculus
  misc_feature 26, 57, 58, 62, 63, 64, 69, 72, 75, 78, 81, 86, 90, 93,97, 101: n = A,T,C or G
gccggaagccctccacgcaccatgcngcagcactctgttcccactgactttggcagnnttgnnnatccnggnccngancanggacntcancanccgntggnacctgcctgactcagactgggacaatgacagcagttctggtccgcctctttgagaggtccaggatcaaggccctggcagatgagcgagaagccgtgcagaagaaaaccttcaccaagtgggtgaactcccacctggcccgggtgacatgccgggtgggagacctgtacagcgacctgcgggacgggcgcaacctcctgaggctcctggaggtgctctcgggagagaccctgccaaaaaaaaaaa

SEQ ID NO: 466 - DNA, 336 bp, Mus musculus
ctggattacttcaaaggggccattcctatcaggaagttggtgagctggtataagacctcagtggagtgttccagggatgccatcgtgtttctgactgtccagggcaagctcatctgtgcagaccccaaagacaaacatgtgaagaaggccatcagattggtgaaaaacccaaggccgtgaccttcccgctgaggcatttggagacgccagggctgctgtccatggtttcaacataaagcggcctgtgaccagcagagcccaagagcagccacagagcagaagtccctgttcccttttttatggactcttatgcactacaggcgaacacaaaaaaaa

SEQ ID NO: 467 - DNA, 543 bp, Mus musculus
  misc_feature 208: n = A,T,C or G
actgaggatgatggtgaatgtagaggaaatctcttcatggttattaataacaaaattgccaacttctcacagagctgtaccctttattcaggagatgatttggtagaagcgcttcccaagccttgccctggctgccccagggacatacctgtagacagcccagagctgaaggaggtgcttggtcattccattgcacagctaaatgcanagaatgaccatcctttctattacaagattgacaccgtgaaaaaagcaacatcacaggtggtagcaggaactaaatatgttattgagttcatagccagagaaaccaaatgctccaaggaaagtaacacagagctggcagaagattgtgagatcaagcaccttggacaaagtctcgactgcaatgctaacgtgtacatgagaccttgggagaacaaagtcgtcccgactgtgaaatgccaagcattagatatgactgaaatggcaagaaggcctccaggtttttctcctttccggagtgtcacagtacaagaaacaaaagaaggaagaactaggctc

SEQ ID NO: 468 - DNA, 445 bp, Mus musculus
  misc_feature 18, 52, 68, 74, 92, 100, 109, 112, 117, 129, 130, 173,185, 193, 200, 202, 223, 225, 226, 257, 273, 277, 346, 374, 403,416, 418, 421: n = A,T,C or G
actcaccgggggccttcngatctcttgatgccagcagagaggagcttgttgnattccatgacttgcanaaaccntattggatgggaaaagtncagaggancctgggttngtnatctncacctatgaggnnagggggaccatgtacaacacttggaaggcatgtctccctccgnctggacaaagancacttggncaatatntnaggagggcccatgacatacantnnccgcctggaagaaatccggttacactttggnagtgaggacagccaanggtntgagcacctccttaatggacaggccttctccggggaggtatgtgcacctgccaaagtgctaaggcttanggccaacttgtaggattccacgcctacntaacagtgaaataacagatgggaaggctnccttgaacctgananatnaaatccgggcctcctttaaaaaaa

SEQ ID NO: 469 - DNA, 303 bp, Mus musculus
  misc_feature 226, 250: n = A,T,C or G
actgaggctgggatgataaagagcgatgatgaagagtatttcattgaacccttggagagagggaagcagatggatgaagaaaatggaaggattcacgttgtctacaagagatcagcagtggagcggcctcccagagatgtatctgaagacgtctaccacaggggtaggcagcttaagtaaccagagttttacaggacagcttgtaaagtaaaccatgaagtgtacntgatataatccctggtataaaatncctccaacatgcaaaatgaaagaaaaccttgattaaacatccaaaaaaaaaaa

SEQ ID NO: 470 - DNA, 472 bp, Mus musculus
  misc_feature 216, 218, 220, 224, 226, 228, 233, 239, 242, 245, 254,256, 264, 267, 270, 279, 287, 290, 291, 294, 297, 303, 306, 317,321, 325, 326, 327, 331, 333, 336, 340, 342, 352, 357, 360, 361,368, 378, 415, 429, 431, 437, 453: n = A,T,C or G
actgaggtggggtctttcacctttggggcatccgcctgcgctttgagaagcagccatctgaagaagaggagtgggctatgccggccagcccgcccagtcccgaagcgggcaccatccgctgcgcttcattctgtgcgggcttccccatcccagagtcgcctgcggccaccgcctatgcccacgctgagcacgcacagtcctggccatccatcaacntnangagngnantggtngcctcngantgngtaatcggnangctgagcnaanagngtgtccttnttgtgggnccnnacnttnttgctnatnttcacacacantacncacnnncgancntgnagananatgctacctnagccnttnnctccctnctttttcggntggtaggaatgagtccccacccccacctgttctattnaaagtggggtgacngnggaganaccctccctatgagantatgcctaactcgcaaggg

SEQ ID NO: 471 - DNA, 210 bp, Mus musculus
  misc_feature 16, 17, 45, 46, 62, 77, 84, 111, 112, 128, 157: n = A,T,C or G
tgaggaggcctgctgnncctgctgctgtgctgctgatcagtatgnngtcgtttcgggagcanagcagttaccaccgnaaaccanctgagcatacccacaggaggtgcacanntcatcccgcatagaanagttcagccctcgccaggcccagatgttncagaattttgggggagcaggtggtggtagtagtggcactggcagcatcatgta

SEQ ID NO: 472 - DNA, 370 bp, Mus musculus
  misc_feature 19, 27, 35, 52, 54, 57, 79, 80, 82, 88, 90, 96, 97, 98,107, 108, 113, 115, 117, 118, 120, 131, 136, 141, 145, 152, 354: n = A,T,C or G
ccacgtctccttagctggntgctgagnagccacangcagaacagcctgttgnanccncaccagaggctatgtcgggctnncnaccttnancatggnnnattccttgnncacgntncnnanagcctaccatngtggnccctngctnaggatgnggacattcactacagtcccggatgaaagagactgagaccctgctacaggagtggtctgggcaacctgagcgagattggtctgaaaaagcagatggcacaggggactgaggctgatcctgtgcaggagggagaagagattgcgaagaagcctggaccattcttgaagcaggggaaggagcccgctcacgcccagaggtttttntagactggcttggggc

SEQ ID NO: 473 - DNA, 109 bp, Mus musculus
  misc_feature 17, 20, 31, 51, 77: n = A,T,C or G
tacagtggtgcggtacnttncttcatgaagnctcatcgctgctatgacctngttcccacaagttccaagttggtggnatttgacacttcgctacaggtaaaaaaaaaaa

SEQ ID NO: 474 - DNA, 518 bp, Mus musculus
gactgaggtggggtctttagtgcagtggctccagcgccatgttcggctccaatcgcggtggcgtgcgtggcggacaagaccagttcaattgggaggacgtgaagactgacaagcagagggagaactacttgggcaactcgctgatggcgccagtaggccgctggcagaagggccgcgacctcacctggtacgcgaaggaccgcgcgccgtgtaccggcccgagccgtgaggaagagctggcagccgtgcgcgaggcggagcgtgaggcgctgctggccgcacttggttacaagaacgtgaggaaacagcccaccggcctgagcaaggaggacttcgtggagatctgcaaacgggaaggaggagaccccgaggagaagggcgtggaccggctcctgggcttgggcagcgccagtggctccgcgggccgtgtggcgctatcccgagaagacaaagaggctgccaaacttgggctgtcagtgttcacgcaccaccgtgtggacagcgaaggcccaagcact

SEQ ID NO: 475 - DNA, 283 bp, Mus musculus
  misc_feature 55, 91, 96, 116, 117, 147, 168, 222, 240, 251: n = A,T,C or G
cctaggtcattgactttattatctggccaacgacacataagaagatgtttcacangacagaggagttctttcccatgaagacagacacgcngatgnggacgacatggacacatcgnnacacacagtggggctggttctacctggcgnaatgtgggaaggggcacatgnttcagccggataccaacattcaatgttcagttagcagtgaagatatcgaaaaangcttcaaaacaaaccctnggggctccatntcctttactaccttgcaaattcaccatacaag

SEQ ID NO: 476 - DNA, 250 bp, Mus musculus
  misc_feature 60, 77, 143, 206, 210, 228: n = A,T,C or G
ctctaaaggccattagatacattcttcctttcaaggattcgtcgcatccagtggattttntggatgcattcagaacnttttacatggatggactatttactgatattacccttcagtgtccttcaggcataatcttccattgncaccgagctgttttagctgcctgcagcaattattttaaggcaatgttcacagctgacatgaangaanaatttaaaagtaaaatanaactgtcgggcatccaccatga

SEQ ID NO: 477 - DNA, 431 bp, Mus musculus
  misc_feature 20, 23: n = A,T,C or G
cttacatgctagctaaacanatnggggtagaacatgttgtggtgtacgtgaacaaggcagacgctgtccaggactcagagatggtggagttagtcgagctggagatccgggagctgctcaccgagtttggctataaaggagaggaaactccagtcatcgtaggctcggctctctgtgcccttgagcaacgtgaccctgagctaggcgtgaagtcagtgcagaagctcctggatgctgtggacacctacatcccagtgcccacccgggacctggacaagcccttcctgctccctgtagagtcagtctactctattcctggccggggcacagtggtgacaggtacattagagcgtggcattttgaagaaaggagatgagtgtgagttgctgggacataacaagaacatccgcactgtggtgacaggcattgaa

SEQ ID NO: 478 - DNA, 334 bp, Mus musculus
  misc_feature 30: n = A,T,C or G
tgagtgcggccaaggattctcccagcaggncaatggccattgcatggacaccaatgaatgcatccagttcccatttgtgtgccctcgagacaaacccgtatgtgtcaacacctatggaagctacaggtgccggaccaataaaagatgcaatcggggctatgaacccaatgaagatggcacagcctgtgtggctcaagtggcctttttaggtgggtactcatcggctgcctttagactctctgagcctctctctcaggcctcgtatctttctctaggcctgggactttgccttcagttatatgcactttaaatcccatcaataaaggaaaaaaaa

SEQ ID NO: 479 - DNA, 111 bp, Mus musculus
gactgagatggggacttaatgttttggttgatattttgactgaagtaccagaacaggatgatgaattgtataatccagagagtgaacaagataaaaatgagaaaaaaaaaa

SEQ ID NO: 480 - DNA, 376 bp, Mus musculus
  misc_feature 186: n = A,T,C or G
gactgaggactctgaaaaggactcttgttcaggcactggtgaggaggatgaagatgaggacgaggatgattacagatatgatgaagatggggatgattacatgattgatgattttgtagtgcgaaatgaggaaggcgatgacgagaatagcaatcaacaaggagaaaatctgactacatcacaacntgaaattagtaaaacagaattctctttgtaagttaaatatgaagcaaggtttctgccttgttcaaatcattatataatgcatcaaagcttactaatatcatcttctaaaacatttggttaggaattatggtttttatgtttgagtattttaaatttgacaataaaagtttattccagtgaaaaaaaaaaa

SEQ ID NO: 481 - DNA, 254 bp, Mus musculus
  misc_feature 17, 28, 33, 51, 62, 74, 91, 97, 115, 157, 184, 189, 190,214, 220, 221: n = A,T,C or G
gctgatttgaagatttncctgagacctnagagnctgtttggattctgggcngataatacagnattttcacaganaaggacgaaatcttgantgatgntgcatccagactttggtnttacatacaggagaaactttccagctattgttgggaaatggntgaaactttgccacaccacctccaatnggatnngtggtgacaagttccccagactgnagcganngaccttaataccggaagctcttccgtggaaccg

SEQ ID NO: 482 - DNA, 502 bp, Mus musculus
actgaggttccttttagtcttctcctgccttgtgctttctgtgttttccaccatcaaggagtacgagaagagctctgagggggccctctacatcttggaaatcgtgactatcgtggtattcggtgttgagtactttgtgaggatctgggctgcaggctgctgttgccggtatcgaggctggaggggcaggctcaagtttgccaggaagccgttctgtgtgattgatatcatggtgctgattgcctccattgctgtgctggctgctggttcccagggcaatgtctttgccacatctgcgcttcggagcttgcggttcttgcaaatcttgcggatgatccgtatggaccggaggggtggcacctggaagctcttgggatcggtagtctacgctcacagcaaggagctggtgactgcctggtacattggcttcctctgcctcatcctggcctcatttctggtgtacttggcagaaaagggtgagaatgaccactttgacacctac

SEQ ID NO: 483 - DNA, 556 bp, Mus musculus
  misc_feature 19, 38, 40, 73, 75, 92, 232, 487, 504: n = A,T,C or G
ccgattttcggacactccncccaatttgggggaccaancngaccccacagccatcacaaggcatgacttcatntngaccaatggggacaacngcctggtcacgctgctgccactgctaaacatggcctacaagttccggacccagagcccagagaccctggagcagctgtacccttgggaatgcttcgtcttctgtctgaccatctttggcaccttcaccaaccagatccanttttttttcacacacatacttagggctgccatacctgggtcaccgtcctgcaggactggcacgtgatcctaccacggaaacaccatcgcatccaccacgtggcgccccacgaaacatacttctgtatcaccacaggctggctcaactaccccctagaggttataggcttctggcggcgcctggaggatctcatccaaggcctgacgggtgagaagccgagagcggacgacatgaaatgggcacagaagatcaagnagcatgtctggactctnggtcgccaaccttcccaaacctgaactgaagccatctgccaaatcccgggaa

SEQ ID NO: 484 - DNA, 545 bp, Mus musculus
gactgaggtttcccagttcgtggcgtttgaggagcttcctgcaggagccccagagctcccccaagatggccccactcgacgcctctccctgcccggccagctgggggcccttacctcccagcccctgcacagacatggctcagatccaggcagttagtgatgctgcccactgtggacatgtggctcaccctgggttcagggcattacctgaccgctctccttggagaccctggtttggaggggagacctgtgcagaagcacctccatccccacccttgggaccagcatctcctcctgcaataccctgggccccactgggaggggcaggacagagctgggcagtggatggggggtctgtggtccccccactgccctgggggcattagctggtctaactgcccggaggcaccggccctgggccttaggcacctcaaggacttttctgctatttactgctcttattgttaaggataataattaaggatcatatgaataattaatgaagatgctgatgactatgaataataaataattatcctgatctc

SEQ ID NO: 485 - DNA, 545 bp, Mus musculus
  misc_feature 82, 145, 228, 512: n = A,T,C or G
tgaggccaagtgaaagagtttggaattgaccctcaaaacatgttcgagttctgggattgggtaggtggccgctattcgctgnggtcagccattggactttccattgctctgcatgtaggttttgaccacttcgagcagctgctgnccggggctcactggatggaccagcacttcctcaagacgcccctggagaagaatgcccccgtcctgctggctctactgggcatntggtacatcaactgctacggctgtgagacccacgccttgctgccctatgaccagtacatgcaccgctttgctgcctatttccagcagggtgacatggagtccaacggaaagtacatcaccaagtccggggcccgtgtggaccaccagacaggccccatcgtgtggggggaaccagggaccaatggtcaacatgcattctaccagctcatccaccaaggcaccaagatgataccctgtgactttctcatccctgtccagacccagcaccccatacggaaaggtcngcatcacaagatcctcctggctaacttcttgga

SEQ ID NO: 486 - DNA, 542 bp, Mus musculus
  misc_feature 469, 478, 481, 514, 522, 526: n = A,T,C or G
atatagaaattccatgcctgcatccagttttcaacagcaaaaactccgtgtttgcgaggtttgttcagcttaccttggtctccatgacaatgaccgtcggcttgcagaccacttcgggggcaagttacacctggggttcattcagatccgggagaagcttgatcagttgaggaaaactgtggctgaaaaacaggaaaaaagaaatcaggatcgattgaggagaagagaggagagagagcgagaagagcgcctgggcaggaggtctggatcaagaacccgagatcgcagaaggtcacgctcccgggatcgtcgacggaggcggtctaggtctacctccagagagcggcggaagttttcccgttccagatcccgagatagatacaggcgccatcgcagccgttcccggagccacagccggggtcaccgccgggcttccagggaccggagtacaaaatacaagtaactactntgactcctnatnaacttcaaccaggagtgagccctttgtctttgngttcccangatntgctaagatctgtcat

SEQ ID NO: 487 - DNA, 541 bp, Mus musculus
  misc_feature 136, 145, 177: n = A,T,C or G
gaactgaggtgggagatgcacattccctcagtggcaggtatttgaaggggaaccgggaaagaaggtgaacttggcagagctgttcaagggcaagaaaggtgttttgtttggagtccctggggcatttacacctggntgttctaanacccacctgcctgggtttgtggagcaagctgnagctctgaaggccaagggagcccaggtggtggcctgtctgagcgttaatgacgtctttgtgattgaagagtggggtcgagcccaccaggcagaaggcaaggttcggctcctggctgaccccactggagcctttgggaaggcgacagacttattattggatgattctttggtgtctctctttgggaatcgtcggctgaaaaggttctccatggtgatagacaacggcatagtgaaggcactgaacgtggagccagatggcacaggcctcacctgcagcctggcccccaacatcctctctcaactctgaggccctggccagatgtcctctgactctcccatctctcccacccggctctaggccaaa

SEQ ID NO: 488 - DNA, 541 bp, Mus musculus
gactgaggattccatttaagatcgggcagcccaagaagcagattgtttccaaaacagtggagagagactttgaaagggagtatggaaaactgcagcagcctggaagagcaaactaaacgcctgcagaaggacatgaagaaaagcaccgatgccgacctggccatgtccaaatctgctgtgaagatatcccaggacctactgtccaacccactgtgtgagcaggaccaggactttctgcacatggtgacagccctagacacagccatgaaacggatggatgccttcaaccaggagaaggtgaaccagatccaaaagacagtgattgaacctctgaaaaagttcagcagcatattcccaagcctcaacatggcggtgaagcggcgggagcaggccttgcaggactacgggaggctgcaggccaaggtggagaagtacgaggaaaaggagaagactggccccgtgctggccaagctccaccaggcccgagaagagctacgtcctgtgcgggaagactttgaggccaagaataaacaactcctgg

SEQ ID NO: 489 - DNA, 541 bp, Mus musculus
  misc_feature 29, 30, 496, 519, 523: n = A,T,C or G
aactgaggaaactcccatgctcaccaagnnagcaaaaacagaagcgcagggtaggaggaggaggagcgacagctccttcttgccgtgcagaagaaggagcaagagcagatgttaaaggaggagagaaagcgggaaatggaggaaaaggtcaaggcagtggaagatcgagcctaagaggagaaagctcagggaagaacgagcatggctattggctcaaggcaaggagctacccccagaactttcccatttggacctgaattctcctatgagggaagggaagaagaccaaggacctctttgaattggatgacgatttcacagccatgtataaagttctagatgtggtgaaggctcacaaggattcctggcccttcttggaacctgtggatgaatcgtatgcccccaactattaccagattattaagatccccatggatatttcaagcatggagaagaaattaaacggaggtttatattgtaacaaggaggaatttgtncatgacatgaagaccatgttcanaaancgccgaaaatacaatggt

SEQ ID NO: 490 - DNA, 539 bp, Mus musculus
  misc_feature 477, 491, 510: n = A,T,C or G
tgaggtggggtctttcagtgacagcctttgatagtggtgaatactactgccaggcccagaatggatatgggacagccatgaggtcagaggctgcacacatggatgctgtggagctgaatgtggggggcatcgtggcagctgtcctggtaacactgattctccttggactcttgatttttggcgtctggtttgcctatagccgtggatactttgaaagaacaaagaaagggactgcaccgggtaagaaggtcatttacagccagcccagtactcgaagtgagggggaattcaaacagacctcgtcgttcctggtgtgacctgctgcggctcctccgttgtccatttgccttactcaggtgctacaggttccagcccctgctggctgtagctgcacaggatgccttcaatgtcttctaggtcccacaggaccccttgcttttattctagctaggatataaatttaaaaacatcatctanttccccctcctctntcccaccctccctcctttncttaccaccattgggtggctccgagactc

SEQ ID NO: 491 - DNA, 538 bp, Mus musculus
  misc_feature 17: n = A,T,C or G
actgaggtattaaagtnctactccaactggccaaccatcccgcaagtgtacctcaacggcgagtttgtggggggctgtgacatccttctgcagatgcatcagaatggggacctagtggaagaactgaaaaagctgggtatccgctctgccctggtagatgagaaggaccaagactccaagtgagggccccgggccctcgccgagcagagcgccctctccatcctcggggccagaaaagccttactgcgtggtggtgccctgttgctgtgatgcttgcgctgggcctcctggcagcaagcgggcaggtgcttttactttagtctctggctcagaattccctctggtgaacgtacaaccactgcactgtaaatcaatgtcgcgatcctgtattgctataaccgtgatctgttcttacgttgtctttattctctgccggccggctttgggagtcacttctgattcttgtttgggttgtagagatggtaaagccccctgatgtttttgtaacttcctgtaaaagggaaatgatggacagagg

SEQ ID NO: 492 - DNA, 538 bp, Mus musculus
  misc_feature 56: n = A,T,C or G
aactgagttctgggccaagacttgtattcaatcgagtaaacggccggcgacccctnaccacctccccatccctcgaggggacccaagagacctacacagtggcccacgaggagaacagtccgatttgtgtccgaagcctggcagcaggtagagctgacaacctggatggtggccctgccgatgagagtggcccacggcctgtgcagtatgtggagagcactcctgatccccggctacagaactttgtacctattgacctcgatgagtggtgggcacagcagtttctggctaaaatcaccaactgttcctagcagctgtccgggaaggaatgttgccatagtggaccctttgccagaagaggaccatggtgcgccgatccaccttgtggcctggctgggtaccggccacacctgaagtgccaacatttggacttttgcacctgttgttcccttggcttggctgtcccaagctgccagagccaggggccaaacctgtctaacctcagtcctgctcactgtgcccagggaccagcccctgt

SEQ ID NO: 493 - DNA, 537 bp, Mus musculus
actgaggagtcggattgtaacaataagtcacttgataatcaaggggaacagaactccaagcaacacttacctcacttttcaaagaaagacggacttagacggaacaaggttcctgggctctgtaggtctagttctatggactctttttctacaaccaagatcaagcccctggtgcctgccagggtcagtggcctgagcaagaagtcaggaagcatgcagacgagaaaacaccatgatgtggagaacaagccaggcctacagacgaagatcagtgagctctggaaaaactttgggtttaaaaaagattctgaaaagcttccttcttgtaagaagcccctgtctccagtcaaggacaatatccaactcactccagagacagaagatgagatctttaacaagcccgagtgtgtacgcgctcagagagcaatatttcactaagtgcagaccgttgcctacaagaaaatcttatcaactggagagatgtattagtctcaatgtcattttttaaatggattacattttgtatataaaaaaaaa

SEQ ID NO: 494 - DNA, 535 bp, Mus musculus
tgagatccagacgcggaagtgtgtcttcataccctgcggctcatccagtctgtggttctagaaccagacgtcttctccaaggtggcctctgagctccagagctccttacccttgcagcgcatcctggccatgtccaagagccgcaactcccacctgcagtctgctgcccaggagctcctggaagacctccgcgccctggactgtaatgtttagatgtgctctaccaccaggagcccggtgaaaatgccagtggtccatcccctcccaggcttctaactccatgttcaaaactagatgtcactgtatgtcatgtgttagagattgcaggccttgagggactagagatggacatgatgggtcagcatcctacctcttcctcctgggagtgcacacttgttaggcagctggttaatcttgccagagcagtgaccaaaaaaatgtcacctttgaatgcatggaaactctcaaagtgttttctcagagtagcttcttgctgccccggaggctgccttgcagcatgggagtttagatcact

SEQ ID NO: 495 - DNA, 534 bp, Mus musculus
  misc_feature 62, 144: n = A,T,C or G
tgtactggtgtctccggtgccgcaccagatgctgccctacatgaacgtgggcacgctgtcgngcactgagctgcagctgctcaaccagcttgcactgtcggcggaagcggctggcaccggcaccatcttcaccgatgagcagcntcgaagccctggagaacctcttccaggagacgaagtacccagacgtgggcactcgggagcagctggccaggaaggtgcaccttcgggaggagaaggtggaggtctggtttaagaaccgccgagccaagtggagacgacagaagcgatcctcctcggaggagtcagaaaacgccgagaagtggaacaagacgtcctcaaaagcctcgccggagaagagggaagaggaaggtaaaagcgatttggactcggacagctgacggccgcggccgcgggccggccggggcgcttgcctgacgcaaggacttgcacagacagtcgatgctacttgcacacaccctgccttgcgggagggggtcgagaaagcaacgaggagctagctgtaaataatgc

SEQ ID NO: 496 - DNA, 531 bp, Mus musculus
  misc_feature 438, 508: n = A,T,C or G
actgagggtcccggtccctcgaatctgggtgggcatagacttgaaggtccagctgcagaagcccctggtactgaacgaggcagacaacacagactggcacctactgcaaggtgacagtgatgtcagagtagagagaaaacgtccggaagaagtggagctgtggggacttaaggaggggacctacctgttccaactgacaagaactgactcagaccaaccagaggaaacagccaacctgactatcactgtgctaactgccaagcagacagaagattattgcctcgcatcctacaaggtgggccgctgccggggctccttcccacgctggtactacgaccccaaggaacagatctgcaaaagttttacttttggaggttgcctgggcaacaagaacaactaccttcgtgaggaagagtgcatgctagcctgcaaggatgngcaaggaatctcaccgaaaaggcaccatccagtgtgctccggcagctgccatgccacccagttccgctgnagcaatggctgctgtatcgatgg

SEQ ID NO: 497 - DNA, 528 bp, Mus musculus
actgagagaaggagcaggctgtaaatcgagctggtattgtccaggaagatgtgcaaccgccagggttgaaagtgtggtctgatccatttggcaggaaatgaggctgtcagcaagtctgatgaggaaagtggacgtctttatcctgtgcactccgcagtggggacaatagatgcctcactgtggcagcatggcatggagagggaactctcatgctgctagccagaccccttgtgatagagactgtgtgcaaagacagtgcttcccttaactccctggagaacctgaacagatgccaccattaggaagtgccttgcggctccattgactttgcaggagcagagccagcctgcaaggctgtttgtggaagatctgctgctcctgcagtctttatcacttccaagctgtgatgtgaacacaagcaacctgtgggctcaaggtccgtggctgctctgacaccttctgaataagcgatttcagtgcaaatggccttgccaagctgcctcgcagggctcttggaggatgtttcag

SEQ ID NO: 498 - DNA, 526 bp, Mus musculus
  misc_feature 26, 32, 197: n = A,T,C or G
tgagacttggattcagccgccgtggnctcagnatgtatgatctgcccaaaaaagaagacgccttactttaccagagcaaggactataatgatgactactatgaggagagttatttgaccaccaagacatacggggagcctgagtctgtgggcatgtccaagagcttccgccagccggggacctcacttgtagatgcntgataccttccatcaccaggtgcgtgatgacattttctcttctttagaagaggaaggcaaggatagggagcgcctcatctatggccaagacagtgcctaccagagcatcgcacactaccgccccatttccaatgtctccaggagctctctgggcttatcatattatcctacatcctccacctcttctgtgtcctcctcttcatcttctccctcttcatggcttacccgacgtgccattcggccagagaagcaggcccctgctgctgctctgggccaggatcgacaggtcccactctggggccagctgttactcttcctggtctttgctg

SEQ ID NO: 499 - DNA, 525 bp, Mus musculus
  misc_feature 23, 322, 476: n = A,T,C or G
actgagacagacggagagctggncttctccccttcctgaccatcatgcatatgcaaatcaagcaagaggacccaaagaaagaaatccttctggccatgctgatggcagacaaggagaagaaaggttacatcatggccgtctgagctgcggtcgaaactcatgaaactgggagagaagcgtcacatcacaaggaagtggatgatcttttcaaggaagcaggcattgaaccaaacggccaagtgaaatatgacactgtttatccagaggatcaccattcctgtgcgagactactaagggagccaagatcaagagagcagatganagccttcggggcctgaaaacctagactgattcctttttgtaagggaagtctagagatgacacaaccactgcatggcataagtaccaataaatgatgtagccatggcattggataactgtaataaggtctgtcttgatttcggtactaacattantctagccatttgcagcatagtctagccctatgatttgtagtcagggttg

SEQ ID NO: 500 - DNA, 524 bp, Mus musculus
  misc_feature 174, 298: n = A,T,C or G
gaggtgtgacaagcagcctgcctataccccaaccactacagaccaccagccgcaccccaactacccagcccagaagtaccattcccggagcagcaaggcaggtttgtggagcagcagctgaggagaagcgagctgtcatcccgctctgagcacagtggagggaccagcacgaanaacctcatgcccaaagagtcccgggagtctcaggcatagatgccttctgggactaggggtgcattgtgagcctgaacacctccctataggcgggagaaacagcccaaaccacaggaagcaacanaaactggagaaccgccacctttagaattcccaacactcaggctctgcccgtcgtcggcttggctgcaggacagagacttgactccatcccagctctggtcttggctgggatggcactaccctgtgagccaaacactttaggtgtaagtaggtacacttgtgacgtcactgacctagcgtacctttccttttcatatctacactgaccttaacttataaaaaaaaaa

SEQ ID NO: 501 - DNA, 523 bp, Mus musculus
  misc_feature 25, 32, 34, 58, 73, 79, 92, 100: n = A,T,C or G
gactgagactggacaggaacacganctgatananagcatgcctctcttggaatggttngctaacaactataanaaattnggagctacactanaaattgtnacagataagtcacaagaaggatcacagtttgtgaaaggatttggtggaattggaggtatcttgcggtaccgagtagatttccagggaatggagtaccaaggaggagatgatgaattttttgaccttgatgactactaggtagtcgacatgggtccggcaaccgtgcctcaccctccagcattcaacccaaggagcatacccgtggtggagtccaacagatccctgccttacaattggagcatttccagaacttaatccgtgagcattggatactgaaaagaaaagtgaaacaaaaccagacccaaccctacactttggtttgtcgtggtgtcagcgcagcagccgacaactaagtctctaaaggccactgtggactcatttaaaaagaatcccagtttttacttttacttgatggtaaaattg

SEQ ID NO: 502 - DNA, 521 bp, Mus musculus
  misc_feature 380: n = A,T,C or G
gactgaggctcctgtgcatgaccgatgagtggttctcggagtacgtctacgaggtggtggtggacaagaagcatgtcccagaagaggtgctggctgtgttagagcaggagcccattgtcctgccggcatgggaccccatgggggctttggctgagtgattgcgtggactgcctcctgctctcctcctcccatgggatcggatggagctgccaaggacagacccggggactgaaaccaaagttacatggatggctgttcccacaggatacagtcagactcttgggtttccagtctcctccaggaacctctttgagaaaaatgctttatgctgaaacaaaatattcataaaggaaaaaataaaggggaaggatcggggcatnctgttgctctcctcatctctgacggctcaggatctcgtagcattttaattggatgtcattgtcctctgtcacagtctttgtgtttgtagtaagacggagtaagatgagcagctgaggtgtacggagaacaaacagtcctgg

SEQ ID NO: 503 - DNA, 520 bp, Mus musculus
  misc_feature 90, 91, 93, 95, 97, 98, 102, 104, 105, 106, 111, 114,116, 119, 120, 125, 126, 131, 137, 138, 152, 158, 162, 181, 194,199, 200, 203, 206, 209, 211, 213, 216: n = A,T,C or G
gactgaggttaccagcttggcaagccggtccgagccctgcataccgctgggatgaacgtcagcacactgcacctctggcagccagccggnngngncnnggcntnnntgtgngcncngannttaannggggnaggcannaggatcatgaatgnatgctncccncagctgcatcaacaatgtnaaaccagcctggnctatnnganacnttntntnaanactggaatttgctgacacaagcaacgcacgtggcgaggagaggcaactcggactacagtgatctgagtgacggctggctggagatcatacgtgccgaagcccctgatactggtgccgacctgcccttgagcagcgtgaatggcgtgtgcccagaagttccagcccggctctccatccgcattctcactgctgaagctggctctgtggaaggggtggctcagcgggagatcctggctgtggagacaaggttttccacagtgacctggcagtaccagtgtgggctcacttgtgaggacaaggccgt

SEQ ID NO: 504 - DNA, 518 bp, Mus musculus
gactgaggtactgaaattgccaaccgtgggggctccaaggcccaggggccacagcaccagccaggctccgagggtcccagctatgccaaaaaaattgcgctctggatcgccggtttgctcggagctggtgggactgtcagcatcgtctatatttttggaaacaaccctgtggatgaaaatggtaccaagattcctgatgaattcgacagcgatccaattctggttcagcagttgcgccggacatacaaatacttcaaagattacagacagatgatcatcgagcccaccagtccctgccttctcccagaccctctgcgggagccctactaccagccaccttatacactggtcctggagcttaccggggtccttctgcacccagagtggtcgctggccactggctggaggtttaagaagcgtccaggcatcgagaccttgttccagcagcttgcccctctgtatgaaatagtcatcttcacatcagagactggcatgactgcatttccactcattgatag

SEQ ID NO: 505 - DNA, 513 bp, Mus musculus
  misc_feature 61, 74, 158, 171, 175, 200, 221: n = A,T,C or G
gagactgccttcccagcttttgccagctttaaagacatgatgcctcagaccacgtgagcanaaagagtttgaangcggggacttccaggatttcaccaggcaggacatgcccactggtggaccggagccaagaaacctcatgtccaagcccgggtttncagtgtggcttcncacngagacccccaaggagggtgcagacngactttggagaatttcaaagngaaaagcccaaaatcagcaagtttgacttcttagtagctaattcccagagcaaaatgaaatccagtgaagaaatgatcaaaagtgagctggcaacctttgacctttctgttcaaggatcacacaagaggagcttgagccttggggataaagaaataagccggtcctctccttccccggccctggagcagcccttcagagaccggtccaacactctgagtgagagggctgcgctgcccgtcatccgagacaagtacaaggatctcactggccgaggtggaggaaaatgagaga

SEQ ID NO: 506 - DNA, 511 bp, Mus musculus
  misc_feature 411, 437, 471, 489, 490: n = A,T,C or G
aatctgtcagaacaatttgaacagtactgggatgaagaggaggaagaatggcacttaaaaaatgctattagagtagacggaaagatttaccatccatcatgttatgaagattatcaaaatacatcttcatttgattgcacaccatctcccagcaagacaccagttgaaaaccctttgaacattatgttgaacattgtcaaaaacgaattgcaagaaccctgtgaaagtcccaaagttaaggaagaacaaattgatgccccaccagcttgatcagaagaaagtgtagcaacacccactgaaattaaaacagaaagtgatacagttgagtcagtttaaataaaatgagaaaagtatgcttttctttttaaacaaaagctgctgttggatctaggtgaagaatttttttatgtnaatatagaaatatctatattaattgnctagctgtggcagggccttcagctatcatttggntaataaatacatttttannatttgcatttcctactggctg

SEQ ID NO: 507 - DNA, 511 bp, Mus musculus
  misc_feature 132, 170, 206, 317, 371, 377, 485: n = A,T,C or G
tgcgccacaaggtggcagcatgaaagcaaggcgcataatgggagaagcttccagaggaccgggactcttgcctttgagcgggtctacactgccaaccagaactgcgtagatgccgtaccccactttccttgnggtactctggactgcaggactacgtttgcagccaagtncctgccgccttcgccggacgtgatgtacctgtttgngaggcaaaaatactttgtcggctatctgggagagagaactcagagcacccctggctacatcttcggcaagcggatcatcctgttcctgttcctcatgtccttcgccggganactcaaccattacctcatcttcttcttcggaagcgactttgagaactacatcanaacggnaagcacgacgatctccccgctgcttctcatcccctgattgctggagacagagaaggacgctcaccagatcaatagagacgcatcataacgcaacgccgcgaaggcttntgctcctcttcaagctgtagatgctg

SEQ ID NO: 508 - DNA, 510 bp, Mus musculus
ggcaggttacctgtttggcagtgacgtggtcgcccagttcaatgcagccaacgacattgatatgatctgccgtgcccaccaattagtgatggaaggctacaagtggcacttcaatgagaccgtgcttactgtgtggtcagcgcctaattactgctaccgctgtggcaatgtggcagccatcttagaactggatgagcacctccagaaagatttcatcatcttcgaggctgcaccccaagagacacgtggcatcccctccaaaaagccagtggccgactatttcctgtgactccttggccccagcccctctggcccttggaccactgtgactgccctctgcccagtcggaggctgggtctgaggggctgcctggctttgcttgccccagggtgcggacttgctctggagaggtggagccttggctcctggcttcctctcctttctcccacttgatccatgaagttttcagtcattttttttttctttttccctttttttccctttcttttt

SEQ ID NO: 509 - DNA, 503 bp, Mus musculus
  misc_feature 128, 132, 145, 153, 173, 174, 218, 225, 231, 254, 257,260, 278, 279, 284, 288, 291, 294, 305, 310, 313, 315, 321, 325,337, 340, 341, 352, 358, 370, 374, 376, 383, 388, 390, 393, 398,402, 405, 416, 417, 442, 457, 461, 473, 482, 486: n = A,T,C or G
tgaggagggttcagagatgggctagatgccggcaaagcacttactcttcaacggggcttcaatcaagggtacaaagagggtgcagacgtcattataaactatggactccttagaggaacactgagggntttntataacctgtaantgatcgantcatttagccacaattctanngaaggtctttaatagaggtactatgcacaaattcaccagtttanaagagtntttcgncaggctggtgaaatggcgtcacnggncangagcacccgactgctctnncgaangccnganggncaaatgccacnttccnccnanggtccnacanccctctctaacnggnnctgactccctnttatgnagcgtgtgaatnccgntncagatgngcttncntanaatanatanatntttttttaaannaaaagagcatgcacatttggaaaanccggcctctcttggnttcnggagcagacatncagtttagntganacagctgggagtaccgg

SEQ ID NO: 510 - DNA, 501 bp, Mus musculus
  misc_feature 19, 409, 484: n = A,T,C or G
ctcccttgcacttcacttntgccttcggggccggcttctgcaccaccgtcatcgcctcccctgttgatgtggtcaagacgagatacatgaactctgccttgggccagtaccacagcgcaggtcactgtgcccttaccatgctccggaaggagggaccccgcgccttctacaaggggttcatgccttcctttctccgcttgggatcctggaacgtagtgatgtttgtcacctatgagcagctcaaaagagccctaatggctgcctaccaatctcgggaggcacctttctgagcctctccatgctgacctggaccctgcttcccagccctgccctgtctttttcttcatcctctgcccagtcccattctcttcccatttcctgcaccccgatttacttcccacctcacctncctgtgcctctgtactgatgactcacagtgaggaggcctgacaccagaccctgagccctcagccctttttacagntaagcccacatcttcat

SEQ ID NO: 511 - DNA, 499 bp, Mus musculus
aagcaaatccaagatctgcgccaatgtgttttgtggagctggcagggaatgtgccgtcacagagaagggggagcccacgtgcctctgcattgagcaatgcaaacctcacaagaggcctgtgtgtggcagtaatggcaagacctacctcaaccactgtgaacttcatagagatgcctgcctcactggatccaagatccaggttgattatgatgggcactgcaaagaaaagaagtctgcgagtccatctgccagcccagttgtctgctatcaagctaaccgcgatgagctccgacggcgcctcatccagtggctggaagctgagatcattccagatggctggttctctaaaggcagtaactacagtgagatcctagacaagtactttaagagctttgataatggcgactctcacctggactccagtgaattcctgaaattcgtggagcagaatgaaacagccatcaacatcaccacttatgcagatcaggagaacaacaaa

SEQ ID NO: 512 - DNA, 498 bp, Mus musculus
  misc_feature 167, 216, 287, 451, 454: n = A,T,C or G
gactgaggatgtacttcttattaatgtggtgattgaacagatgatttgtgatactgaccctgagctcggaggtgctgttcagttaatgggacttcttcgtactctaattgatccagagaacatgttggctacaactaataaaactgaaaagagtgagttcttaaatntcttctacaaccattgtatgcatgttctcactgctccacttttgaccantacgtcagaagacaaatgcgagaaagataatatgcttggatctaacacaaccaacacaatttgccctgatnattatcaaacagcacagctgcttgccttaattttagagttactcacattttgtgtggaacatcacacataccacataaaaaactacatcatgaacaaggacttgctaagaagagtcttggtgttgatgaattcaaagcacacttttctggccttgtgtgctctncgntttatgaggcgaataattggcctgaaagatgaattttataatcg

SEQ ID NO: 513 - DNA, 498 bp, Mus musculus
  misc_feature 377, 460, 480: n = A,T,C or G
cacgagctgtctgaactggtgtactctgcctcgaacctgctggtgctgctcaatgacgggatcctgaggaaggagcttcgaaaaaagttgcctgtgtcgctgtcccagcagaagttgcgtgacatggctgagtgtacttggagtgtgtggaggtgttcatggagatgggggcatgccaaggtgtggggcgaagtaggccgttggctcgtcattgctctcatccagttggccaaggccgtcctgcgcatgctcctgttgatctggttcaaggctggcattcagacatcaccccccattgttccactggacagggaaactcaggcacagcccttggatggtgaccacaatccgggcagccaggagccatcatatgtggngaaacggtcacacagagtggtgcgaaccctccagaacagcccatccctgcactcacggtactggggagcccctcagcagcggnaaattcggcagaaacagcancaggaggaactgagcata

SEQ ID NO: 514 - DNA, 491 bp, Mus musculus
tgagaccttttacttgtcctagttgacttgtgtgaaaatggcgggaagcgtcctactgcaagtagcagtgctagtttcaccaaagaacaagcagacactattcgcagaatccgaaatagtaaggacagctgggagatgctaggagtcagacctggagcctcaagggaggaagttaataaggcctaccggaagctggctgtgctgcttcacccggacaagtgtgtagcaccaggcagtgaagatgccttcaaagcagttgtgaatgcccggacagccctcctgaaaaacatcaagtagaggcagaggaaactacaagcgagctcaagtgcaaacagactccccttgaggtgaaataaccaacatggagttttccttcacgaatgctcactgttgtgttccctcatgtgtaagttaatagtatgggtgcctgttaccatgttgtggccatttctcagtgagatgaccgcttttgtgtgagagataaccccaaa

SEQ ID NO: 515 - DNA, 489 bp, Mus musculus
  misc_feature 183, 206: n = A,T,C or G
tgaggtgacgaaaactggtcttgtggtggtgaaaacatgaagatcattggtcttcactgctccagtgaagacttacatactgggcaaattgctctcattaagcatgggtccaggctgaaaaactgtgatctttatttttcaagaaaaccatgttctgcttgtttgaaaatgatagtgaatgcntggagttaaccgaatttcttacntggccttctgacccagaaataagtttgctcactgaagcttctagttctgaagatgcaaagctagatgccaaagccgcagaaagattgaagtcaaacagccgggcccatgtgtgtgtcttactccagccgttggtgtgttacatggtgcagtttgtggaggaaacctcttacaaatgtgactttattcagaaaactgcaaaagcgttgccgggtgctgacactgatttttattctgaatgtaaacaagaaagaataaaagagtatgaaatgttaaaaaaaaaaa

SEQ ID NO: 516 - DNA, 481 bp, Mus musculus
gactgaggtttctgtaaagcaagcttccttcctttgctggaatttgcctatacttctgtactaagttttgacttttgtagcatggctgatgtagccgtcttagctcgccacctttttatgtcagaagttttagaaatttgtgaaagtgtacataaactaatggaagaaaaacagcttacagtgtataagaaaggagaagttcaaactggttgcatctactcaggacttagcagcacacaatggaactacaacaccacctggcactaggaatgaagcaaccacaactttgtccggtgaacttgggcattgtgaaattgtactgctggtaaatggagaattgccagaggcagagcagaatggggagccagagcagcagcctgcacctcaggcttctccagaggctgaagctagtgtgtcccctgtagaaggtatacctgagccccaccctgaaatgggaactgcaagcttagcaaaaaaaaaa

SEQ ID NO: 517 - DNA, 481 bp, Mus musculus
  misc_feature 35, 76: n = A,T,C or G
gaggttcttccagatgcttccagccctcagcagantgccatcagcagtccacacccggctggtttgcacctccctntgctggactttgagctgcgcgccaaaagaaagatgcacctgatggaggtgcatttctttaaccgacatacagttgaaggaaagactggtctcctgatggatgtctgcaatgattaagagggaatgcttctggtaccctttccctcggatgcccgtgaagggctcgggcatcaccagccactgtcccagcccgggctctaagtctgacccaagccgaagtgcctgcgccccgctatgcgctccatcaccactagagggagtaaagagcgggcgtcttccctgtgctctgtcctccatagccttgcctaggaaagaaagatccctccaagtcaaagtgagaggaggtctaactgggaagatgatcaatggtgcggcgggtagggaacaagtgaggtaatgggagggc

SEQ ID NO: 518 - DNA, 481 bp, Mus musculus
  misc_feature 58, 139, 210, 292, 305, 309, 334, 361, 370, 380, 431: n = A,T,C or G
gactgagagctggcattcctaccatcctttatagtgatgctacaggcacaaagaggantggataaaaacattggtgagcaactcaatagagcatatgaagccttccgacaggcatgcatggatagagattcagcagtangagagctacagcaaaagactgagaactatgaacaaagaatactgccgagcaacaggaacagctgtcatttnaacaaaacctaattgacaggctgaaatcacagctacttctcgtggattctagtcgagataacagttatggctatgtaccttngcttgaagacagngacngaaggaagaataatttgacccttgntgaaccacatgataaagtgaaactagnaacactganagataagcantcaaaggtgagacgacaagaagtttcttctggaaaagaatccgccaagggnctcaacatccctctgcatcacgaaagggataatatagagaagactttctg

SEQ ID NO: 519 - DNA, 480 bp, Mus musculus
  misc_feature 463: n = A,T,C or G
ggaaagataccagctcctgcgtggggcgacagaagcaggtgtctcacccacgggctgtgatggagctcccgaggactcgctggcccagctgcttcgtgtgctgcaagaccttcgagaagcccacggctccagcctggccagcccagcagccagagagccccatcaccttctagagctacagacgtgaggtgtccgtgaggtgtccatcgagcccctgttaggtgtttgcagatcctagacatgctcaggatccttgtcccagtcaactgttctggggagggctgtgacccagcatagtcttctccatgtctgcgagggacaaccgtgacacctgcttccttgggagaaccaactgctcttgtagtccccatgacccatattcttcacccaggagaacaggctgtccacctcacattgctttgccactgtctggcctcctaggtcggatgccacagtggtttgntgtcataaataaatgct

SEQ ID NO: 520 - DNA, 476 bp, Mus musculus
  misc_feature 35, 36, 47, 62, 86, 194, 273, 287, 323, 326, 335, 351,352, 379, 387, 404, 416, 423, 435, 453, 454, 458: n = A,T,C or G
atatgcggctctgctgcaggcggctcagccatggnngggtcaggggntgaaaaagaggccgnggactggagcccagcccctccgangtatgaggaataccggccacccgcgctggatgccatccgcctgcctcgctatgccctgtatctgctgatggccgctatcctggcggtggccgtggcctatgccattgntggccacctcatcaaagatctagctcatgacctggatgactgggcctttggccctaagcctgaccaggaggaggaaggntcccggaagctgcnggccagcttggcatgcagaggacctggaagaactanatnttaagctgnccctggcctggcgtgnngaggatgaccctgcccgtgctcctcgncgcacctncattgccttcaaggacncgccggcgcaanccaactnttgggaggctgnactgacctggaggagtgnnatgngtgacactggcgggcctg

SEQ ID NO: 521 - DNA, 474 bp, Mus musculus
  misc_feature 453, 454: n = A,T,C or G
gcaggtagctggaagcaggaagactctccaggtggccgggaggatggcttcactgctgagcacctggctgcagaggccatggcagccgatatggacccctggctggtatttgatgcccgtaccacacctgccacagagctggatgcctggttggccaagtacccaccatctcaagttactcgctatggggacccaggttcacccaactctgaacctgtgggctggattgcagcctatgggcagggttacacccccaactcaggggatgtacaagggctgcaggcggcctgggaggctctgcagaccagtgggcgacccatcacaccaggtaccctgcgccagctggccatcacccaccatgtgctctccggcaagtggctgattcacctgtcacctggcttcaagctggaccacgcctgggctggcattgcccggcctgtagttgagggccgnntccaggtggccaagaaggga

SEQ ID NO: 522 - DNA, 470 bp, Mus musculus
actgagcagcgttcatcttcccgaattggacatgctacaaagggttttaactggtatggtaccgaacgacttatccggaaacatttggctacacgaggcatcccaacttacatatactactttcccagaagtaaggcccgccattcacagcctgcatcctcctcctcagcaccactggttccctagcaaccagcccagcattgggtccagttttcagcaaccagttagattcttgctcccagattgactgttagttttcaggaaacaaggctatctttggtcatattctctgctctggaatttctgggctagtctttcctgaattctaattttgttgctctggaagtgttaactgtgatccagtacctgtaatacaggaacacacaatgtatatggggaagccctttggcttcagattgcctatgtttgaataataataataatagatggcctattgttaaaaaaaaaaa

SEQ ID NO: 523 - DNA, 469 bp, Mus musculus
  misc_feature 52, 92, 233, 290, 325, 332, 352, 353, 367, 401, 408,422, 423, 424, 432, 438, 452: n = A,T,C or G
ctcaggatgagcgtgtttcccgttcttatcttgccctggcaactgaaaccgnggacatgttccatctcctcaccaagcaagtccagaagccnttcctgaggccagagcttggtccccgattagcagccatgctgaactttaacctgcagcagctctgcggacccaagtgccgggacctgaaagttgaaaacccagagaagtatggggtttgagccaaagaagctgttggaccnactgacaggatatttatttacaattggactgtgctcgctttgctaaagccatcgccnatgaccagcgatcctacagccaaggaacttgtttngaagaantcatttcaaagatgcagganngcgggaatcaaatncaccatcgcaaaagagaagttaaaacttcttgcntagaaangtggagggaaatcnnnggtaaatnaatgcnctggccaaatatanactacagcgaatgcccc

SEQ ID NO: 524 - DNA, 466 bp, Mus musculus
gagcactgagctcttgtcgaagatttgagttctccatcttcagtgcctcaacttgctttaacaactgatcatatgaagctgcagccatccttggctacccttggacctataaggttaaaaaggattttgaaattcaccactaagaatggagtagcctgcttgttttgttttgaagaaagagacaaaggcagcaaagctctccagacaggaggagtggtgcttattcgcaggcttgggctccaaacgccatggtcccagcagcaacagttcctaagaggatggaacatggggtctgagctgaagagaaacagggttcaagcaaaaggcagagcctcagtcccatcggaagagatctgcagtgctacaggtaagctctctatctggtgagactgtttcaaggttaaagatgacctaactcctcctgtatgtcaaacttacagaatacaaccactgataaagaaaaaaa

SEQ ID NO: 525 - DNA, 451 bp, Mus musculus
  misc_feature 195, 406, 422: n = A,T,C or G
tttgtgaacacagctctggaatacaaagaaacgtaccgctccaaaaactccatctggtttcacgaagtatttatcgttcacatcatcctgccctcaagcttcaaagaccccaactaaggacaccatttcagcagttctcttctctaactcacctttcattacataaattgaaactttctccaattaaatatggcntaccagccccgcaggaacttttggccagcaaggttagctgcaagactcttaaaacttcgatatatcatactgggatctgctgttggaggtggctatacagccaaaaaaaccttcgatgaatggaaagatatgataccagaccttagtgactataagtggattgtgcctgactttatatgggaaattgatgagtatattgatttgggtttgnatcaatcaacattaanaatagctctgggtttcgagggatggcagt

SEQ ID NO: 526 - DNA, 450 bp, Mus musculus
tgaggtggagagctctacttccggtttcactcctagtggagctggtgcctcagtatccatgattgccagtagaaaaccaacagatggcgcttcctcatcaaattgtgtgactgatatttcccatcttgtcagaaagaagaggaaaccagaggaagagagtccccggaaggatgatgcaaagaaagctaaacaagagccagaggtgaatggaggcagtggggatgctgtctccagtggaaaggaagtttcagaaaacatggaggccgaggctgagaatcaggctgagagccaaacagcagaggggaccgtggagtctgcagctacaattaaaagcactgcatgttaagaggcgagcagaaccttcctcctgagggaaagtggttttgtacataatgtattttttcacttttggggattctttttgtataactccaataaagattgtaggca

SEQ ID NO: 527 - DNA, 445 bp, Mus musculus
  misc_feature 274, 336, 361, 385, 401: n = A,T,C or G
cctaaacccctccgtttctgtcaaccgtaacagtagtccagctgtcccagctcctaccccagaaggagtccaggctgtgaacaccacaaagaaggcctcaggcaccactgcccagagctcctcctctgagagtgaggacggggacgaggacttgattcctgccacacaaccctccacctatgctctcagaaccagtgtgacgacgcctgcagtaccaggaggaagcgaggtgggacacacaccccctctgagtgctacgtgacatgtgtatagnatatctatttttttaagcgacttgcctctctcccttcaaatccaccgtgcccagaggcctgnggacctccctccatgttatctgcanagccagcaaggtcccgggtgtggntcatcccacaccctgngacccctggcgcagcctgaagccttgccccatcaatttttccag

SEQ ID NO: 528 - DNA, 444 bp, Mus musculus
  misc_feature 26, 146, 151, 152, 155, 156, 157, 158, 163, 167, 171,172, 174, 189, 201, 203, 219, 255, 256, 258, 262, 289, 301, 305,309, 312, 337, 341, 352, 356, 364, 372, 375, 377, 380, 382, 383,387, 400, 406, 418, 421: n = A,T,C or G
ctgtcttcctctgcttcaactcgganaagacgagagtaagaggaatctggagctgccacgcctctcagaaacctccataaaggaccgcatggccaagtaccaggctgcagtgtccaagcagagcagcctcagccagctacacacangagcnngannnnagcgngagnaganntnatagcaagggagcanaagtgagattgntnctgcaggtccgggagnagctgcgcccgtccattcagggattgaattaagganntngggnctgacaatagggtagaggcctttttgnattcgtagagantaangctnttntggagaggaagaacgtgtattaccnttangggactggttntgcntgaaaatntttattcnttnangcntnnaatnttcagtattccangtcttnccaaccccctancgncaagaatcaagagcacatatatg

SEQ ID NO: 529 - DNA, 443 bp, Mus musculus
  misc_feature 21, 34, 66, 111, 127, 132, 173, 192, 196, 277, 288, 356,386, 419: n = A,T,C or G
cagcactcgtcgtcgtgaagngaaatgatccgtnacagagagcaggaggagctgaggcgacagcangagggcggctttaaaccaaactatatggaaaatagagatcagganatgagaatgggtgatntgggnccccttggagcaataaacatgggagatgcatttagcccagnacctgctggtacccaaggncctnctccaatgatgggtatgaatatgaacaacagaggaactatacctggcccaccaatgggtcctgggcctgccatgggaccanaaggagctgcnaatatgggaactccaatgataccagataatggcgcagtgcacaatgataggtttccccaaggacctcnatcccagatgggttcacccatgggaaatanaacaggttctgaaactccccaggcaccgatgancggtgtagggcctgtaagtggtgg

SEQ ID NO: 530 - DNA, 441 bp, Mus musculus
  misc_feature 44, 48, 343, 348, 351, 364, 368, 369, 373, 376, 382,387, 407: n = A,T,C or G
ggcttctactggaatccccaccattagttttttgcggggtccanaaanccaaacgcttaccctcaagagcatttgtatgttatggtgaatgccaagcttggagaagaatcaaaagaacctctctctgatgaagatgaggaagacaatgatgatgttgaacccatttctgaatttcgatttgtgcccagtgataagtcagcattggaggcgatgttcacagcaatgtgtgagtgccaggccttgcatccagaccctgaggatgaagactcggacgactatgatggagaggagtatgatgtggaggcacacggtcagtgactcgggccttgcggctttcctgttncattntangcatgtagttgtnctannggcntangatgantgtcncccatgtaagatggcagtanataaagggaatgatccgatagcgaaaaaaaaaaa

SEQ ID NO: 531 - DNA, 429 bp, Mus musculus
  misc_feature 260: n = A,T,C or G
aactgagatgcagaaacagtggtatgtataaaaatatagcatgctttcttgatctcacacaccttaaacaccgcgagactttaaatcttgctggatggaactgccactcaccgctagaaatcggggatcaacagcaaactctggatgaccctgtaaccacatctccagctccgcccggcgaggggcctcatcacccaccagcaaagtaccatccaccttattgatgacagggatccgggtctccaggtccagatcaaggngattaggctcttccatgcactccacctccagctgcaacccagaatccacccccatgagcacatacacttgtttggaaggaggaggagagggaagcagggccaatggtagtcatgaggaaacttcccaactgccttcaattactgataataaaaagaccttatctagtca

SEQ ID NO: 532 - DNA, 428 bp, Mus musculus
  misc_feature 17, 28, 46, 52, 72, 73, 78, 79, 90, 100, 123, 139, 142,174, 211, 356: n = A,T,C or G
gagctcgttctatggtncttaccacatncagttcggttttctccangacatncaccagtacctcaatcttgnncttgnngctgaagaganagtcatcgtncacccagaggacatatttagtantcacctgtgagatggncangttcctcccagcaaaccagccctgcaggcagnatggggttggatcagcagttgtcacctcgggggcttnttgaattcccagtgaccaggctgcctcccaccatgggtgaacttggaatctgtgggattctgtagttcagacaaggctgacgactacagtctgcctcagagagacatggcagggacagtgactttctggatcagaggctgagacnaagactggaaagtccttccttagtccagtgacctctatgaatagactggagtcccctctcatcttacagtca

SEQ ID NO: 533 - DNA, 422 bp, Mus musculus
  misc_feature 45, 51, 55, 61, 80: n = A,T,C or G
gttctccagccctcagtggaaccagcacagacaaccgccccaggnatgatncagnatgcgnaccaggcaggcttgcttcnacagcaggaagaactagacaggaaagctgccgagctggaacgcaaggagcgagagttgcagaacactgcagcgaatttgcatgtgcgagacaacaactggccgccactcccctcatggtgccctgtgaaaccctgcttctatcaggacttctccacggagatccctgctgactaccagcggatttgcaagatgctttactatctctggatgttgcattcagtgactctgtttctaaacctgcttgcgtgcctggcctggttcacaagtgacgcagccaatggaacagcctttggcctctccatcctttggtttctgatcttcactccctgtgccttcctttg

SEQ ID NO: 534 - DNA, 422 bp, Mus musculus
  misc_feature 357, 405: n = A,T,C or G
ggcccatgcagtttctgggcatggttatctctcatgacatgctgctgggacgctggcgcctgtctttagaactgttcggcagagtattcatggaagatgttggtgcagagcctggatcgattttaactgaattgggtggttttgaggtaaaagaatcaaagttccgtagagagatggaaaaactgagaaaccagcaatcaagagatttatcactagaggtcgatcgcgatcgggatcttctcattcagcagactatgagacagcttaacaaccattttggccaaagatgacagcagaacaacataatttacactgtgatttatcttttttgctgagggggaaaaatgtaaaatgttnctgaaaattcactgctgcctttggggaaagtgcttcagcaaaggctcntgtatagagggaatagg

SEQ ID NO: 535 - DNA, 421 bp, Mus musculus
  misc_feature 65, 79, 101, 118, 119, 173, 190, 206, 209, 225, 226,227, 235, 249, 254, 259, 267, 268, 279, 286, 300, 304, 315, 332,345, 350, 351, 357, 364, 367, 376, 398, 402, 404: n = A,T,C or G
tacaggaacaaccttatactaccagcctggcctcctgtatggcgggcctgtggagcacgactgcngtgtcctccgtggnattggctattacctggaagctntgctttgcctggctccnnttatgaaacacccattaaaaatcgttctgcgaggagtgaccaatgaccaggggngacccttcggttgatgntcttaaagcgacagcnctncctctactgaaacagnnntgggatcnatggagaatcattngagcntaaanatcttgcnntcggggaatgncccctgnaggagggggcgaancgcnttttttcctgncccgtgaggaacgtccngaagtctgtcagntcacnnacccangaaaaancancgggatcanaggaatggctgtatattagttngcgncntcacctcacatggtaaa

SEQ ID NO: 536 - DNA, 417 bp, Mus musculus
acagtgtagacgggcgcccccggtgttgtctcagtcacgcgtcacaagtcgcctgcagctgcccgaagagccaagagtcagtattccggccagcttcacgaagtcagagagatgatgaaccaggtggaaggccagcagaagaacctcgtgcatgccattgagtccctgccaggctcgggccccctcactgccttggaccaggacctgcttctcctgaaggctacctctgccgccaccctcagctgtcttggggagtgcctcagcctgcttcagcagagtgtgcgccaggcagcgccacccagccacaagccaggcgcctcagagaccatcctgggatggcatgggcccacgtcacattccacagaccaactgaaaaatgggaccctcggctctctaccctccgccagtgccaacata

SEQ ID NO: 537 - DNA, 414 bp, Mus musculus
  misc_feature 40, 42, 79, 84, 107, 108, 113, 138, 158, 178: n = A,T,C or G
gatgttcatcagctcagtgcgatgtttgaactgaccccangngtatgcttccagtcatggagaaactctggccttgcanagcangaagaagctctatgctgaaagcnnctgcngacaccaaaggacgacaggaactgncaaaggaagaaaaggctcgngaactcttcctgcaggcagnggaggaagaacaaaatggagctctctatgaagccatcaagttctaccgtagggcgatgcagctggtgccagacattgagttcaagatcacttacacccggtctccagacggcgacggcgttgggagcggctacatcgaagagaacgaggacgccagcaagatggccgatctcctgtcgtacttccagcagcagctcacgttgcaggagtctgtgctcaaactctgtcagcctgagt

SEQ ID NO: 538 - DNA, 403 bp, Mus musculus
  misc_feature 119, 121, 122, 216, 366, 372, 374: n = A,T,C or G
gttcttctgacctttggagttcatcgaatgggatgagccagcctggttttggtggaattctggggacctccacatcccacatgtctcagtccagtagttatggcagccttcattcacangnncgcttgagttatcctccacactcagttgtcaccaacagacataaacacaagtcttccgcctatgtccagcttccaccgtggtagtaccagcagntcaccatatgttgccgcctcacatactcctcccatcaatggatcagatagcatcctaggaaccagagggaatgctgctggaagctcacagacgggtgatgcacttgggaaggccttggcatctatttattcccctgaccacacaagcagnagttantncatctaaccccatcaacaccagagggata

SEQ ID NO: 539 - DNA, 390 bp, Mus musculus
tcaggaatccgagcagcatacaggccggcgccggtgatgtacatcacagccatgaggaagaaccagcccatctggcccaccgtggtggccttgacaaagccctcagcgatagtaaagtgcatggtgggtacaacaccactcaagccaagtcccaggaacactcctgaacagagcagacactgaccgtcaacaggaagcagctacaggaaagcactgctgtggatctggaactgatgtacttacgaaaagcaactgctttctcatgaactatacatgtttaaaagtatttaggacctcttttgacctccctgggtacctgaacatatatgacagacacagattcacacatatacagataaaataaatgaaacagggagctaaaaaaaaaaa

SEQ ID NO: 540 - DNA, 390 bp, Mus musculus
ccttcttaaagatcgaagtctgaagcccatgaagatcgccgagagtgacattgatgtcaagctgagcatcttctgtgagcaagacaggatactccaggacttggaagacaagatccgagctctcaaggagaataaagatcagctagaatcctgtgctggaggtgctgcacaggcagacagaacagtaccgagaccagccacagcacctggagaagatcacttgccagcagaggctcctgcaggaggacctcgtccacatccgtgcggagctctgcagagagtccacggagatggaaaatgcatggaacgagtacctgaagctggagaaggatgtggagcagctaaagcagaccctgcaggagcaacacaggagagccttttttttccagg

SEQ ID NO: 541 - DNA, 389 bp, Mus musculus
  misc_feature 23, 69, 98, 108, 109, 135, 146, 153, 156, 161, 162, 187,188, 201, 209, 211, 224, 249, 250, 260, 307, 316, 317, 323, 330,337, 343, 350, 354, 356, 368: n = A,T,C or G
aaggtgttcttggtgaggaaggntacaggatcagacgctggtcagctctacgccatgaaggtcctgaanaaagccaccttgaaagtgctgagaccggntcagatttannatggagagagacatcctggcagaggngaatcaccctntcattgncangcctnnattatgcctttcagacccgagggcnngctctactctgancctggacntncctgcggggaggngacctcttcaccaggcctttccaannaggcagatgntcaccggaggaggatgttaagatcctaccctggccgagctggccttngcttctagnncacccnccatggnaatgggnataatntacaggnatcngnagccagagaatntcctcccggacgaagagggac

SEQ ID NO: 542 - DNA, 382 bp, Mus musculus
  misc_feature 354: n = A,T,C or G
agatgtacagcagtggtacaagggcttcattaaggactgccccagcgggcagctggatgccggtggcttccagaagatctacaagcagttcttcccatttggagaccccaccaagttcgccacgtttgttttcaacgtcttcgacgagaacaaggatggcaggattgagttctccgagttcatccaggcactgtcggtgacctcaagggggaccctggatgaaaagctgcgatgggccttcaagctttatgacttggataacgatggctacatcaccaggaacgagatgctcgacatagtggacgccatttaccagatggtggggaacaccgtggagctcccagaagaagagancacaccagagaagagggtggaccggatc

SEQ ID NO: 543 - DNA, 368 bp, Mus musculus
  misc_feature 48, 69, 71, 76, 77, 91, 111, 121, 158, 172, 176, 182,183, 194, 207, 231, 238, 262, 274, 279, 283, 300, 301, 306, 311,312, 313, 317, 345, 350: n = A,T,C or G
gagtgctggctctcgtcctcgcagccaccaccccgaaaacacactcgntcctgggctgtgctgaaggcntngctgnngctcctgctgctgngtgtagcaggagggctggtngtatgccggntgacagggctgctccagcagcccctctgcaccagcgngaacaccatctacnacantgcggnncagggcctgcntcatcatgagatnctccagagggtcctgcaaaccgantcgcagnagtgagctcaccctgagcacccgntgactcccagcnttggntctntggattcctatggaatnngggtnctgcnnnacanaacctatttttagcgttaagactctaantgccntcatcacatgtaatgctg

SEQ ID NO: 544 - DNA, 363 bp, Mus musculus
  misc_feature 223, 227, 248, 254, 260, 266, 267, 275, 283, 284, 293,298, 299, 344: n = A,T,C or G
ggacccttcctgcgcatctccttcaattcctatgagctgggctccctgcaagttgaggacgaagcaagccagcctttctgtgctgtgaagatgaaggaggcactcagcacagagcgagggaagacactggtacagaagaagcccaccatgtatcctgagtggaagacaacgttcgacgcccacatctatgaaggccgtgttatccagattgtgctgatgcggncagntgaagaccccgtgtctgaggncacggngggcgngtcagnnctggctgngcgctgcnngaagaacancggcnnggctgagttctggctggatctgcagcctcaggccaaggcgcctgntgtgtgcgcagcctttcct

SEQ ID NO: 545 - DNA, 362 bp, Mus musculus
gactgagataatttttatggtgggacgaggatacctatctccagatctcagtaaggtacggagtaactgtccaaaagccatgaagagattaatggcagagtgcctcaaaaagaaaagagacgagagaccactctttccccaaattctcgcctccattgagctgctggcccgctcattgccaaaaattcaccgcagtgcatcagaaccttccttgaatcgggctggtttccaaacagaagattttagtctgtatgcttgtgcttctccgaaaacacccatccaagcagggggatatggtgggtttccagtccactgaaacaccctgagtgagacattaaagtaaggtagcaacaaaaacaaaa

SEQ ID NO: 546 - DNA, 360 bp, Mus musculus
  misc_feature 210, 212, 229, 261, 273: n = A,T,C or G
agcccagagctcctctgtggagccctgaggaagaggcccagcttcaggaactatacctcgcccacaaggatgtggaaggtcaagatgtagtggaaaccatattggcgcacctgaaagtcgttcctcgaacacgcaagcaggtcatccaccacctggtccggatgggcctggccgacagcgtcaaggagttccagaagaggaaagggaccnanattgttttgtggacggnggaccaggagctggagttacagcgtttctttnaggagttccggnactctgatgatgttcttggtcaaatcatgaagaatatcacagccaaacgttcacgggctcgagtagtggacaaactgttggccctgg

SEQ ID NO: 547 - DNA, 355 bp, Mus musculus
  misc_feature 283: n = A,T,C or G
gagagtatatgatactgcactaacacttgcctgtgctggtggccatgaggaactggtacaaacactactagagagaggagctagtattgaacatcgagacaagaaaggttttactccactcatcttggctgctacagctggtcatgttggtgttgtagagatattgctggacaatggtgcagacattgaagcccagtcagaaagaaccaaggacacaccactgtccttggcttgttctgggggcaggcaggaggtggtggagttgttgttagctcgcggtgcnaataaagaacacaggaatgtttctgattatacacctctaagcctggctgattctggtggctatgcgaacatc

SEQ ID NO: 548 - DNA, 355 bp, Mus musculus
  misc_feature 236, 264, 311, 313: n = A,T,C or G
cgcacgatgggtacggtactgcctatgcatgagcagagctatgactcctatgacaacagctacagcaccccagcacaaagtgcagctgattactacgattatgggcatggactcagcagaggacgcttatgactcctatgggcaagaggaatggactaactcaagacataaggctccttcggcgaggacggcgaagggcgtctacagagaccagccatatggcagatactgattgnactgtctgatgttgtgaaatagccaatntccaccgtcctgtatactgttcaaagtaatttttttctatgaccaancnctttttaaataaatcaaaatgcttaaaacactgaaaaaaaaa

SEQ ID NO: 549 - DNA, 350 bp, Mus musculus
  misc_feature 27, 29, 57, 68, 104, 123, 183, 215, 245, 281, 283, 290,317, 328: n = A,T,C or G
caggaggaacgtggaggagctcatgtngnacattaagaagacagcgaacaaagttcnctccaagctanagagcattgagcagagcatcgagcaggaggaaggtntgaaccgctcatcagccgntctgaggatccggaagacgcagcactccacgctgtcccgaaagtttgtggaggtcatgtncgagtacaacgccactcagtcagactaccganaacgctgcaaagggcacatccagaggcagntggagatcaccggccggaccacagaccagtgaggantnggaaganatgctggagagtgggaaccctgccatntttgcctctgngatttatttaggtctggcgaga

SEQ ID NO: 550 - DNA, 347 bp, Mus musculus
  misc_feature 31, 114, 116, 146, 154, 157, 160, 161, 184, 186, 188,193, 196, 220, 257, 292, 303, 313: n = A,T,C or G
agtttgggcaacgaaacatgaagccttgtanagaatgcacatgatctactggccaaattggcatgggatttttctcctgaacaacttgatcatctttttgattgctttaaggcnantatgtaacgcagcattgatattaccatgtnatatgcanatnganntgattcattagaacttacagagnangnattanccnacaaatacgttacataactcgtangacacttctctgatatgaaaggacatttatctagttngaaagactttgccagtgttttctttctacaaactngactttcattntcatgatagngctaatagccctaataaagactcttgttcaaaca

SEQ ID NO: 551 - DNA, 344 bp, Mus musculus
  misc_feature 301, 319: n = A,T,C or G
acgggccctgggtgtctgcctggccaagcagtcacaagcagatgacccggacaaactagcatgtggccttaagaaaagaagtcagaagaggaaccccagcccatcagttgtgccttcttggacagaccagcctgtagcagatagccatggaaagtcacgggccacagggggcagcagcctcagaaatgaagcatggtcagagcaaggccagcctcctgcatcatggtggcttcaaagtgcttcagtctctgaagggatcagtggggaggagttcagccccagcagcctccctcggcaaggntgtggctctgtcccctgntccttcagatgagcaactagcccgg

SEQ ID NO: 552 - DNA, 342 bp, Mus musculus
  misc_feature 21, 31, 37, 50, 54, 66, 79, 82, 89, 91, 97, 178, 181,182, 188, 190, 193, 205, 209, 237, 297: n = A,T,C or G
gactgagatgagatgaagcanatggctgccntgtggngcaacagcttgtngagnccagagaactgnttgaacagatggnttnggaagtncnagaaantccaccccaaagtcgaggaatgtatagcaacaggatgagaagctacaagcaagaaatggggaaactggaaacggattttantgnnattagnancgnctacagggaccnaagnacggaatgagctcctaggggatgctggncattcctcctgagaaccagagggcacatcatgctggataacacggagaggctggaaaggncgtctcggagactagaggctgggtaccagatagcagtggagaccg

SEQ ID NO: 553 - DNA, 328 bp, Mus musculus
  misc_feature 17, 18, 19, 51, 55, 78, 243: n = A,T,C or G
gctgggcctgatgctgnnnttcatctgaagacctaggaagtgagaagctgnccantcttgtctgcccctctcaacagntattcttccctgggggcagggcaggccgtctgctgggacaattggggaaaagaacctctccctggaacttctttttcatgtttacaggagagcagagagtatgtgcaggaaggagcccatggggaggggttgctcctgtgtgtaggtgcgtgcccatgtgtttgnggggaccggaggacatttgggtgcttctctgccttagtgccttggctggtgagcttagctctagatgttctttaaaaagaaaaaa

SEQ ID NO: 554 - DNA, 328 bp, Mus musculus
tgagacctgttggaggacctgtcggagagccgcgagtgcgtgaactgcggctccatccagacgccactgtggagacgagacggcaccggtcattacctgtgcaatgcatgcggtctctacagcaagatgaatggcctcagcaggcccctcatcaagccacagaagcgcgtgccttcatcacggcggcttggactgtcctgtgccaactgtcacaccacaaccactaccttatggcgtagaaatgctgagggtgagcctgtgtgcaatgcttgcgggctctatatgaaactccatggggtgcctcgaccacttgctatgaaaaaaaaaa

SEQ ID NO: 555 - DNA, 327 bp, Mus musculus
gactgaggcacacactgataaagaagctctgccagctgagggagagcgagccggagctggcccagctgctcgtggatcagatctatgagaatgccatgatagcagcaggactcgttgatgacccccgggccatggtcggccgcctgaacgaccttttggtcaaggtcctggagaaacactgacagccaagacactggatttagtgtcaacccaggtcttctcgggtgataatggacctgcctggggaggcaggacttaatacacaaacagtgccaccaactgcttgagctcagctttatttacttcaattaaacagtatttcttagt

SEQ ID NO: 556 - DNA, 315 bp, Mus musculus
actgaggctcttggtctggaagcggtggatctgtcagcattcagctctgcagccgagttggagtcgctgggtttggagaggctcaagtgtgaactgatggtccttggactgaagtgtggggggaccctgcaggagcgcgcagccaggctcttctctgtcagaggcctcacaaaggaactcattgacccagctttatttgccaagccttccaaggggaagaagaagtgaatttccttggcacacttcctccgctgtgacagacttgtggcctgtgttccttttgatactaaaagccaattttttaataaaaaaaaa

SEQ ID NO: 557 - DNA, 305 bp, Mus musculus
gactgagaccggatgcagagtttgaatccagatcccaaagcccggtatacaagcatctatggcgccctcaagaggatcatgcacactgaaggcttctggaggcccctgcggggcctgaaccgtgatgatgatgggtgcagggcccgcgcacgccatgtattttgcctgctatgaaaacatgaaaaggactttaaatgacgttttcagccaccaaggaaacagccatctagctaatggtattttgaaagcgttttgtctggagctgggaagctcttctctctggggctttccttcccccggaccca

SEQ ID NO: 558 - DNA, 305 bp, Mus musculus
  misc_feature 28, 33, 81, 107, 130, 171, 221, 232, 241, 243, 247, 248,260: n = A,T,C or G
acagcgaacagccatccatcaagccatnaaatntctctttcccaggactagagacaaagacagaggatagagaggggcggnaatacatttgtagcctaccatgcagntggcaaaaaggctttggcaaatncaagaaaacattcttggccaaaatcccggggaagttactgncactttgttctctacaaggaaaacaaagacaccatggatgccatcaaggntctatccaagnacctgcggntnaagnnaaacatattatnctatatgggaaccaatgccaaaagggcaaacacaggccaggagat

SEQ ID NO: 559 - DNA, 304 bp, Mus musculus
  misc_feature 28, 38: n = A,T,C or G
tgagcttcagtgtttctgcccctctgtncaaaggatanttttacctgtgagacagatggtctttgctttgtctcagtcactgagaccacagacaaagttatacacaatagtatgtgtatagctgaaattgacctaattcctcgagacaggccatttgtatgtgcaccatcttcaaaaacaggggcagttactacaacatattgctgcaatcaggaccactgcaataaaatagaactcccaactacaggtaagttgttgcattagcacccttccctttcttcttctaagatgctaaaaaaaaaaa

SEQ ID NO: 560 - DNA, 303 bp, Mus musculus
  misc_feature 273, 285: n = A,T,C or G
tgcccagagctattccatccggcagcagcagcagtggatctgagatgccaggcgccaacccaggggacccctctgcccctagcccaggatctccctgagggctggacttgcaacaggaagcaggcagaagggggtggaagcacagagcaacttgttgctttcgggacccaaagactcttccggggatccagcatgttgccttccatcctctttctgcctgtgccccaatgggaagcaccccctatcccacctctgtcccaactgtacccctgnttaactttttgnaaatgataaaaaaaataa

SEQ ID NO: 561 - DNA, 300 bp, Mus musculus
  misc_feature 47, 49, 82, 84, 85, 122, 154, 165, 173, 201, 202, 228,233, 238, 241, 244, 247, 251, 258, 260, 280: n = A,T,C or G
ggcaaccccagtaccgaggaacgaatccagcagggacatgaggattngnctcctcatccactttcaggcgcatctgatggcngnnaccaacctcagcctttctagccaccgtggagccagtntatatgcaacaaattggaaatgctaagatatnactaagctcgnctggagtnttgttgaatggaagccttaatacctgcnngtactacacctgtcttccttaatggnaaatnctctnatncanggncacnactggantnatccttaccggactgaatgngggaaatacacagacagatg

SEQ ID NO: 562 - DNA, 299 bp, Mus musculus
attgagtggcttgaagaaaagcaacagcaatttgagaatcttgaccagcaacttcggaaacttcatgccagtgttgaagccttggtctgtcatagaaaaggtttgttagccgtgttaaaatgcgtattctgaagtgactgtagaaaggaagaacaacagtctcagaggatgccctgctgcctgggctgcacgggggcagctgaagcagacctgccaggagtctgacgctaggctgtctcaaaacagaaaaagaggatttgttcaatatcatttaaatttttccactgtaaaaaaaaaaa

SEQ ID NO: 563 - DNA, 298 bp, Mus musculus
  misc_feature 19, 44, 46, 91, 101, 103, 108, 129, 146, 165, 171, 181: n = A,T,C or G
attgaggatccctgctganccagtttctcagtctacccttctgntnatcagtgggatgttagccgcaatcaaccttatatcgatgatgaanggtctgggtncnatggnccgtcttctgctgaccctagngtcagactgcgaatgcnccagcagaggagtgggggnacctgnctacatgaanatctagcttcacgttctgaagtcccaggaaccaatttctaatgatcaaaaggtttcagatgatgataaagaaaaaggggagggagctcttccaactggaaaatctaaaaagaaaaaa

SEQ ID NO: 564 - DNA, 296 bp, Mus musculus
  misc_feature 16, 18, 181, 182, 183, 189, 193, 194, 195, 198, 200,202, 209, 219, 226, 227, 233, 234, 236, 257, 266, 280: n = A,T,C or G
caaagagttcctttgntncgtgtatctttacagaacacaacaggaattgaaaatgcatcagaacactgagtctgtggcagtcactgagactctggctgaggtacctgaacacgtgcttcgaggacttccagaggaagtaagacttttaccatctgcggtcgacaagactcggattggtgtnnnacccanatannncantntnagaaggnagatgccttngatcatnngatggnnanaatgaactaagagatcccagnttaggaatncttgtatacatgancagaggtgatttaccc

SEQ ID NO: 565 - DNA, 291 bp, Mus musculus
  misc_feature 25, 82, 96, 120, 124, 143, 162, 236, 237, 246, 257, 270,273: n = A,T,C or G
tgcttctacctggaagttggacagnatatcccaggaccgggacttcatcatgaccttcaacacatccctgcatcgctcctgntggatggagaatgntccaggctgcctggtgactcctgntctnaactctcgcctggccctgnaggaccaccacgtcatctnggtcactggctgcctgcttgattatccttacattgaagccctcagtagtgccctgcagatcttcctagctctgnncggcttcgngtttgcctgtnatgtgagcaaagnatncctgcaagaggaggaccg

SEQ ID NO: 566 - DNA, 290 bp, Mus musculus
  misc_feature 128, 157, 178, 246, 250, 263, 267: n = A,T,C or G
taaagccagggcatggtgccagccctttcatttcaagtacgactgaaggagaaaattttgagcagacgccattgagacgaacattcaagtctaaggtccttgcccgataccctgagaatgtagactgnaatccctttgatcaggatgcagtaggaangctgtgcatgcccaaagggcntggcattcaagacccaggctgatcccagggagccacagttccatgcctttattatcacaagggagganggtnctcggacatttgngctntgctcttaccttttatgaagaag

SEQ ID NO: 567 - DNA, 290 bp, Mus musculus
  misc_feature 45: n = A,T,C or G
gaatttgatgtctctggactaactgacactgatattagaccgaanttcaatttaaaaccaaacacaaaaggaattactctacagtcaataacaggcagtcagcgtttagatgtagaagagttccagaatgatgaagaagacttgagtccacctgtgatggaatataatatcgatgtgaaatcaaacactgagatacagttagcagagataaataaagatgtagcctcgatacctagtgaatcttcaacagaatctgttaaagatcttcaggaggtcaaaagtaagacaca

SEQ ID NO: 568 - DNA, 289 bp, Mus musculus
  misc_feature 44, 50, 142, 225, 236, 250: n = A,T,C or G
catacggcggctttctatgtaaagtttgcagagggtcagagcantgcctncacgaccatcttcgggtgaactgtggggcatccacttgatgcccccacgaatcctagtggaatgtttactccccaatggaatgatagtgacntttagaatgcctccgtgaggccacactcgtcaccatcaaacatgaactgttcagagaggccaggaaataccctctccatcagnttctgcaagangaaactttttacanaacaaaaatccccaaaatacatacaaatgatggagacat

SEQ ID NO: 569 - DNA, 289 bp, Mus musculus
  misc_feature 228, 234, 242, 270: n = A,T,C or G
gagcagtttgtattctactaccacatggaccccttcaggcccatgagcggacggttcaaagaccgggtggtctgggacggaaaccccgagcgatatgacgtctccatcttgctctggaagctgcagtttgacgacaatgggacatacacctgccaggtgaagaatccacctgatgttgatggtctggttgggacgatccggctcagcgttgtgcacactgtgcccttntctganatctactncctggccgaggccatttggctctgcgcncgcactgatgattatcgta

SEQ ID NO: 570 - DNA, 286 bp, Mus musculus
actgagggagagacttcgaaggaaggcctgacttcacatcctgagccttctctgagccgagcctagagggaactcccatgctcctctgcagcagcaagaagccctacccctgcttccctgccaaggtcctgggcagctcttaccttgacttgttcatttgaggttacgtgcacagaaaacaatctctgtgaatccctgggatggaaacatccggaaacagataccaccaatgacacggccatctttaattaaagcaagggttttgtgtttcctaaaaataaataaa

SEQ ID NO: 571 - DNA, 272 bp, Mus musculus
  misc_feature 104, 110, 116, 118, 141, 171, 174, 178, 187, 207, 213,222, 223, 226, 234, 243, 246, 247, 249: n = A,T,C or G
ggaacaccaagcatcaaatagtggtaccttatcccagagcaatgcagttggttcggccttcacacaagacacaagatctgtaaaaccacagctagcccaaggtngatcangccctnanttaggccctttgagagaaagtcnaaccatggaccaagcggtacagaccgcctnggnccanttacctgcnccagcacctgttgggagaangagccntgtaccagnnagncctttgcnacctaccantcnnanagcaatagacaaccaagagcaca

SEQ ID NO: 572 - DNA, 270 bp, Mus musculus
  misc_feature 18, 52, 72, 96, 143, 148: n = A,T,C or G
caaagaagttcagtatantcccacctatgagaccatgttcgctcctgagttnggaccagaaaatccctttcnaacacagcaaatgggtgcccctanaaatatgctttctgggtatgcagagccagcacacatcaatgacttcnatggntttgaacagcaaagaaggacatttgctacatacgggtatgcactggacccttcgttagataatcatcaggtgtctgctaagtatattggctctgtggaagaagcggagaagaaccaaggcct

SEQ ID NO: 573 - DNA, 268 bp, Mus musculus
tggaagtttgaaagacagtttgatcaagaccacaatctggacctggcagagaaagacttcactgtgaacactgtggcaggggccatgaagagttttttctcagagctaccagaccccctggtaccatacagcatgcagattgacttggtggaagctcacaaaatcaacgacagggaacagaagcctgcatgcgtctgaaggaagtgctgaagaagttcccgaaggaaaaccatgaagtcttcaaatatgtcatctcccacctgaacaa

SEQ ID NO: 574 - DNA, 267 bp, Mus musculus
  misc_feature 16: n = A,T,C or G
acgcccaagagaatgnttctgttacccgctatgaaattcccaacatatcctgttccccactactcattcttttagtaaatgacagaagctcattcctattgaacaacaatgcagtacaatgcagaatgttagagaaaaagcctttttatcctgctttctttgaacacatacttactttgatcaaaattatttgtaaagaacatctttcctactttttgattttaacaaatgcaaattcagttctctaaaacttgaaaacaaacaaaa

SEQ ID NO: 575 - DNA, 260 bp, Mus musculus
  misc_feature 202: n = A,T,C or G
aactgagtaccttcacgaaaatgggatcatacatcgggacttaaagccggagaatgttcttttatcatctcaggaagaggattgtctaatcaagatcactgactttgggcagtccaagatcttgggggagacctccttgatgagaaccttatgtggtacgcccacttacctggctcctgaggttcttgtctccaacgggacntgctgggtacagccgtgctgtggactgctggagtttaggagttattcttttcatctgc

SEQ ID NO: 576 - DNA, 259 bp, Mus musculus
gactgagggcacattccagcagatgtggatctccaaacaggaatacgaggagggagggaagcagtgcgtggagcggaagtgcccctgaagttgccccctcccccaaatacccgcccaccccatccacagagaaacgccagaggggctgttctaccagccagaaatccaaaagcgctgaactctccctttcccattatcctcttctctcttacccacatcttcttagactgttatgttcctgagttgaaagaagaaaaaa

SEQ ID NO: 577 - DNA, 250 bp, Mus musculus
gactgagagtcgtcatttttgaacttccacgagtctgactgcgaactgagaggctcggcgccctgtgactcgctcctctctctcaacactgaaaagattttgagccaggccaagtctattgcagagcagaagagattcccatttgctaccgacaatgacagcaccaatgaagaattagccgattgcttatgtcctggtgggcagcggtctctacgatgaagccataagacacttttccacgatgctccag

SEQ ID NO: 578 - DNA, 245 bp, Mus musculus
  misc_feature 43, 71, 133, 134, 158, 165, 171, 180, 224: n = A,T,C or G
cttccagggcatcaggcgatggcccagtatctgtccagagcantgacacagagatgcaagatggaggccgngcagcacgtgatgtcagaagacacccagatgtcacacaggatctggccaaagttccagactnntacacacacgggtcgctccttcancggagtncacatncacagagcncggtcatgctttggcttcacaactgtgaaggccgtatacatcangacggagcacagaaaaaaaaa

SEQ ID NO: 579 - DNA, 244 bp, Mus musculus
  misc_feature 31, 73, 185, 196, 200, 203, 211, 215, 217, 220, 223: n = A,T,C or G
ctggaagagaagactgagaaaagcttgggcngagcttggactcctctgctgagggcaaaggggccttgaaganggagagagaggtgcaccagaaactgctggctgacagccacagcctggtgatggacctgccgctggcagatccatcaccgagagaagaactggaaccgggagaaggtggagcntgcttggagcngtcntgnacagcaancggnangantgngggcgacatgaaggaggagct

SEQ ID NO: 580 - DNA, 242 bp, Mus musculus
  misc_feature 25, 47, 54: n = A,T,C or G
atgaaagaggtgatcaaggagcagngtggagaggtgatgatatttgnactggcncaccatgtgcagtcgtttctcagcgagcataacaagcccccaccaaagtctttccatgaagaaatgctggaaaggcaggctcaggagaagcagcagaggttgctggaggccaggcggaaggaggagcaggagcaacgtgaaatcctccacgagattcagagaaggaaagaggagatcaaggaagagaa

SEQ ID NO: 581 - DNA, 236 bp, Mus musculus
  misc_feature 19, 20, 80, 95, 113, 129, 131, 133, 143, 165, 168, 187,210, 215: n = A,T,C or G
ccagtcagaagctcttcgnnagagtattgaagttctgccaaggtgtactgattttgaagatatcactctgcaacgtgcanagaatgtggttttgnccgaagattccaaatctnaggctaatcagaaagncngnaacgtgctgngtatgagaataactattcacanggnttctgaagtgcgttttctnttcatttgaggactacacagggnactanacaggtttccgtatccaggca

SEQ ID NO: 582 - DNA, 234 bp, Mus musculus
attattccattgatgtgtttattgccttttatatcacaacaagactctttttgtactaccatactctggctaacaccagagcgtatcatcagagtaggagagcgaggatttggttccctatgttttctttctttgaatgcaatgttaatggcacagtacctaatgaatattgttggccattttctaaaccagcaataatgaaaagactaattggatgaagactctctctctgta

SEQ ID NO: 583 - DNA, 232 bp, Mus musculus
  misc_feature 171, 177: n = A,T,C or G
actgaggttgcttgtcaagtgtcccacagatgaaaaaaatctccacgtcttacgaagaaagaaggaagcaggccacggctattgttctcctgggagtgataggggcagagtttggagctgaaattgaaccgccgaagctgctgaccaggcctcgaagctccagtcaaaatnccttgnaaggcttttggctttgaccagcgggaggcttccaacttacttccttggccaaaac

SEQ ID NO: 584 - DNA, 217 bp, Mus musculus
tgagaacaggccccatctcctcgatctacagactacacttggaaacacaaacaaatataaatatctgaagacccacgtgggacctgaagaatggcctattacatggatacatgggcggcagctcccgccgaaagaccagccaacaattctcttgaagaacattatagatggcaaattcccattaaacacaatgtcttcgaaattttaaaaaaaaaaa

SEQ ID NO: 585 - DNA, 199 bp, Mus musculus
  misc_feature 27: n = A,T,C or G
gactgaggagctggtgaccttcgaggntgtggctgtgcacttcaccatggaggagtgggctctgctggatctatcccaaaaggagctctacaaagacgtgatgaaggagatattgagaaatgtggtttccataggagacataaggaaacaccaaaagattgacaataaataccaaaattctgggagaaaactaaaaaaa

SEQ ID NO: 586 - DNA, 194 bp, Mus musculus
  misc_feature 21, 94, 168, 176: n = A,T,C or G
aattgcctactttttgttcancgattggcagaagagtccaggacaaatgcttgtgaaagtaaatctcgtatttaccacgtactggctgcagcanaggtaattctgaagaagagcaaaggttaggtgtcaacatgtgcttaagcataatatgatgtgttaatttaagtngtaacatnataaaacacttttgtatg

SEQ ID NO: 587 - DNA, 193 bp, Mus musculus
actgaggggaaacaggaatgtcactctgacgctctcctggaacgttgtaccaaacgctggaattctgcctcttgtgacaggatcgggacatgtgtctgtgccatttccagatacatacgaaataaccaagagttactaaattattctgaatatgaagcatatttttatacatgttgaattgcaaaaaaaaaaa

SEQ ID NO: 588 - DNA, 191 bp, Mus musculus
  misc_feature 119, 126, 128, 131, 132, 133, 142, 150, 158, 164: n = A,T,C or G
gagcaggtggccctgagccggacccaggtgtgtgggatcctgagggaagagttgtaccagggtgatgccttccaccaagctgatacacacatatttatcatcatgggtgcatcgggggncctgccntngannncgatgtatnctaccatntggatggnctgttncgggagtggccttctacctgaagaaaa

SEQ ID NO: 589 - DNA, 187 bp, Mus musculus
agccgctatcgtctttatcaaggagccatcctcccaggatgcacttcaagggcattgtcccctggtgaggggagcaggggagagaaaatggtagccatttttacattgttttgtatagtatttattgattcaggaaacaaacacaaaattctgaataaaattacttggaaactgccaaaaaaaaaaa

SEQ ID NO: 590 - DNA, 186 bp, Mus musculus
aaagattgacccagaaaacacctctccgagtccttcaccgaaggcctttggctgtgagaactcgggccattcactctatgaagacacactacctggatgagcatcattttcgtctgcatttgaagacccaagctggaacagttttaaaccataaagtagccatcaatttaatgtcaaaaaaaaaaa

SEQ ID NO: 591 - DNA, 184 bp, Mus musculus
  misc_feature 16, 19, 37, 75, 91: n = A,T,C or G
gaggtaaacattgcancgnaattttctgaaaaatgcngatgagctagtcaaacagaaaattcatccaacattttnttattagtggctatcnccttgcctgcaaggaagcggcatgcccaagagaatagttaatgcaaaaattgcttgtcttgacttcagcctgcagaaaacaaaaatgaaaaaa

SEQ ID NO: 592 - DNA, 183 bp, Mus musculus
tgagagcagagtagaagagatgatttagaagccttaggccacatgttcatgtacttcctgagaggcagtcttccttggcaaggcttaaaggctgacacattaaaagagaggtatcagaaaattggagacacaaaacgagccacaccaatagaagtgttgtgtgagaacttcccagaaaaggcg

SEQ ID NO: 593 - DNA, 177 bp, Mus musculus
  misc_feature 40, 84, 92, 97, 122, 129, 137, 138, 156, 158: n = A,T,C or G
acggaacgttcaccatctgtcacaagacggaggtcatgangaacaccctcaacccggatgtggcaaacacttcatacatccccntgagggcntctgngcccccggagactatgacaggaccntcaaggnggaggtgnntaactgggggggtgacgncngctatgccctccattgggg

SEQ ID NO: 594 - DNA, 172 bp, Mus musculus
actgaggacttcctagtttggccacctacttgtcccagatacctcatccgtgttcttggataccatttccgacttccatctcctgctgtttctggtcaccaatgaagtcatgccgctgcaggacagtatcagcctgctgctagaagccgtgaggaccagaaagaaataaaaa

SEQ ID NO: 595 - DNA, 158 bp, Mus musculus
  misc_feature 19, 22, 23, 27, 31, 51, 52, 55, 70, 85, 92, 94: n = A,T,C or G
taacattgaagaatactgngtnnaggnaatnttacctgtttttttgggacnnggntgaacgctgttattncttcttgaatgcttntgttggncncagctcactaaaaaggagcctgaatatgggagactggcgcagaatttgcttatgaaccctacaa

SEQ ID NO: 596 - DNA, 157 bp, Mus musculus
  misc_feature 16, 69, 93, 124: n = A,T,C or G
aactgctctgctgtgnactctccaggcgtacactcggcaccccttgtttgagggagggttatgtgcccnatgtaaggataagttcctggagtncctcttcctgtatgatgatgatggacaccanagttactgcaccatcttgctgttcccggggggt

SEQ ID NO: 597 - DNA, 145 bp, Mus musculus
  misc_feature 17: n = A,T,C or G
ggaccgttcgtaggctnggaagcaaagacaagttgttgaaacgagaaggagatgatcgagtggaaccagggacccaaggatatgaatcattcagttatggtggggttgatccttacatgtgggaaccccaagagcttggtgagat

SEQ ID NO: 598 - DNA, 144 bp, Mus musculus
gacctggaccgtctgattgaccagaagctaaaggattacgaggacctcagggaagacatgagaggaaaggaacatgaagtgaagaaccttctagagaaggggaaagctgaacagcagaccgccgaccaactcctagctcgagcg

SEQ ID NO: 599 - DNA, 142 bp, Mus musculus
  misc_feature 96, 98, 99, 110, 115: n = A,T,C or G
gactgaggagctgtggccctcaaggaccttgtaattaaagaaaatgcattgcatgaattggatgtaccattcaaagttaaagttggtcatataggnanncttaaacttanaattncatggaaaaaccttcatactcaaccag

SEQ ID NO: 600 - DNA, 141 bp, Mus musculus
gactgaggagttgaagcagaagatggggtaaatttcttcacaccaaaaacaagctttacacagattttgatgaaattcgacaagaaattgaaaatgaaactgaaagaatttcaggaaataataagggggtaaaaaaaaaaa

SEQ ID NO: 601 - DNA, 140 bp, Mus musculus
gactgagaatattatggctcttcgaatcatttactttcaagtacacctgctacagattattttctgcaacagaaagactacttcgtcattttccttctgattttgcttcaagtcataataaactttatatttaagtagaa

SEQ ID NO: 602 - DNA, 137 bp, Mus musculus
  misc_feature 39, 54, 60, 65, 67, 68, 74, 93, 99: n = A,T,C or G
caacgccccaccaggggaccttcctacggcctgagccgngaggtgcagcgcgangattgngaagnanncctganagcggatctggagcagatncttatncagtggatcaccactcagtgccgcgaggacgtgggcca

SEQ ID NO: 603 - DNA, 119 bp, Mus musculus
ctgagggtaactgggatacttctggcagtgaactgagtgaaggggaattggaaaagcgcagaagaacccttttggagcaactggatgatgatcaataaattataccaaatataaaaaaa

SEQ ID NO: 604 - DNA, 104 bp, Mus musculus
taccttgatgcaattagaagaaacaaatacccagaagacaagcggcctgacagccatgatccttgcggctgctgtaactgcatgaaggcgcaaaaggaaaaaaa

SEQ ID NO: 605 - DNA, 560 bp, Mus musculus
  misc_feature 194, 205, 217, 224, 226, 260, 320, 431, 533, 541: n = A,T,C or G
gactgaggactcagtgacgctttgaggttgtggctgtgaacttcacccaggaggagtgggctctgctggacacgtcccagaggaacctttacagagaagttgtcggggagatctttatgaacttggcctctgtgggggagagatgggaccagaatattgaagactggtacagaaatcagggcaggatcctaagnagccatattgnggagagacttantgacagnanacaaggtagccattgttgggaaaccatcagccanattcttgaccatgatctgaagaagaaaagctctcctggagtaaatctctgtgagtgcagngtgtgtggaccagccttcgtgccttgttcatccgggaagcccagcagtgcgcaggcgcacgctggacaggacgtacgtgactgcaaatgcaagcgatgtgagatagccttntgtaattactccttccaagtcaatggcaggtctcactgtgaagacaagcccaacgggtgtcaggaacgcgaggaagacttcctttctttccccagccttggngactttgntataactcgtactggggtt

SEQ ID NO: 606 - DNA, 557 bp, Mus musculus
  misc_feature 27: n = A,T,C or G
aactgaggcttggtgaaacccacaggnctgaagctttcctctctcgcctggcggtcccgcccaggcacggagcacctttggcatcccggcaggtggctgctctccccattcccccggtccactccaagatgggttagtgcttggccttctgcagaatctggctaccttgagccagttccctgtcacatccaccttcagcggcaagcagcaggcgcagttggtgtcagtcctctgcctccggtgcagcaccgcggccatgggcggctcagcttccagccagctggacgagggcaagtgcgcctacatccgagaatgaagggttgagggttggtgacacagctcactgggaaaggcactccctgaggccctcattccgattcttagagtccacctgatgaagaagagaactgactctcacaagttatccctgacttccacatggtacaatgctccccccacaaaccaaccaaccaaccaaccaaccaaccaaccaaccaacgtaatgcaagatttaaaagaaggcctagggaaatggcccagcaggtaaccatgctgtg

SEQ ID NO: 607 - DNA, 556 bp, Mus musculus
  misc_feature 182, 186, 198, 205, 207, 208, 209, 213, 216, 221, 226,230, 234, 249, 271, 389, 471, 485: n = A,T,C or G
actgaggcgggctggcagaagaggtggacgacaaggttcttcatgctgcatttatcccctttggagacatcacagatattcaaattcctctggactatgaaacagaaaaacaccgagggtttgcttttgttgagtttgagttggcagaggatgcggcagcagctatcgacaacatgaatgantctngagcatctttgnacggacnannngtgncantttgnccaanccantganaattaaagagggctnttccaggccagtctggtcagangatgactggttgaagaagttttctgggaagactcttgaggagaataaggaagaagaaggacccgagcctcccaaagctgaagcccaggagggagagcccactgcaaaaaaagcccggncaaatcctcaggtgtacatggacatcaagattgggaacaagccagcaggccgcattcagatgctcctacggtctgatgtggngcccatgaccgcanaaaacttccgctgcctgtgcacccatgaaaagggctttggcttcaagggaagcagcttccaccgcatcatc

SEQ ID NO: 608 - DNA, 554 bp, Mus musculus
  misc_feature 366, 392, 416, 419, 429, 470, 491, 506, 521: n = A,T,C or G
tgaggctgcgtggcttccgtaccctgcagccatatgctgagaggatccccatggtggcctcagatggtgtcaccatcaacttcacctcccagatctccctcacaggcccgggtgtgcaagtgtactacagcttgtacaaccaatcagacccctgccctggtgagttcctctgctctgtgaatggactgtgtgtccctgcgtgtgacgggatcaaggactgccccaatggcctggatgagagaaactgtgtctgcagagccatgttccagtgccaagaggacagcacgtgcatttcactgcctagagtctgtgaccggcagcccgactgtctcaatggcagtgacgaagaacagtgccaagaaggantgccctgtgggacattcactttccantgtgaggaccggagctgtgtgaanaancccaacccanagtgtgacggccagtcagattgcagagacggctcacatgancaacactgtgactgtggcctncagggcctctccagncgtattgtgggcggnaccgtgtcctccgagggtgagtggccatacagg

SEQ ID NO: 609 - DNA, 549 bp, Mus musculus
  misc_feature 71, 89, 93: n = A,T,C or G
gactgaggagggagctatggatttgctgcgggagctgaagaatatgcctattacattgcacttgctccagnccacccgtgttgggatgnctgncaatgctctgcgcaagcagagttcagatgaggagctcattgcacttgccaagtccctcatcaagtcctggaagaagctcttggatgtttctgatggcaaatccaggaatcaagggaggggcacacctttgcctacatcatcatcaaaggatgcctcaaggactacggatctcagctgcaagaagccagatccacctaggaccccatccactccaaggatcactacatttccccaagtgcccatcacctgtgatgctgtacgaaacaaatgccgagagatgctgactttggccctgcaaactgaccacgaccacgtggccgttggtgtgaactgtgagcatctgtcatctcagatcgaggagtgcatcttcctggacgtgggaaatactgacatgaagtacaagaatcgggtgcggagccgaatctctaacctgaaagatgccaagaaccctgggtc

SEQ ID NO: 610 - DNA, 547 bp, Mus musculus
actgagacagcagcagattggtgacaagaccgttcccatcccactgcggcgcctcccgagaggagtccggaacctttctgctgtgctgtcagcccggacatgctgaggcggagaccggaagccccttgtcggttccgggttttctgggctactacgatggctgatgagtttcgagtggccgtggcagtaccagcttcccgcccttctttacgttacagccgaacgtggacacccggcagaagcagctggccgcctggtgctctctggttctgtccttctgccgcctgcacaaacagtccagcatgacggtgatggaagcccaggagagcccgcttttcaacaacgtcaagctacagcggaaacttcctgtggagtcaattcagattgtattagaagaactgagaaagaaagggaacctcgagtggttggataagaataagtctagcttcctgatcatgtggcggaggccagaagaatgggggaaactcatttaccaatgggtttccaggagtggccagaataactctgtgtttactctgtatgaact

SEQ ID NO: 611 - DNA, 546 bp, Mus musculus
  misc_feature 25: n = A,T,C or G
gactgaggtggggtctttacttggntcggtcggcgaggctcacgcgagtgaagggcactgaggaaggaagaaggactcagagcatggcagaaaacagagaaccccgctgtgccatcgaggctgagctggaccctgtggagtacacccttcggaagcgacttccccaccgcttgccccggaggcccaacgacatttatgtcaacatgaagactgactttaaggcccagctagctcgctgccaaaagctcctggacggaggcactagggggcagaacgcatgcactgaaatctacattcatggcttgggcctggccatcaaccgggccatcaacatcgccctgcagctgcaggcgggcagctttgggtccctacaggtggccgccaatacctccaccgtggagctggtggacgagttggagccagagactgactctcgggagcccctgacgagggtcaggaacaactcagccatccacatccgagtcttcagggtcacgcccaagtaactgaagggaggctgacttggaccccaccaaggcttctgag

SEQ ID NO: 612 - DNA, 545 bp, Mus musculus
  misc_feature 298, 310, 318, 320, 321, 322, 323, 338, 341, 342, 351,355, 360, 371, 378, 384, 392, 397, 398, 410, 414, 421, 424, 427,433, 470, 471, 475, 484, 509, 526: n = A,T,C or G
actgagatgagtctgcatgtcttaaatgacgaaacgtgcccaatgaaaagagttcacagtgccgtgatttccagtttttgccaccagaacttaccggcagatcatctgttctttgtctgtcacagaaagaaaatgtaccaccccagagccaggctaaggccacaaatgtgaccgttccagacaccacctcgggatccacagacacacaggatcttaagtcctaacatgaccaataagcgtgaggctccttttggattacaaaatgaccactgtgtcttcttacagaaagaaaaccatnggcctttagccncagtggangnnnntcctgcggttcatanggnnactgagatnctangagcncagagcgagtnaccgganggaatnttgacttnctcannctttttagcttncacnattcttntgnacngtgagntggtgaccccttctatcgagccagtgctgcagccttnncatnagggactanaacctgtcttggattgtgagctggngacccctcctgttgagncagttgctggagcctacgt

SEQ ID NO: 613 - DNA, 543 bp, Mus musculus
  misc_feature 336, 389, 395, 414, 449, 452, 491, 513: n = A,T,C or G
actgagactccgttgggtaccccaaaagagaatcgcacgtccacaccggtgcggagtccagggggatccacaatgatgaaagcagtggtccatgccgtggacggcatcgctgagaacgggatccagcctctaagttcctccgaggtggacgaactcattcacaaggccgatgaggtcacactgagcgaggcagggtccacagctgggccagcggagcctcggggactcgcagaggatgtcaccaggaccacgccgtccagaagggagatcacaggagttgaggctcaaccaggagaggccacattaggcccaccaggcatccagcccggtcaggancccccggtcaccatggtcttcatgggttatcagaacgtggaagatgaagcatnaaaccnagaaggtactgggcctgcnggacaccatcaaggctgaactggtggtgattgaanacncggccacgcccagggagcccgcacctctcaatggcagcnctgctgagctcccggccaccanggaggagaaccagacggggcccacgattcc

SEQ ID NO: 614 - DNA, 532 bp, Mus musculus
  misc_feature 16: n = A,T,C or G
tgggctttttggcagncaagaaccagaacagggccagcgggtctctggaattcttgttaaaagaaactttaattaccatatactctctccctgtgacctctccaattacactgacctggccatgagcactgtgaaacagacccaggccatcccgtacaccgggcccttttacctgctctactaccagctccagaaattgacgggtgatgtagaagaattagaaattcaagaaaagcctgctctgaaggtgttcaaaagcatcactgtggtgcaggagccgggcatggtggttctggagtggctggcgaacccctctaacgacatgtatgcagacacagtgaccaccgtgatattggaagttcagtcaaatccaaagataaggaaaggtgcagtacagaaggtctctaagaagctagagatgcacgtttacagcaagaggctggaggtcatgctccaggacatatttggagaagactgtgtcagtgtgaaagatgactccgtcctcagtgtcacagtggacgggaaggcagtg

SEQ ID NO: 615 - DNA, 527 bp, Mus musculus
gactgagtgttatcagaaagcccgccagcttcccattactgtgacatcggaggctggagaaaatgatgaaactgtccagactactctcaagtttacatacagtgaaaagtaccctgatgagactccactttatgaaatattctcccaggaaaatctagaagacaatgatgtctcagacattttaaaattacttggcattgcaggccgaggagaatctcggcatggtgatgatcttcaccctcgtgacagctgttcaagaaaagttaaatgaaatagttgatcagatcaaaacaagaagagaggaagaaaagaagcagaaagaaaaggaagcagaagaagcagagaagaaactgttccatggcacccctgttacgattgagaatttcttgagttggaaggccaaatttgatgcagaactcctggaaattaaaaagaaacgaatgaaagaagaggagcaagcagggaaaaataaactaagtgggaaacagttatttgaaacagatcacaatcttgacacatctgatacc

SEQ ID NO: 616 - DNA, 520 bp, Mus musculus
gactgagagcgagaaggcttgctgcactccagccacatctcagaggagctgaccacgaccacagagatcatgatgggccggtttgagcgggacgccttcgatactcttttcgaccacgccccggacaagctcaaccttgtgaagaagtctctcatcacgtttgtgaacaaacacctgaataagctaaacttggaggtgactgacctggagacccagtttgcagatggtgtgtacctagttctacttctgggccttcttgaagactactttgtcccccttcacaacttctacctgactcctgacagctttgaccagaaggtgcacaacgtggcctttgcctttgaactgatgctggatggaggcctcaagaagcccaaggcacgccctgaagatgtggtgaacttggacctcaagtccactctgcgggtcctttacacgctgttcaccaagtacaaggatgtggagtgatggagcaactgatcctccagggacaagtgctaggccagaaaaatgccactcc

SEQ ID NO: 617 - DNA, 519 bp, Mus musculus
  misc_feature 29, 266, 268, 281, 287, 289, 314, 318, 322, 328, 329,330, 333, 338, 341, 344, 346, 362, 364, 368, 370, 371, 373, 390,391, 393, 406, 408, 423, 425, 430, 433, 442, 446, 450, 458, 460,470, 478, 479, 482, 493, 496: n = A,T,C or G
actgaggtagctgagacctatttccaggnaagaggactatgagaaagcaatgaaatttattcagcttgaaaggctgtatcatgagcaattgcttgcaaatctttctgccattcaggaacaatgggaaacaaaatggaaagctgtccagccacgtacagtgacgcctctgagaaattcagaaaagggatttaatggtgaagattttgaacagcttgctaaaatttgcacaactcaccaagaccctctcttgtccaagcttaagacancncctgtggaaccgncaccananaggaaaagccttgcaagggcgatantgtntgangaggcnnnggngacanaancancngccaaagagccagccntncctngnncnctccagagatggccacnngntctctgtgctctntnatattagaagtggtngntcggnaancctggaccntacntaanctttttcngngccgcctttngtatgccnncanagtctgccaanccnacatggggctcttcggcttctga

SEQ ID NO: 618 - DNA, 518 bp, Mus musculus
  misc_feature 346, 394, 409, 482: n = A,T,C or G
actgaggttctctggcagagccattactgaagtcttggacctctatgctacatctcgagagagacggtttcgtttatttgcatctatagaatgtgaaggacagttattcatgactccatatgattttattctggctgttacaacagatgagcccaaatgaattaagtcagatgctctcagaaactccaccagtttggaagggatcatcaaagctttttcgaaatcttaaagaaagaggtgtgatttcttacacagaatatctttttcttttatgtattttaacaaagccccatgcaggttttagaatagctttcaacatgtttgacacgtgacggaaatgagatgntggataaaaaagaatttttggtgcttcaggagatattcaggaaaaagnatgaaaagagagaanctcctggagatgaagaaaaacgtgccatgctgcgtcttcaactttatggataccattctcctactaatagcgnccttaaaacagatgctggggaacttgtctcccctga

SEQ ID NO: 619 - DNA, 515 bp, Mus musculus
  misc_feature 25, 38, 45, 73, 135: n = A,T,C or G
gactgagagacctgctgtcatgccnatcccatacgcantactgancaagactcttctacgacaaatcctaagntttcaacattacagctggtcttcatgttccaccccactctcccagaccagccgttataccanagaacaagattatcgacctaaaacagcaggaaggaaaacacctaccttggcatccccagtcccaggaacaccttttctccgccctacccaccaagttccccttgtccctcatgttcctattgttcgacctgcacaccagcttcacccaggattagtccaaaggttgatagcccaaggggtacatccacagcatcttccaagtttgctccaagctggtgtgcttcctcctgggatagacatggctcctttacaggggctatctggcccactcctgggtcaacccttgtaccctttggtctctgctgctagtcaccctctcctaaaccctcgtcctgggacccctctgcatttggcggtgatgcagcagcagctacagcgct

SEQ ID NO: 620 - DNA, 512 bp, Mus musculus
tgagacggccatggctaccctccagcagcagcctaccagcaagtactccagcctgctctgcctgggcaggtccttcctggggcaagggcaagaggcccacgccctgtgcagaaggtcatcctgaatgactccagcccccaagaccaagaagagagacctgcacagagagacttctctttcccgaggctcccgagggaccagcctctaccgcccaccatctaatggagtggaagcccctgaggagtccttggaccttcctgcagagctgagaccacatggtccccaggctccatccctagctgccgtgcctagaccccctagcaaccccttagcccgaggaactctaagaaccagcaatttgccagaagaattacggaaagtctttatcacttattctatggacacagccatggaggtggtgaactttgtgaacttactgttggtgaacggcttccaaactgcgattgacatatttgaggatagaatccggggtattgatatcattaaatgcc

SEQ ID NO: 621 - DNA, 510 bp, Mus musculus
  misc_feature 30, 207, 392, 404, 437: n = A,T,C or G
actgaggtttctacccagtcaccacaccgnctgcttggtctctgcagtgcaccactgctttgatggtcccacaggtgaactggcccacgctttcttcccaccccacggtggcattcactttgatgacagcgagtactgggtcttgggccccacacgctacagttggaagaaaggtgtttggctcacaaacctggtgcactgtggcancccatgagattggccatgcactgggcctgatgcactcacagcaagatcaggcgctcatgcacctcaatgccacattgcgaggctggaaggcactgtcccaggatgaactgtgggggttacaccgactctatggctgcctggaccggatttttgtgtgtgcatcctgggcacgaaagggattttgngatgtccgccanaggctcatgaagaggctctgccccaaaagctgngatttctgttatgaattcccatttcccacggtggccaccaccacatcacccaccagaacgaaaacaaggttgg

SEQ ID NO: 622 - DNA, 505 bp, Mus musculus
  misc_feature 21, 26, 27, 30, 32, 35, 40, 59, 60, 68, 73, 74, 75, 78,80, 81, 89, 93, 94, 95, 99, 108, 110, 114, 123, 125, 130, 134, 426: n = A,T,C or G
atgtgaagaggacatcagcangcagnngcncnctnctgancggaacccacatgtgacanntgtgaggnggcannnaangnnggcactgngcgnnnatgnaaccatctnantagntaccgtgcnangatgngacnttaatgcaggtaagcctccaagccacgacatgtttgattttcttcatctcacaggagggggctatgcaaagatctgcaaacccctccatgaactgatcatgcagttgcttggaggaaacaccggaggagaagcagaccaccattgcttgactggcccatcagtgttacagcatcccgcttcctcagatcagttagaccactggcactacagaatcctttcttaaggcactttgcattgtacaatgttcctggatgcttgtatgtttgaaggggaggagggatttaaattggncctgtatataaaggttttgtttgagaggagaaaaattagccaaggaagattcatttgaaaccagaaggggactttttag

SEQ ID NO: 623 - DNA, 505 bp, Mus musculus
tgaggcagtggcagcccagccccagccccagccccagcaatgtgccccatttctccaccatgttcttctatggctcaaatacctaaagcaacccaacctgaggtggatcttcctcaaagttctggaaacttcaaaaaagaagagctggctacacgcctgtcccaggccattgcaggtggggatgagaaagcagcggcccaagtggcagcagtcctggcgcagcatcatgtggctctcaatgtccagctcatggaggcctggttcccaccaggtcccataaggctacaagtcacagttgaagacgccacatcggttttgtcctcttcatcgtctgcccatgtctcactgaagatccacccacattgctccattgcagcccttcaggatcaggtgttctcagagttcggtttcccaccagctgtgcagcgctgggtcattgggcggtgcctgtgtatgcctgaacgaagccttgcttcctatggggtctctcaagatggagaccctg

SEQ ID NO: 624 - DNA, 500 bp, Mus musculus
  misc_feature 126, 406, 437, 455, 458, 460, 463, 474, 484: n = A,T,C or G
tgagcttccggagtatgggtccacgggcgctgcccacgccgacagtgagtatgagagaagaatgatgtccgtgtacagtcgcgtcctagaggaggtggagtcgcttaaccggaagtatgccccagnttcctacatgcacacagcatgcctctgcaatgccatcatcgctctgctgaaagttcctctttcatttcagagatactttttccagaaactacagtctaccagcatcaagcttgctctgtcaccttctccccggagccctgcagagcccattgctgtgcagaataaccagcagctggcactaaaggtagaaggcgttgttcagcacggatctaaaccaggcctcctccgcagagttcagtctgtgtgcctgaatgtttcctccacactgcagagtaaatcnggccaagactacaagattcccattgatagcnctgaccaatgagatggancananggntgagccacatnaatgactacntcaccacccagtctcc

SEQ ID NO: 625 - DNA, 500 bp, Mus musculus
gactgaggggccttcgagattgagatcaatggacagctggtgttctccaagctggagaatggagggtttccttatgagaaagacctcatggaagccatccgaagagccagcaatggagaacctgtagaaaagatcaccaacagccggcctccctgtgtcatcctgtgactaagaaatgattcttggaagcttggttctggggtttaaacctttccttgtggttacgctaggaatccctctacctttagcccctcacttggccctaggtaactaagaccctgacttcactttgcctcttagggcacaaggagggactagacatttctgtggccaaaggcagaaaagagatgttctctgtgaacatcaccatcgctctcccgggatctgtgccctctgcagtttggtcctctccccagcgtggcaatacctttcagatgccacggttgctataacttattttccctgaatcctgggcagtgtcggctcttggcaataaagag

SEQ ID NO: 626 - DNA, 497 bp, Mus musculus
gaggtggacagtggaattcggatgaccccatccccagaaaggatctggagcaaggtgtggtgggggcccatggcttgctctgccgcctctctgaccgtgtggacaagaaacttctggatgccgcaggagccaacctcagagtcatcagcaccttgtctgtgggggtcgaccacttggctttggatgaaatcaagaagcgcgggatccgggtgggctacacgccaggtgtcctgacagatgccactgcagaactcgccgtctccctcctcctcaccacctgccgccggttgccggaggccatagaggaagtgaagaacggcggctggagctcctggagcccattatggatgtgcggctacggactctcgcagagcactgttggcattgtggggctggggcgcataggtcaggccatcgctcgacgactgaaaccattcggtgtccagagatttctttacacggggcgccagcccaggcctcaggaagcagccgagttt

SEQ ID NO: 627 - DNA, 497 bp, Mus musculus
  misc_feature 177, 232, 250, 351, 362, 394, 420, 451: n = A,T,C or G
tggaatatgagagattttaaccagtttatcaaggctaatgagaagtggggtcgtgatgatattgaaaatatagccagagaagtagaaggaaaaactccagaagaagtcattgaatactcagctgtgttctgggaaagatgcaatgagcttcaggacatagagaagattatggctcanattgaaaggggagaggcaagaattcaaagaagaataagtatcaagaaagcactanatacaaagattggacggnacaaagcaccctttcatcagctaagaatatcatatggtactaacaaaggaaaaaactatacctgaagaagaggatcgttttctcatttgtatgctgcacangcttggcttcnacaaggaaaatgtgtatgatgaattacgacantgcatccgaaactcccctcagtttanatttgattggtcttctcaagtccagaactgngatggagctccagaggaggtgtcataccttaattactttgattgaa

SEQ ID NO: 628 - DNA, 494 bp, Mus musculus
  misc_feature 138, 227, 413, 448, 454, 469, 473: n = A,T,C or G
tatgaaggactcaggacaccggctcatgccaaaactcatcccatcacaaagtcattttacatcaaaaatggcgttttccctaaccctcggcgatcagatgacatcatgtgacttgacttggaaaaggcctcagcggcntcctttcccccgtcaccatctggaccagccagatgggagatatttcggcccagccctacctcactccatgttctgctgagccctgcctncttgagtgagatggacctgaagaagtgaccacgggcatacacctctggggtgtactgtcatcctatgttggggctcctgtctacacacagagaactccgctgcttgcaaagctgtcgcgtcctaccatccgtcactagcaatgctgtttcttttcctggggtgatccaatgctgcatgagcaggcnctggacagaggaggcgcttgtgagcctgcaaggcncgtggncgagcaccaatcctnttangcctattcttctgcattagac

SEQ ID NO: 629 - DNA, 488 bp, Mus musculus
  misc_feature 32, 34, 35, 48, 52, 55, 60, 62, 67, 73, 108, 172, 181: n = A,T,C or G
gcgctcatatttacttacttgtgggcactctnanngtgtcggactggncgcntangatgncnccatntgagcnggagcagcctcatggtcacagatgctcacagagcngcagagcagcctgctggagacgctgcagagaggcgcaggacgggttggcgacagcgcagctacngactgcgcngagctcggccacgagaaggactcgctgcagcggcagctcagcatcgctctgcctcaggagtttgcggctctgacgaaggagcttaacttatgtcgggaacagcttctggagagggaagaagagatcgcagagctgaaggcggagcgaaacaacacccggctccttctggaacaccttgagtgcctggtgtccaggcacgaacgttccctgcgaatgactgtggtgaaacgccaagcccagtcccctggaggggtctcctctgaggtggaggtgctcaaggctctaaaatccctctttgaacaccaca

SEQ ID NO: 630 - DNA, 487 bp, Mus musculus
  misc_feature 165, 168, 172, 173, 180, 182, 183, 195, 197, 209, 234,243, 259, 267, 274, 282, 295, 298, 341, 346, 358, 402, 406, 412,432, 443, 471: n = A,T,C or G
gactgaggaatctgtgggctcctgtggccgacacacatgagggtcatatcaccagcgatatgcagctttctacctacttagaccctgccctggagctgggtccccgtaatgtactgctgttcctacaggacaagctaagcattgaggatttcacagcatactggnggngtgnntggaaananncaggacagtgcnantttctaacctgnagaatgccctggacttggccccctnctcactggngcttcctgctgtggantggtatgnaataagnactctgancacctacctacanganaagcttggggctagccccttgcatgtggatctaggtaccttanagganctgaagctcaangccagccttcctgccctgctgctcatccgtctgccctacacagncagntccggnctgatggcgcccagggaggncctcacaggtnacgatgagggcatcggacaggcactgancacactcatgtatgaa

SEQ ID NO: 631 - DNA, 469 bp, Mus musculus
actgagcttcccctctgccccatgagaaacatcttcaagaggaaccaggagccaaatgtggctccggccaccaccactgccacaatgccccttgcacccgtcgcacctgccgacaactctacagagagcacgggtcctggggagagccaagaagacatgttcgccaagctgaaggagaaattcttcaatgagatcaacaagatcccctgtgagtggccctgtggccaggagggttactgggctagcctggaaagcctatgtcaggccagaggaggcggggagccactgggctgggatctgctggcttctttctttgggtctcagtttggatttccaccccagaggcatggcttgggagtgtgatctggaattgaggaagccaccaagggagtttatgttacacatcccaagctatagaagccaagttcaatttcaagtacaacctgaaatccctcctcaaaaaaaaaaa

SEQ ID NO: 632 - DNA, 461 bp, Mus musculus
  misc_feature 262, 317, 368, 374, 379, 384, 445: n = A,T,C or G
ccgacagttatttatgcaaaacagctgaaagagagattggaggcttttacaagagattttcttcctcacatgaaagaggaagaagaggtttttcagcccatgttaatggaatattttacctacgaggagcttaaagatatcaaaaagaaagtgattgcacagcacatgctcacagaaggacacggcagagctcctcagagggctcagtctgtggaaccaagctgaggagcgacagaaagttctcaagtactctgtggatganaaggcagatacagaagcggaagtgtcagaacactccacaggtataacccatcttnctcctgaggtaatgctgtccattttcagttaccttaatcctcaagaattgngtcggngtagncaagncagtactaagcggtctcagctggtaaaaacaggatctttgtggaaacatctttaccctgtncattgggcaagaggtg

SEQ ID NO: 633 - DNA, 457 bp, Mus musculus
  misc_feature 16, 36, 51, 58, 59, 66, 71, 75, 77, 95, 101, 102, 112,136, 141, 400, 421: n = A,T,C or G
gctaaatgctggcccntggggggactacctgctganattcttcttgggccnttatcanntgcgtcnttggnggcnangaaggagattgatccagngcctgnnctttattctnatggacaggggactgacacaccgnacgtncccaatgagagtctaggagaggcagcctgtgctggcaattgtggaacgcttgaccaacctcatcaaggaagtctttccagacactaaagtctatgctgctctgggaaatcatgacttccaccctaagaaccagttcccagcacagagcaaccgcatctataaccaggtggcagagctgtggagaccctggcttagtaacgaatcctacgctctcttcaaaagaggtgcccttctattctgagaagctgccgggtcccancagggcggggcgagttgtggncctcaacaccaatctgtcgatctcgcaagcacaaag

SEQ ID NO: 634 - DNA, 453 bp, Mus musculus
  misc_feature 29, 137, 147, 179, 281, 321, 360, 377, 437: n = A,T,C or G
ttgtacttcctccagtcacacaatgccgncgccttcggacatcagtgaaggtggccatagaatggcctggcgcctaccctaaactcatggagattgaccagaaaaaaccactgtctgccattataaaggaagtctgngatgggtggnctcttgccaaccatgaatattttgcactgcancacgctgatagttcaaacttctacatcacagaaaagaatcgcaacgaaataaaaaatggcacgatccttcgattaaccacatctccagctcagaatgcccancagctccatgaacgaatacagtcatcaagtatggatgccnagttggaagccctgaaggacttggccagcctctcccggnatgttacctttgctcangagttcataaacctgggatggtggagaattcccaagccacagcccttttgtctcgggganctcccttcttcacaga

SEQ ID NO: 635 - DNA, 451 bp, Mus musculus
  misc_feature 19, 190, 192, 408: n = A,T,C or G
aaacagcaggatacattanttgtttggtcagaagctgaaaactatgatctggctctaagttttcaagaaaaagctggcttgtgatgaaatttgggaaaaaatttgtcaagttcaaggtaaggacccatcagtagaagtcacacaggacctcattgatgaatctgaagaagaacgatttgaagaaatgccngnggctagtcatctaattgacctgcccgcatgtgaactcagtaaacttgaagagattgctgacttagttacctcagttctctcctcacctatccgtagggaaaagctggctctggccttggaaaatgaaggctatatcaaaaaactactacagctattccaagcttgtgagaacctagaaaacactgaaggcttacaccatctgtatgaaattattanaggaatcttattcctaaataaggcaacacctttttgaggcaat

SEQ ID NO: 636 - DNA, 448 bp, Mus musculus
tcccgcttacgcagacctcatccatagcagtggctggagccgtgattggagctgtcctggccctcttcatcatcaccgtctttgtgactgtgttgctgacgcctcggaaaaagagaccgtcctatcttgacaaagtaatcgaccttccacctacacataagccaccccctgtatatgaagaacgaattccttctctccctcagaaagaccttcttggccaggtacgtgctctcgaagacacttaaagtcaccagaactagcttctaaacaccttgcccagctgttgtggcacacatctgcaaacctagcacctgagagagaggctggagcaggaccaggagtttaaagtcagccttggcgacactgagtttgagacctgcctgggctactggagaccccattgcaaaatttgaagtgaataaattttttaaaaatccaaaaaaaaaaa

SEQ ID NO: 637 - DNA, 445 bp, Mus musculus
  misc_feature 156, 165, 168, 170, 187, 191, 192, 197, 198, 225, 232,235, 265, 270, 276, 286, 288, 289, 297, 310, 313, 321, 328, 359,368, 395, 396, 411, 425: n = A,T,C or G
cagttttgccagtgctcggggttagacctgatctttggtctaaatgcgttactacgaaccccagacttacggtggaacagctccaacgcccagcttctccttgactactgctcttccaagggttataacatctcctgggaactgggcaatgagccnaacagcgtnctngnagaaagcttacattctnatcnnttggnnagcagttaggagaagactttggggagntgcatanacntctacaaaggtcagctttccaaaatgcaanacatntatggncctgacatcngnncagcctcnagggaagacagtntanctgctgangagtttnctgaaggctggcggagaagtgatcgactctnttacatggnataactattacttgaatggccgcatcnntcccaaagaagaaanttctgagctctgangtgctggacacttttattct

SEQ ID NO: 638 - DNA, 439 bp, Mus musculus
  misc_feature 18, 28, 39, 87, 282, 382, 386: n = A,T,C or G
tatatacctgaggagaanaggattcccnagcctaccggncccctgcgccctccccggccagcgtgaacctattacagatgtggcagncatcgcctagggcacatgggcgaggaggtgccccagggctatacatgcattcagacttctgcgggtggccaccccttggagctcagtgctgggctcctgggtggaactcaaccagtcatctgctaccgcagggggcgtgacaagccccccctcgttgagctgggggtgttatatgaagggaaggaacgacccatncttgggttccaagtgcttgacacgacaccgtatagccactcagccaatctggccccttcaggccccgggcacccccgcacctacctcatgtatcggcggncagnaaaaggggcagggcctgcatgccctgggcatcactgacctctgcctggttctg

SEQ ID NO: 639 - DNA, 439 bp, Mus musculus
  misc_feature 133, 146, 182, 220, 221, 236, 245, 295, 298, 310, 311,327, 330, 418, 420: n = A,T,C or G
actgagcatcacaatggggctgcctctgagccctgcagctgactctgcccgctccgcaaggcacgccctgtctctcatcgccacagccagaccacccgccttcatcaccaccatagctaaggaggtgggcagncttcatcacgtgntccatcgcccctttcagaggagttcctccgggccgnaactctggggtcaggacccagcagcagaatccttatanntcaagcaaagcaagntaaccgttnacggcatgaaatgaatggatcatgtgtgcacgcaccctgagttccgcatntgngggagtgtgctnncgtgctcctttctgtnctnagaatgaacacacttagtgacaatacctttcagtatgaagtgcaataggtctatgagaagtagaagatattaaaggtattcacactgntnaagtgtaagcaaaaaaaaa

SEQ ID NO: 640 - DNA, 438 bp, Mus musculus
  misc_feature 39, 56, 76, 213, 296, 302, 308, 328, 344, 345, 348, 360,367, 368, 387, 403: n = A,T,C or G
ccatcgggcaggcgggaaacccggaagaaggacgggatngctgtgagtgagattgngagtgaagcgcctggaagcngcagaaccctgctctgcaggatggacccagccctgtctgctgtccggctcaccgtgcaggaagccattcacaccctttcatcctcagaggatgctggccatatcctttctaccctggggactctgaaacgatacctnggtggaaccgaggatccagttcttcctgaggagaaggaggagtttgccactgcccacttctcagccgtccttagatgtctggncagcangctgancccaggttggctggagctgncccccggtggtgcagnntgnagcggttttggnagagctnnttcctggatggccctccanaccaagccttcctggngctgatggaagccattgcgagcactgctggcgcca

SEQ ID NO: 641 - DNA, 437 bp, Mus musculus
  misc_feature 228, 332, 343, 345, 368, 370, 390, 399, 417, 419: n = A,T,C or G
tgtttctcagcctcagcaaaaccatggtatccttgttagtaatattacagagtcattgtctaccaaatcatgtggagcaccgcgacctgtcaatggagttgttaacaggtaatgttttagctcttctacttctaaaacgtaatactcttataaatgtggaatttgtagcccctagttattcccagccaagattggaaaaaccctaccactcattcatactttgtatgngggtatactataaagtatactttatttagtatgttcatgcaatcccttgtacctaccattgtctaaaagggactggccatggagtaaaacaaagtcctacaaantatttgaaggntnttaataggatccatgagcatatnantacatattatccttagaatncattatttnatagaaactgtgactgcntnattaatatttgcaaaagt

SEQ ID NO: 642 - DNA, 434 bp, Mus musculus
  misc_feature 43, 56, 60, 158, 159, 160, 162, 163, 171, 176, 177, 179,183, 184, 185, 195, 201, 214, 216, 229, 241, 243, 262, 283, 289,297, 309, 333, 335, 367, 378, 392, 397, 405, 406, 409, 416: n = A,T,C or G
gcaggaaccgagcatcacagaagaagaaaactcggaggacgantctgacgagaagngctntggctacggtgaccacagaagcattagctgaaaatgtgaattctacccacaccaacgacacctcaaaccaggtggaatttatattaatgggggcgaannnannggcaagancagannancctnnnattcatggcnctcatngcgactctactgngngggcaagagaccnaagcaagagccntntagccaaggatctcaaagngccttgcagacacatgaactnggaggngaaacccngaaagtccctanttttgaagaggacacaccctctgncnatggaaatagagatggaagagaccgataaaanggatgaacagngtgaacagaaacgncgacnatgagtgnntanctacttngaaggaccaaaaggaacc

SEQ ID NO: 643 - DNA, 433 bp, Mus musculus
  misc_feature 25, 79, 83, 88, 94, 103, 104, 120, 123, 124, 135, 157,241, 251, 291, 294, 309, 321, 337, 347, 364, 366, 381, 399, 415: n = A,T,C or G
gtacccatgccccaccctggctctnagtgggcacagctcggacaggcttgaactttgaccttggggacaaggtcaggtnccgntgctnctcctnaaatatggnnttgactggctctgcanagnngggagtgtcanagcaatggagtgtggagtgggncggaacccatttgccgacagccttactcttacgacttccctgaggatgtagcatctgccctagacacctccctcaccaacctgnttggagccancaatcccacccagaaccttctgacaaaaagtttgggccgnaanatcataatccagcgntcgggtcacctnaacctatatttgctgnttgatgcttntcagagagtgacagaanangaatttgacatcttnaagaagagtgccgaactnatggtggagagggtcntaaacttaggggtacatg

SEQ ID NO: 644 - DNA, 432 bp, Mus musculus
  misc_feature 343, 365, 391, 394, 416: n = A,T,C or G
ctcttttggcccttgtggtaaatacaacgcgatggtgctggaactgctgggaccgagtttggaggatttgtttgacctctgtgatagaacattttctcttaaaacagttctcatgatagctatacagcctgatttctcgtatggagtatgtccactcaaagaacttgatatacagagatgtgaagcctgagaacttcttaataggacggccaggaaacaaagcccagcaagtcattcatatcatagattttggtttggcaaaggaatatattgatccagagacaaagaaacatataccatacagagaacacaaaagccttaccggaaccgccagatacatgancataaacacacatgtaggaacnagagcagagtagaagagatgatttanaanccttacgccacatgctcatgtncttcctgcgaggcccg

SEQ ID NO: 645 - DNA, 432 bp, Mus musculus
  misc_feature 312, 356: n = A,T,C or G
accccgagcgccaagtgacacccggcccatcgcgccggaaggatattctcggcgcggagctgctcgatggtggcctcgcagcgccggagcttgctcctgcagcgtccgcatcgcccaccccgttctcgtcgtcgtcgtcgtcctcttcctcctcgtcgtccttgaaccgggtgtgaacgggcttcggcgtcgactcctctggtgggtcaaacgcctcgaagagctctaggtcgccaaaatccacaccggccgccatcttgggctgtgggaaagcttggagggcagggggcgaaggttggaggccagagccgntctccctggaggcttttctccacacacaggcctccaaagccgtntaggggcgggtcctggacggagactacaactcccagaatgccaaggccgccctcatgcctctttccgaatgggcca

SEQ ID NO: 646 - DNA, 427 bp, Mus musculus
  misc_feature 229, 230, 235, 254, 256, 262, 275, 281, 283, 291, 293,294, 296, 297, 304, 309, 318, 319, 321, 323, 324, 327, 328, 331,332, 333, 342, 351, 352, 356, 361, 365, 373, 376, 384, 404, 407: n = A,T,C or G
gactgaggtaggagtataatggctgtccaggtgctgcggcagatggtctacttcctactgagtctgttttctctggtgcaaggtgcacacagtggcagcccccgagaagacttccgcttctgtggccagcggaaccagacccaacagagcaccctccactatgatcaatcttcagagcctcacatctttgtgtggaacacagaggagaccctcacaattcgtgcctgtnngctgncagtggtgaccgtgttccnanctgggnatagttgtatgantcctcnanacacacantnnannctgtgtncctgngactgtganntncnnatnntgnnntagtggggntcttgatgnncttngctanctgntgtatacnttnggactgtnacttgcacagctgtgctcantangagagatggccatgctcccg

SEQ ID NO: 647 - DNA, 411 bp, Mus musculus
  misc_feature 17, 42, 51, 69, 71, 72, 83, 86, 87, 90, 102, 105, 122,160, 180, 187, 193, 195, 199, 204, 238, 242, 249, 252, 258, 264,272, 281, 286, 291, 299, 301, 304, 323, 333, 337, 343, 357: n = A,T,C or G
aagaagcgagcagaagnacagggagaagaagaggaagaacancaagacgangctgtggacgaacttgtnannggatagtcagnggnnaangcagtggcgggngcnccaggccgaggctctgnccggcccctcactggatcagtggcacagatcatctggngaggacaatgatggcccagncctgacngatgancngaantctcntatgcaggccatgaaacccatgacaaaggaggantggnatgcccnacngagcgntattcnaaaggtgntggacccanagacnggacncacaaggntnatnaagggagacggcgaggttntagaggaaantttnaccaangaacgacacatatngatctgcaagctagccacccccaggggatgggctggcctttcagatgcgaacag

SEQ ID NO: 648 - DNA, 409 bp, Mus musculus
  misc_feature 328, 360, 369: n = A,T,C or G
ggtttgaagaccatgaaggcagcaaagctggccaagatgaagataccacccggtgagatgttcctgtcagaagtcaacaagtattctaaatttgatgaaaatggtctgcctacccacgacaccgaaggcaaagacctgagcaagggccaggccaagaagctgaagaagctctttgaagcccaggagaaacctgtacaaggagtatctgcaaatgttacagaatggcagcctccagtgatgccacggcagcccaggaggggcgcacctgccagcctggctgctatggactaggccacagctactgaggctccgtgccctgatcatgttnacagctgttctcacacctgaatcaggcagagnggtagcagntctgggacactgataataaattcttctgaacggtgaaaaa

SEQ ID NO: 649 - DNA, 403 bp, Mus musculus
  misc_feature 180, 301, 378, 381: n = A,T,C or G
tgtgggtgatgctgttgcagccgagctgctgaacattctccgtggactcttccctgccacaccttgcccatcagccgagataaaatggaagactcctacaaggataggacttcactgatgaagggtgccaaggacattgccaaagaggtgaagaagcagacggtgaagaaggtgaatcangcagtggaccgggcccaggatgaatacacccagcggtcctacagtcggttccaggatgaagaggacgatgatgactactacccacctggcgaaacctacagtggggaggccattgatgacnaaggctcaagtgaggccactgagggtcacgacgaggaggatgagatctatgaaggggagtaccagggcatccccagncangaaccaagggaaggacagcatc

SEQ ID NO: 650 - DNA, 401 bp, Mus musculus
tgaggttccaggtgacaggggtatttatgaagaactgggactcgctggatgagcagggcgtttgtgctgctgacaaggactgtgaagatgcgtataaagtttgtcagatcttagacatccctttccaccaggtgtcctatgtgaaggagtattggaatgatgtgttcagtgactttttgaacgagtatgagaaaggaaggactccaaaccccgacatcaactgcaataagcatatcaagttcagttgcttttatcactatgctgtggacaatcttggagcagatgccgttgccactggccactacgccaggacctcactggaggatgaggaagtctttgagcaaaagcacactaagaagcctgacgggctgttcagaaatcgatttgaagtcagaaaccgc

SEQ ID NO: 651 - DNA, 401 bp, Mus musculus
  misc_feature 151, 155, 192, 225, 244, 296, 299, 301, 304, 305, 322: n = A,T,C or G
actgaggtacaagtagctctgggggttccgttccaagcccagccgacggagagtctcctcctcgcccccctccagtagctggtaaaagacgtggaagttccgctctccgtgattttggtgcaccacccgggacttttccaggagccacgtnctgntgctttaaccttgaaaacacccagaaccaacctctgntcatcacctctacgtctagcaccctgatctganaggccgtcatgccatctgncaggtagctacattacatgggcctcctaaccggttcccctgcttggctgtgntcntntgnnatctactcccaacgacnggcagaatgaccttttgagtaggaaagttacatcactacacgactgctcaaaaccctctgatagttccttttcacttag

SEQ ID NO: 652 - DNA, 399 bp, Mus musculus
  misc_feature 17, 26, 46, 57, 70, 73, 109, 121, 146, 149, 167, 176,190, 196, 201, 205, 228, 230, 231, 232, 237, 240, 242, 248, 250,252, 260, 272, 287, 294, 308, 311, 319, 326, 356, 381: n = A,T,C or G
aaagggtctggggcatngctcatggnatccagatgccctcagtgcntatgtgcccanctgtttggcagcncgnactgtccagttctcactggccctggcactgtgtatngagtgtgtgcangaactgccaccttccacctgctgcnggntgtgcctgctagttctgnctacactcncccaccaaccctcnggcacngctgnatancagcttctggagcctcaagctgntnnncctgntangnctctgngncntagccttntgcatccctgangaggatctcttcccnggctggnattatattggcatntgnggaggctncacattnatcctattacaggtggtgcttatcacagtntttgcccagccctggaacaagaatnggtagactggcgcagctc

SEQ ID NO: 653 - DNA, 389 bp, Mus musculus
  misc_feature 16, 17, 153, 167: n = A,T,C or G
tggaaaataagcgagnngccttttttcctccattacatcaattttgcatcaatccaaaaaaccctgttacagtaattcgcggtcttgcgtggagctcttaagttagatcttggacttttctctaccaaaactttggtagaggctaacaatgancatatcgtagaagngaggacccaattgttacagccagcagatgaaaactgggatccctctggaacaaagaaaatctggcgatatgaaaataaaagttctcatactacaattgctaaatatgcacagtaccaggcctgctccttccaggaatcattgagagtaagtttgttttacattcactttgggaaaaagaccattaagagaattttaataaagtatttcacttgtacagggaa

SEQ ID NO: 654 - DNA, 387 bp, Mus musculus
attgaggcgtggccagtcgactgctggggccatcgttcgagccttacctgttgccggaactgaccagatatgactgtgaggtgaactgtgcccgtgcctggaagctccacactcctgcaggggagagaccttctcagagctctggaccaggccacctgagccagggcctctggccgggcatgcccctgcctgccccgccgtcttgacctgccagcttcacttccatctgtgttgctattaggtatctctaacaccagcacacttcttacgagatgtactcaacctggcctactggccagatcaccaagcagtggcctttatctgacatgctcactttattatccatgttttaaaaatacatagttgttgtacctgcaaaaaaaaaaa

SEQ ID NO: 655 - DNA, 386 bp, Mus musculus
actgaggctggctttgaactcactctgtaactgaagaacttataaactgattctgactccctcaagggctgatactgcaggcctgggtcattacatacagtgccgaggactggatccagggcttctttcatgctggactcttttccaaagccagtgatctcacagtctgaaccaaacggagcatcagtaaaccttgggggcaggcagatggtctgtatggacctggatggctgtgcacattggcccgtgctggatgcccacgctgtgcatgttactggcttgcctgaattccaggaacttcccttgtggtctaaactggctgaagtttctgttgagagtgataaataagaaagcaaatgcatatgatttcattattaaaaaaaaaa

SEQ ID NO: 656 - DNA, 381 bp, Mus musculus
tgagctgctagctgggaaagaaggtggctgtggctgactatgtggaaccttctccccgaggcaccaagtggggccttggtggcacctgtgtcaacgtgggttgcatacccaagaagctgatgcatcaggctgcacgtgctggggggcatgatcagagatgctcaccactatggctgggaggtggcccagcctgtccaacacaactggtgagagagaagcccttgccctctgcaggtcccattgaaaggagaggtttgctctctgccactcatctgtaaccgtgagcttttccacccggcctcctctttgatcccagaataatgactctgagacttcttatgtatgaataaatgcctgggccaaaaaccttaaaaaaaaaaa

SEQ ID NO: 657 - DNA, 378 bp, Mus musculus
  misc_feature 25, 291, 304, 311: n = A,T,C or G
ttgcgacctcatcaggcagtgcggngtcataaatccagagaccaagaagatctgtacacgcctattgacttgcaagatccactcagtgcaccagcgcagggaggtcccaggcagggccaaggactttgatgtgctagtggccgaactgaaggccagctcccgaaaaggagagtctcccaaggagaagagcccagcacacaaagagccagctctggagcgcccatcccaggagcctcccactgcagtccagggtgtggcagaggtggctgcccccaacaccaccttctcttntctgtctaagcanacctatncgtactgtgcactccccaggctgctgccgctttttataactttatattatttttttttaagaaaaaa

SEQ ID NO: 658 - DNA, 362 bp, Mus musculus
  misc_feature 302, 311, 313, 317, 346: n = A,T,C or G
tgagagctgttggggatggtggcagtatttccaaaggaagaatatcttctctaaaagcacgggctgttttgattgacatggaagaaggcgtagtgaatgaaattctccaaggcccactgagagacgtgtttgatagcaaacagctcatcactgatatctctggctcgggaaataattgggctgtgggtcacaaagtttttggatgtctttaccgggaacagattttagagaaactgcggaagtcggcagagcagtgcgattgcttacagtgcttcttcatcatccactccatgggcggaggnaccagcccntnccantcagcttccttcaaggaggagcccattcncaaacagcccctattg

SEQ ID NO: 659 - DNA, 357 bp, Mus musculus
  misc_feature 319: n = A,T,C or G
ttggtaaaatattttgtttatttggaccaaagaagaatcctgaagctcacatgaatgttagccgagattattaaacactgggattatccaagcgaagaatatgaggttgtgactgatgatggttacattcttccaattaaccgaattcctcatgggaagaacaatgctaatagttcagccccaaagatggtagtattttgtcagcatggcttgcttgcaacacctggtgcatgggtttccaatccgcctgtcaacagcctggccttcatcctagcagatgctgggtatgatgtgtggatgggaagcagcagaggaagtncctgggcaaagaaacacgtggccctcaacccagattct

SEQ ID NO: 660 - DNA, 346 bp, Mus musculus
gagtgccacccggaagcgcggtttccccaggcagacgcgtttgaaacacttcttgcagaagaagctctctggcagctgcgcaccctcaacaggtttcgcatcccagccttcgagccctcgagtcggaggaagacctgacacccagcctccagcgtcacacctctgtagaaccccagacaacaaagccactgcagacagcgccagaggcgacgaaagatggctgtcagcttctgttaacgatgacgtggatacggatgaggagtcagcacagctcagagggtccaaaagaatcggtgacggcttcccgtttgatgaagaaagccccagtaaattccgtatggtagga

SEQ ID NO: 661 - DNA, 334 bp, Mus musculus
  misc_feature 32, 152, 156, 188, 208, 216, 240, 243, 248, 254, 269,305: n = A,T,C or G
agtagaagaagatgaacccacagcatttggcngaagataaagaagaggaggatgtctccagtgaaccagaagcttcactccagtgcagacacaactattctgtttgtaaaaggagaagattttccggcaaacaacatcgtgaagttcctggntggntttacaaacatggggacagaagattttattgntgaatcgctagatgcctcgntccgttancctcaggactaccagttttacatncanaattncacagntcttcctctaaacantgtagtgccaccccagcggcaggctcccttttgagnattccttcatccctgcagagccgatggga

SEQ ID NO: 662 - DNA, 333 bp, Mus musculus
  misc_feature 38, 39, 41, 45, 46, 133: n = A,T,C or G
tgctgccgtttaataaatcctccctgttagctgtgaannanctgnnatattctcaatagcctcctgttcctttcaagtgagctcacaaattctctatgagctgcagaaaggccaagcgctcatcacctttganaaggaagaagtcgcacaaaatgtgatatcgatggggaatcatgtcgtgcagatggaaggcaccccagtgaaggtctctgcacacccagtcccactaaacacaggcgtcagattccaggttcatgtggacatttctaaaatgaagatcaatgttacgggaattcccgatgagctgtctgaggagcaaacaagggacaaact

SEQ ID NO: 663 - DNA, 326 bp, Mus musculus
gactgaggtatcatgagctcattacaaaatatgggaaattggagcctttggcagaagtggacctaagaccccagagcagtgcgaaagtagaagttcacttcaatgaccaagtggaggaaatgagcatccgcctggaccagacagtagccgaactgaagaaacagctaaagaccctggtgcagttgcccaccagcagcatgcttctctactactttgaccacgaggccccgtttggcccagaggagatgaagtacagctcccgggcactgcactcctttggcattagggatggagataaaatttttgtggaatcaaaaacaaaataa

SEQ ID NO: 664 - DNA, 311 bp, Mus musculus
  misc_feature 30, 212, 249, 267, 274, 293: n = A,T,C or G
agggctgggagcctgcaccctgactggagntaccctgatcttcttgctactccccagaagtctggagagctgtggacacatctgagatttcaccccctgttgtccgcctgggggaccctgtcctggcctcttgcaccatcagcccaaactgcagcaaactggaccaacaggcaaagatcttatggagactgcaagatgagcccatccagggnggggacagacagcatcatctgcctgatgggacccaanagtccctcatcactctgnctcactngaactacacccaggccttnctcttctgcttaaacgcc

SEQ ID NO: 665 - DNA, 309 bp, Mus musculus
  misc_feature 24, 29, 33, 41, 43, 47, 61, 62, 65, 73, 78, 79, 85, 93,96, 124, 134, 144, 156, 289: n = A,T,C or G
agtgggtttgctgctgatgtacancctgngcanggccgaantnctcnttggggaactctcnnggncgggcagntatanngagctnttatatgnganacattcggacttttttactaggaaatangaaaaagacntctacgtggnggagcacagtancaggctgcctcagcgtgggaagccctgtttatttccggtcaggtgctcaggagtttggagagcccggatggcggaaacttgttcataacagacccccaaccatgaggccggagggagagaagctggctgcctncaccttgaaagtgaaagaaa

SEQ ID NO: 666 - DNA, 308 bp, Mus musculus
  misc_feature 16, 31, 35, 51, 103, 105, 142, 144, 149, 170, 185, 196,198, 207, 226, 234, 239, 254, 265, 266, 278, 288: n = A,T,C or G
cgcaccccacggtgcnccattacgtaatgtncctncaccagaaataatggngttgccggtatgaaagccgaggccgagccagtggcacagccatggactcacntnacccagagcttgaaggctcttcactgcgcccaccacngnctggngctccacacccagctgcgtanccaactccgcgctgnccagaccgtcntnagttacctncagacggcgtagtaacagntacaaaangggantatccgcctatggcntactcccagcnntgcttggcgtgngcaggccagncggacctgacataggggacc

SEQ ID NO: 667 - DNA, 302 bp, Mus musculus
  misc_feature 108, 181, 191, 211, 250, 272, 275: n = A,T,C or G
gtccttgacacccgatcatgtatgacagttttgacaacacctatcaggcaacaattggcattgacttcttatcaaaaacaatgtacttggaggatagaaccgtgcgantgcaattatgggacacagcaggtcaagagcggttcaggagcttgattcctagctacattcgagactccactgnggcagttgtngtttatgatatcacaaatgntaactcattccagcaaactacaaaatggattgatgatgncagaacagagagaggaagtgangtnatcatcatgctagcaggaaataaaaca

SEQ ID NO: 668 - DNA, 297 bp, Mus musculus
gactgagattccatgtattcagttatcctgagctcacgtacagcagaggatacccccgagtttcaccgccatctcttactaggattccaggatccctttcaaggcttctacctggagcaactgcccacagttctgccttaagagtgcctgagagacctggcatcgtcacagcctgccctccaaatcatcacctcactctgccaggcacaccaggtcttcctccccttcctcgccgcaccaaagaaatcaaaacccaaactgaggtatcagaaacatgtttttttttttttttttaaa

SEQ ID NO: 669 - DNA, 290 bp, Mus musculus
  misc_feature 137, 146, 147, 148, 149, 150, 151, 152, 153, 154, 155,156, 157, 196: n = A,T,C or G
actgagtgggccatgcagtgttggccatcaatggcatggcacgtgaatggcaagtacacggctgacgggaaagaagtgctggagtatctgggtaaccctgcgaattacccggtttccatccgattcgggcccccccnggggggggnnnnnnnnnnnncccgcccttaccttcttaatggaagaaaggcctttaatngcccttgggcccttcggaatggtttcccacttccggcttcttttgggccatttggggttccccagcttatcttcccgggaacaagggggccaag

SEQ ID NO: 670 - DNA, 287 bp, Mus musculus
  misc_feature 64, 66, 74, 80, 89, 92, 107, 159, 163, 169, 186, 203,205, 208, 214, 251, 260, 261: n = A,T,C or G
actggaagattgctagctactttaagaggacatgctgctgaaatatcagacatggctgccctananaaggattntgaggnggttggtgnganttgtgataaaatgantccgtgtctggtgtcttcgaacctgtgcacctttggctgttcttcagggacntagngcatcntattacatcactacagntctcaccattgtgcagngnctnaaaganatacctgttttctacaggggccggactggcactattngcattttgnnttttggaatgctggaacccttaaaat

SEQ ID NO: 671 - DNA, 286 bp, Mus musculus
  misc_feature 22, 41, 67, 92, 174, 255: n = A,T,C or G
ctggagttcctgtgttcttccnaaatgcattgctggctganagtgtgagcatgtgcttacagactgngccaacagagggagccagtaacagnactgaattaccacagggaacaaaagatgagccttaccggccatcatgataaccagaaaatttaccaggacttggtgggccangtgaaccacctactaagtaacgcctcccaagaaaccgaagaaccacctaccaaagcggtagttaccaaccgtgaatgcgcnaagacccaaaatactcaccatgcaaggaaaa

SEQ ID NO: 672 - DNA, 282 bp, Mus musculus
  misc_feature 63, 142, 145, 148, 189, 212: n = A,T,C or G
tccccagagctggaaggtggctctggccgctgccatggtgagtggtgtcgccatagtcgtggnaatgacaccctttgatgtagccagcacaagactttataaccaacccactgatactcgaggcaagggcctcatgtaccgnggnatnttggacgctctgctgcaaacagcgcggacagagggcttttntggcatgtacaagggtataggtncctcctattttcgcctcggcccccacactattctctccctcttcttctgggatcagctgcgctcctttta

SEQ ID NO: 673 - DNA, 263 bp, Mus musculus
gctctgagtgggcagtctcacttggcaataaaagatgacaagaagcaccatgatgcagagcgccacagaggtgagaggccgggcaagccagattaagctttgctcccatactcagacagtgctgcagagctgtgcctcgctgtggaaagcccaagccccacacagagagcaaacagtgagataacaggagctgtctgtcgcaaagcttgctttccactgacactcagttcaccctgccactctgcaaggtttgccttgggact

SEQ ID NO: 674 - DNA, 258 bp, Mus musculus
  misc_feature 173, 176, 202, 203, 210, 239, 241: n = A,T,C or G
ggccgacgcgcagaagcaaactgatgagcttggtcaacagcacagaagggaaagtggacaaagtcctcatgagaaacctctttatcggtcatttccacacacctaaacaccagcgtcacgaagtccttcggctcatggggagcatcctgggtgttaaaagggaagagatggancanctgtttgcacaatatcaaggaggtgnnactcggnggatggctggatggctggtcaggaggatntnaaaagtgagcccaatac

SEQ ID NO: 675 - DNA, 231 bp, Mus musculus
tgaggtaacatgaagagtgaggaatctaacaaagcaaaattcttctgagatggactatttagaaaatgccactgtaatagatgaatctgctttgacacctgagcagaggctaggcttgaaacaagcagaagaacgtctagaaagagatcacatcttccgacttgaaaaagtatgctatgctatgttagttacctgagggagaaatatatgctttatgacttttcctacact

SEQ ID NO: 676 - DNA, 221 bp, Mus musculus
  misc_feature 20, 28, 34, 83, 86, 97, 133, 161, 163, 168, 199, 200: n = A,T,C or G
ctttatcattcagtgtacanatgagcangaatgngaacatcccacagttggcggacagtttgtttgaaagaactactaacagnagntgggtggtggncttcaaatcactcattacaactcatcatttgatggngtatggaaatgagcgtttcattcagtantnggctncaagaaacacattgttttaacttaagcaatnnttttggataaaagtggattgc

SEQ ID NO: 677 - DNA, 221 bp, Mus musculus
  misc_feature 143, 148: n = A,T,C or G
aagaggatgatgagaagtctgacagtgaggatggagacctggataagcagatgggcaatctcaatggcgaggaagctgacaaactagatgagaggctttggggagatgatgaggaagaagatgaagacggtgacggtagagcntgaanagacaggaccaggggtggatgaggaagattctgaacttgatgctaaagatgacagcgttggatgctggaaatt

SEQ ID NO: 678 - DNA, 211 bp, Mus musculus
gctggctttgaacctgaagtcttctggtcccaacctcttgagcccctgttaggattacaggcagcacaccggccctggctccctctggtattttaattgagactaaattccattggaactgcttatggatagagggtggcttgacattctcttaagagtgccgagctgtttttcacccaagtaaaaagtcttgtctttgcaaaaaaaaaaa

SEQ ID NO: 679 - DNA, 200 bp, Mus musculus
gactgagatttttgcttcagtgtcttgaggatcttgatgccaatctacgaaaattaaattctcgtctgtttgtgattcggggacagccagctgatgtatttcccaggcttttcaaggaatggaacatcacgtaaacctctcaattgagtatgattctgagccttttgggaaggaacgagatgcagctatcaaaaaaaaaa

SEQ ID NO: 680 - DNA, 197 bp, Mus musculus
  misc_feature 29, 176: n = A,T,C or G
gactgaggatgaagatataacacttcagntaacagagagtgaagataatgaggacgatgacatggtcgatgttatctggcggcagttaatatcaagctgcccttggctttctgaacttgatgaaaatgcaactgaaggtgttattaaagtgtggctggaaagttgtggacagaatnttcagcctgtaaaaaaaaaaa

SEQ ID NO: 681 - DNA, 194 bp, Mus musculus
  misc_feature 46, 60, 86, 122, 139, 144, 166, 167, 169: n = A,T,C or G
caagcatgcacaaacgaagtaccgtgtcctgaaaattgatagaacnttaaccgagggacnaggaggtaatcgacgacaggcacgtntacacacaacaagaagtgagggaacttcttggccgnctggatctgggaaacanaacanagatgagccagaaaggatcctnngngttgttttagagccgtactcagtat

SEQ ID NO: 682 - DNA, 189 bp, Mus musculus
  misc_feature 33, 37, 40, 44, 47, 50, 60, 72, 74, 75, 84, 114, 131,147, 151: n = A,T,C or G
agcatataggcacgctttcactaaccttacatncagnacnagantanagntgaaaatgcngaaactcaattnannacaattggntggccatattatgaagttttaaaggatgantgaaagaaggcagaggnatgatgatgttctgancaanggacttccagattggcgacagcctgtattctactacag

SEQ ID NO: 683 - DNA, 187 bp, Mus musculus
  misc_feature 21, 31, 32, 50, 66, 69, 81: n = A,T,C or G
ggctccaccagcaggcatggngctggactgnncccaagcctggctattgngcctgcccatagctgnggngtacaggctcgntggccctcttcacctccatcctgcacaatgtgttcctgctgtactacgtggacaccttcgtctcagtgtataagatcaataaagtgtccttctgggttggagagaa

SEQ ID NO: 684 - DNA, 174 bp, Mus musculus
gactgaggatgaagtaagggaaaatatagctcgtggaatggcaatcctgggccccacattcactcttgacgctcttgtggaatgccttgtgattggagttggcaccatgtcaggcgtccgccagctggagatcatgtgctgcttcggctgcatgtcagtgttgaaaaaaaaaaa

SEQ ID NO: 685 - DNA, 171 bp, Mus musculus
  misc_feature 19, 78, 109, 114: n = A,T,C or G
tgatcacaagtattcaaangaaggcattgtatggcagctcatgagggatctctgtgtccagggccacagctgtttcgnaagtccagcactgagcgacattacagatagngtggncacctcctcccagcatccctggaataaagtctactttatcatttgcataaacaaaaa

SEQ ID NO: 686 - DNA, 170 bp, Mus musculus
  misc_feature 26: n = A,T,C or G
cccccccgcaccttttgtggagttgnccgagccgcttctttgcacttcctatggatattctctgttgcaaggcatcaaaaaaggactgaggaatgtggaacaccaggggttctggtttgcctgcagaaaaaagaagaatgtcatttaaataaacatggttgttttcttga

SEQ ID NO: 687 - DNA, 169 bp, Mus musculus
  misc_feature 36, 64, 70, 121, 126, 127: n = A,T,C or G
aacatgaaccagcgcacagcatgttggacggatggngaggcccgtacagggtgagaagttctcnttctgnatcctgacacctgacaaggaacattttatcagggcagaatccaaggagatnatcanngggtggttagagatgctcatggttgtatccttcgaaccaaca

SEQ ID NO: 688 - DNA, 169 bp, Mus musculus
  misc_feature 88, 146, 149, 153: n = A,T,C or G
ctggtggactactccaagaaaccttgtgaacaaggaggtgatgcagatgctggcggagctggccaagtccagaggcagtggaggctgnacgggacaacatgttcagtggttccaagcatcaactacaccgaggatctgggcggtgntgnatgnggcccttcgaaaccgg

SEQ ID NO: 689 - DNA, 166 bp, Mus musculus
  misc_feature 54, 64, 87, 124: n = A,T,C or G
gtcctggccttcacgggctgtggcgggctacatcacctacctgtccatcgactnggctctgttngtggagacgctgggcttcctggnctgtgcatgactgaagccatgctgggggtgccacagnatgaacctgaaactacttggcaccgctccacggagggcatga

SEQ ID NO: 690 - DNA, 163 bp, Mus musculus
tgtgtcaaaacccaaagctggcgctcaagcaacagcccgccttatatcttagacctgctgcctgacacctaccagcacctccgcactgtcttgtcaagatatgaggggaagatggagacgcttggagaaaatgagtatttcagggtgttcatggaaaaaaaaa

SEQ ID NO: 691 - DNA, 162 bp, Mus musculus
  misc_feature 125, 139, 141: n = A,T,C or G
gcaccatcatccaacaagcgacccagcaacagtacaccacccccaacacaactcagcaaaatcaagtactcagggggaccccagtattgtcaagaaagaacgacgccaaagctccttccccatangcacagaactgaananagagggactctgagaaaaaga

SEQ ID NO: 692 - DNA, 160 bp, Mus musculus
  misc_feature 28, 30, 62, 111: n = A,T,C or G
actgtcccacaacctacttcagacgctngncatcgggctcctggcgaacctttcagcactgntggagctggacttaagcaacaataggatttctacattagaagaaggcgngtttgctaatttatttaatttaagtgaaataaacctgagtgggaaccca

SEQ ID NO: 693 - DNA, 149 bp, Mus musculus
  misc_feature 23, 24, 27: n = A,T,C or G
tttcctggattcatcaccaaggnntgnacctggaatggcggcagtaactatcaggcaaaccgacagacttctaactacaattcagccaaaatgtctactccaattgacaagtccttgcgaaaatttgaaaataaaatcaatctaaataa

SEQ ID NO: 694 - DNA, 141 bp, Mus musculus
  misc_feature 56, 63, 70, 73, 124: n = A,T,C or G
aaataccatcttgtccgagcaatattggagtcaatagcttttcaggaacaaacagnctgtacnacatgcntgnagagagaaatccagattcccgtcacgaacatccgggcagatggaggcgtgngtaacaacgctttcgtc

SEQ ID NO: 695 - DNA, 135 bp, Mus musculus
  misc_feature 33, 62, 67, 71, 74, 92, 107, 111: n = A,T,C or G
atgagagcctggactatgacaacagcgagaatnagctgttcctggaggaggaaagaccaatnaaccncacnganttccggacagtggagatnatgcgctgggttatnatgngccctcattggaatcaaaaaaaaa

SEQ ID NO: 696 - DNA, 110 bp, Mus musculus
  misc_feature 67, 71, 77, 79: n = A,T,C or G
gatagatttaaaggggaaccccatggcagaaagaagacagctgtttgcagagttgagggctcaaganctgnatcgcntncgactctccacctacagaacagcatgcaggc

SEQ ID NO: 697 - DNA, 107 bp, Mus musculus
gactgaggcagagaaggtgctgcagtttgacccaggcaaccaagaatgtgacggctctgctgatggaagcccgggacctggaagcccgggtcatcatcctttctgca

SEQ ID NO: 698 - DNA, 102 bp, Mus musculus
  misc_feature 40, 43: n = A,T,C or G
aagtttacagtcacaccgtcgcccatctatcatatgtcgntgngacattacttccacttgggtttcatattctgtaaatcgaacaaagtcaaacccttctga

(19) United States
(12) Patent Application Publication
(10) Pub. No.: US 2006/0031947 A1
(43) Pub. Date: Feb. 9, 2006

(54) Novel mutated mammalian cells and animals

(76) Inventors: Alejandro Abuin, The Woodlands, TX (US); Brian Zambrowicz, The Woodlands, TX (US); Arthur T. Sands, The Woodlands, TX (US)

Correspondence Address: Lance K. Ishimoto;LEXICON GENETICS INCORPORATED, 8800 Technology Forest Place, The Woodlands, TX, 77381, US

(21) Appl. No.: 11/021,492

(22) Filed: Dec. 23, 2004

Related U.S. Application Data
(63) Continuation of application No. 10/202,489, filed on Jul. 23, 2002, now abandoned.
(60) Provisional application No. 60/307,670, filed on Jul. 25, 2001.

(51) Int. Cl. A01K 67/00; C07H 21/04; C12N 5/06; C12N 15/867

(52) U.S. Cl. 800/8; 536/23.5; 435/456; 435/354

(57) ABSTRACT

Novel mutated mammalian cells are provided that have been characterized by identifying the sequence of the genes that have been mutated. Preferably, novel mutated cells are murine ES cells that stably incorporate retroviral gene trap constructs in the specifically identified genes. The novel mutated cells and animals are useful in functional genomic analysis, and in the discovery and development of new therapeutic and diagnostics agents and methods.

The present application claims priority to U.S. Provisional Ser. No. 60/307,670 which was filed Jul. 25, 2001. The present application incorporates U.S. Pat. Nos. 6,080,576, 6,136,566, 6,139,833, and 6,207,371 and U.S. application Ser. Nos. 08/728,963, 09/443,282, and 09/276,533 and their respective disclosures herein by reference in their entirety.

### 1.0. FIELD OF THE INVENTION

The present invention is in the field of molecular genetics. The application discloses novel mutated cells that are generated by a process involving the insertion of at least a portion of a genetically engineered viral vector into the chromosome. The specifically disclosed recombinant vector allows for the rapid identification of the gene that has been mutated by using nucleotide or amino acid sequence information to identify the gene that has been mutated by the vector. When mutated embryonic stem cell clones are produced, such cells can be used to produce mutant animals capable of germline transmission of the described mutated genes.

### 2.0. BACKGROUND OF THE INVENTION

Most mammalian genes are divided into exons and introns. Exons are the portions of the gene that are spliced into mRNA and encode the protein product of a gene. In genomic DNA, these coding exons are often divided by noncoding intron sequences. Although RNA polymerase transcribes both intron and exon sequences, the intron sequences must be removed from the transcript so that the resulting mRNA can be translated into protein. Accordingly, all mammalian, and most eukaryotic, cells have the machinery to splice exons to produce mRNA. Gene trap vectors have been designed to insert into the introns of genes in a manner that allows the cellular splicing machinery to splice vector encoded exons to cellular mRNAs. Commonly, gene trap vectors contain selectable marker sequences that are preceded by strong splice acceptor sequences and are not preceded by a promoter. Thus, when such vectors integrate into a gene, the cellular splicing machinery splices exons from the trapped gene onto the 5′ end of the selectable marker sequence. Typically, such selectable marker genes can only be expressed if the vector encoding the gene has integrated into an intron. The resulting gene trap events are subsequently identified by selecting for cells that can survive selective culture.

Gene trapping has generally proven to be an efficient method of mutating large numbers of genes. The insertion of the gene trap vector creates a mutation in the trapped gene, and also provides a molecular tag for ease of identifying the gene that has been trapped. When ROSAβgeo was used to trap genes it was demonstrated that at least 50% of the resulting mutations resulted in a phenotype when examined in mice. This indicates that the gene trap insertion vectors are useful mutagens. Although a powerful tool for mutating genes, the potential of the method has historically been limited by the difficulty in identifying the trapped genes. Methods that have been used to identify trap events rely on the fusion transcripts resulting from the splicing of exon sequences from the trapped gene to sequences encoded by the gene trap vector. Common gene identification protocols used to obtain sequences from these fusion transcripts include 5′ RACE, cDNA cloning, and cloning of genomic DNA surrounding the site of vector integration. However, these methods have proven labor intensive, not readily amenable to automation, and generally impractical for high-throughput.

More recently, vectors have been developed that rely on a new strategy of gene trapping that uses a vector that contains a selectable marker gene preceded by a promoter and followed by a splice donor sequence instead of a polyadenylation sequence. These vectors do not provide selection unless they integrate into a gene and subsequently trap downstream exons that provide a polyadenylation sequence. Integration of such vectors into the chromosome results in the splicing of the selectable marker gene to 3′ exons of the trapped gene. These vectors provide a number of advantages. They can be used to trap genes regardless of whether the genes are normally expressed in the cell type in which the vector has integrated. In addition, cells harboring such vectors can be screened using automated (e.g., 96-well plate format) gene identification assays such as 3′ RACE (see generally, Frohman, 1994, PCR Methods and Applications, 4:S40-S58). Using these vectors it is possible to produce large numbers of mutations and rapidly identify the mutated, or trapped, gene by DNA sequence analysis.

### 3.0. SUMMARY OF THE INVENTION

The subject invention provides numerous isolated mammalian mutant cell clones that are each characterized by the insertion of a mutagenic genetically engineered polynucleotide sequence into a gene identifiable as corresponding to one or more of the OMNIBANK gene trapped sequences (GTSs) disclosed in Sequence Listing.

The subject invention further contemplates a mutated cell, and particularly a mutated ES cell, and the animals derived from such ES cell that stably maintain a genetically engineered mutation in a gene identifiable as corresponding to one of the disclosed GTSs.

### 4.0. DESCRIPTION OF THE SEQUENCE LISTING AND FIGURES

The Sequence Listing is a compilation of nucleotide sequences obtained by sequencing clonal lines of gene trapped murine ES cells.

FIGS. 1A-1C present a diagrammatic representation of representative gene trap vectors used to generate the described sequences.

FIG. 2 provides an index to the Sequence Listing and the corresponding database accession numbers for the genes that have been mutated according to the present invention.

### 5.0. DETAILED DESCRIPTION OF THE INVENTION

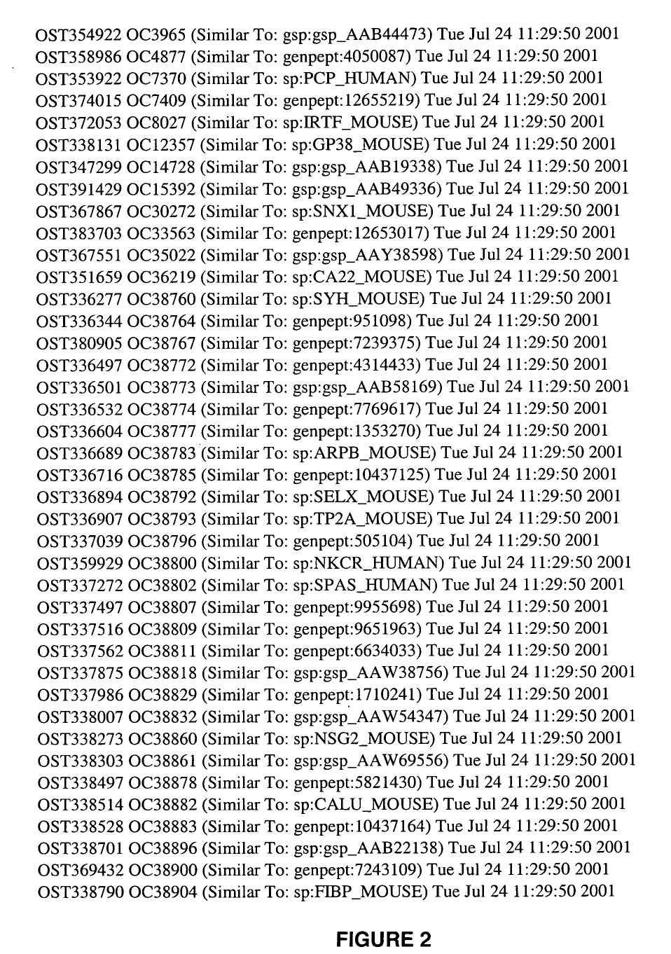

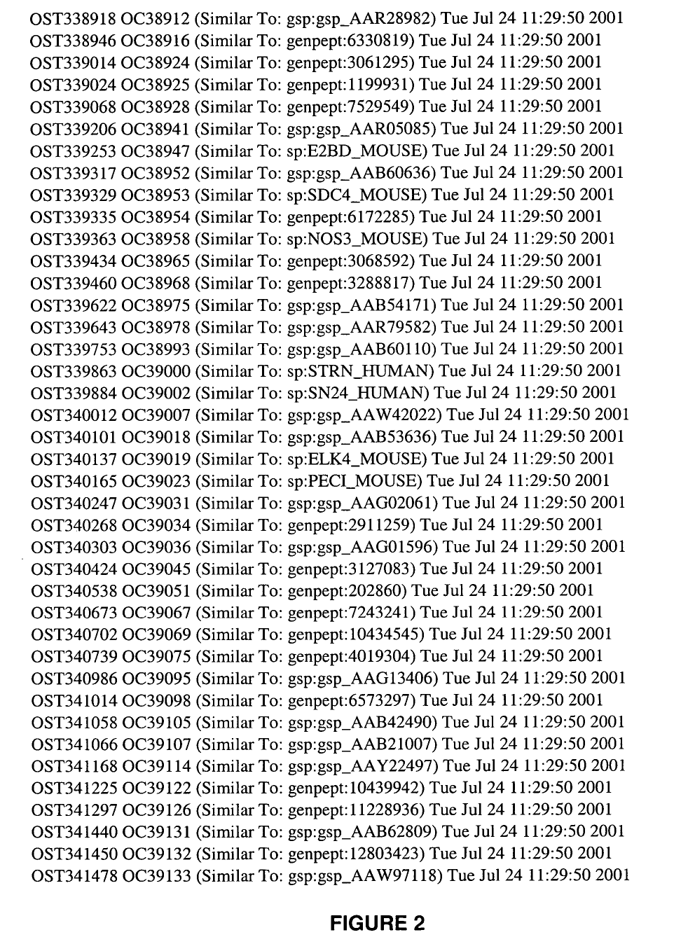

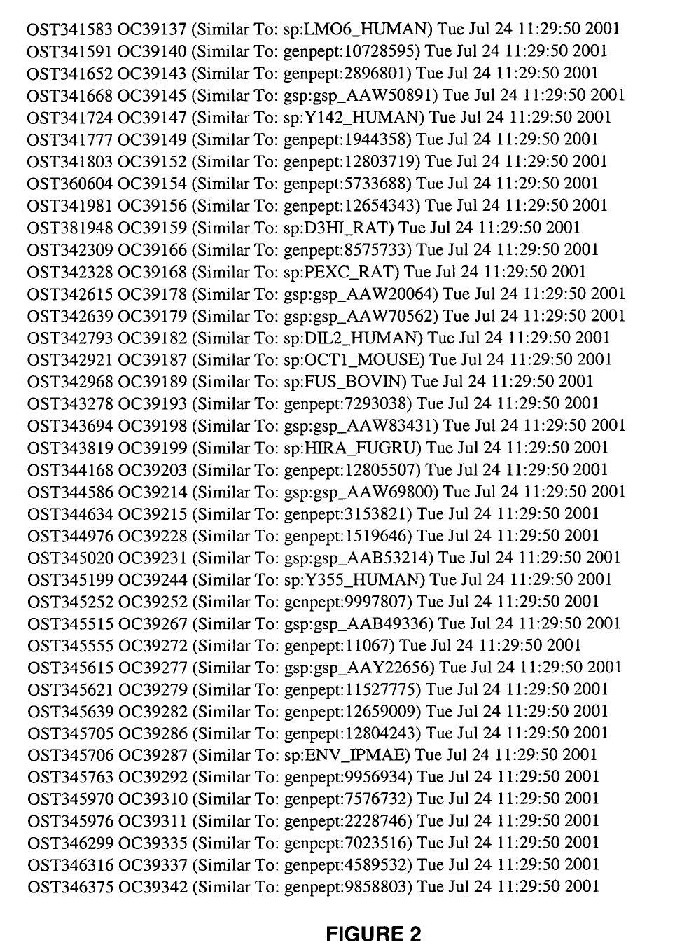

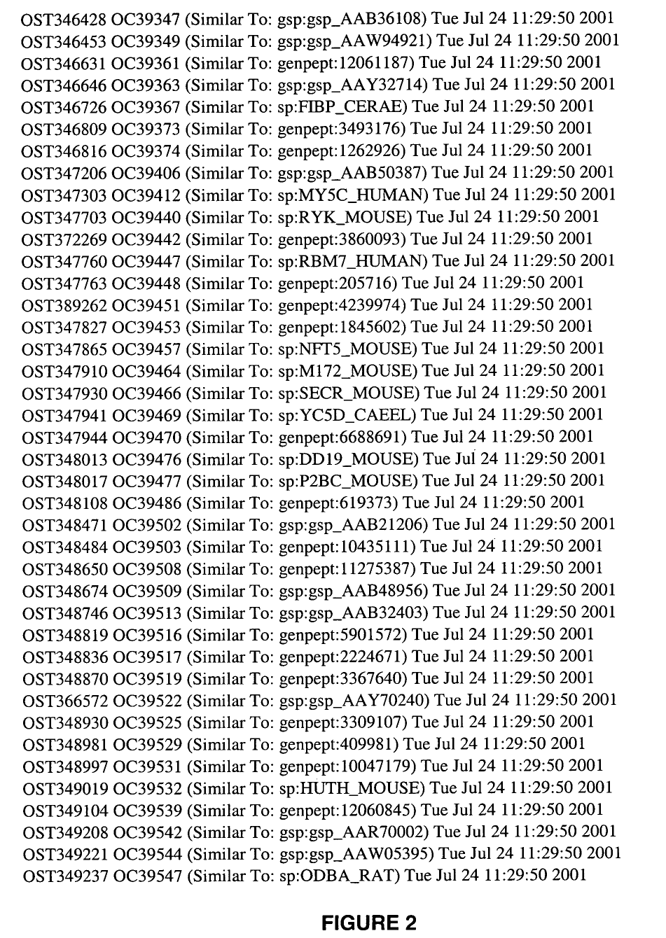

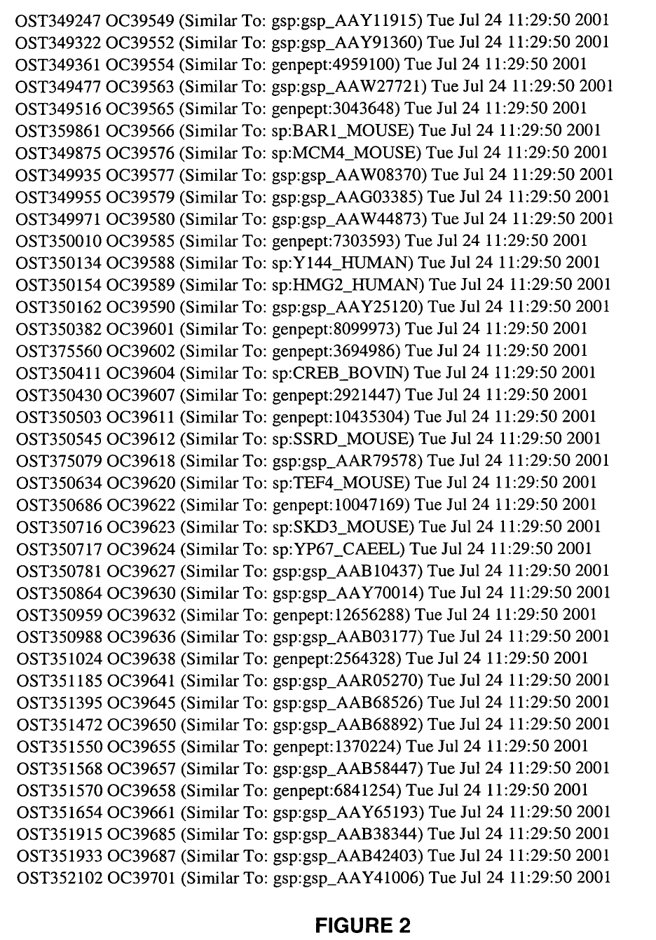

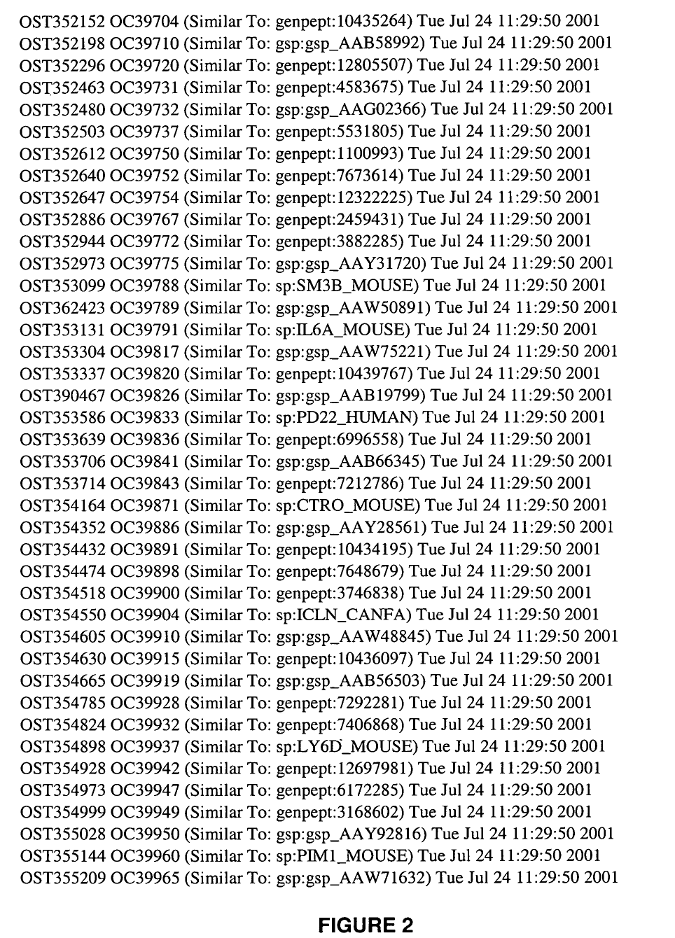

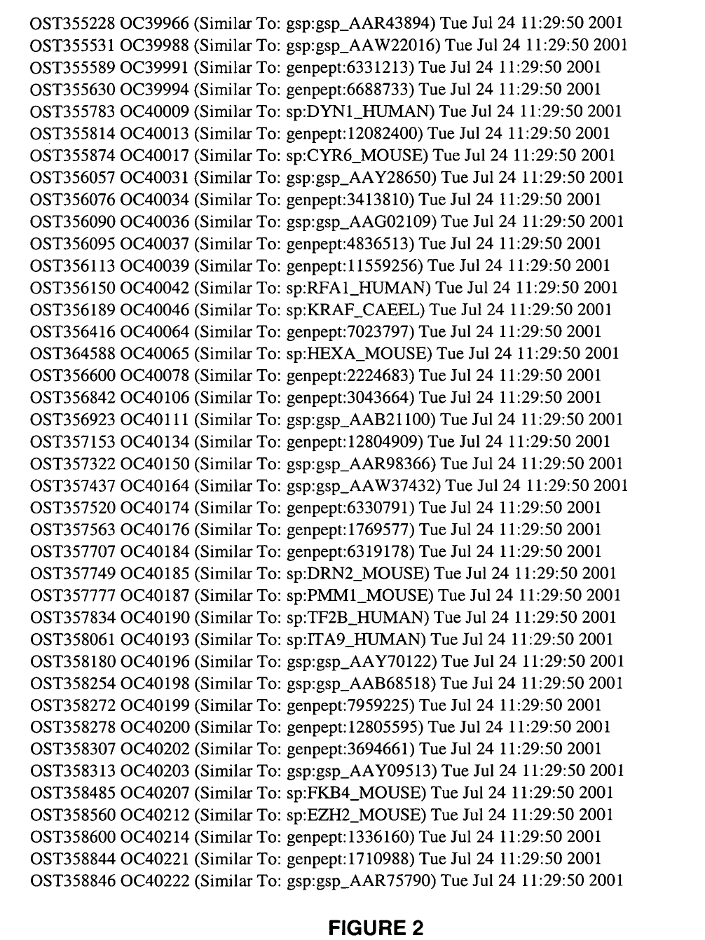

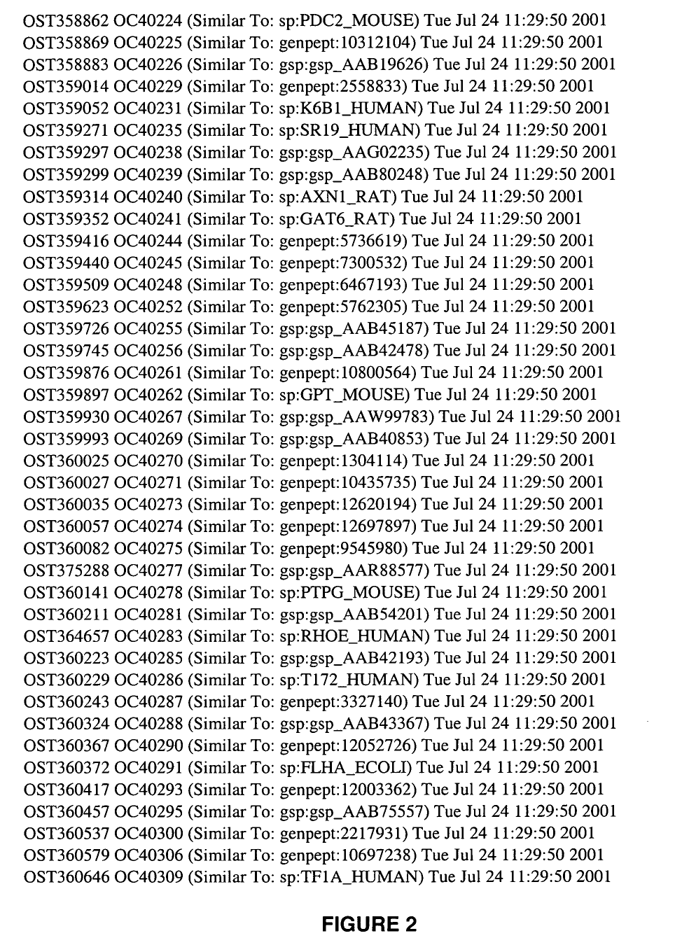

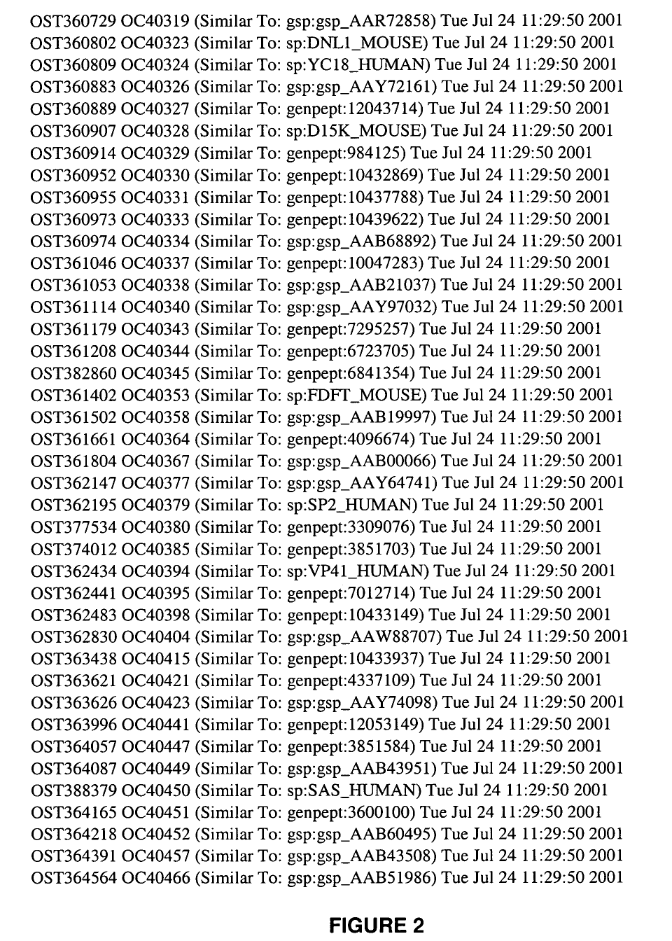

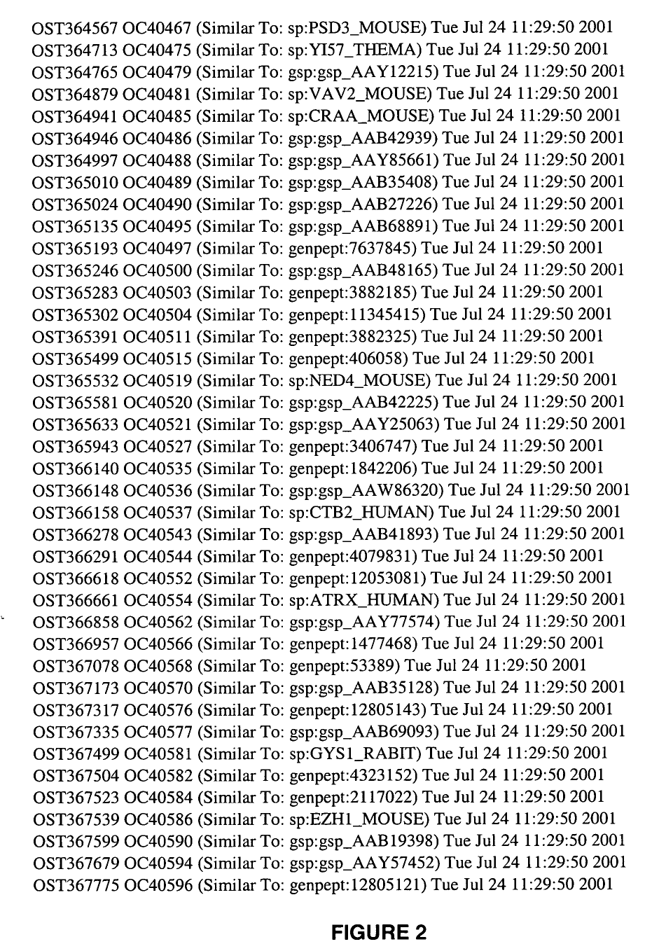

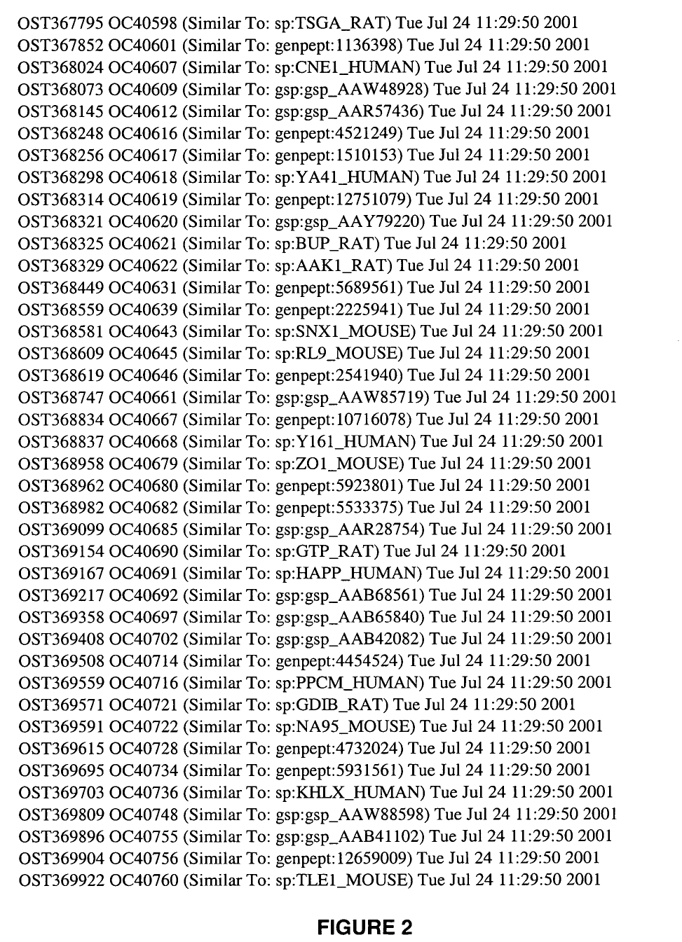

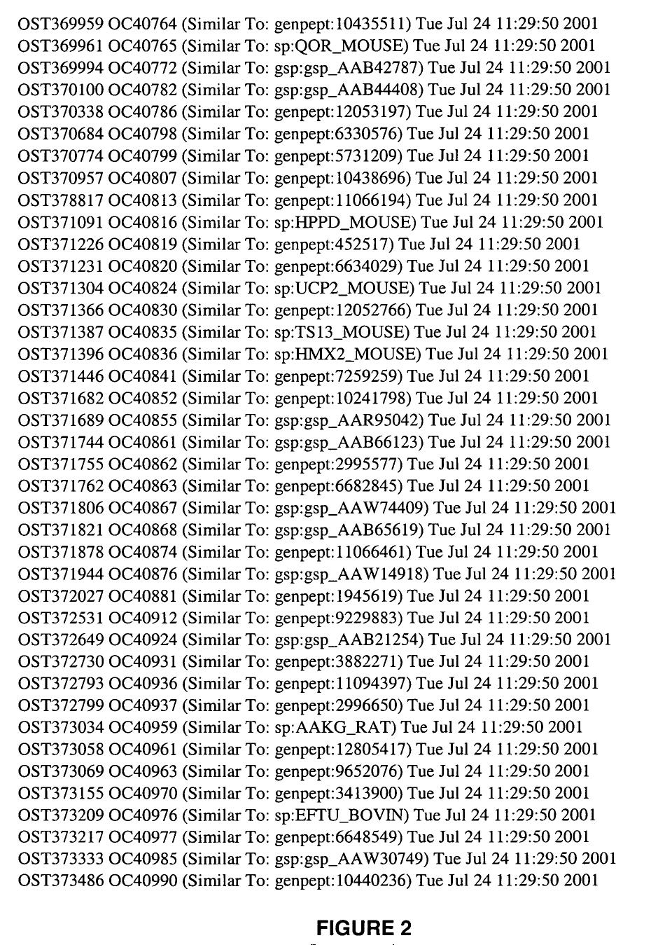

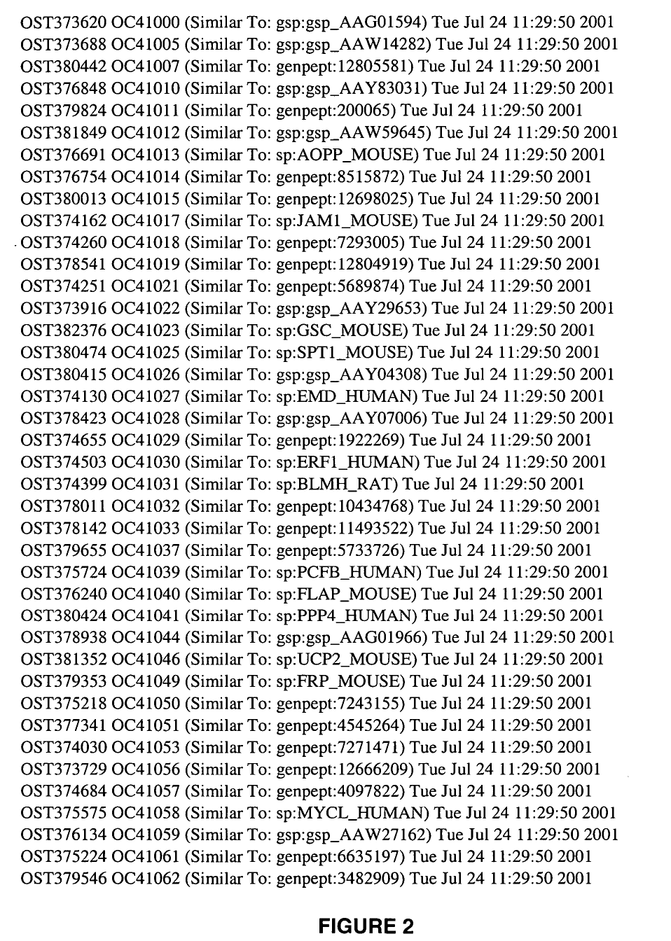

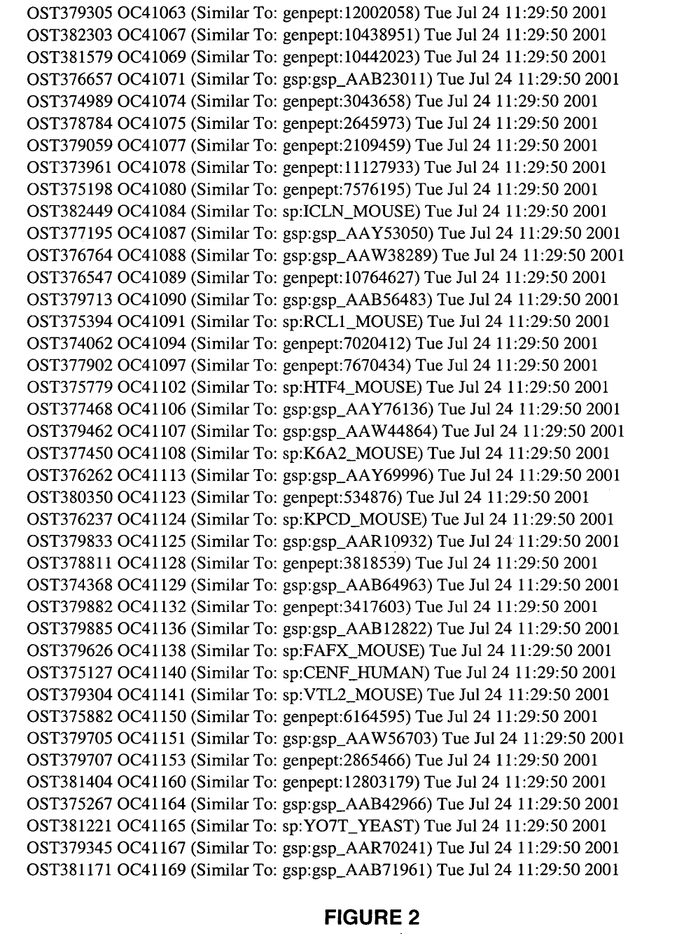

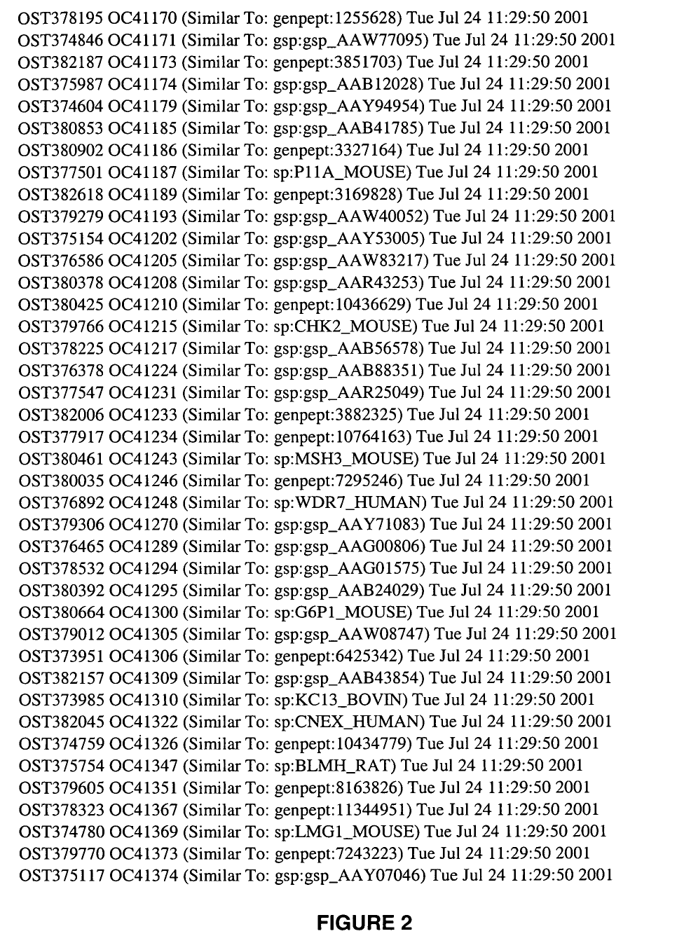

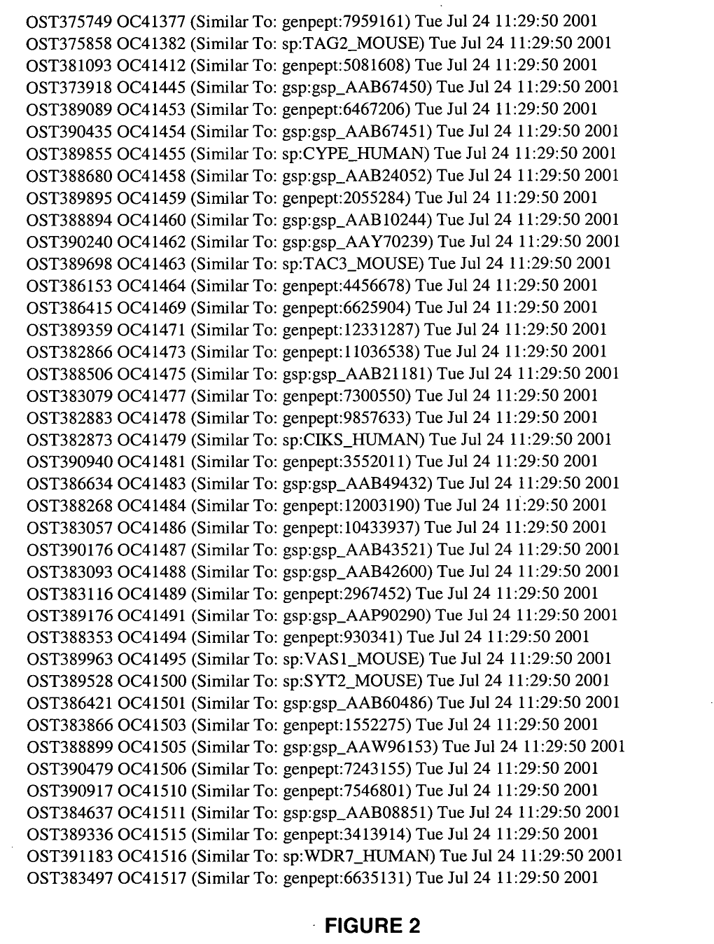

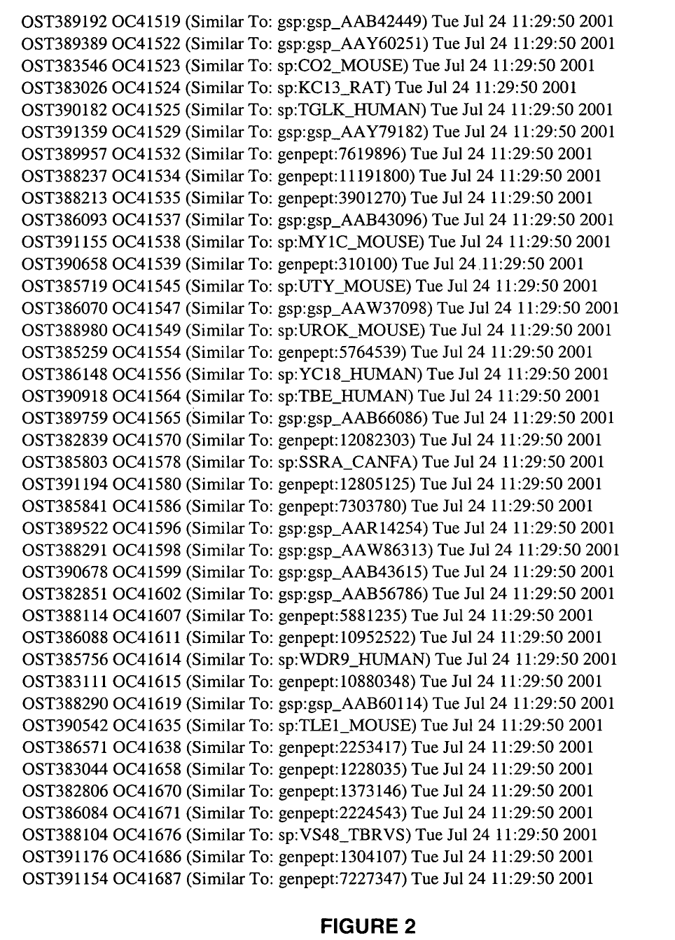

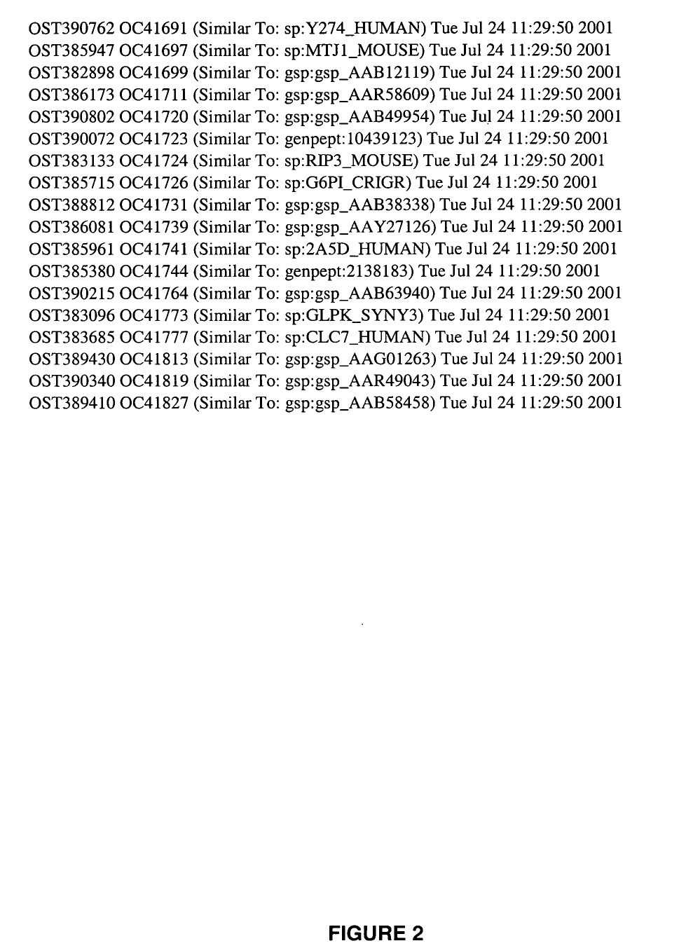

The current invention relates to novel mutated mammalian cells that are each characterized by the insertion of a recombinant (i.e., genetically engineered) mutagenic polynucleotide sequence into a gene identifiable as corresponding to one of the GTSs of SEQ ID NOS:1-698 (i.e., represents the genetic locus that encodes one or more exon sequence(s), or a portion thereof, of the described GTSs). For the purposes of the present invention, the term “identifiable” is to be construed as indicating that a mammalian cell, and preferably, a murine ES cell, has been mutated by the insertion of a polynucleotide sequence of recombinantly manipulated origin at a genetic locus that normally comprises polynucleotide sequence, and/or post-spliced exonic sequence, that is at least partially described in one of the GTSs of Sequence Listing. One method of determining whether one of the described mutated mammalian cells has a mutation in a gene of interest is by comparing the polynucleotide sequence (or a corresponding amino acid sequence) of the GTS identifying the mutated locus to the full length sequence of the gene. Alternatively, such searches can be conducted by comparing the described GTS sequence to a well known database (such as, but not limited to GENBANK) using established computer algorithms including, but not limited to, BLASTX, FASTA, BLASTN, BLASTP, TBLASTN, and TBLASTX using the default parameters used, for example, at the National Center for Biotechnology Information web site (www.ncbi.nlm.nih.gov). The GTSs reported in the Sequence Listing have been compared to such a database (GENBANK), and the accession numbers of the genes that have been mutated are presented in FIG. 2. Accordingly, an additional aspect of the subject invention includes mutated mammalian, preferably murine, cells, or isolated cell lines, that have at least one engineered mutation in a gene identified by a GENBANK or GENESEQ (for example) accession number in FIG. 2.

As used herein, the terms “mutated” or “mutation” mean that the genetic locus has been altered by a process involving the integration or incorporation of a genetically engineered polynucleotide sequence into the genome of the cell with the result that the subsequent level of activity of the product normally encoded by the locus is altered (i.e., reduced, increased, or substantially ablated). In those instances where the mutation substantially completely disrupts the expression or activity of the product normally encoded by the locus (i.e., a null mutation), a cell that is heterozygous for the mutated allele will typically produce about one half of the product of a nonmutated cell (via a gene dosage effect).

The term “recombinantly manipulated” shall mean that such compositions comprising such molecules or polynucleotides have been genetically engineered using molecular biology methodologies in vitro or ex vivo (see generally, Sambrook et al., 1989, Molecular Cloning, A Laboratory Manual, Cold Spring Harbor Press, N.Y.; and Ausubel et al., 1989, Current Protocols in Molecular Biology, Green Publishing Associates and Wiley Interscience, N.Y.).

Where the specifically exemplified mammalian cells, i.e., embryonic stem cells (Lex-1 cells from murine strain A129), are mutated by a process involving the insertion of at least a portion of a genetically engineered vector sequence into the gene of interest, the mutated embryonic stem cells can be microinjected into blastocysts that are subsequently introduced into pseudopregnant female hosts and carried to term using established methods such as those described in, for example, “Mouse Mutagenesis”, 1998, Zambrowicz et al., eds., Lexicon Press, The Woodlands, Tex., and periodic updates thereof, herein incorporated by reference. The resulting chimeric animals are subsequently bred to produce offspring capable of germline transmission of an allele containing the engineered mutation in the gene of interest.

An alternative method of producing mutated cells and animals in the specifically exemplified genes involves the process of gene targeting by homologous recombination using methods such as those exemplified in U.S. application Ser. No. 09/171,642, which is herein incorporated by reference in its entirety. Mutations produced using such methods include, but are not limited to, knockout mutations, “knockin” mutations (where a human gene, for example, is used to replace its murine ortholog), conditional, point mutations, and mutations that activate gene expression. Some of the mutations described above (conditional mutations, point mutations, etc.) can be produced via processes that involve the substantial removal of vector encoded sequences (often recombinase mediated) subsequent to the incorporation of the recombinantly manipulated sequences into the genome.

The described mutated cells/animals can also be used to predict drug action for compounds (small molecules, antisense, antibodies, etc.) that inhibit the activity or expression of the endogenous counterparts of the described mutated genes/gene products.

### 5.1. Mutated Mammalian Cells of the Present Invention

The presently described mutated cells have genetically engineered mutations in genes identifiable as corresponding to, or normally comprising, at least a portion of a sequence reported in the Sequence Listing as SEQ ID NOS: 1-698. Additional embodiments of the present invention are cells comprising engineered mutations in homologs, paralogs, orthologs, etc., of the mutated genes disclosed in the Sequence Listing. Such homologs, paralogs, and orthologs include genes having sequences that hybridize to one or more of the disclosed GTSs of SEQ ID NOS: 1-698 under stringent, or preferably highly stringent, conditions. Hybridization conditions also provide an alternative means of identifying the mutated genes corresponding to the GTSs reported in the Sequence Listing. Typically, such genes will be identifiable because a disclosed GTS, or portion thereof, will hybridize to the gene under stringent conditions.

By way of example and not limitation, high stringency hybridization conditions can be defined as follows: Prehybridization of filters containing DNA to be screened is carried out for 8 h to overnight at 65° C. in a buffer containing 6×SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 48 h at 65° C. in prehybridization mixture containing 100 g/ml denatured salmon sperm DNA and 5-20×10⁶ cpm of ³²P-labeled probe (alternatively, as in all hybridizations described herein, approximately 42, 44, 46, 48, 50, 52, 54, 56, 58, 62, 64, 66, 68, 70, or about 72 degrees or more can be used). The filters are then washed in approximately 1× wash mix (10× wash mix contains 3M NaCl, 0.6M Tris base, and 0.02M EDTA, alternatively, as with all washes described herein, 2×, 3×, 4×, 5×, 6× wash mix, or more, can be used) twice for 5 minutes each at room temperature, then in 1× wash mix containing 1% SDS at 60° C. (alternatively, as in all washes described herein, approximately 42, 44, 46, 48, 50, 52, 54, 56, 58, 62, 64, 66, 68, 70, or about 72 degrees or more can be used) for about 30 min, and finally in 0.3× wash mix (alternatively, as in all final washes described herein, approximately, 0.2×, 0.4×, 0.6×, 0.8×, 1×, or any concentration between about 2× and about 6× can be used in conjunction with a suitable wash temperature) containing 0.1% SDS at 60° C. (alternatively, approximately 42, 44, 46, 48, 50, 52, 54, 56, 58, 62, 64, 66, 68, 70, or about 72 degrees or more can be used) for about 30 min. The filters are then air dried and exposed to x-ray film for autoradiography. In an alternative protocol, washing of filters is done at 37° C. for 1 h in a solution containing 2×SSC, 0.01% PVP, 0.01% Ficoll, and 0.01% BSA. This is followed by a wash in 0.1×SSC at 50° C. for 45 min before autoradiography. Another example of hybridization under highly stringent conditions is hybridization to filter-bound DNA in 0.5 M NaHPO₄, 7% sodium dodecyl sulfate (SDS), 1 mM EDTA at 65° C., and washing in 0.1×SSC/0.1% SDS at 68° C. (Ausubel F. M. et al., eds., 1989, Current Protocols in Molecular Biology, Vol. I, Green Publishing Associates, Inc., and John Wiley & Sons, Inc., New York, at p. 2.10.3).

Additional examples of GTS variants include polynucleotides, or complements thereof, that are capable of binding to the disclosed GTSs under less stringent conditions, such as moderately stringent conditions (e.g., washing in 0.2×SSC/0.1% SDS at 42° C. (Ausubel et al., 1989, supra)). Moderately stringent conditions can be additionally defined, for example, as follows: Filters containing DNA are pretreated for 6 h at 55° C. in a solution containing 6×SSC, 5× Denhart's solution, 0.5% SDS and 100 μg/ml denatured salmon sperm DNA. Hybridizations are carried out in the same solution and 5-20×10⁶ cpm ³²P-labeled probe is used. Filters are incubated in hybridization mixture for 18-20 h at 55° C. (alternatively, as in all hybridizations described herein, approximately 42, 44, 46, 48, 50, 52, 54, 56, 58, 62, 64, 66, 68, 70, or about 72 degrees or more can be used in combination with a suitable concentration of salt). The filters are then washed in approximately 1× wash mix (10× wash mix contains 3M NaCl, 0.6M Tris base, and 0.02M EDTA, alternatively, as with all washes described herein, 2×, 3×, 4×, 5×, 6× wash mix, or more, can be used) twice for 5 minutes each at room temperature, then in 1× wash mix containing 1% SDS at 60° C. (alternatively, as in all washes described herein, approximately, 42, 44, 46, 48, 50, 52, 54, 56, 58, 62, 64, 66, 68, 70, or about 72 degrees or more can be used) for about 30 min, and finally in 0.3× wash mix (alternatively, as in all final washes described herein approximately 0.2×, 0.4×, 0.6×, 0.8×, 1×, or any concentration between about 2× and about 6× can be used in conjunction with a suitable wash temperature) containing 0.1% SDS at 60° C. (alternatively, approximately 42, 44, 45, 48, 50, 52, 54, 56, 58, 62, 64, 66, 68, 70, or about 72 degrees or more can be used) for about 30 min. The filters are then air dried and exposed to x-ray film for autoradiography.

In an alternative protocol, washing of filters is done twice for 30 minutes at 0.60° C. in a solution containing 1×SSC and 0.1% SDS. Filters are blotted dry and exposed for autoradiography.

Other conditions of moderate stringency that may be used are well-known in the art. For example, washing of filters can be done at 37° C. for 1 h in a solution containing 2× SSC, 0.1% SDS. Another example of hybridization under moderately stringent conditions is washing in 0.2×SSC/0.1% SDS at 42° C. (Ausubel et al., 1989, supra). Such less stringent conditions may also be, for example, low stringency hybridization conditions. By way of example and not limitation, procedures using such conditions of low stringency are as follows (see also Shilo and Weinberg, 1981, Proc. Natl. Acad. Sci. USA 78:6789-6792): Filters containing DNA are pretreated for 6 h at 40° C. in a solution containing 35% formamide, 5×SSC, 50 mM Tris-HCl (pH 7.5), 5 mM EDTA, 0.1% PVP, 0.1% Ficoll, 1% BSA, and 500 μg/ml denatured salmon sperm DNA. Hybridizations are carried out in the same solution with the following modifications: 0.02% PVP, 0.02% Ficoll, 0.2% BSA, 100 μg/ml salmon sperm DNA, 10% (wt/vol) dextran sulfate, and 5-20×10⁶ cpm ³²P-labeled probe is used. Filters are incubated in hybridization mixture for 18-20 h at 40° C. (alternatively, as in all hybridizations described herein, approximately 42, 44, 46, 48, 50, 52, 54, 56, 58, 62, 64, 66, 68, 70, or about 72 degrees or more can be used). The filters are then washed in approximately 1× wash mix (10× wash mix contains 3M Na Cl, 0.6M Tris base, and 0.02M EDTA, alternatively, as with all washes described herein, 2×, 3×, 4×, 5×, 6× wash mix, or more, can be used) twice for five minutes each at room temperature, then in 1× wash mix containing 1% SDS at 60° C. (alternatively, as in all washes described herein, approximately 42, 44, 46, 48, 50, 52, 54, 56, 58, 62, 64, 66, 68, 70, or about 72 degrees or more can be used) for about 30 min, and finally in 0.3× wash mix (alternatively, as in all final washes described herein, approximately, 0.2×, 0.4×, 0.6×, 0.8×, 1×, or any concentration between about 2× and about 6× can be used in conjunction with a suitable wash temperature) containing 0.1% SDS at 60° C. (alternatively, approximately 42, 44, 46, 48, 50, 52, 54, 56, 58, 62, 64, 66, 68, 70, or about 72 degrees or more can be used) for about 30 min. The filters are then air dried and exposed to x-ray film for autoradiography. In yet another alternative protocol, washing of filters is done for 1.5 h at 55° C. in a solution containing 2×SSC, 25 mM Tris-HCl (pH 7.4), 5 mM EDTA, and 0.1% SDS. The wash solution is replaced with fresh solution and incubated an additional 1.5 h at 60° C. Filters are then blotted dry and exposed for autoradiography. If necessary, filters are washed for a third time at 65-68° C. and reexposed to film. Other conditions of low stringency that may be used are well known in the art (e.g., as employed for cross-species hybridizations). Preferably, GTS variants identified or isolated using the above methods will also encode a functionally equivalent gene product (i.e., protein, polypeptide, or domain thereof, encoding or otherwise associated with a function or structure at least partially encoded by the complementary GTS).

Low stringency conditions are well known to those of skill in the art, and will vary predict ably depending on the specific organisms from which the library and the labeled sequences are derived. For guidance regarding such conditions see, for example, Sambrook et al., 1989, Molecular Cloning, A Laboratory Manual, Cold Springs Harbor Press, N.Y.; and Ausubel et al., 1989, Current Protocols in Molecular Biology, Green Publishing Associates and Wiley Interscience, N.Y.

The identification of homologs, heterologs, or paralogs of SEQ ID NOS: 1-698 in other, preferably-related, species can be useful for developing additional animal model systems that are closely related to humans for purposes of drug discovery. Genes at other genetic loci within the genome that encode proteins that have extensive-homology to one or more domains of the gene products encoded by SEQ ID NOS: 1-698 can also be identified via similar techniques. In the case of cDNA libraries, such screening techniques can identify clones derived from alternatively spliced transcripts in the same or different species.

Techniques useful to disrupt a gene in a cell, and especially an ES cell that may already have a disrupted gene, are disclosed in U.S. Pat. Nos. 6,136,566, 6,139,833 and 6,207,371, and copending U.S. patent application Ser. No. 08/728,963, each of which are hereby incorporated herein by reference in their entirety. Thus, it is also within the scope of the current invention to disrupt a gene that encodes a polynucleotide of the current invention.

### 5.2. Uses of the Described Mutated Genes and Animals

The described mutated cells and animals are used to investigate and define the cellular and biological functions of the mutated genes. Producing a scientific model that accurately accounts for the large number of genes, proteins, and macromolecules within a single cell has thus far proved beyond the capabilities of existing computer technology. It should thus not be surprising that the far more complex task of modeling the various intricacies, cross and direct redundancies, and interrelated functions of the various metabolic and catabolic processes that occur within a single cell has also proven largely intractable to algorithmic methods of modeling and prediction. Even if one assumes that computer modeling of inherently chaotic/heuristic processes will rapidly mature in the near future, such methods, at best, can only provide predictions that subsequently require practical validation. Several decades of empirical data have proven that mutant phenotypes provide a valuable source of such validation. The mutated diploid mammalian cells of the present invention will initially exist as mutated diploid cells that are heterozygous (except where genes on the X or Y chromosomes are mutated) for the mutations identified in the Sequence Listing. As such, via a “gene dosage” effect, the mutated cells can typically be characterized by the fact that they produce about one half of the mutated transcript/activity relative to cells having two nonmutated or wild type copies of the corresponding gene.

When mutant animals are produced from the mutated cells, heterozygous animals capable of germline transmission of the mutated allele can be bred to produce embryos or offspring that are homozygous for the mutant allele. Such animals or embryos are a rich source of tissues and cells that do not express physiologically relevant amounts of the mutated genes or activities encoded thereby. Accordingly, an additional embodiment of the present invention are mutant cells and animals that have homozygous mutations in genes identifiable as corresponding to the GENBANK, or other database accession, numbers provided in FIG. 2, or are identifiable as a homologs, paralog, or orthologs of a sequence provided in the Sequence Listing.

In addition to providing important information regarding the functional role of a given gene in its nonmutated state (i.e., learning about the function of the gene by discerning the effects of reducing or ablating the activity normally encoded by the gene), the described mutated cells and animals can be used as disease models, or in assays for compounds or genes (via gene delivery or transgenic methods) that compensate for the mutant phenotype and that can be used to treat diseases and disorders related to the observed phenotype. Alternatively, such products and genes can also be used to enhance desirable, if not normal, symptoms related to the observed phenotypes.

Another use for the presently described knockout cells and animals is the generation of high affinity antibodies against mammalian proteins. Given that the described knockout animals will never have seen the proteins expressed by the disrupted genes, the mutated animals can recognize mammalian orthologous proteins (including highly homologous human proteins) as foreign and mount an immune response against such proteins, whereas nonmutated animals might not. Such mammalian antibodies can be “humanized” using readily available means and used as therapeutic agents.

The gene replacement/delivery therapies described above should be capable of delivering gene sequences to the cell types within patients that express the peptide or protein having the desired activity.

The examples below are provided to illustrate the subject invention. These examples are provided by way of illustration and are not included for the purpose of limiting the invention in any way whatsoever.

### 6.0. EXAMPLES

### 6.1. Generation of a Library of Mutated Mouse ES Cells Defined by GTS Sequences

VICTR retroviral vectors (e.g., VICTR 3, 20, BTK, etc.), described in detail in U.S. Pat. Nos. 6,080,576, 6,136,566, 6,139,833, and 6,207,371 were used to generate a library of gene trapped ES cell clones that represent a portion of the described GTSs. A plasmid containing a VICTR cassette was constructed by conventional cloning techniques and designed to employ the features described above. Namely, the cassette contained a PGK promoter directing transcription of an exon that encodes an exon that ends in a canonical splice donor sequence. Sequences were added to the transcript containing the exon to allow for the annealing of nested PCR and sequencing primers. The vector backbone was based on pBluescript KS+ from Stratagene Corporation.

The plasmid construct was linearized by digestion with ScaI, which cuts at a unique site in the plasmid backbone. The plasmid was then transfected into the mouse ES cell line AB2.2 by electroporation using a BioRad Genepulser apparatus. After the cells were allowed to recover, gene trap clones were selected by adding puromycin to the medium at a final concentration of 3 μg/ml (or other selectable agents such as, for example, G418 were use as applicable depending on the vector construct employed in the mutagenesis). Positive clones were allowed to grow under selection for approximately 10 days before being removed and cultured separately for storage and to determine the sequence of the disrupted gene.

Total RNA was isolated from an aliquot of cells from each of 18 gene trap clones chosen for study. Five micrograms of this RNA was used in a first strand cDNA synthesis reaction using the “RS” primer. This primer has unique sequences (for subsequent PCR) on its 5′ end and nine random nucleotides or nine T (thymidine) residues on it's 3′ end. Reaction products from the first strand synthesis were added directly to a PCR with outer primers specific for the engineered sequences of puromycin and the “RS” primer. After amplification, an aliquot of reaction products were subject to a second round of amplification using primers internal, or nested, relative to the first set of PCR primers. This second amplification provided more reaction product for sequencing and also provided increased specificity for the specifically gene trapped DNA.

The products of the nested PCR were visualized by agarose gel electrophoresis, and seventeen of the eighteen clones provided at least one band that was visible on the gel with ethidium bromide staining. Most gave only a single band, which is an advantage in that a single band is generally easier to sequence. The PCR products were sequenced directly after excess PCR primers and nucleotides were removed by filtration in a spin column (Centricon-100, Amicon). DNA was added directly to dye terminator sequencing reactions (purchased from ABI) using the standard M13 forward primer, a region for which was built into the end of the puro exon in all of the PCR fragments.

Subsequent studies have used both VICTR 3 and VICTR 20. Like VICTR 3, VICTR 20 is exemplary of a family of vectors that incorporate two main functional units a sequence acquisition-component having a strong promoter element (phosphoglycerate kinase 1) active in ES cells that is fused to the puromycin resistance gene (or other exon sequence) that is followed by a synthetic consensus splice donor (SD) sequence and lacks an operatively positioned polyadenylation sequence downstream from the SD sequence (PGKpuroSD); and 2) a mutagenic component that incorporates a splice acceptor sequence fused to a selectable and/or colorimetric marker gene and followed by a polyadenylation sequence (for example, SAβgeopA, SAneopA, SAIRESneopA, or SAIRESβgeopA).

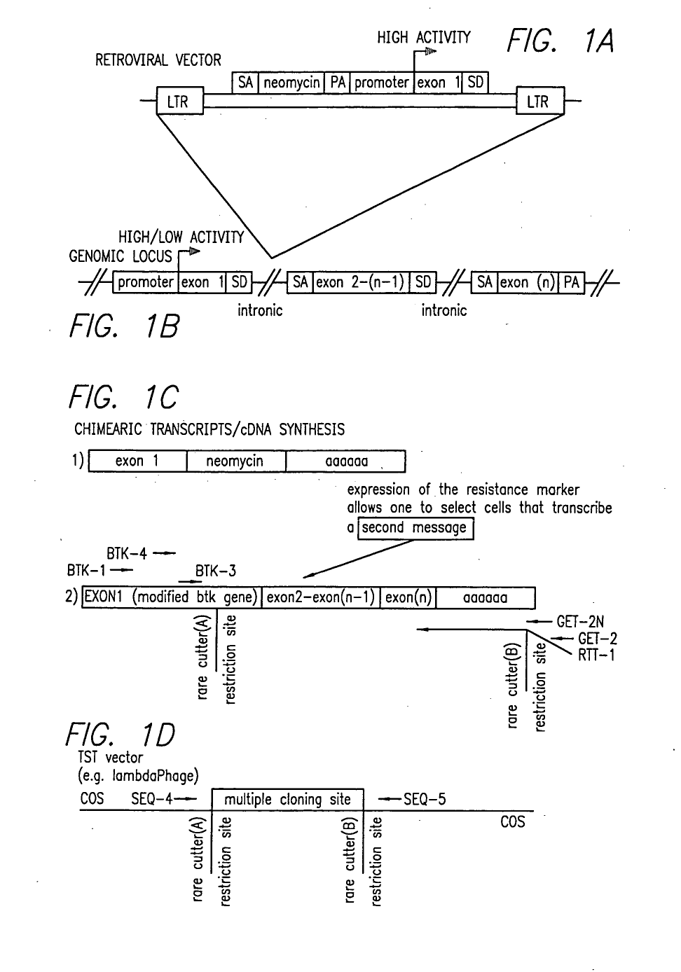

Also like VICTR 3, stop codons have been engineered into all three reading frames in the region between the 3′ end of the selectable marker and the splice donor site. A diagrammatic description of the structure and functions of VICTRs 3 and 20 is provided in FIG. 1.

When VICTRs 3, 20, and certain variations thereof, such as the vectors and methods described in U.S. application Ser. Nos. 09/276,533, and 60/095,989 (the disclosures of which are herein incorporated by reference in their entirety), were used in the commercial scale application of the presently disclosed invention, many mutagenized ES cell clones were rapidly engineered and obtained. Sequence analysis obtained from these clones has identified a wide variety of sequences. Each of the sequences presented in SEQ ID NOS: 1-698 identify novel mutations in the coding regions of mammalian genes that are identifiable as corresponding to the sequences presented in the Sequence Listing. Alternatively, the described mutated cells are described by the database (GENBANK, GENSEQ, etc.) accession numbers for the corresponding genes that have been mutated (see FIG. 2). The described mutated cells, and preferably ES cells, provide a valuable resource for defining, evaluating, or validating the biological function or disease/pharmaceutical relevance of each of these genes.

The cloned 3′ RACE products resulting after the target ES cells were infected with one of the described gene trap vectors were purified using conventional column chromatography, (e.g., S300 and G-50 columns), and the products were recovered by centrifugation. Purified PCR products were quantified by fluorescence using PicoGreen (Molecular Probes, Inc., Eugene Oreg.) as per the manufacturer's instructions.

Dye terminator cycle sequencing reactions with AmpliTaq® FS DNA polymerase (Perkin Elmer Applied Biosystems, Foster City, Calif.) were carried out using approximately 7 pmoles of sequencing primer, and approximately 30-120 ng of 3′ template. Unincorporated dye terminators were removed from the completed sequencing reactions using G-50 columns as described above. The reactions were dried under vacuum, resuspended in loading buffer, and electrophoresed through a 6% Long Ranger acrylamide gel (FMC BioProducts, Rockland, Me.) on an ABI Prism® 377 with XL upgrade as per the manufacturer's instructions. The sequences of the resulting amplicons, or GTSs, are described in SEQ ID NOS: 1-698.

Suitable ES clones identified by the corresponding sequence tags can be microinjected into blastocysts, implanted into pseudopregnant females and used to generate chimeric animals that subsequently bred to product offspring capable of germline transmission of the mutated allele. Typically, animals heterozygous for the mutated allele are initially bred to obtain progeny that are homozygous for the mutated allele.

The reduction or absence of the endogenous protein product in the resulting mutated mouse genetically mimics/predicts the action of a drug that specifically antagonizes the nonmutated form of the protein, or in this case “drug target.” By understanding the medical impact of drug action on mammalian physiology prior to incurring the expense of drug development, a rational method of allocating resources and/or evaluating the market potential of the resulting drug products is provided. Moreover, even in those instances where no medically relevant phenotype is uncovered after a comprehensive medical evaluation of the mutated animals (including results after suitable medical challenges and stresses), substantial value is obtained. For example, where the knockout animal does not present a relevant phenotype, even a drug that essentially perfectly antagonizes the corresponding drug target (i.e., the naturally occurring form of the mutated protein) would more likely than not fail during clinical evaluation. Given the substantial costs of drug development and testing, the ability to deprioritize drug discovery programs directed to drug targets for which mutated animals indicate will have little clinical relevance provides substantial cost savings opportunities for drug development companies. When such cost savings are extended to the industry as a whole, one gains an appreciation for the commercial relevance and utility of the described invention.

All publications and patents mentioned in the above specification are herein incorporated by reference. Various modifications and variations of the described methods and systems of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the above-described modes for carrying out the invention that are obvious to those skilled in the field of molecular biology or related fields are intended to be within the scope of the following claims.

What is claimed is:

1. A genetically engineered mammalian cell that has been mutated by a process comprising the insertion of a recombinantly manipulated polynucleotide sequence into a gene in said genetically engineered mammalian cell wherein said gene is identifiable as corresponding to at least one of SEQ ID NOS: 1-698.

2. The genetically engineered mammalian cell of claim 1, wherein said cell is murine.

3. A cell according to claim 2, wherein said cell is an embryonic stem cell.

4. The genetically engineered mammalian cell of claim 1, wherein said polynucleotide sequence is present on a viral vector.

5. A cell according to claim 4, wherein said viral vector is a retroviral vector.

6. A cell according to claim 4, wherein said viral vector additionally comprises regions of targeting DNA that facilitate gene targeting by homologous recombination.

7. An isolated murine embryonic stem cell line comprising an engineered retroviral gene trap vector in at least one gene comprising a polynucleotide sequence identifiable as corresponding to any one of SEQ ID NOS: 1-698.

8. A method of generating antibodies against a human protein comprising introducing said human protein into a mouse having a genetically engineered mutation in a murine gene identifiable as corresponding to any one of SEQ ID NOS: 1-698, wherein said mouse produces antibodies against said human protein.
